(19) United States
(12) Patent Application Publication
(10) Pub. No.: US 2025/0049854 A1
(43) Pub. Date: Feb. 13, 2025

(54) CELLS COMPRISING A SUPPRESSOR OF GENE EXPRESSION AND/OR A SYNTHETICPATHWAY ACTIVATOR AND/OR AN INDUCIBLE PAYLOAD

(71) Applicant: Arsenal Biosciences, Inc., South San Francisco, CA (US)

(72) Inventors: Aaron Cooper, Berkeley, CA (US); Joseph Choe, South San Francisco, CA (US); Sofia Kyriazopoulou Panagiotopoulou, South San Francisco, CA (US); Marian Sandoval, Oakland, CA (US); Gavin Shavey, South San Francisco, CA (US); Stephen Santoro, Daly City, CA (US); Luke Cassereau, San Bruno, CA (US); Brian Hsu, Foster City, CA (US); Jason Hall, South San Francisco, CA (US); Natalie Bezman, Foster City, CA (US); Thomas Gardner, South San Francisco, CA (US); Anzhi Yao, South San Francisco, CA (US)

(21) Appl. No.: 18/919,328

(22) Filed: Oct. 17, 2024

Related U.S. Application Data
(63) Continuation of application No. PCT/US2023/065998, filed on Apr. 20, 2023.
(60) Provisional application No. 63/384,600, filed on Nov. 21, 2022.
(60) Provisional application No. 63/376,499, filed on Sep. 21, 2022.
(60) Provisional application No. 63/376,531, filed on Sep. 21, 2022.
(60) Provisional application No. 63/369,656, filed on Jul. 27, 2022.
(60) Provisional application No. 63/333,076, filed on Apr. 20, 2022.

(51) Int. Cl. A61K 35/17; A61K 39/00; A61P 35/00; C07K 14/54; C07K 14/715; C07K 16/30; C07K 16/40; C12N 9/22; C12N 15/11; C12N 15/113

(52) U.S. Cl. CPC A61K 35/17; A61K 39/4631; A61K 39/464419; A61K 39/46444; A61K 39/464463; A61K 39/464468; A61P 35/00; C07K 14/5443; C07K 14/7155; C07K 16/30; C07K 16/40; C12N 9/22; C12N 15/111; C12N 15/1138; A61K 2239/13; A61K 2239/21; A61K 2239/22; C12N 2310/14; C12N 2310/20; C12N 2310/531

(57) ABSTRACT

Provided herein are systems comprising one or both of cytokines and/or synthetic pathway activators. Also provided herein are systems comprising one or more suppressors of gene expression, and one or both of cytokines and/or synthetic pathway activators. Also provided are systems of chimeric priming receptors that bind ALPG and/or ALPP, chimeric antigen receptors that bind MSLN, and at least one of one or more suppressors of gene expression, and/or one or both of cytokines and/or synthetic pathway activators; cells expressing such systems; and methods of use thereof.

### CROSS REFERENCE TO RELATED APPLICATIONS

This application is a continuation of International Application No. PCT/US2023/065998 filed Apr. 20, 2023, which claims the benefit of U.S. Provisional Application No. 63/333,076, filed Apr. 20, 2022, U.S. Provisional Application No. 63/369,656, filed Jul. 27, 2022, U.S. Provisional Application No. 63/376,531, filed Sep. 21, 2022, U.S. Provisional Application No. 63/376,499, filed Sep. 21, 2022, and U.S. Provisional Application No. 63/384,600, filed Nov. 21, 2022 each of which are hereby incorporated in their entirety by reference.

### SEQUENCE LISTING

The instant application contains a Sequence Listing which has been submitted via Patent Center and is hereby incorporated by reference in its entirety. Said copy, created on Oct. 15, 2024 is named ANB-214USWOC1_SL.xml, and is 358,279 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them.

After the T cells are engineered with the CARs, the resulting CAR-T cells are introduced into patients to attack tumor cells. CAR-T cells can be either derived from T cells in a patient's own blood (autologous) or derived from the T cells of another healthy donor (allogencic). Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some concerns and limitations to CAR T cell-based immunotherapy. Some CAR T cells may engage with normal cells expressing low levels of target antigens, leading to off target toxicity. Thus, additional therapies are required that reduce off-target toxicity.

### SUMMARY

This disclosure generally relates to systems and methods for enhancing the function of CAR-expressing immune cells (e.g., through use of a logic gate comprising a CAR and a priming receptor along with a synthetic pathway activator (SPA) that enhances immune cell stimulation by inducing constitutive cytokine signaling). Altogether, the systems and methods disclosed herein provide improved efficacy and antigen-specific targeting of CAR-immune cells.

Disclosed herein, in various embodiments is a system comprising system comprising: a first chimeric polypeptide comprises a priming receptor; a second chimeric polypeptide comprises a chimeric antigen receptor (CAR); and a cytokine.

Disclosed herein, in various embodiments is a system comprising system comprising: a first chimeric polypeptide comprising a priming receptor; a second chimeric polypeptide comprising a chimeric antigen receptor (CAR); and a third chimeric polypeptide comprising a synthetic pathway activator (SPA).

Disclosed herein, in various embodiments is a system comprising system comprising: a first chimeric polypeptide comprising a priming receptor; a second chimeric polypeptide comprising a chimeric antigen receptor (CAR); a third chimeric polypeptide comprising a synthetic pathway activator (SPA); and a cytokine.

Disclosed herein, in various embodiments is a system comprising (a) a first chimeric polypeptide comprising a priming receptor, (b) a second chimeric polypeptide comprising a chimeric antigen receptor (CAR), (c) a suppressor of gene expression, and a (d) one or both of (i) third chimeric polypeptide comprising a synthetic pathway activator (SPA) and/or (ii) a cytokine. In some embodiments, the priming receptor comprises, from N-terminus to C-terminus, a first extracellular antigen-binding domain; a first transmembrane domain comprising one or more ligand-inducible proteolytic cleavage sites; and an intracellular domain comprising a human or humanized transcriptional effector.

In some embodiments, the first extracellular antigen-binding domain specifically binds to Alkaline Phosphatase, Germ Cell (ALPG/P). In some embodiments, the first extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 1, 39, 40, 41, or 42, CDR-H2 comprises the sequence set forth in SEQ ID NO: 2, 43, 44, 45, or 46, CDR-H3 comprises the sequence set forth in SEQ ID NO: 3, 47, or 48, CDR-L1 comprises the sequence set forth in SEQ ID NO: 4, 49, or 50, CDR-L2 comprises the sequence set forth in SEQ ID NO: 5 or 51; and CDR-L3 comprises the sequence set forth in SEQ ID NO: 6 or 53. In some embodiments, the VH chain sequence comprises the sequence set forth in SEQ ID NO: 7. In some embodiments, the VL chain sequence comprises the sequence set forth in SEQ ID NO: 8. In some embodiments, the first extracellular antigen-binding domain comprises the sequence set forth in SEQ ID NO: 9. In some embodiments, binding of ALPG/P by the first extracellular antigen-binding domain results in cleavage at the one or more ligand-inducible proteolytic cleavage sites within the intracellular domain.

In some embodiments, the priming receptor further comprises a first hinge domain positioned between the first extracellular antigen-binding domain and the first transmembrane domain. In some embodiments, the first hinge domain comprises a CD8a or truncated CD8a hinge domain. In some embodiments, the first hinge comprises the sequence as set forth in SEQ ID NO: 18.

In some embodiments, the first transmembrane domain comprises a Notch 1 transmembrane domain. In some embodiments, the first transmembrane domain comprises the sequence as set forth in SEQ ID NO: 19.

In some embodiments, the intracellular domain comprises an HNF1a/p65 domain or a Gal4/VP64 domain. In some embodiments, the intracellular domain comprises the sequence as set forth in SEQ ID NO: 23.

In some embodiments, the priming receptor further comprises a stop-transfer-sequence between the first transmembrane domain and the intracellular domain. In some embodiments, the stop-transfer-sequence comprises the sequence as set forth in SEQ ID NO: 20. In some embodiments, the priming receptor comprises a sequence as set forth in SEQ ID NO: 24.

In some embodiments, the CAR comprises, from N-terminus to C-terminus, (a) a second extracellular antigen-binding domain; (b) a second transmembrane domain; (c) an intracellular co-stimulatory domain; and (d) an intracellular activation domain.

In some embodiments, the second extracellular antigen-binding domain specifically binds to mesothelin (MSLN), and the second extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: (a) CDR-H1 comprises the sequence set forth in SEQ ID NO: 10, 54, 56, 57, or 71, (b) CDR-H2 comprises the sequence set forth in SEQ ID NO: 11, 58, 59, 60, 61, or 308, (c) CDR-H3 comprises the sequence set forth in SEQ ID NO: 12, 62, or 63, (d) CDR-L1 comprises the sequence set forth in SEQ ID NO: 14, 64, 65, 66, or 67, (c) CDR-L2 comprises the sequence set forth in SEQ ID NO: 15, 68, 69, or 70, and (f) CDR-L3 comprises the sequence set forth in SEQ ID NO: 16 or 72. In some embodiments, the VH chain sequence comprises the sequence set forth in SEQ ID NO: 13. In some embodiments, the VL chain sequence comprises the sequence set forth in SEQ ID NO: 17. In some embodiments, the second extracellular antigen-binding domain comprises the amino acid sequence set forth in SEQ ID NO: 30.

In some embodiments, the CAR comprises a second hinge domain. In some embodiments, the second hinge domain comprises a CD8a or truncated CD8a hinge domain. In some embodiments, the second transmembrane domain comprises a CD8a transmembrane domain. In some embodiments, the intracellular co-stimulatory domain comprises a 4-1BB domain. In some embodiments, the intracellular activation domain comprises a CD33 domain. In some embodiments, the CAR comprises a sequence as set forth in SEQ ID NO: 31 or 32.

In some embodiments, the SPA is an activator of STAT phosphorylation, optionally STATI, STAT3 and/or STAT5 phosphorylation. In some embodiments, the SPA comprises an extracellular domain linked to an intracellular signaling domain. In some embodiments, the intracellular signaling domain comprises an intracellular signaling region derived from a cytokine receptor. In some embodiments, the intracellular signaling domain comprises a polypeptide sequence derived from an interleukin receptor.

In some embodiments, the cytokine receptor comprises interleukin-6 signal transducer (IL6ST). In some embodiments, the extracellular domain conveys constitutive activity to the intracellular signaling domain. In some embodiments, the extracellular domain comprises a dimerization region, optionally wherein the dimerization region comprises at least one of a cysteine residue and a leucine zipper. In some embodiments, the dimerization region forms a homodimer.

In some embodiments, the SPA comprises a leucine zipper-gp 130 (L-gp130). In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74. In some embodiments, the SPA comprises the amino acid sequence of SEQ ID NO: 74. In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75. In some embodiments, the SPA comprises the amino acid sequence of SEQ ID NO: 75.

In some embodiments, the extracellular domain comprises a polypeptide derived from a cytokine and mimics receptor agonism. In some embodiments, the SPA comprises a membrane-bound interleukin-15 (mbIL-15). In some embodiments, the SPA comprises an amino acid sequence at least 90%, at least 91%, at least 92%, at least 93%, at least 94%, at least 95%, at least 96%, at least 97%, at least 98%, or at least 99% % identical to the sequence set forth in SEQ ID NO: 76. In some embodiments, the SPA comprises the amino acid sequence of SEQ ID NO: 76.

In some embodiments, the SPA comprises a CD34-interleukin-7 receptor (C7R). In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 77. In some embodiments, the SPA comprises an amino acid sequence of SEQ ID NO: 77. In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 78. In some embodiments, the SPA comprises an amino acid sequence of SEQ ID NO: 78.

In some embodiments, the cytokine is a secreted cytokine. In some embodiments, the cytokine is an interleukin. In some embodiments, the cytokine comprises at least one of interleukin (IL)-2, Super-2, IL-12, IL-12/23p40, IL-7, IL-15, IL-21, and IL-18. In some embodiments, the cytokine is IL-2. In some embodiments, the cytokine is Super-2. In some embodiments, the cytokine is IL-12. In some embodiments, the cytokine is IL-12/23p40. In some embodiments, the cytokine is IL-7. In some embodiments, the cytokine is IL-15. In some embodiments, the cytokine is IL-21. In some embodiments, the cytokine is IL-18.

In some embodiments, the cytokine comprises a non-native signal peptide. In some embodiments, the non-native signal peptide comprises a signal peptide from at least one of CD44, CD3E, CD5, IGTAL, IL-2, GMCSF, chymotrypsinogen, trypsinogen, IgK, IgKVIII, IgE, OSM, IgG2H, BM40, secrecon, and tPA. In some embodiments, the non-native signal peptide comprises or consists of the amino acid sequence set forth in SEQ ID NO: 100, 102, 104, 106, 108, 110, 112, 114, 116, 118, 120, 122, 124, 126, 128, or 130. In some embodiments, the cytokine comprises the amino acid sequence set forth in SEQ ID NO: 86, 88, 90, 92, 94, 96, 98 or 132.

In some embodiments, the suppressor of gene expression is an sgRNA or an shRNA. In some embodiments, the suppressor of gene expression is an sgRNA. In some embodiments, the sgRNA suppresses the expression of a gene selected from PTPN2, RASA2, SOCS1, ZC3H12A, and CISH. In some embodiments, the sgRNA suppresses the expression of PTPN2. In some embodiments, the sgRNA suppresses the expression of RASA2. In some embodiments, the sgRNA suppresses the expression of SOCS1. In some embodiments, the sgRNA suppresses the expression of ZC3H12A. In some embodiments, the sgRNA suppresses the expression of CISH. In some embodiments, the sgRNA comprises a nucleic acid sequence set forth in any one of SEQ ID NOs: 160-164.

In some embodiments, the suppressor of gene expression is an shRNA. In some embodiments, the shRNA suppresses the expression of a gene selected from RASA2, SOCS1, ZC3H12A, TGFBR1, and CISH. In some embodiments, the shRNA suppresses the expression of RASA2. In some embodiments, the shRNA suppresses the expression of SOCS1. In some embodiments, the shRNA suppresses the expression of ZC3H12A. In some embodiments, the shRNA suppresses the expression of TGFBR1. In some embodiments, the shRNA suppresses the expression of CISH. In some embodiments, the shRNA comprises a nucleic acid sequence set forth in any one of SEQ ID NOs: 165-172.

In some embodiments, the system comprises two or more suppressors of gene expression. In some embodiments, the system comprises an shRNA that suppresses the expression of TNFRSF6 (Fas) and an additional suppressor of gene expression. In some embodiments, the system comprises an shRNA that suppresses the expression of TNFRSF6 (Fas), an shRNA that suppresses the expression of TGFBR2 and an additional suppressor of gene expression. In some embodiments, the system comprises an shRNA that suppresses the expression of TNFRSF6 (Fas), an shRNA that suppresses the expression of PTPN2 and an additional suppressor of gene expression.

In some embodiments, the system comprises an sgRNA that suppresses CISH expression and a cytokine that is IL-2. In some embodiments, the system comprises an sgRNA that suppresses PTPN2 expression and a cytokine that is IL-2. In some embodiments, the system comprises an sgRNA that suppresses RASA2 expression and a cytokine that is IL-2. In some embodiments, the system comprises an sgRNA that suppresses SOCS1 expression and a cytokine that is IL-2. In some embodiments, the system comprises an sgRNA that suppresses ZC3H12A expression and a cytokine that is IL-2. In some embodiments, the system comprises an shRNA that suppresses RASA2 expression and a cytokine that is IL-2. In some embodiments, the system comprises an sgRNA that suppresses PTPN2 expression and a cytokine that is IL-21. In some embodiments, the system comprises an sgRNA that suppresses ZC3H12A expression and a cytokine that is IL-21. In some embodiments, the system comprises an sgRNA that suppresses CISH expression and an SPA that is C7R. In some embodiments, the system comprises an sgRNA that suppresses PTPN2 expression and an SPA that is C7R. In some embodiments, the system comprises an sgRNA that suppresses RASA2 expression and an SPA that is C7R. In some embodiments, the system comprises an sgRNA that suppresses SOCS1 expression and an SPA that is C7R. In some embodiments, the system comprises an sgRNA that suppresses ZC3H12A expression and an SPA that is C7R. In some embodiments, the system comprises an sgRNA that suppresses CISH expression and an SPA that is L-gp130. In some embodiments, the system comprises an sgRNA that suppresses RASA2 expression and an SPA that is L-gp130. In some embodiments, the system comprises an sgRNA that suppresses ZC3H12A expression and an SPA that is L-gp130. In some embodiments, the system comprises an shRNA that suppresses RASA2 expression, a cytokine that is IL-2, and an SPA that is L-gp130 In some embodiments, the system comprises an sgRNA that suppresses RASA2 expression, a cytokine that is IL-2, and an SPA that is L-gp130. In some embodiments, the system comprises an shRNA that suppresses RASA2 expression, a cytokine that is IL-15, and an SPA that is L-gp130. In some embodiments, the system comprises an sgRNA that suppresses RASA2 expression, a cytokine that is IL-15, and an SPA that is L-gp130.

In some embodiments, the priming receptor and the CAR are capable of binding to a same target cell. In some embodiments, the target cell is a human cell. In some embodiments, the target cell is a cancer cell. In some embodiments, the cancer cell is a solid cancer cell or a liquid cancer cell. In some embodiments, the cancer cell is ovarian cancer, fallopian cancer, primary peritoneal cancer, uterine cancer, mesothelioma, cervical cancer, or pancreatic cancer.

Also disclosed herein, in various embodiments, is one or more recombinant nucleic acids comprising at least one nucleic acid fragment comprising a nucleotide sequence encoding the system disclosed herein.

Also disclosed herein, in various embodiments is one or more recombinant nucleic acids comprising at least one nucleic acid fragment comprising: a nucleotide sequence encoding a priming receptor comprising a first extracellular antigen-binding domain; a nucleotide sequence encoding a chimeric antigen receptor comprising a second extracellular antigen-binding domain; and a nucleotide sequence encoding a cytokine.

Also disclosed herein, in various embodiments is one or more recombinant nucleic acids comprising at least one nucleic acid fragment comprising: a nucleotide sequence encoding a priming receptor comprising a first extracellular antigen-binding domain; a nucleotide sequence encoding a chimeric antigen receptor comprising an second extracellular antigen-binding domain; and a nucleotide sequence encoding a synthetic pathway activator.

Also disclosed herein, in various embodiments is one or more recombinant nucleic acids comprising at least one nucleic acid fragment comprising: a nucleotide sequence encoding a priming receptor comprising a first extracellular antigen-binding domain; a nucleotide sequence encoding a chimeric antigen receptor comprising an second extracellular antigen-binding domain; a nucleotide sequence encoding a synthetic pathway activator; and a nucleotide sequence encoding a cytokine.

Also disclosed herein, in various embodiments is one or more recombinant nucleic acids comprising at least one nucleic acid fragment comprising: a nucleotide sequence encoding a priming receptor comprising a first extracellular antigen-binding domain; a nucleotide sequence encoding a chimeric antigen receptor comprising an second extracellular antigen-binding domain; a nucleotide sequence of a suppressor of gene expression; and one or both of: a nucleotide sequence encoding a synthetic pathway activator and/or a nucleotide sequence encoding a cytokine. In some embodiments, the first extracellular antigen-binding domain specifically binds to ALPG/P. In some embodiments, the second extracellular antigen-binding domain specifically binds to MSLN. In some embodiments, the recombinant nucleic acid comprises two or more nucleic acid fragments.

In some embodiments, the recombinant nucleic acid further comprises an inducible promoter operably linked to the nucleotide sequence encoding the CAR. In some embodiments, the recombinant nucleic acid further comprises a constitutive promoter operably linked to the nucleotide sequence encoding the priming receptor. In some embodiments, the recombinant nucleic acid further comprises a constitutive promoter operably linked to the nucleotide sequence encoding the synthetic pathway activator. In some embodiments, the priming receptor and the synthetic pathway activator are under the control of the same constitutive promoter. In some embodiments, the recombinant nucleic acid further comprises an inducible promoter operably linked to the nucleotide sequence encoding the chimeric antigen receptor and a constitutive promoter operably linked to the nucleotide sequence encoding the priming receptor and the nucleotide sequence encoding the synthetic pathway activator.

In some embodiments, the nucleic acid comprises, in a 5′ to 3′ direction, the constitutive promoter; the nucleotide sequence encoding the synthetic pathway activator; the nucleotide sequence encoding priming receptor; the inducible promoter; and the nucleotide sequence encoding chimeric antigen receptor. In some embodiments, the nucleic acid comprises, in a 5′ to 3′ direction, the inducible promoter; the nucleotide sequence encoding chimeric antigen receptor; the constitutive promoter; the nucleotide sequence encoding priming receptor; and the nucleotide sequence encoding the synthetic pathway activator.

In some embodiments, the recombinant nucleic acid further comprises an inducible promoter operably linked to the nucleotide sequence encoding the cytokine.

In some embodiments, the recombinant nucleic acid further comprises: (a) an inducible promoter operably linked to the nucleotide sequence encoding the chimeric antigen receptor and the nucleotide sequence encoding the cytokine; and (b) a constitutive promoter operably linked to the nucleotide sequence encoding the priming receptor.

In some embodiments, the nucleic acid comprises, in a 5′ to 3′ direction, (a) the constitutive promoter; (b) the nucleotide sequence encoding the priming receptor; (c) the inducible promoter; d) the nucleotide sequence encoding the chimeric antigen receptor; and (c) the nucleic acid sequence encoding the cytokine.

In some embodiments, the nucleic acid comprises, in a 5′ to 3′ direction, (a) the constitutive promoter; (b) the nucleotide sequence encoding the priming receptor; (c) the inducible promoter; (d) the nucleotide sequence encoding the cytokine; and (e) the nucleic acid sequence encoding the chimeric antigen receptor.

In some embodiments, the nucleic acid comprises, in a 5′ to 3′ direction, (a) the inducible promoter; (b) the nucleotide sequence encoding the chimeric antigen receptor; (c) the nucleic acid sequence encoding the cytokine; (d) the constitutive promoter; and (e) the nucleotide sequence encoding the priming receptor.

In some embodiments, the nucleic acid comprises, in a 5′ to 3′ direction, (a) the inducible promoter; (b) the nucleotide sequence encoding the cytokine; (c) the nucleic acid sequence encoding the chimeric antigen receptor; (d) the constitutive promoter; and (e) the nucleotide sequence encoding the priming receptor.

In some embodiments, the recombinant nucleic acid further comprises: (a) a first inducible promoter operably linked to the nucleotide sequence encoding the chimeric antigen receptor; (b) a second inducible promoter operably linked to the nucleotide sequence encoding the cytokine; and (c) a constitutive promoter operably linked to the nucleotide sequence encoding the priming receptor.

In some embodiments, the nucleic acid comprises, in a 5′ to 3′ direction, (a) the constitutive promoter; (b) the nucleotide sequence encoding the priming receptor; (c) the first inducible promoter; (d) the nucleotide sequence encoding the chimeric antigen receptor; (e) the second inducible promoter; and (f) the nucleic acid sequence encoding the cytokine.

In some embodiments, the nucleic acid comprises, in a 5′ to 3′ direction, (a) the constitutive promoter; (b) the nucleotide sequence encoding the priming receptor; (c) the second inducible promoter; (c) the nucleic acid sequence encoding the cytokine; (d) the first inducible promoter; and (c) the nucleotide sequence encoding the chimeric antigen receptor.

In some embodiments, the nucleic acid comprises, in a 5′ to 3′ direction, (a) the first inducible promoter; (b) the nucleotide sequence encoding the chimeric antigen receptor; (c) the second inducible promoter; (d) the nucleic acid sequence encoding the cytokine; (c) the constitutive promoter; and (f) the nucleotide sequence encoding the priming receptor.

In some embodiments, the nucleic acid comprises, in a 5′ to 3′ direction, (a) the first inducible promoter; (b) the nucleotide sequence encoding the chimeric antigen receptor; (c) the constitutive promoter; (d) the nucleotide sequence encoding the priming receptor; (e) the second inducible promoter; and (f) the nucleic acid sequence encoding the cytokine.

In some embodiments, the nucleic acid comprises, in a 5′ to 3′ direction, (a) the second inducible promoter; (b) the nucleic acid sequence encoding the cytokine; (c) the first inducible promoter; (d) the nucleotide sequence encoding the chimeric antigen receptor; (e) the constitutive promoter; and (f) the nucleotide sequence encoding the priming receptor.

In some embodiments, the nucleic acid comprises, in a 5′ to 3′ direction, (a) the second inducible promoter; (b) the nucleic acid sequence encoding the cytokine; (c) the constitutive promoter; (d) the nucleotide sequence encoding priming receptor; (c) the first inducible promoter; and (f) the nucleotide sequence encoding chimeric antigen receptor.

In some embodiments, the first inducible promoter and the second inducible promoter are identical.

In some embodiments, the nucleotide sequence encoding the priming receptor comprises the sequence set forth in SEQ ID NO: 35. In some embodiments, the nucleotide sequence encoding the chimeric antigen receptor comprises the sequence set forth in SEQ ID NO: 36. In some embodiments, the nucleotide sequence encoding the synthetic pathway activator comprises the sequence set forth in SEQ ID NO: 79, 80, 81, 82, or 83. In some embodiments, the nucleotide sequence encoding the cytokine comprises the sequence set forth in SEQ ID NO: 87, 89, 91, 93, 95, 97, or 99. In some embodiments, the suppressor of gene expression comprises a nucleic acid sequence set forth in any one of SEQ ID NOs: 160-172.

In some embodiments, the nucleic acid further comprises a 5′ homology directed repair arm and a 3′ homology directed repair arm complementary to an insertion site in a host cell chromosome. In some embodiments, the recombinant nucleic acid further comprises a nucleotide sequence encoding a self-excising 2A peptide (P2A). In some embodiments, the P2A is at the 3′ end of the nucleotide sequence encoding chimeric antigen receptor. In some embodiments, the P2A is at the 3′ end of the nucleotide sequence encoding priming receptor.

In some embodiments, the recombinant nucleic acid further comprises a woodchuck hepatitis virus post-translational regulatory element (WPRE). In some embodiments, the WPRE is at the 3′ end of the nucleotide sequence encoding chimeric antigen receptor and at the 5′ end of the nucleotide sequence encoding priming receptor or wherein the WPRE is at the 3′ end of the nucleotide sequence encoding priming receptor and at the 5′ end of the nucleotide sequence encoding chimeric antigen receptor. In some embodiments, the recombinant nucleic acid further comprises an SV40 polyA element.

In some embodiments, the nucleic acid is incorporated into an expression cassette or an expression vector. In some embodiments, the expression vector is a non-viral vector.

Also disclosed herein, in various embodiments, is an expression vector comprising the recombinant nucleic acid disclosed herein. In some embodiments, the 5′ and 3′ ends of the recombinant nucleic acid comprise nucleotide sequences that are homologous to genomic sequences flanking an insertion site in a genome of a primary cell. In some embodiments, the insertion site is located at a T Cell Receptor Alpha Constant (TRAC) locus or a genomic safe harbor (GSH) locus.

Also disclosed herein, in various embodiments, is an immune cell comprising: the system disclosed herein; at least one recombinant nucleic acid disclosed herein; and/or the vector disclosed herein. In some embodiments, the immune cell is a primary human immune cell. In some embodiments, the immune cell is an allogeneic immune cell. In some embodiments, the immune cell is an autologous immune cell. In some embodiments, the primary immune cell is a natural killer (NK) cell, a T cell, a CD8+ T cell, a CD4+ T cell, a primary T cell, or a T cell progenitor. In some embodiments, the primary immune cell is a primary T cell. In some embodiments, the primary immune cell is a primary human T cell. In some embodiments, the primary immune cell is virus-free.

Also disclosed herein, in various embodiments, is a primary immune cell comprising at least one recombinant nucleic acid comprising: a nucleic acid sequence encoding a priming receptor comprising a first extracellular antigen-binding domain; a nucleic acid sequence encoding a chimeric antigen receptor comprising a second extracellular antigen-binding domain; and a nucleic acid sequence encoding a cytokine and/or a synthetic pathway activator; wherein the recombinant nucleic acid is inserted into a target region of the genome of the primary immune cell, wherein the primary immune cell does not comprise a viral vector for introducing the recombinant nucleic acid into the primary immune cell. In some embodiments, the first extracellular antigen-binding domain specifically binds to ALPG/P. In some embodiments, the second extracellular antigen-binding domain specifically binds to MSLN.

Also disclosed herein, in various embodiments, is a viable, virus-free, primary cell comprising a ribonucleoprotein complex (RNP)-recombinant nucleic acid complex, wherein the RNP comprises a nuclease domain and a guide RNA, wherein recombinant nucleic acid comprises: a nucleic acid sequence encoding a priming receptor comprising a first extracellular antigen-binding domain that specifically binds to ALPG/P; a nucleic acid sequence encoding a chimeric antigen receptor comprising a second extracellular antigen-binding domain that specifically binds to MSL; and a nucleic acid sequence encoding a cytokine and/or a synthetic pathway activator that constitutively activates cytokine signaling; wherein the 5′ and 3′ ends of the recombinant nucleic acid comprise nucleotide sequences that are homologous to genomic sequences flanking an insertion site in the genome of the primary cell. In some embodiments, the first extracellular antigen-binding domain specifically binds to ALPG/P. In some embodiments, the second extracellular antigen-binding domain specifically binds to MSLN.

Also disclosed herein, in various embodiments, is a population of cells comprising a plurality of immune cells disclosed herein.

Also disclosed herein, in various embodiments, is a pharmaceutical composition comprising the immune cell disclosed herein or the population of cells disclosed herein, and a pharmaceutically acceptable excipient.

Also disclosed herein, in various embodiments, is a pharmaceutical composition comprising the recombinant nucleic acid disclosed herein or the vector disclosed herein, and a pharmaceutically acceptable excipient.

Also disclosed herein, in various embodiment is a method of editing an immune cell, comprising: providing a ribonucleoprotein complex (RNP)-recombinant nucleic acid complex, wherein the RNP comprises a nuclease domain and a guide RNA, wherein the recombinant nucleic acid comprises the recombinant nucleic acid disclosed herein, and wherein the 5′ and 3′ ends of the recombinant nucleic acid comprise nucleotide sequences that are homologous to genomic sequences flanking an insertion site in the genome of the immune cell; non-virally introducing the RNP-recombinant nucleic acid complex into the immune cell, wherein the guide RNA specifically hybridizes to a target region of the genome of the primary immune cell, and wherein the nuclease domain cleaves the target region to create the insertion site in the genome of the immune cell; and editing the immune cell via insertion of the recombinant nucleic acid disclosed herein into the insertion site in the genome of the immune cell. In some embodiments, non-virally introducing comprises electroporation.

In some embodiments, the nuclease domain comprises a CRISPR-associated endonuclease (Cas), optionally a Cas9 nuclease. In some embodiments, the target region of the genome of the cell is a T Cell Receptor Alpha Constant (TRAC) locus or a genomic safe harbor (GSH) locus. In some embodiments, the recombinant nucleic acid is a double-stranded recombinant nucleic acid or a single-stranded recombinant nucleic acid. In some embodiments, the recombinant nucleic acid is a linear recombinant nucleic acid or a circular recombinant nucleic acid, optionally wherein the circular recombinant nucleic acid is a plasmid.

In some embodiments, the immune cell is a primary human immune cell. In some embodiments, the immune cell is an autologous immune cell. In some embodiments, the immune cell is an allogeneic immune cell. In some embodiments, the immune cell is a natural killer (NK) cell, a T cell, a CD8+ T cell, a CD4+ T cell, a primary T cell, or a T cell progenitor. In some embodiments, the immune cell is a primary T cell. In some embodiments, the immune cell is a primary human T cell. In some embodiments, the immune cell is virus-free. In some embodiments, the method further comprises obtaining the immune cell from a patient and introducing the recombinant nucleic acid in vitro.

Also disclosed herein, in various embodiments, is a method of treating a disease in a subject comprising administering the immune cell disclosed herein or primary cells disclosed herein or the pharmaceutical composition disclosed herein to the subject. In some embodiments, the disease is cancer. In some embodiments, the cancer is a solid cancer or a liquid cancer. In some embodiments, the cancer is ovarian cancer, fallopian cancer, primary peritoneal cancer, uterine cancer, mesothelioma, cervical cancer, or pancreatic cancer.

In some embodiments, the administration of the immune cell enhances an immune response in the subject. In some embodiments, the enhanced immune response is an adaptive immune response. In some embodiments, the enhanced immune response is an innate immune response. In some embodiments, the enhanced immune response is an increased expression of at least one cytokine or chemokine. In some embodiments, the at least one cytokine or chemokine is IL-2 or IFNγ. In some embodiments, the enhanced immune response is an increased lysis of target cells. In some embodiments, the method further comprises administering an immunotherapy to the subject concurrently with the immune cell or subsequently to the immune cell.

Also disclosed herein, in various embodiments, is a method of modulating the activity of an immune cell comprising: obtaining an immune cell comprising the system disclosed herein; the recombinant nucleic acid disclosed herein; and/or the vector disclosed herein; and contacting the immune cell with a target cell expressing ALPG/P and MSLN, wherein binding of the priming receptor to ALPG/P on the target cell induces activation of the priming receptor and expression of the chimeric antigen receptor, wherein binding of the chimeric antigen receptor to MSLN on the target cell modulates the activity of the immune cell, and wherein the cytokine and/or the synthetic pathway activator also modulates the activity of the immune cell.

In some embodiments, the modulation of the immune cell activity comprises enhancing an immune response. In some embodiments, the enhanced immune response is an adaptive immune response. In some embodiments, the enhanced immune response is an innate immune response. In some embodiments, the immune cell activity is an increased expression of at least one cytokine or chemokine. In some embodiments, the at least one cytokine or chemokine is IL-2 or IFNγ. In some embodiments, the immune cell activity is lysis of target cells.

Also disclosed herein, in various embodiments, is a system comprising: (a) at least one of a CAR, an SPA, and a cytokine; and (b) a suppressor of RASA2 expression. In some embodiments, the SPA comprises a leucine zipper-gp130 (L-gp130). In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74. In some embodiments, the SPA comprises the amino acid sequence of SEQ ID NO: 74. In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75. In some embodiments, the SPA comprises the amino acid sequence of SEQ ID NO: 75.

In some embodiments, the cytokine is IL-2. In some embodiments, the cytokine comprises the amino acid sequence set forth in SEQ ID NO: 86. In some embodiments, the cytokine is IL-15. In some embodiments, the cytokine comprises the amino acid sequence set forth in SEQ ID NO: 96. In some embodiments, the suppressor of RASA2 expression is an shRNA or an sgRNA. In some embodiments, the suppressor of RASA2 expression is an shRNA. In some embodiments, the shRNA comprises the nucleic acid sequence of SEQ ID NO: 165. In some embodiments, the suppressor of RASA2 expression is an sgRNA. In some embodiments, the sgRNA comprises the nucleic acid sequence of SEQ ID NO: 161.

In some embodiments, the system further comprises an shRNA that suppresses the expression of TNFRSF6 (Fas). In some embodiments, the system further comprises an shRNA that suppresses the expression of TGFBR2. In some embodiments, the system further comprises an shRNA that suppresses the expression of PTPN2.

Also disclosed herein, in various embodiments, are one or more recombinant nucleic acids comprising at least one nucleic acid fragment comprising a nucleotide sequence encoding the system disclosed herein.

Also disclosed herein, in various embodiments, is an expression vector comprising the recombinant nucleic acid sequence provided herein.

Also disclosed herein, in various embodiments, is an engineered immune cell comprising: the system disclosed herein; the recombinant nucleic acid sequence disclosed herein; or the expression vector disclosed herein.

Also disclosed herein, in various embodiments of an engineered immune cell, is an improvement comprising: (a) at least one of a CAR, an SPA, and a cytokine; and (b) a suppressor of RASA2 expression. In some embodiments, the SPA comprises a leucine zipper-gp130 (L-gp130). In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74. In some embodiments, the SPA comprises the amino acid sequence of SEQ ID NO: 74. In some embodiments, the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75. In some embodiments, the SPA comprises the amino acid sequence of SEQ ID NO: 75.

In some embodiments, the cytokine is IL-2. In some embodiments, the cytokine comprises the amino acid sequence set forth in SEQ ID NO: 86. In some embodiments, the suppressor of RASA2 expression is an shRNA or an sgRNA. In some embodiments, the suppressor of RASA2 expression is an shRNA. In some embodiments, the shRNA comprises the nucleic acid sequence of SEQ ID NO: 165. In some embodiments, the suppressor of RASA2 expression is an sgRNA. In some embodiments, the sgRNA comprises the nucleic acid sequence of SEQ ID NO: 161.

In some embodiments, the engineered immune cell further comprises an shRNA that suppresses the expression of TNFRSF6 (Fas). In some embodiments, the engineered immune cell further comprises an shRNA that suppresses the expression of TGFBR2. In some embodiments, the engineered immune cell further comprises an shRNA that suppresses the expression of PTPN2.

In some embodiments, the engineered immune cell is a primary human immune cell. In some embodiments, the engineered immune cell is an allogeneic immune cell. In some embodiments, the engineered immune cell is an autologous immune cell. In some embodiments, the primary immune cell is a natural killer (NK) cell, a T cell, a CD8+ T cell, a CD4+ T cell, a primary T cell, or a T cell progenitor. In some embodiments, the primary immune cell is a primary T cell. In some embodiments, wherein the primary immune cell is a primary human T cell. In some embodiments, the primary immune cell is virus-free.

Also disclosed herein, in various embodiments, is a population of cells comprising a plurality of engineered immune cells disclosed herein.

Also disclosed herein, in various embodiments, is a pharmaceutical composition comprising the engineered immune cell disclosed herein or the population of disclosed herein and a pharmaceutically acceptable excipient.

Also disclosed herein, in various embodiments, is a method of treating a disease in a subject comprising administering the engineered immune cell disclosed herein or the pharmaceutical composition disclosed herein to the subject. In some embodiments, the disease is cancer. In some embodiments, the cancer is a solid cancer or a liquid cancer. In some embodiments, the cancer is ovarian cancer, fallopian cancer, primary peritoneal cancer, uterine cancer, mesothelioma, cervical cancer, or pancreatic cancer. In some embodiments, the administration of the engineered immune cell enhances an immune response in the subject. In some embodiments, the enhanced immune response is an adaptive immune response. In some embodiments, the enhanced immune response is an innate immune response. In some embodiments, the enhanced immune response is an increased expression of at least one cytokine or chemokine. In some embodiments, the at least one cytokine or chemokine is IL-2 or IFNγ. In some embodiments, the enhanced immune response is an increased lysis of target cells. In some embodiments, the method further comprises administering an immunotherapy to the subject concurrently with the engineered immune cell or subsequently to the engineered immune cell.

Also disclosed herein, in various embodiments, is a method of inhibiting a target cell in a subject comprising administering the engineered immune cell disclosed herein or the pharmaceutical composition disclosed herein to the subject, wherein the engineered immune cell inhibits the target cell. In some embodiments, the target cell is a cancer cell.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows a schematic of an exemplary logic gate protein expression system employing a synthetic pathway activator (SPA).

FIG. 2A shows the fold change of T cells expressing various pro-survival receptors cultured in cytokine-free media over a 6-day period. The percentage of logic gate-expressing T cells (LG T cells) in culture is defined by the number of cells expressing the Myc tag on the primeR. FIG. 2B shows the absolute cell count of the cells from FIG. 2A expressed as fold change from the first day of culture.

FIG. 3 shows phosphorylation of STAT1, STAT3, and STAT5 proteins under non-stimulated conditions in T cells expressing two types of synthetic pathway activators, as compared to T cells expressing an inert truncated EGFR molecule on the surface (EGFRt).

FIG. 4A shows a comparison of baseline pSTATI levels across LG Ts expressing various types of synthetic pathway activators or constitutively secreted cytokine molecules. FIG. 4B shows a comparison of baseline pSTAT3 levels across LG Ts expressing various types of synthetic pathway activators or constitutively secreted cytokine molecules. FIG. 4C shows a comparison of baseline pSTAT5 levels across LG Ts expressing various types of synthetic pathway activators or constitutively secreted cytokine molecules.

FIG. 5A shows primeR surface expression in T cells expressing the indicated proteins cultured alone or with antigen-negative tumor cells. FIG. 5B shows catalytic CAR surface expression in T cells expressing the indicated proteins cultured alone or with antigen-negative tumor cells.

FIG. 6A shows cytotoxic activity of LG Ts expressing pro-survival modules or an inert EGFRt molecule when cocultured with antigen-positive cells. FIG. 6B shows cytotoxic activity of LG Ts expressing pro-survival modules or an inert EGFRt molecule when cocultured with antigen-negative cells.

FIG. 7A shows continual analysis over a 72 hr period of tumor cell killing by LG Ts expressing pro-survival molecules at 4 different Effector: Target ratios. FIG. 7B shows continual analysis over a 72 hr period of T cell expansion during coculture with antigen-positive tumor cells at 4 different Effector: Target ratios.

FIG. 8A shows cell counts of logic gate T cells expressing indicated pro-survival receptors cultured with ALPG/MSLN-expressing RPMI cells over a 10 day period. FIG. 8B shows a representative well image of logic gate T cells expressing truncated EGFR at day 10.

FIG. 8C shows a representative well image of logic gate T cells expressing a gp130-based SPA at day 10.

FIG. 9 shows quantification of flow cytometry staining of various T cell phenotype markers on pre-challenge LG Ts expressing various pro-survival molecules.

FIG. 10 shows cytotoxicity and T cell expansion throughout a 15-day repetitive stimulation assay wherein LG Ts were challenged every 2-3 days with K562 tumor cells.

FIG. 11A shows summary metrics depicting total tumor cell expansion throughout a 31-day repetitive stimulation assay (in presence or absence of IL-2-supplemented media) with data from two donors shown (left and right panels). FIG. 11B shows summary metrics depicting total T cell expansion throughout a 31-day repetitive stimulation assay (in presence or absence of IL-2-supplemented media) with data from two donors shown (left and right panels).

FIG. 12 shows cell viability of T cells expressing indicated pro-survival receptors cultured in cytokine-free media over a 6-day period following 6 consecutive rounds of tumor challenge.

FIG. 13A shows quantification of flow cytometry staining of various T cell phenotype markers on ICTs after 6 rounds of tumor challenge in the absence of IL-2-supplemented media.

FIG. 13B shows quantification of flow cytometry staining of various T cell phenotype markers on ICTs after 6 rounds of tumor challenge in the presence of IL-2-supplemented media.

FIG. 14A shows comparison of cell phenotype in ICTs stimulated with tumor cells in the absence of IL-2. FIG. 14B shows comparison of cell phenotype in ICTs stimulated with tumor cells in the presence of IL-2.

FIG. 15A shows comparison of cell phenotype change over time in T cells expressing truncated EGFR stimulated with tumor cells in the absence of IL-2. FIG. 15B shows comparison of cell phenotype change over time in T cells expressing truncated EGFR stimulated with tumor cells in the presence of IL-2. FIG. 15C shows comparison of cell phenotype change over time in T cells expressing a gp 130-based SPA stimulated with tumor cells in the absence of IL-2. FIG. 15D shows comparison of cell phenotype change over time in T cells expressing a gp130-based SPA stimulated with tumor cells in the presence of IL-2.

FIGS. 16A-16B shows phosphorylation of STAT3 (FIG. 16A) and STAT1 (FIG. 16B) proteins under non-stimulated conditions in T cells expressing L-gp130 with or without knockdown of FAS and PTPN2. T cells expressing EGFRt are used as a control.

FIG. 17A shows relative expansion of target cells following incubation with LG T cells expressing EGFRt or L-gp130 with or without knockdown of FAS/PTPN2. FIG. 17B shows relative counts of SPA-expressing LG T cells expressing the indicated surface markers, as measured by flow cytometry.

FIG. 18A is a heatmap of differential gene expression in LG T cells expressing an SPA before and after challenge with a repetitive stimulation assay. FIG. 18B is a heatmap of differential expression of selected markers of T cell exhaustion in LG T cells before and after challenge with a repetitive stimulation assay.

FIG. 19 depicts the accessibility of genetic loci of selected markers (TIGIT and TOX) of T cell exhaustion in LG T cells as measured by ATAC-seq before and after challenge with a repetitive stimulation assay.

FIG. 20 diagrams the SPA interrogation assay design in mice using CAR/primeR logic gate T cells expressing the indicated SPAs.

FIG. 21 diagrams the SPA interrogation assay design in mice using CAR/primeR logic gate T cells expressing the indicated SPAs.

FIG. 22A shows tumor burden in mice treated with CAR/primeR LG T cells expressing the indicated SPAs. FIG. 22B shows levels of LG T cells in mice treated with CAR/primeR logic gate T cells expressing the indicated SPAs. FIG. 22C shows the cell counts of LG T cells expressing the indicated SPAs at the experimental endpoint. Cell counts are normalized to EGFRt control.

FIG. 23A shows tumor burden in mice treated with CAR/primeR logic gate T cells expressing the indicated SPAs. FIG. 23B shows levels of logic gate T cells in mice treated with CAR/primeR logic gate T cells expressing the indicated SPAs.

FIGS. 24A-24C depict the effects of SPA-expressing LG T cells in a murine xenograft model of renal cell carcinoma. FIG. 24A diagrams the experimental design. FIG. 24B depicts tumor burden in mice engrafted with LG T cells expressing the indicated SPAs. FIG. 24C depicts the total counts of LG T cells expressing the SPAs in whole blood at indicated time points following LG T cell engraftment.

FIG. 25A diagrams an assay testing inducible cytokine secretion in response to logic gate activation. FIG. 25B shows activation of inducible IL-2 secretion in response to logic gate activation.

FIG. 26A diagrams an assay testing CAR-T cell mediated cytotoxicity in response to logic gate activation with or without inducible cytokine expression. FIG. 26B shows CAR-T cell mediated cytotoxicity at indicated effector: target (E: T) ratios in the absence of antigen stimulation. FIG. 26C shows CAR-T cell mediated cytotoxicity at E: T ratios in the presence of priming and cytolytic antigen stimulation.

FIG. 27A diagrams a flow cytometry analysis of leakiness of the Logic Gate in un-stimulated LG T cells. FIG. 27B shows expression of the primeR and CAR in the absence of antigen stimulation in LG T cells with the nucleic acid encoding the cytokine “payload” situated upstream of the nucleic acid encoding the CAR. FIG. 27C shows expression of the primeR and CAR in the absence of antigen stimulation in LG T cells with the nucleic acid encoding the cytokine “payload” situated downstream of the nucleic acid encoding the CAR.

FIG. 28 shows expansion of T cells expressing Logic Gates in the presence of indicated cytokines.

FIG. 29 shows expansion of T cells expressing Logic Gates in the presence of indicated cytokines.

FIG. 30 shows secretion of indicated inducible cytokines compared to control in Logic Gate T cells stimulated with PrimeR and CAR antigens.

FIG. 31 shows expansion of T cells expressing Logic Gates plus indicated inducible cytokines.

FIG. 32A details the construction of inducible cytokines with non-native signal peptides to allow tunable secretion. FIG. 32B shows the levels of IL-7 secretion in Logic Gate T cells with indicated signal peptides.

FIG. 33A shows tumor volume over time in mice treated with Logic Gate T cells expressing indicated accessory molecules. FIG. 33B shows titers of Logic Gate T cells expressing indicated accessory molecules at indicated time points following tumor injection. FIG. 33C shows titer of Logic Gate T cells expressing indicated accessory molecules 15 days following tumor injection.

FIGS. 34A-34C are schematics detailing the workflow of the high-throughput screen of combinations of synthetic pathway activators (SPAs) and cytokines. FIG. 34A details the cell editing process for the production of LG T cells expressing the SPA and cytokine combinations. FIG. 34B details the process of normalizing the edited cells for the screen. FIG. 34C details the continuous stimulation assay used to screen edited T cells for effects on target cell killing and T cell proliferation.

FIGS. 35A-35C show the results of the screen of LG T cells expressing the indicated SPA and cytokine combinations based on decreased counts of target cells (lower number indicates increased target cell killing). FIG. 35A shows results of combinations with boxes colored based on the expressed SPA. FIG. 35B shows results of combinations with boxes colored based on the expressed cytokine. FIG. 35C shows results of the top 20 tested combinations compared to indicated positive controls.

FIGS. 36A and 36B show graphs detailing combinations that perform better that single-module LG T cells. FIG. 36A shows combinations that perform better that single-module LG T cells based on decreased target cell count. FIG. 36B shows combinations that perform better that single-module LG T cells based on increased T cell expansion.

FIGS. 37A and 37B show graphs detailing combinations that showed no additive effects relative to single-module LG T cells. FIG. 37A shows a graph detailing the effects of the combination of L-gp130 with an sgRNA targeting PTPN2 on T cell expansion compared to the effects of L-gp130 or the sgRNA alone. FIG. 37B shows a graph detailing the effects of the combination of C7R with an sgRNA targeting CISH on target cell count compared to the effects of C7R or the sgRNA alone. FIG. 37C shows an ANOVA analysis of the combination of L-gp130 and IL-2 in LG T cells across various backgrounds. FIG. 37D shows an ANOVA analysis of the combination of L-gp130 and IL-12 in LG T cells across various backgrounds.

FIG. 38 is a graph showing results of a continuous stimulation assay screen of LG T cells expressing indicated cytokine and sgRNA/ribonucleoproteins and cultured in the presence of indicated cytokines.

FIGS. 39A-39C detail a continuous stimulation assay screen of LG T cells expressing various shRNAs, inducible cytokine payloads, sgRNAs, and SPAs cultured in the presence or absence of TGF-β. FIG. 39A details the tested combinations. FIG. 39B shows results of indicated combinations including inducible IL-2 expression. FIG. 39C shows results of indicated combinations including inducible IL-12 expression.

FIGS. 40A-40C detail a continuous stimulation assay screen of LG-T cells expressing various shRNAs, inducible cytokine payloads, sgRNAs, and SPAs. FIG. 40A shows results of a screen of LG T cells in the absence of TGF-β (left panel) with selected results of indicated combinations (right panel). FIG. 40B shows results of a screen of LG T cells in the presence of TGF-β (left panel) with selected results of indicated combinations (right panel). FIG. 40C shows the top performing cytokine, SPA, and sgRNA combinations (left panel) or SPA and sgRNA combinations with a control inducible payload (right panel).

FIG. 41A shows the mean tumor volume in mice after treatment with the indicated ICT cells. FIG. 41B shows the total T cells at 7, 14, 21, and 42 days post T cell injection.

FIG. 42 shows serum levels of IL2 and IFNγ on days 14, 21, and 42 after in vivo injection with the indicated ICT cells.

FIG. 43A shows TCR expansion and percent tumor lysis after incubation of T cells with exogenous IL-2 or IL-15 or delayed addition of exogenous IL-2 or IL-15. FIG. 43B shows percent TCR target cell growth after incubation with the indicated cytokine.

FIG. 44 shows that addition of IL-15 expression to the ICT cells expressing L-gp130 (SPA.I) resulted in increased killing by the T cells.

### DETAILED DESCRIPTION

### Definitions

Terms used in the claims and specification are defined as set forth below unless otherwise specified.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). For example, an antibody that “selectively binds” or “specifically binds” an antigen is an antigen-binding moiety that binds the antigen with high affinity and does not significantly bind other unrelated antigens. Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the extracellular antigen-binding domain specifically binds to Alkaline phosphatase, Germ Cell type (ALPG). In some embodiments, the extracellular domain includes an antigen-binding moiety that binds to Alkaline phosphatase, Placenta (ALPP).

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (KD). The kinetic components that contribute to the dissociation equilibrium constant are described in more detail below. Affinity can be measured by common methods known in the art, including, but not limited to, surface plasmon resonance (SPR) technology (e.g., BIACORE®) or biolayer interferometry (e.g., FORTEBIO®).

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDRI in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at bioinf.org.uk/abs/abnum/, and described in Abhinandan and Martin, Immunology, 2008, 45:3832-3839, incorporated by reference in its entirety.

TABLE A
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

As used herein, the term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CHI domain including one or more cysteines from the antibody hinge region.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

The “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; 5,587,458.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen MM, De Haard HJ (2007). “Properties, production, and applications of camelid single-domain antibody fragments”. Appl. Microbiol Biotechnol. 77 (1): 13-22).

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table D.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of cubacterial and archacal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of cubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24;110 (39): 15644-9; Sampson et al., Nature. 2013 May 9;497 (7448): 254-7; and Jinek, et al., Science. 2012 Aug. 17;337 (6096): 816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3 + cell. T cells can be CD4⁺, CD8⁺, or CD4⁺ and CD8⁺. The T cell can be any type of T cell, CD4 +/CD8+ double positive T cells, CD4+ helper T cells (e.g., Th1 and Th2 cells), CD8+ T cells (e.g., cytotoxic T cells), peripheral Including but not limited to blood mononuclear cells (PBMC), peripheral blood leukocytes (PBL), tumor infiltrating lymphocytes (TIL), memory T cells, naive T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin ⁻. In some cases, human hematopoietic stem cells are identified as CD34⁺, CD59⁺, Thy 1/CD90⁺, CD38^{lo}/⁻, C-kit/CD117⁺, lin⁻. In some cases, human hematopoietic stem cells are identified as CD34⁻, CD59⁺, Thy1/CD90⁺, CD38^{lo}/⁻, C-kit/CD117⁺, lin⁻. In some cases, human hematopoietic stem cells are identified as CD133⁺, CD59⁺, Thy1/CD90⁺, CD38⁺/⁻, C-kit/CD117⁺, lin⁻. In some cases, mouse hematopoietic stem cells are identified as CD34^{lo}/⁻, SCA-1⁺, Thy1⁺/^{lo}, CD38⁺, C-kit⁺, lin⁻. In some cases, the hematopoietic stem cells are CD150+CD48-CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, e.g., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g., DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (e.g., a naturally occurring polypeptide) or fragment thereof or a variant polypeptide (e.g., a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene-recombinant DNA—that has been cloned in a system that supports expression of the gene and translation of messenger RNA (see expression system). The recombinant protein can be a therapeutic agent, e.g. a protein that treats a disease or disorder disclosed herein. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (e.g., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g., DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked—in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

It must be noted that, as used in the specification and the appended claims, the singular forms “a,” “an” and “the” include plural referents unless the context clearly dictates otherwise.

### Logic Gate Systems

As used herein, a “logic gate,” “circuit,” “circuit receptor,” “system” or “system receptor” refers to a two part protein expression system comprising a priming receptor and a chimeric antigen receptor. The system can be encoded on at least one nucleic acid inserted into a cell, where the priming receptor is expressed in the cell. The intracellular domain of the priming receptor is cleaved from the transmembrane domain upon binding of the priming receptor to its target antigen. The intracellular domain is then capable of translocating into a cell nucleus where it induces expression of the chimeric antigen receptor. A synthetic pathway activator can also be employed to enhance expansion and activity of logic gate-expressing T cells (LG T cells).

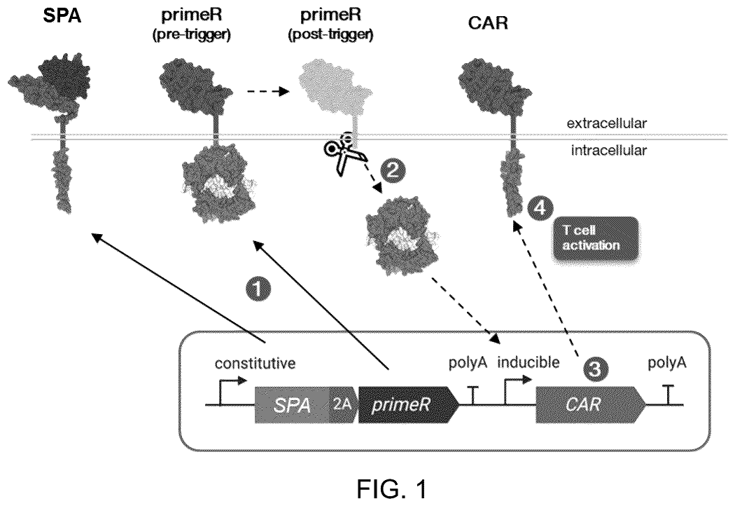

An overview of an exemplary logic gate system employing a synthetic pathway activator is shown in FIG. 1. In various embodiments, the system comprises 4 steps leading to T cell activation: (1) the synthetic pathway activator (SPA) and priming receptor (primeR) are constitutively expressed; (2) the primeR is triggered, resulting in cleavage of the intracellular domain; (3) the cleaved primeR intracellular domain induces expression of the CAR; and (4) the CAR is activated, resulting in T cell activation.

In one aspect, provided herein are systems comprising a priming receptor that binds to ALPG/P, a chimeric antigen receptor that binds to MSLN, and a cytokine and/or a synthetic pathway activator that activates cytokine signaling, wherein the transcription factor of the intracellular domain of the priming receptor is capable of inducing expression of the CAR. Such systems are alternatively termed “logic gates” or “circuits.” In some aspects, the system is encoded by nucleic acid transgenes inserted into an immune cell. The system can be encoded on a single nucleic acid insert or fragment that comprises both transgenes, or can be encoded on two nucleic acids that encode the system transgenes individually. The priming receptor, SPA, and CAR, of the system can be placed in any order on the single nucleic acid. For example, the priming receptor can be at the 5′ end, the SPA can be immediately after the SPA of the priming receptor, and the CAR can be at the 3′ end; or the SPA can be at the 5′ end, the priming receptor can be immediately after the SPA, and the CAR can be at the 3′ end; or the CAR can be at the 5′ end, and the SPA can be after the CAR and the priming receptor can be at the 3′ end; or the CAR can be at the 5′ end, and the priming receptor can be after the CAR and the SPA can be at the 3′ end.

A constitutive promoter can be operably linked to the nucleotide sequence encoding the priming receptor and/or the SPA. The nucleic acids encoding the SPA and priming receptor can be under the control of a single promoter. An inducible promoter can also be operably linked to the nucleotide sequence encoding the CAR. The nucleic acids encoding the CAR and SPA can be under the control of a separate inducible promoters (e.g., a first inducible promoter and a second inducible promoter). The first and second inducible promoters can be identical or different. In some embodiments, when the system is encoded on a single nucleic acid insert or fragment that comprises both transgenes, the nucleic acid can comprise, in a 5′ to 3′ direction, the constitutive promoter; the nucleotide sequence encoding priming receptor; the inducible promoter; and the nucleotide sequence encoding chimeric antigen receptor. Alternatively, the nucleic acid can comprise, in a 5′ to 3′ direction, the inducible promoter; the nucleotide sequence encoding chimeric antigen receptor; the constitutive promoter; and the nucleotide sequence encoding priming receptor. The SPA can be present upstream or downstream of the priming receptor and/or CAR.

### Priming Receptors

Provided herein are priming receptors comprising an extracellular antigen-binding domain that specifically binds Alkaline Phosphatase, Placental/Germ Cell (ALPG/P); ALPP: NCBI Entrez Gene: 250, UniProtKB/Swiss-Prot: P05187, SEQ ID NO: 176; ALPG: NCBI Entrez Gene: 251, UniProtKB/Swiss-Prot: P10696, SEQ ID NO: 177). In some embodiments, the priming receptor comprises an extracellular antigen-binding domain that specifically binds Alkaline Phosphatase, Placental (ALPP). In some embodiments, the priming receptor comprises an extracellular antigen-binding domain that specifically binds Alkaline Phosphatase, Germ Cell (ALPG). As used herein, “Alkaline Phosphatase, Placental/Germ Cell (ALPG/P)” refers to both Alkaline Phosphatase, Placental (ALPP) and Alkaline Phosphatase, Germ Cell (ALPG). An antigen binding domain that specifically binds ALPG/P is capable of specifically binding ALPG and/or ALPP.

In some embodiments, the priming receptor comprises a sequence as set forth in SEQ ID NO: 24. In some embodiments, the priming receptor comprises a sequence as set forth in SEQ ID NO: 25.

In certain aspects of the present disclosure, the priming receptor is a synthetic receptor based on the Notch protein. Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synthetic notch priming receptor replaces the natural Notch intracellular fragment with one that causes a gene encoding a protein of choice, such as a CAR, to be transcribed upon release of the intracellular fragment from the priming receptor.

Notch receptors have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robol, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Linker Notch” receptors comprise a heterologous extracellular ligand binding domain, a linking polypeptide lacking substantial sequence identity with a Notch receptor (e.g., a synthetic (GGS), polypeptide sequence), a TMD, and an ICD. “Hinge Notch” receptors comprise a heterologous extracellular ligand binding domain, a hinge sequence comprising an oligomerization domain (e.g., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

### Priming Receptor Extracellular Domain

The priming receptor disclosed herein comprises an extracellular domain that specifically binds Alkaline Phosphatase, Placental/Germ Cell (ALPG/P). In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

### Priming Receptor CDRs, VH, VL Domains

In some aspects, the priming receptor extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 1, 39, 40, 41, or 42, CDR-H2 comprises the sequence set forth in SEQ ID NO: 2, 43, 44, 45, or 46, CDR-H3 comprises the sequence set forth in SEQ ID NO: 3, 47, or 48, CDR-L1 comprises the sequence set forth in SEQ ID NO: 4, 49, or 50, CDR-L2 comprises the sequence set forth in SEQ ID NO: 5 or 51; and CDR-L3 comprises the sequence set forth in SEQ ID NO: 6 or 53. In some embodiments, the VH chain sequence comprises the sequence set forth in SEQ ID NO: 7. In some embodiments, the VL comprises the sequence set forth in SEQ ID NO: 8. In some embodiments, the extracellular domain comprises the sequence set forth in SEQ ID NO: 9

In some embodiments, the priming receptor extracellular antigen-binding domain CDR-H3 has at least about 50%, 75%, 80%, 85%, 90%, or 95% identity with a CDR-H3 of SEQ ID NO: 3, 47, or 48, the CDR-H2 has at least about 50%, 75%, 80%, 85%, 90%, or 95% identity with a CDR-H2 of SEQ ID NO: 2, 43, 44, 45, or 46, the CDR-H1 has at least about 50%, 75%, 80%, 85%, 90%, or 95% identity with a CDR-H1 of SEQ ID NO: 1, 39, 40, 41, or 42, the CDR-L3 has at least about 50%, 75%, 80%, 85%, 90%, or 95% identity with a CDR-L3 of SEQ ID NO: 6 or 53, the CDR-L2 has at least about 50%, 75%, 80%, 85%, 90%, or 95% identity with a CDR-L2 of SEQ ID NO: 5 or 51, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 3, 47, or 48, with up to 1, 2, 3, 4, 5, 6, 7, or 8 amino acid substitutions; the CDR-H2 is a CDR-H2 of SEQ ID NO: 2, 43, 44, 45, or 46, with up to 1, 2, 3, 4, 5, 6, 7, or 8 amino acid substitutions; the CDR-H1 is a CDR-H1 of SEQ ID NO: 1, 39, 40, 41, or 42, with up to 1, 2, 3, 4, or 5 amino acid substitutions; the CDR-L3 is a CDR-L3 of SEQ ID NO: 6 or 53, with up to 1, 2, 3, 4, or 5 amino acid substitutions; the CDR-L2 is a CDR-L2 of SEQ ID NO: 5 or 51, with up to 1, 2, 3, or 4 amino acid substitutions; and the CDR-L1 is a CDR-L1 of SEQ ID NO: 4 with up to 1, 2, 3, 4, 5, or 6 amino acid substitutions.

In some embodiments, a priming receptor extracellular antigen-binding domain provided herein comprises one to three CDRs of a VH domain as set forth in SEQ ID NO: 7. In some embodiments, an antigen-binding domain provided herein comprises two to three CDRs of a VH domain as set forth in SEQ ID NO: 7. In some embodiments, an antigen-binding domain provided herein comprises three CDRs of a VH domain as set forth in SEQ ID NO: 7. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a priming receptor extracellular antigen-binding domain provided herein comprises a VH sequence having at least about 50%, 60%, 70%, 80%, 90%, 95%, or 99% identity to an VH sequence set forth in SEQ ID NO: 7. In some embodiments, an antigen-binding domain provided herein comprises a VH sequence provided in SEQ ID NO: 7, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a priming receptor extracellular antigen-binding domain provided herein comprises one to three CDRs of a VL domain as set forth in SEQ ID NO: 8. In some embodiments, an antigen-binding domain provided herein comprises two to three CDRs of a VL domain as set forth in SEQ ID NO: 8. In some embodiments, an antigen-binding domain provided herein comprises three CDRs of a VL domain as set forth in SEQ ID NO: 8. In some aspects, the CDRs are Kabat CDRs. In some aspects, the Kabat VH CDRs are provided in the sequences set forth as SEQ ID NOs: 40, 44, and 3, and the Kabat VL CDRs are provided in the sequences set forth as SEQ ID NOs: 4, 5, and 6. In some aspects, the CDRs are Chothia CDRs. In some aspects, the Chothia VH CDRs are provided in the sequences set forth as SEQ ID NOs: 1, 2, and the Chothia VL CDRs are provided in the sequences set forth as SEQ ID NOs: 4, 5, and 6. In some aspects, the CDRs are AbM CDRs. In some aspects, the AbM VH CDRs are provided in the sequences set forth as SEQ ID NOs: 39, 43, and 3, and the AbM VL CDRs are provided in the sequences set forth as SEQ ID NOs: 4, 5, and 6. In some aspects, the CDRs are Contact CDRs. In some aspects, the Contact VH CDRs are provided in the sequences set forth as SEQ ID NOs: 41, 45, and 47, and the Contact VL CDRs are provided in the sequences set forth as SEQ ID NOs: 49, 51, and 53. In some aspects, the CDRs are IMGT CDRs. In some aspects, the IMGT VH CDRs are provided in the sequences set forth as SEQ ID NOs: 42, 46, and 48, and the IMGT VL CDRs are provided in the sequences set forth as SEQ ID NOs: 50, 52, and 6.

In some embodiments, a priming receptor extracellular antigen-binding domain provided herein comprises a VL sequence having at least about 50%, 60%, 70%, 80%, 90%, 95%, or 99% identity to an VL sequence set forth in SEQ ID NO: 8. In some embodiments, an antigen-binding domain provided herein comprises a VL sequence provided in SEQ ID NO: 8, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

Table B provides the CDR sequences of the VH and VL of an illustrative ALPG/P antigen binding domain according to the indicated numbering schemes.

TABLE B

| SEQ ID | | Numbering | |
|---|---|---|---|
| NO | Name | scheme | Sequence |
| 1 | CDR-H1 | Chothia | GFSLTSY--- |
| 39 | | AbM | GFSLTSYGVS |
| 40 | | Kabat | -----SYGVS |
| 41 | | Contact | ----TSYGVS |
| 42 | | IMGT | GFSLTSYG-- |
| 2 | CDR-H2 | Chothia | -----WEDGS--------- |
| 43 | | AbM | ---VIWEDGSTN------- |
| 44 | | Kabat | ---VIWEDGSTNYHSALIS |
| 45 | | Contact | WIGVIWEDGSTN------- |
| 46 | | IMGT | ----IWEDGST-------- |
| 3 | CDR-H3 | Chothia | --PHYGSSYVGAMEY |
| 3 | | AbM | --PHYGSSYVGAMEY |
| 3 | | Kabat | --PHYGSSYVGAMEY |
| 47 | | Contact | ARPHYGSSYVGAME- |
| 48 | | IMGT | ARPHYGSSYVGAMEY |
| 4 | CDR-L1 | Chothia | RASENIYSYVA-- |
| 4 | | AbM | RASENIYSYVA-- |
| 4 | | Kabat | RASENIYSYVA-- |
| 49 | | Contact | ------YSYVAWY |
| 50 | | IMGT | ---ENIYSY---- |
| 5 | CDR-L2 | Chothia | ----NAKSLAS |
| 5 | | AbM | ----NAKSLAS |
| 5 | | Kabat | ----NAKSLAS |
| 51 | | Contact | LLIYNAKSLA- |
| 52 | | IMGT | ----NA----- |
| 6 | CDR-L3 | Chothia | QHHYVSPWT |
| 6 | | AbM | QHHYVSPWT |
| 6 | | Kabat | QHHYVSPWT |
| 53 | | Contact | QHHYVSPW- |
| 6 | | IMGT | QHHYVSPWT |

### Transmembrane Domain

As described above, the priming receptor comprises a transmembrane domain (TMD) comprising one or more ligand-inducible proteolytic cleavage sites.

In some embodiments, the TMD comprises a Notch1 transmemebrane domain. In some embodiments, the transmembrane domain comprises the sequence as set forth in SEQ ID NO: 19.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSFIR, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors ILIR1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBOI, SORCS3, SORCS1, SORLI, SDC1, SDC2, SPN, TYR, TYRP1, DCT, YASN, FLT1, CDH5, PKHD1, NECTINI, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the priming receptor comprises a Notch cleavage site, such as S2 or S3. Additional proteolytic cleavage sites suitable for the compositions and methods disclosed herein include, but are not limited to, ADAM10, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO:182), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO:183), where cleavage occurs after the lysine residue. Another example of a protease cleavage site that can be included in a proteolytically cleavable linker is a thrombin cleavage site, e.g., Leu-Val-Pro-Arg (SEQ ID NO:184). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

### Intracellular Domain

In some embodiments, the priming receptor comprises one or more intracellular domains from or derived from a transcriptional regulator and/or a DNA-binding domain. In some embodiments, the intracellular domain comprises a Gal4/VP64 domain. In some embodiments, the intracellular domain comprises the sequence as set forth in SEQ ID NO: 23.

Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” refers to the domain of a transcription factor that interacts with transcriptional control elements and/or transcriptional regulatory proteins (e.g., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (cAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

In some embodiments, the priming receptor comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNFla, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Casof, Cas7, Cas8al, Cas8a2, Cas8b, Cas8c, Cas10, Cas10d, CasF, CasG, CasH, Csy1, Csy2, Csy3, Cscl (or CasA), Csc2 (or CasB), Csc3 (or CasE), Csc4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx 10, Csx 16, CsaX, Csx3, Csz1, Csx 15, Csf1, Csf2, Csf3, Csf4, and Cu196, and TALES.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (e.g., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

### Juxtamembrane Domain

The ECD and the TMD, or the TMD and the ICD, can be linked to each other with a linking polypeptide, such as a juxtamembrane domain. “SynNotch” or synthetic notch receptors comprise a heterologous extracellular ligand -binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robol, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand -binding domain, a linking polypeptide lacking substantial sequence identity with a Notch receptor (for example, without limitation, having a synthetic (GGS)ₙ polypeptide sequence), a TMD, and an ICD. “Hinge Notch” receptors comprise a heterologous extracellular ligand -binding domain, a hinge sequence comprising an oligomerization domain (e.g., a domain that promotes dimerization, trimerization, or higher order multimerization with a synthetic receptor and/or an existing host receptor), a TMD, and an ICD.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligimerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker does not comprise a protease cleavage site or a glysosylation site.

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO:185).

Hinge polypeptide sequences can also be derived from a CD8a hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8 a hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robol JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

### Stop-Transfer Sequence

In some embodiments, the priming receptor further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in priming receptors is described in WO2021061872, hereby incorporated by reference in its entirety. Non-limiting exemplary STS sequences include APLP1, APLP2, APP, TGBR3, CSFIR, CXCL16, CX3CL1, DAG1, DCC, DNER, DSG2, CDH1, GHR, HLA-A, IFNAR2, IGFIR, ILIR1, ERN2, KCNEI, KCNE2, CHL1, LRPI, LRP2, LRP18, PTPRF, SCNIB, SCN3B, NPR3, NGFR, PLXDC2, PAM, AGER, ROBOI, SORCS3, SORCS1, SORLI, SDC1, SDC2, SPN, TYR, TYRPI, DCT, VASN, FLTI, CDH5, PKTFDI, NECTINI, KL, IL6R, EFNB1, CD44, CLSTN1, LRP8, PCDHGC3, NRG1, LRPIB, JAG2, EFNB2, DLLI, CLSTN2, EPCAM, ErbB4, KCNE3, CDH2, NRG2, PTPRK, BTC, EPHA4, ILIR2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

In some embodiments, the stop-transfer-sequence comprises the sequence as set forth in SEQ ID NO: 20.

### Chimeric Antigen Receptors

In another aspect, provided herein are chimeric antigen receptors comprising an extracellular antigen-binding domain that specifically binds to mesothelin (MSLN; NCBI Entrez Gene: 10232; UniProtKB/Swiss-Prot: Q13421, SEQ ID NO: 178). The CAR may be a human CAR, comprising fully human sequences, e.g., natural human sequences.

In some embodiments, the chimeric antigen receptor includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand -binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some aspects, the chimeric antigen receptor includes an extracellular portion comprising an antigen binding domain described herein and an intracellular signaling domain. In some embodiments, an antibody or fragment includes an scFv, a VH, or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD8a or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

### Chimeric Antigen Receptor CDRs, VH, VL Domains

In some aspects, the priming receptor extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 10, 54, 56, 57, or 71, CDR-H2 comprises the sequence set forth in SEQ ID NO: 11, 58, 59, 60, 61, or 308, CDR-H3 comprises the sequence set forth in SEQ ID NO: 12, 62, or 63, CDR-L1 comprises the sequence set forth in SEQ ID NO: 14, 64, 65, 66, or 67, CDR-L2 comprises the sequence set forth in SEQ ID NO: 15; and CDR-L3 comprises the sequence set forth in SEQ ID NO: 16 or 72. In some embodiments, the VH chain sequence comprises the sequence set forth in SEQ ID NO: 13. In some embodiments, the VL comprises the sequence set forth in SEQ ID NO: 17. In some embodiments, the antigen-binding domain comprises the sequence set forth in SEQ ID NO: 30.

In some embodiments, the priming receptor extracellular antigen-binding domain CDR-H3 has at least about 50%, 75%, 80%, 85%, 90%, or 95% identity with a CDR-H3 of SEQ ID NO: 12, 62, or 63, the CDR-H2 has at least about 50%, 75%, 80%, 85%, 90%, or 95% identity with a CDR-H2 of SEQ ID NO: 11, 58, 59, 60, 61, or 308, the CDR-H1 has at least about 50%, 75%, 80%, 85%, 90%, or 95% identity with a CDR-H1 of SEQ ID NO: 10, 54, 56, 57, or 71, the CDR-L3 has at least about 50%, 75%, 80%, 85%, 90%, or 95% identity with a CDR-L3 of SEQ ID NO: 16 or 72, the CDR-L2 has at least about 50%, 75%, 80%, 85%, 90%, or 95% identity with a CDR-L2 of SEQ ID NO: 15, 68, 69, or 70, and the CDR-L1 has at least about 50%, 75%, 80%, 85%, 90%, or 95% identity with a CDR-L1 of SEQ ID NO: 14, 65, 66, or 67. In some embodiments, the CDR-H3 is a CDR-H3 of SEQ ID NO: 12, 62, or 63, with up to 1, 2, 3, 4, 5, 6, 7, or 8 amino acid substitutions; the CDR-H2 is a CDR-H2 of SEQ ID NO: 11, 58, 59, 60, 61, or 308, with up to 1, 2, 3, 4, 5, 6, 7, or 8 amino acid substitutions; the CDR-H1 is a CDR-H1 of SEQ ID NO: 10, 54, 56, 57, or 71, with up to 1, 2, 3, 4, or 5 amino acid substitutions; the CDR-L3 is a CDR-L3 of SEQ ID NO: 16 or 72, with up to 1, 2, 3, 4, or 5 amino acid substitutions; the CDR-L2 is a CDR-L2 of SEQ ID NO: 15, 68, 69, or 70, with up to 1, 2, 3, or 4 amino acid substitutions; and the CDR-L1 is a CDR-L1 of SEQ ID NO: 14, 65, 66, or 67 with up to 1, 2, 3, 4, 5, or 6 amino acid substitutions.

In some embodiments, a priming receptor extracellular antigen-binding domain provided herein comprises one to three CDRs of a VH domain as set forth in SEQ ID NO: 13. In some embodiments, an antigen-binding domain provided herein comprises two to three CDRs of a VH domain as set forth in SEQ ID NO: 13. In some embodiments, an antigen-binding domain provided herein comprises three CDRs of a VH domain as set forth in SEQ ID NO: 13. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a priming receptor extracellular antigen-binding domain provided herein comprises a VH sequence having at least about 50%, 60%, 70%, 80%, 90%, 95%, or 99% identity to an VH sequence set forth in SEQ ID NO: 13. In some embodiments, an antigen-binding domain provided herein comprises a VH sequence provided in SEQ ID NO: 13, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a priming receptor extracellular antigen-binding domain provided herein comprises one to three CDRs of a VL domain as set forth in SEQ ID NO: 17. In some embodiments, an antigen-binding domain provided herein comprises two to three CDRs of a VL domain as set forth in SEQ ID NO: 17. In some embodiments, an antigen-binding domain provided herein comprises three CDRs of a VL domain as set forth in SEQ ID NO: 17. In some aspects, the CDRs are Kabat CDRs. In some aspects, the Kabat VH CDRs are provided in the sequences set forth as SEQ ID NOs: 10, 11, and 12, and the Kabat VL CDRs are provided in the sequences set forth as SEQ ID NOs: 14, 69, and 16. In some aspects, the CDRs are Chothia CDRs. In some aspects, the Chothia VH CDRs are provided in the sequences set forth as SEQ ID NOs: 71, 308, and 12, and the Chothia VL CDRs are provided in the sequences set forth as SEQ ID NOs: 14, 15, and 16. In some aspects, the CDRs are AbM CDRs. In some aspects, the AbM VH CDRs are provided in the sequences set forth as SEQ ID NOs: 54, 58, and 12, and the AbM VL CDRs are provided in the sequences set forth as SEQ ID NOs: 14, 68, and 16. In some aspects, the CDRs are Contact CDRs. In some aspects, the Contact VH CDRs are provided in the sequences set forth as SEQ ID NOs: 56, 60, and 62, and the Contact VL CDRs are provided in the sequences set forth as SEQ ID NOs: 66, 70, and 72. In some aspects, the CDRs are IMGT CDRs. In some aspects, the IMGT VH CDRs are provided in the sequences set forth as SEQ ID NOs: 57, 61, and 63, and the IMGT VL CDRs are provided in the sequences set forth as SEQ ID NOs: 67, DT, and 16.

In some embodiments, a priming receptor extracellular antigen-binding domain provided herein comprises a VL sequence having at least about 50%, 60%, 70%, 80%, 90%, 95%, or 99% identity to an VL sequence set forth in SEQ ID NO: 17. In some embodiments, an antigen-binding domain provided herein comprises a VL sequence provided in SEQ ID NO: 17, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

Table C provides illustrative MSLN antigen binding domain CDR sequences of the VH of SEQ ID NO: 13 and the VL of SEQ ID NO: 17, according to the indicated numbering schemes.

TABLE C

| SEQ ID | | Numbering | Sequence |
|---|---|---|---|
| NO | Name | scheme | |
| 71 | CDR-H1 | Chothia | GYSFTGY |
| 54 | | AbM | GYSFTGYTMN |
| 10 | | Kabat | GYTMN |
| 56 | | Contact | TGYTMN |
| 57 | | IMGT | GYSFTGYT |
| 308 | CDR-H2 | Chothia | TPYNGA |
| 58 | | AbM | LITPYNGASS |
| 11 | | Kabat | LITPYNGASSYNQKFRG |
| 60 | | Contact | WIGLITPYNGASS |
| 61 | | IMGT | ITPYNGAS |
| 12 | CDR-H3 | Chothia | GGYDGRGFDY |
| 12 | | AbM | GGYDGRGFDY |
| 12 | | Kabat | GGYDGRGFDY |
| 62 | | Contact | ARGGYDGRGFD |
| 63 | | IMGT | ARGGYDGRGFDY |
| 14 | CDR-L1 | Chothia | SASSSVSYMH |
| 14 | | AbM | SASSSVSYMH |
| 14 | | Kabat | SASSSVSYMH |
| 66 | | Contact | SYMHWY |
| 67 | | IMGT | SSVSY |
| 15 | CDR-L2 | Chothia | DTSKLAS |
| 68 | | AbM | DTSKLAS |
| 69 | | Kabat | DTSKLAS |
| 70 | | Contact | RWIYDTSKLA |
| — | | IMGT | DT |
| 16 | CDR-L3 | Chothia | QQWSGYPLT |
| 16 | | AbM | QQWSGYPLT |
| 16 | | Kabat | QQWSGYPLT |
| 72 | | Contact | QQWSGYPL- |
| 16 | | IMGT | QQWSGYPLT |

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (e.g., comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the CAR comprises a CD8a TMD. In some embodiments, the CD8a TMD comprises the sequence set forth in SEQ ID NO: 27.

### CAR Hinge

In some embodiments, the CAR further includes a spacer, which may be or include at least a portion of an immunoglobulin constant region or variant or modified version thereof, such as a hinge region, e.g., a CD8a hinge, an IgG4 hinge region, and/or a CHI/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8a hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8a hinge. In some embodiments, the CD8a hinge comprises the sequence set forth in SEQ ID NO: 26.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16. In some embodiments, the CAR comprises a CD3-zeta activation domain comprising the sequence set forth in SEQ ID NO: 29.

In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD8a transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain. In some embodiments, the 4-1BB co-stimulatory domain comprises the sequence as set forth in SEQ ID NO: 28.

In some embodiments, the CAR comprises a sequence as set forth in SEQ ID NO: 30, 31, or 32. In some embodiments, the CAR comprises a sequence as set forth in SEQ ID NO: 31.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. Sce WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, e.g., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3-and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g., containing only the VH region), VH domains, and scFvs, described herein, a spacer such as a CD8a hinge, a CD8a transmembrane domain, a 4-1BB intracellular signaling domain, and a CD3 zeta signaling domain. In some embodiments, the CAR includes an antibody or fragment, including sdAbs and scFvs described herein, a spacer such as a CD8a hinge, a CD8a transmembrane domain, a 4-1BB intracellular signaling domain, and a CD3 zeta signaling domain.

Transgenes expressing the priming receptor and CAR system may be introduced into cells, such as a T cell, using, for example, a site-specific technique. With site specific integration of the transgenes (e.g., priming receptor and CAR), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered cells have applications to immune-oncology. The priming receptor and CAR, for example, can be selected to target different specific tumor antigens. Examples of cancers that can be effectively targeted using such cells are blood cancers or solid cancers. In some embodiments, immune cell therapy can be used to treat solid tumors.

### Synthetic Pathway Activators

In various aspects, systems disclosed herein employ one or more “synthetic pathway activators” (SPAs). CAR-expressing immune cells can be limited by the necessity for in vivo expansion following infusion. To achieve robust expansion, T cells require three signals: antigen-stimulation, co-stimulation, and cytokine-induced stimulation. Activation of CARs is sufficient to induce the first two signals, but cannot recapitulate cytokine signaling. Furthermore, the tumor microenvironment is often immunosuppressive and devoid of pro-inflammatory cytokines. SPAs can thus be used to stimulate robust in vivo expansion and enhance desirable properties (e.g., increased survival, persistence, and potency) of T cells expressing priming receptors and/or CARs as described herein.

### SPA Structure

In various embodiments, SPAs mimic activation of interleukin signaling. Interleukin receptors are cytokine receptors that signal through Signal Transducer and Activator of Transcription (STAT) transcription factors (e.g., STAT3 and STAT5). Interleukin receptors typically function by dimerization in response to ligand binding. Once dimerized, receptors can bind janus-associated kinases (JAKs) to induce JAK cross-phosphorylation and downstream “JAK/STAT” signaling. Accordingly, induced receptor agonism or ligand -independent dimerization of receptors can be utilized to induce constitutive receptor activity and thus, constitutive cytokine signaling.

In various embodiments, SPAs comprise interleukin receptors or functional fragments thereof. In some embodiments, SPAs comprise or are derived from interleukin receptor intracellular signaling domains or functional fragments thereof. In some embodiments, SPAs comprise or are derived from interleukin-6 signal transducer (IL6ST) polypeptides or functional fragments thereof. In some embodiments, SPAs comprise or are derived from interleukin-7 receptor (IL-7R) polypeptides or functional fragments thereof. In some embodiments SPAs comprise or are derived from interleukin-15 (IL-15) polypeptides or functional fragments thereof.

In various embodiments, one or more structural alterations can be made to confer constitutive activity to a SPA or functional fragment thereof. In some embodiments, structures or mutations can be added to induce SPA multimerization. In some embodiments, one or more amino acids can be mutated to a cysteine to allow formation of one or more disulfide bond(s), e.g., between two receptor monomers. In some embodiments, one or more amino acids can be inserted into a wild-type receptor polypeptide to promote dimerization, e.g., through formation of one or more disulfide bond(s).

In some embodiments, an exogenous polypeptide is operatively linked to a cytokine receptor or functional fragment thereof to cause their multimerization. In some embodiments, a leucine zipper polypeptide is operatively linked to a cytokine receptor or functional fragment thereof. In some embodiments, the leucine zipper polypeptide is a c-Jun leucine zipper. In some embodiments, an exogenous scaffold is operatively linked to a cytokine receptor or functional fragment thereof. In some embodiments, the exogenous scaffold is a CD34 ectodomain.

In some embodiments, SPAs can comprise a ligand agonist (e.g., a cytokine, e.g., an interleukin) that allows constitutive activation of the SPA. In some embodiments, the cytokine receptor and a soluble agonist are expressed simultaneously. In some embodiments, the cytokine receptor and a membrane-bound agonist are expressed simultaneously.

In various embodiments, SPAs are anchored to the cellular membrane. In some embodiments SPAs comprise an extracellular domain, a transmembrane domain, and an intracellular signaling domain. In some embodiments, SPAs comprise a transmembrane domain of an interleukin receptor.

### Exemplary SPAs

In some embodiments, the SPA comprises a leucine zipper-gp 130 (referred to interchangeably herein as “L-gp130” or “gp130”) or an L-gp130 intracellular signaling domain. L-gp130 comprises a homodimer, with each monomer comprising (a) an extracellular domain comprising an inserted cysteine residue that forms a disulfide linkage with another monomer and a c-Jun leucine zipper; and (b) an IL6ST transmembrane domain and intracellular signaling domain. The cysteine residue and the leucine zipper on each polypeptide can induce the formation of stable homodimers that mimic constitutive IL-6R activation. Additional details on the construction of L-gp130 are described in Stuhlmann-Lacisz et al. Mol Biol Cell. 2006 July; 17 (7): 2986-95 and in WO2020200325, which are hereby incorporated by reference in their entirety.

In some embodiments, the L-gp130 intracellular signaling domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 74. In some embodiments, the L-gp130 intracellular signaling domain comprises the amino acid sequence set forth in SEQ ID NO: 74. In some embodiments, L-gp130 comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 75. In some embodiments, L-gp130 comprises the amino acid sequence set forth in SEQ ID NO: 75.

In some embodiments, the L-gp130 intracellular signaling domain is encoded by a nucleic acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 79. In some embodiments, the L-gp130 intracellular signaling domain is encoded by the nucleic acid sequence set forth in SEQ ID NO: 79. In some embodiments, L-gp 130 is encoded by a nucleic acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 80. In some embodiments, L-gp130 is encoded by the nucleic acid sequence set forth in SEQ ID NO: 80.

In some embodiments, the leucine zipper domain comprises the sequence as set forth in Seq ID NO: 179. In some embodiments, the leucine zipper domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 179.

In some embodiments, the L-gp130 transmembrane domain comprises the gp130 transmembrane domain. In some embodiments, the L-gp130 transmembrane domain comprises the sequence as set forth in Seq ID NO: 180. In some embodiments, the L-gp130 transmembrane domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 180.

In some embodiments, the SPA comprises a membrane-bound interleukin-15 (mbIL-15). mbIL-15 comprises an IL-15 polypeptide linked to a linked to the IL15-receptor a (IL15Ra), thus allowing constitutive receptor activation. See also, e.g., U.S. Pat. No. 9,629,877, which is hereby incorporated by reference in its entirety.

In some embodiments, mbIL-15 comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 76. In some embodiments mbIL-15 comprises the amino acid sequence set forth in SEQ ID NO: 76. In some embodiments, mbIL-15 is encoded by a nucleic acid sequence at least 90%, at least 91%, at least 92%, at least 93%, at least 94%, at least 95%, at least 96%, at least 97%, at least 98%, or at least 99% identical to the sequence set forth in SEQ ID NO: 81. In some embodiments, mbIL-15 is encoded by the nucleic acid sequence set forth in SEQ ID NO: 81.

In some embodiments, the SPA comprises a CD34-interleukin-7 receptor (C7R). C7R comprises a homodimer, with each monomer comprising (a) an extracellular domain comprising a CD34 ectodomain, (b) a transmembrane domain comprising an inserted cysteine residue that forms a disulfide linkage with another monomer, and (c) an IL-7R intracellular signaling domain. The CD34 ectodomain and the inserted cysteine residue on each polypeptide allow the formation of a stable homodimer that mimics constitutive IL-7R activation. Additional details on the construction of C7R are described in Shum et al. Cancer Discov. 2017 November; 7 (11): 1238-1247 and in US Publication No. 20190183939, which are hereby incorporated by reference in their entirety.

In some embodiments, the C7R intracellular signaling domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 77. In some embodiments, the C7R intracellular signaling domain comprises the amino acid sequence set forth in SEQ ID NO: 77. In some embodiments, C7R comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 78. In some embodiments, C7R comprises the amino acid sequence set forth in SEQ ID NO: 78. In some embodiments, the C7R extracellular domain and transmembrane domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 181. In some embodiments, the C7R extracellular domain and transmembrane domain comprises the amino acid sequence set forth in SEQ ID NO: 181.

In some embodiments, the C7R intracellular signaling domain is encoded by a nucleic acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 82. In some embodiments, the C7R intracellular signaling domain is encoded by the nucleic acid sequence set forth in SEQ ID NO: 82. In some embodiments, C7R is encoded by a nucleic acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence set forth in SEQ ID NO: 83. In some embodiments C7R is encoded by a nucleic acid sequence set forth in SEQ ID NO: 83.

### Cytokines

In various embodiments, the systems disclosed herein employ one or more cytokines. CAR-expressing immune cells can be limited by the necessity for in vivo expansion following infusion. To achieve robust expansion, T cells require three signals: antigen-stimulation, co-stimulation, and cytokine-induced stimulation. Activation of CARs is sufficient to induce the first two signals, but cannot recapitulate cytokine signaling. Furthermore, the tumor microenvironment is often immunosuppressive and devoid of pro-inflammatory cytokines. Cytokines can thus be used to stimulate robust in vivo expansion and enhance desirable properties (e.g., increased survival, persistence, and potency) of T cells expressing priming receptors and/or CARs as described herein.

In various embodiments, cytokines used in the systems disclosed herein can be members of the interleukin (IL) family of cytokines. Interleukins activate receptors that signal through Signal Transducer and Activator of Transcription (STAT) transcription factors (e.g., STATI, STAT3 and STAT5). Once activated, interleukin receptors can dimerize and bind janus-associated kinases (JAKs) to induce JAK cross-phosphorylation and downstream “JAK/STAT” signaling. Accordingly, induced cytokine expression can be utilized to induce receptor activity and thus cytokine signaling.

In some embodiments, cytokines used in the system disclosed herein are secreted into the extracellular milieu upon expression. In some embodiments, cytokines used in the system disclosed herein are membrane-bound. Membrane-bound (“mb”) cytokines can comprise a non-native polypeptide that tethers the cytokine to the cellular membrane upon expression. Membrane-bound cytokines can improve activation of cytokine signaling, e.g., by increasing the proximity of the cytokine to its receptor.

### Exemplary Cytokines

Any cytokine that confers advantageous effects to CAR-expressing immune cells can be employed in the system disclosed herein. In various embodiments, cytokines employed in the system disclosed herein can promote memory T cell persistence and/or proliferation (e.g., IL-7, IL-15, and IL-23). In various embodiments, cytokines employed in the system disclosed herein can promote effector cell function (e.g., IL-2 and IL-12). In various embodiments, cytokines employed in the system disclosed herein can reduce immune cell exhaustion (e.g., IL-21 and IL-23). In various embodiments, cytokines employed in the system disclosed herein can promote activation of endogenous immunity (e.g., IL-18). In various embodiments, cytokines employed in the system disclosed herein can reduce an inflammatory response (e.g., IL-10 and TGF-β). In various embodiments, cytokines used in the system disclosed herein are selected from IL-2, Super-2, IL-7, IL-21, IL-12, IL-12/23p40, IL-15, IL-18, IL-10, and TGF-β.

In some embodiments, the cytokine used in the system disclosed herein is IL-2. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 86. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 87.

In some embodiments, the cytokine used in the system disclosed herein is IL-7. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 88. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 89.

In some embodiments, the cytokine used in the system disclosed herein is IL-21. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 90. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 91.

In some embodiments, the cytokine used in the system disclosed herein is IL-12. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 92. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 93.

In some embodiments, the cytokine used in the system disclosed herein is IL-12/23p40. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 94. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 95.

In some embodiments, the cytokine used in the system disclosed herein is IL-15. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 96. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 97.

In some embodiments, the cytokine used in the system disclosed herein is IL-18. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 98. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 99.

In some embodiments, the cytokine used in the system disclosed herein is Super-2. Further details on Super-2 are given in U.S. Pat. No. 10,150,802, which is hereby incorporated by reference in its entirety. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 132. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 133.

In some embodiments, the cytokine used in the system disclosed herein is IL-10. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 134. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 135.

In some embodiments, the cytokine used in the system disclosed herein is TGF-β. In some embodiments, the cytokine used in the system disclosed herein comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 136. In some embodiments, the nucleic acid encoding the cytokine used in the system disclosed herein comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 137.

In some embodiments, one or more than one cytokine is expressed in the system disclosed herein. In some embodiments, two or more cytokines are expressed in the system disclosed herein. In some embodiments, three or more cytokines are expressed in the system disclosed herein. In some embodiments, four or more cytokines are expressed in the system disclosed herein. In some embodiments, five or more cytokines are expressed in the system disclosed herein. In some embodiments, six or more cytokines are expressed in the system disclosed herein. In some embodiments, seven or more cytokines are expressed in the system disclosed herein.

### Signal Peptides

In various embodiments, cytokines used in the system disclosed herein are operably linked to a non-native signal peptide. Different signal peptides can yield variant levels of secretion of expressed proteins (e.g., cytokines). Further details on signal peptides and their influence on protein secretion can be found in Lumangtad LA, Bell TW. The signal peptide as a new target for drug design. Bioorg Med Chem Lett. 2020 May 15;30 (10): 127115, which is hereby incorporated by reference in its entirety. Selection of specific signal peptides can therefore allow rheostat tuning of cytokine secretion based on desired levels of downstream signaling. For example, for cytokines that are expressed with low efficiency, a signal peptide yielding highly efficient secretion can be selected to improve overall activation of cytokine signaling. In another example, for cytokines that are toxic at high levels, a signal peptide yielding reduced efficiency of secretion can be selected to reduce the toxic effects of the cytokine.

In some embodiments, the non-native signal peptide comprises a signal peptide from at least one of CD44, CD3E, CD5, IGTAL, IL-2, GMCSF, chymotrypsinogen, trypsinogen, IgK, IgKVIII, IgE, OSM, IgG2H, BM40, secrecon, and tPA.

In some embodiments, the cytokine used in the system disclosed herein is linked to a CD44 signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 100. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 101.

In some embodiments, the cytokine used in the system disclosed herein is linked to a CD3E signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 102. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 103.

In some embodiments, the cytokine used in the system disclosed herein is linked to a CD5 signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 104. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 105.

In some embodiments, the cytokine used in the system disclosed herein is linked to a IGTAL signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 106. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 107.

In some embodiments, the cytokine used in the system disclosed herein is linked to a IL-2 signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 108. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 109.

In some embodiments, the cytokine used in the system disclosed herein is linked to a GMCSF signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 110. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 111.

In some embodiments, the cytokine used in the system disclosed herein is linked to a chymotrypsinogen signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 112. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 113.

In some embodiments, the cytokine used in the system disclosed herein is linked to a trypsinogen signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 114. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 115.

In some embodiments, the cytokine used in the system disclosed herein is linked to a IgK signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 116. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 117.

In some embodiments, the cytokine used in the system disclosed herein is linked to a IgKVIII signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 118. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 119.

In some embodiments, the cytokine used in the system disclosed herein is linked to a IgE signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 120. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 121.

In some embodiments, the cytokine used in the system disclosed herein is linked to a OSM signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 122. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 123.

In some embodiments, the cytokine used in the system disclosed herein is linked to a IgG2H signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 124. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 125.

In some embodiments, the cytokine used in the system disclosed herein is linked to a BM40 signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 126. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 127.

In some embodiments, the cytokine used in the system disclosed herein is linked to a Secrecon signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 128. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 129.

In some embodiments, the cytokine used in the system disclosed herein is linked to a tPA signal peptide. In some embodiments, the non-native signal peptide comprises an amino acid sequence having at least 75%, at least 80%, at least 85%, at least 90%, at least 95%, at least 96%, at least 97%, at least 98%, at least 99%, at least 99.5%, at least 99.9%, or 100% identity to the amino acid sequence set forth in SEQ ID NO: 130. In some embodiments, the nucleic acid encoding the non-native signal peptide comprises a nucleic acid sequence having at least 75%, at least 80%, at least 85%, at least 90%, at least 95%, at least 96%, at least 97%, at least 98%, at least 99%, at least 99.5%, at least 99.9%, or 100% identity to the nucleic acid sequence set forth in SEQ ID NO: 131.

### Suppressors of Gene Expression

In various embodiments, one or more suppressors of gene expression can be used in a system described herein to yield desirable effects on the logic-gate expressing T cells. A suppressor of gene expression can be used, for example, to suppress activity of genes that have inhibitory effects on T cell properties, such as expansion or target cell killing. Suppressors of gene expression can function via any mechanism known in the art. Suppressors of gene expression can function, for example, by knock-out of the genomic sequence, suppression of gene transcription, or suppression of protein translation (“knock-down”). Examples of suppressors of gene expression include, but are not limited to, sgRNAs, shRNAs, siRNAs, TALENs, and zinc-finger nucleases (ZFNs).

In some embodiments, a suppressor of gene expression used in a system disclosed herein is an sgRNA or an shRNA. In some embodiments, the suppressor of gene expression is an sgRNA. In some embodiments, the sgRNA suppresses the expression of a gene selected from PTPN2, RASA2, SOCS1, ZC3H12A, and CISH. In some embodiments, the sgRNA suppresses the expression of PTPN2. In some embodiments, the sgRNA suppresses the expression of RASA2. In some embodiments, the sgRNA suppresses the expression of SOCS1. In some embodiments, the sgRNA suppresses the expression of ZC3H12A. In some embodiments, the sgRNA suppresses the expression of CISH.

In some embodiments, the sgRNA comprises a nucleic acid sequence set forth in any one of SEQ ID NOs: 160-164. In some embodiments, the sgRNA comprises the nucleic acid sequence set forth in SEQ ID NO: 160. In some embodiments, the sgRNA comprises the nucleic acid sequence set forth in SEQ ID NO: 161. In some embodiments, the sgRNA comprises the nucleic acid sequence set forth in SEQ ID NO: 162. In some embodiments, the sgRNA comprises the nucleic acid sequence set forth in SEQ ID NO: 163. In some embodiments, the sgRNA comprises the nucleic acid sequence set forth in SEQ ID NO: 164.

In some embodiments, the suppressor of gene expression is an shRNA. In some embodiments, the shRNA suppresses the expression of a gene selected from RASA2, PTPN2, SOCSI, ZC3H12A, CISH, TNFRSF6 (Fas), TGFBR1, and TGFBR2. In some embodiments, the shRNA suppresses the expression of RASA2. In some embodiments, the shRNA suppresses the expression of PTPN2. In some embodiments, the shRNA suppresses the expression of SOCS1. In some embodiments, the shRNA suppresses the expression of ZC3H12A. In some embodiments, the shRNA suppresses the expression of CISH. In some embodiments, the shRNA suppresses the expression of TNFRSF6 (Fas). In some embodiments, the shRNA suppresses the expression of TGFBRI. In some embodiments, the shRNA suppresses the expression of TGFBR1. In some embodiments, the shRNA comprises a nucleic acid sequence set forth in any one of SEQ ID NOs: 165-172. In some embodiments, the shRNA comprises the nucleic acid sequence set forth in SEQ ID NO: 165. In some embodiments, the shRNA comprises the nucleic acid sequence set forth in SEQ ID NO: 166. In some embodiments, the shRNA comprises the nucleic acid sequence set forth in SEQ ID NO: 167. In some embodiments, the shRNA comprises the nucleic acid sequence set forth in SEQ ID NO: 168. In some embodiments, the shRNA comprises the nucleic acid sequence set forth in SEQ ID NO: 169. In some embodiments, the shRNA comprises the nucleic acid sequence set forth in SEQ ID NO: 170. In some embodiments, the shRNA comprises the nucleic acid sequence set forth in SEQ ID NO: 171. In some embodiments, the shRNA comprises the nucleic acid sequence set forth in SEQ ID NO: 172.

### Exemplary Combinations

Combinations of suppressors of gene expression with an SPA and/or a cytokine can be used in a system described herein to support activation of logic gate-expressing T cells. In some embodiments, the SPA is L-gp130 and the cytokine is IL-2. In some embodiments, the SPA is L-gp 130 and the cytokine is mbIL-15. In some embodiments, the SPA is C7R and the cytokine is IL-2. In some embodiments, the SPA is C7R and the cytokine is mbIL-15. In some embodiments, the system comprises an sgRNA that suppresses CISH expression and a cytokine that is IL-2. In some embodiments, the system comprises an sgRNA that suppresses PTPN2 expression and a cytokine that is IL-2. In some embodiments, the system comprises an sgRNA that suppresses RASA2 expression and a cytokine that is IL-2. In some embodiments, the system comprises an sgRNA that suppresses SOCS1 expression and a cytokine that is IL-2. In some embodiments, the system comprises an sgRNA that suppresses ZC3H12A expression and a cytokine that is IL-2. In some embodiments, the system comprises an sgRNA that suppresses PTPN2 expression and a cytokine that is IL-21. In some embodiments, the system comprises an sgRNA that suppresses ZC3H12A expression and a cytokine that is IL-21. In some embodiments, the system comprises an sgRNA that suppresses RASA2 expression and a cytokine that is IL-15. In some embodiments, the system comprises an shRNA that suppresses RASA2 expression and a cytokine that is IL-2. In some embodiments, the system comprises an shRNA that suppresses RASA2 expression and a cytokine that is IL-15. In some embodiments, the system comprises an sgRNA that suppresses CISH expression and an SPA that is C7R. In some embodiments, the system comprises an sgRNA that suppresses PTPN2 expression and an SPA that is C7R. In some embodiments, the system comprises an sgRNA that suppresses RASA2 expression and an SPA that is C7R. In some embodiments, the system comprises an sgRNA that suppresses SOCS1 expression and an SPA that is C7R. In some embodiments, the system comprises an sgRNA that suppresses ZC3H12A expression and an SPA that is C7R. In some embodiments, the system comprises an sgRNA that suppresses CISH expression and an SPA that is L-gp130. In some embodiments, the system comprises an sgRNA that suppresses RASA2 expression and an SPA that is L-gp130. In some embodiments, the system comprises an sgRNA that suppresses ZC3H12A expression and an SPA that is L-gp130.

In some embodiments, the system comprises an shRNA that suppresses the expression of TNFRSF6 (Fas) and an additional suppressor of gene expression. In some embodiments, the system comprises an shRNA that suppresses the expression of TNFRSF6 (Fas), an shRNA that suppresses the expression of TGFBR2 and an additional suppressor of gene expression. In some embodiments, the system comprises an shRNA that suppresses the expression of TNFRSF6 (Fas), an shRNA that suppresses the expression of PTPN2 and an additional suppressor of gene expression.

### Recombinant Nucleic Acids and Vectors

In some embodiments, the present disclosure contemplates recombinant nucleic acid inserts that comprise one or more transgenes encoding the priming receptors, CARs, cytokines, or SPAs as described herein. In some embodiments, the nucleic acids are recombinant nucleic acids. In some embodiments, the insert encodes a priming receptor transgene. In some embodiments, the insert encodes a chimeric antigen receptor transgene. In some embodiments, the insert comprises a priming receptor transgene and a chimeric antigen receptor transgene. In some embodiments, the insert comprises a priming receptor transgene and a cytokine transgene. In some embodiments, the insert comprises a cytokine transgene and a chimeric antigen receptor transgene. In some embodiments, the insert comprises a priming receptor transgene, a cytokine transgene, and a chimeric antigen receptor transgene. In some embodiments, the insert comprises a priming receptor transgene and a SPA transgene. In some embodiments, the insert comprises a SPA transgene and a chimeric antigen receptor transgene. In some embodiments, the insert comprises a priming receptor transgene, a SPA transgene, and a chimeric antigen receptor transgene. In some embodiments, the insert comprises a SPA, a cytokine transgene, and a chimeric antigen receptor transgene. In some embodiments, the insert comprises a SPA, a priming receptor transgene, a cytokine transgene, and a chimeric antigen receptor transgene.

The insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and -mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7 (2): 403-412 (2015)).

The insert can also comprise a WPRE element. WPRE elements are generally described in Higashimoto, T., et al. Gene Ther 14, 1298-1304 (2007); and Zufferey, R., et al. J Virol. 1999 April; 73 (4): 2886-92., both of which are hereby incorporated by reference.

### Recombinant Cells

Also provided herein are recombinant immune cells comprising at least one DNA template non-virally inserted into a target region of the genome of the cell, wherein DNA template encodes the priming receptor and CAR system as described herein, optionally also the cytokine, optionally also the SPA, optionally also the gene expression suppressor molecule. Also provided herein are recombinant immune cells comprising the priming receptor that specifically binds Alkaline Phosphatase, Placental/Germ Cell (ALPG/P), the chimeric antigen receptor that specifically binds MSLN, and the cytokine and/or the synthetic pathway activator that activates cytokine signaling. Also provided herein are engineered immune cells comprising the priming receptor that specifically binds Alkaline Phosphatase, Placental/Germ Cell (ALPG/P), the chimeric antigen receptor that specifically binds MSLN, and the cytokine. The cell can further comprise a gene expression suppressor such as an RNAi molecule (e.g., shRNA) or an sgRNA for CRISPR-based knockout of a target gene.

A cell comprising a DNA template insert at a target locus or safe harbor site as described in the present disclosure can be referred to as an engineered cell. In some embodiments, the immune cell is any cell that can give rise to a pluripotent immune cell. In some embodiments, the immune cell is a primary immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an hematopoietic stem cell, an adaptive immune cell, an innate immune cell, a T cell or a T cell progenitor. Non-limiting examples of immune cells that are contemplated in the present disclosure include T cell, B cell, natural killer (NK) cell, NKT/INKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g., deliberately immortalized cell lines, cancer cell lines, etc.).

Also provided herein are populations of cells comprising a plurality of the immune cell. In some embodiments, the genome of at least 20%, 30%, 40%, 50%, 60%, 70%, 80%, 90%, 95%, 99% or greater of the cells comprises the priming receptor and CAR system with the optional cytokine, SPA, and/or gene suppressor as described herein.

### Method of Treating Cancer

In another aspect, the invention provides methods of treating an immune-related condition (e.g., cancer) in an individual comprising administering to the individual an effective amount of a composition comprising a system comprising a priming receptor that specifically binds to ALPG/P, a chimeric antigen receptor that specifically binds to MSLN, and optionally, a cytokine and/or a synthetic pathway activator that activates cytokine signaling. The system can further comprise a gene expression suppressor such as an RNAi molecule (e.g., shRNA) or an sgRNA for CRISPR based knockout of a target gene. In another aspect, the invention provides methods of enhancing an immune response in an individual comprising administering to the individual an effective amount of a composition comprising a system comprising a priming receptor that specifically binds to ALPG/P, a chimeric antigen receptor that specifically binds to MSLN, and a cytokine and/or a synthetic pathway activator that activates cytokine signaling.

In some embodiments, the methods provided herein are useful for the treatment of an immune-related condition in an individual. In one embodiment, the individual is a human.

In some embodiments, the methods provided herein (such as methods of enhancing an immune response) are useful for the treatment of cancer and as such an individual receiving the system described herein has cancer. In some embodiments, the cancer is a solid cancer. In some embodiments, the cancer is a liquid cancer. In some embodiments, the cancer is immunoevasive. In some embodiments, the cancer is immunoresponsive. In particular embodiments, the cancer is ovarian cancer, fallopian cancer, primary peritoneal cancer, uterine cancer, mesothelioma, cervical cancer, or pancreatic cancers. In particular embodiments, the cancer is ovarian cancer.

In some embodiments, the treatment results in a decrease in the cancer volume or size. In some embodiments, the treatment is effective at reducing a cancer volume as compared to the cancer volume prior to administration of the antibody. In some embodiments, the treatment results in a decrease in the cancer growth rate. In some embodiments, the treatment is effective at reducing a cancer growth rate as compared to the cancer growth rate prior to administration of the antibody. In some embodiments, the treatment is effective at eliminating the cancer.

In some embodiments, MSLN and ALPG or ALPP is expressed at a higher level in the cancer as compared to a non-cancer cell. Levels of MSLN, ALPG, and ALPP can be assessed by any technique known in the field, including, but not limited to, protein assays or nucleic assays such as FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, qPCR, RT-qPCR, multiplex qPCR or RT-qPCR, RNA-seq, microarray analysis, SAGE, MassARRAY technique, and FISH, and combinations thereof.

### Method of Modulating Autoimmune Disease

In another aspect, provided herein are methods of treating an immune-related condition (e.g., graft-versus-host) in an individual comprising administering to the individual an effective amount of a composition comprising a system comprising a priming receptor, a chimeric antigen receptor, and a cytokine. In another aspect, provided herein are methods of suppressing an immune response in an individual comprising administering to the individual an effective amount of a composition comprising a system comprising a priming receptor, a chimeric antigen receptor, and a cytokine. The cytokine can be a suppressive cytokine such as IL-10.

In some embodiments, the methods provided herein are useful for the treatment of an immune-related condition in an individual. In one embodiment, the individual is a human.

In some embodiments, the methods provided herein (such as methods of suppressing an immune response) are useful for the treatment of autoimmune disease. In some embodiments, the autoimmune disease is graft-versus-host disease. In some embodiments, the autoimmune disease is transplant rejection. In some embodiments, the autoimmune disease is rheumatoid arthritis. In some embodiments, the autoimmune disease is inflammatory bowel disease. In some embodiments, the autoimmune disease is type-I diabetes.

### Method of Immune Modulation

Methods of administration of a cell comprising a system comprising a priming receptor that specifically binds to ALPG/P, a chimeric antigen receptor that specifically binds to MSLN, and a cytokine and/or a synthetic pathway activator that activates cytokine signaling as described herein can result in modulation of an immune response. Modulation can be an increase or decrease in an immune response. In some embodiments, modulation is an increase in an immune response.

In one aspect, administration of a cell comprising a system comprising a priming receptor that specifically binds to ALPG/P, a chimeric antigen receptor that specifically binds to MSLN, and a cytokine and/or a synthetic pathway activator that activates cytokine signaling as described herein can result in induction of pro-inflammatory molecules, such as cytokines or chemokines. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a system comprising a priming receptor that specifically binds to ALPG/P, a chimeric antigen receptor that specifically binds to MSLN can induce multiple anti-tumor immune mechanisms that lead to tumor destruction, and a cytokine and/or a synthetic pathway activator that activates cytokine signaling.

In another aspect, provided herein are methods of increasing an immune response in an individual comprising administering to the individual an effective amount of a cell comprising a system comprising a priming receptor that specifically binds to ALPG/P, a chimeric antigen receptor that specifically binds to MSLN, and a cytokine and/or a synthetic pathway activator that activates cytokine signaling. In some embodiments, the method of increasing an immune response in a subject comprises administering to the subject a cell comprising a system comprising a priming receptor that specifically binds to ALPG/P, a chimeric antigen receptor that specifically binds to MSLN, and a cytokine and/or a synthetic pathway activator that activates cytokine signaling.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a composition comprising a system comprising a priming receptor that specifically binds to ALPG/P, a chimeric antigen receptor that specifically binds to MSLN, and a cytokine and/or a synthetic pathway activator that activates cytokine signaling.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a system comprising a priming receptor that specifically binds to ALPG/P, a chimeric antigen receptor that specifically binds to MSLN, and a cytokine and/or a synthetic pathway activator that activates cytokine signaling. In some embodiments, the immune response is enhanced by administration of cell comprising a system comprising a priming receptor that specifically binds to ALPG/P, a chimeric antigen receptor that specifically binds to MSLN, and a cytokine and/or a synthetic pathway activator that activates cytokine signaling.

In another aspect, the present application provides methods of genetically editing a cell with a system comprising a priming receptor that specifically binds to ALPG/P, a chimeric antigen receptor that specifically binds to MSLN, and a cytokine and/or a synthetic pathway activator that activates cytokine signaling, which results in the modulation of the immune function of the cell. The modulation can be increasing an immune response. In some embodiments, the modulation is an increase in immune function. In some embodiments, the modulation of function leads to the expression of an MSLN CAR. In some embodiments, the modulation of function leads to the activation of a cell comprising the system.

In some embodiments, the cell is a natural killer (NK) cell, a T cell, a CD8+ T cell, a CD4+ T cell, a primary T cell, or a T cell progenitor.

In some embodiments, the modulation of function of the cells comprising the priming receptor and CAR system as described herein leads to an increase in the cells' abilities to stimulate both native and activated T-cells, for example, by increasing cytokine or chemokine secretion by the cells expressing the priming receptor and CAR system. In some embodiments, the modulation of function enhances or increases the cells' ability to produce cytokines, chemokines, CARs, or costimulatory or activating receptors. In some embodiments, the modulation increases the T-cell stimulatory function of the cells expressing the priming receptor and CAR system, including, for example, the cells' abilities to trigger T-cell receptor (TCR) signaling, T-cell proliferation, or T-cell cytokine production.

In some embodiments, the increased immune response is secretion of cytokines and chemokines. In some embodiments, the priming receptor and CAR system induces increased expression of at least one cytokine or chemokine in a cell as compared to an isotype control cell. In some embodiments, the at least one cytokine or chemokine is selected from the group consisting of: IL-2 and IFNg. In some embodiments, the cytokine or chemokine is IL-2. In some embodiments, the cytokine or chemokine is IFNg. In some embodiments, the cytokine or chemokine secretion is increased a between bout 1-100-fold 1, 5, 10, 20, 30, 40, 50, 60, 70, 80, 90, 100, 1-10, 10-20, 20-30, 30-40, 40-50, 50-60, 60-70, 70-80, 80-90, or 90-100 fold as compared to an untreated cell or a cell treated with an isotype control antibody. In some embodiments, the chemokine is IL-2 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the cytokine is IFNg and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody.

In some embodiments, the enhanced immune response is anti-tumor immune cell recruitment and activation.

In some embodiments, the cell expressing the priming receptor and CAR system induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g., for therapeutic purposes).

Currently available genome editing tools include zinc finger nucleases (ZFN) and transcription activator-like effector nucleases (TALENs) to incorporate genes at safe harbor loci (e.g., the adeno-associated virus integration site 1 (AAVS1) safe harbor locus). The DICE (dual integrase cassette exchange) system utilizing phiC31 integrase and Bxbl integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector.

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

The plasmid can be introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit system receptors do not express CAR until the priming receptor binds to its cognate ligand and releases the cleavable transcription factor.

Initially, a T cell is activated. The T cell may be obtained from a patient. Thus, the present disclosure provides methods in which immune cells, such as T cells, are harvested from a patient. Then, the plasmid that encodes the CAR and priming receptor are introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The T cells are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the size of the DNA template is greater than about 200 nucleotides, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the DNA template is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10 kb or any size of DNA template in between these sizes. For example, the size of the DNA template can be about 4.5 kb to about 10 kb, about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the DNA template is large enough and in sufficient quantity to be lethal as naked DNA. In some embodiments, the DNA template encodes a heterologous protein or a fragment thereof. In some embodiments, the DNA template encodes at least one gene. In some embodiments, the DNA template encodes at least two genes. In some embodiments, the DNA template encodes one, two, three, four, five, six, seven, eight, nine, ten, or more genes.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L.H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains. In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4+ T cell. In some embodiments, the T cell is a CD8+ T cell. In some embodiments, the T cell is a CD4+CD8+T cell. In some embodiments, the T cell is a CD4-CD8-T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

### Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBCI or TRBC2 gene.

Methods for editing the genome of a T cell, specifically, include a method of editing the genome of a human T cell comprise inserting a nucleic acid sequence or construct into a target region of a genomic safe harbor (GSH).

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 KB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVSI site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (Sec Sadelain, M., et al. (2012). Safe harbours for the integration of new DNA in the human genome. Nature reviews Cancer, 12 (1), 51-58, the relevant disclosures of which are herein incorporated by reference in their entirety).

The AAVS1 (also known as the PPP1R12C locus) on human chromosome 19 is a known SHS for hosting transgenes (e.g., DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. An exemplary AAVS1 target gRNA and target sequence are provided below:

AAVS1-gRNA sequence:
(SEQ ID NO: 186)
ggggccactagggacaggatGTTTTAGAGCTAGAAATAG
CAAGTTAAAATAAGGCTAGTCCGTTATCAACTTGAAAAA
GTGGCACCGAGTCGGTGCTTTTTTT
AAVS1 target sequence:
(SEQ ID NO: 187)
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. Examples of additional integration sites are provided in Table D.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 KB, about 125 kB, about 150 KB, about 175 kB, about 200 kB, about 225 kB, about 250 KB, about 275 kB, about 300 KB, about 325 kB, about 350 KB, about 375 kB, about 400 kB, about 425 kB, about 450 kB, about 475 kB, about 500 KB, about 525 kB, about 550 KB away from the safe harbor locus (integration site).

In some embodiments, the present disclosure contemplates inserts that comprise one or more transgenes. The transgene can encode a therapeutic protein, an antibody, a peptide, or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, a T cell having “enhanced therapeutic properties” has an enhanced, improved, and/or increased treatment outcome when compared to a typical, unmodified and/or naturally occurring T cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kilobasepairs (kb) to about 10 kilobasepairs (kb). In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kbp (kilo basepairs) or the sizes in between. In some embodiments, the insert size is greater than 4.5, 4.8, 5, 6, 7, 8, 9, 10, 11, 12, 13, 14, 15, 16, 17, 18, 19, 20 kbp or the sizes in between. In some embodiments, the insert size is within the range of 4.5-15 kbp or is any number in that range. In some embodiments, the insert size is within the range of 4.8-8.3 kbp or is any number in that range. In some embodiments, the insert size is within the range of 5-8.3 kbp or is any number in that range. In some embodiments, the insert size is within the range of 5-15 kbp or is any number in that range. In some embodiments, the insert size is within the range of 4.5-20 kbp or is any number in that range. In some embodiments, the insert size is 5-10 kbp. In some embodiments, the insert size is 4.5-10, 5-10, 6-10, 7-10, 8-10, 9-10 kbp. In some embodiments, the insert size is 4.5-11, 6-11, 7-11, 8-11, 9-11, or 10-11 kbp. In some embodiments, the insert size is 4.5-12, 6-12, 7-12, 8-12, 9-12, 10-12, or 11-12 kbp. In some embodiments, the insert size is 4.5-13, 6-13, 7-13, 8-13, 9-13, 10-13, 11-13, or 12-13 kbp. In some embodiments, the insert size is 4.5-14, 6-14, 7-14, 8-14, 9-14, 10-14, 11-14, 12-14 or 13-14 kbp. In some embodiments, the insert size is 4.5-15, 6-15, 7-15, 8-15, 9-15, 10-15, 11-15, 12-15, 13-15, or 14-15 kbp. In some embodiments, the insert size is 4.5-16, 6-16, 7-16, 8-16, 9-16, 10-16, 11-16, 12-16, 13-16, 14-16 or 15-16 kbp. In some embodiments, the insert size is 4.5-17, 6-17, 7-17, 8-17, 9-17, 10-17, 11-17, 12-17, 13-17, or 14-17, 15-17 or 16-17 kbp. In some embodiments, the insert size is 4.5-18, 6-18, 7-18, 8-18, 9-18, 10-18, 11-18, 12-18, 13-18, 14-18, 15-18, 16-18 or 17-18 kbp. In some embodiments, the insert size is 4.5-19, 6-19, 7-19, 8-19, 9-19, 10-19, 11-19, 12-19, 13-19, 14-19, 15-19, 16-19, 17-19, or 18-19 kbp. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occuring nucleotides, non-naturally occuring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, etc. In some embodiments, the the insert is a human sequence. In some embodiments, the insert is chimeric. In some embodiments, the insert is a multi-gene/multi-module therapeutic cassette. A multi-gene/multi-module therapeutic cassette referst to an insert or cassette having one or more than one receptor (e.g., synthetic receptors), other exogenous protein coding sequences, non-coding RNAs, transcriptional regulatory elements, and/or insulator sequences, etc.

In some embodiments, the nucleic acid sequence is inserted into the genome of the T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

Examples of integration sites contemplated are provided in Table D.

TABLE D
sgRNA sequences

| | | | | | Median |
|---|---|---|---|---|---|
| | | | | | (% |
| | | | | | Modified), |
| | | sgRNA | | | summarized |
| | | start | sgRNA | Integra- | from 2 |
| sgRNA | | coor | Target | tion | donors, 2 |
| ID | sgRNA Sequence | GRCH38 | Loci | Site | primersets |
| sgRNA_1 | GCACCTGAATACCACGCCTG | chr16: | APRT | APRT | 79.28 |
| | (SEQ ID NO: 188) | 88811818 | | | |
| sgRNA_2 | CGCCTGCGATGTAGTCGATG | chr16: | APRT | APRT | 78.60 |
| | (SEQ ID NO: 189) | 88811551 | | | |
| sgRNA_3 | CAGGACGGGCGAGATGTCCC | chr16: | APRT | APRT | 85.25 |
| | (SEQ ID NO: 190) | 88811640 | | | |
| sgRNA_4 | CTGAATCTTTGGAGTACCTG | chr15: | B2M | B2M | 78.51 |
| | (SEQ ID NO: 191) | 44715425 | | | |
| sgRNA_5 | GGCCACGGAGCGAGACATCT | chr15: | B2M | B2M | 94.75 |
| | (SEQ ID NO: 192) | 44711550 | | | |
| sgRNA_6 | AAGTCAACTTCAATGTCGGA | chr15: | B2M | B2M | 70.97 |
| | (SEQ ID NO: 193) | 44715515 | | | |
| sgRNA_7 | GCTTGGAGGCCTGATCAGCG | chr19: | CAPNS1 | CAPNS1 | 89.34 |
| | (SEQ ID NO: 194) | 36141111 | | | |
| sgRNA_8 | CTTATCTCTTCGCAGCGAGG | chr19: | CAPNS1 | CAPNS1 | 91.09 |
| | (SEQ ID NO: 195) | 36142301 | | | |
| sgRNA_9 | CACACATTACTCCAACATTG | chr19: | CAPNS1 | CAPNS1 | 71.98 |
| | (SEQ ID NO: 196) | 36142676 | | | |
| sgRNA_10 | TTCCGCAAAATAGAGCCCCA | chr3: | CBLB | CBLB | 91.55 |
| | (SEQ ID NO: 197) | 105746019 | | | |
| sgRNA_11 | TGCACAGAACTATCGTACCA | chr3: | CBLB | CBLB | 91.43 |
| | (SEQ ID NO: 198) | 105751622 | | | |
| sgRNA_12 | GCAATAAGACTCTTTAAAGA | chr3: | CBLB | CBLB | 76.18 |
| | (SEQ ID NO: 199) | 105853470 | | | |
| sgRNA_13 | CAAAGAGATTACGAATGCCT | chr1: | CD2 | CD2 | 89.80 |
| | (SEQ ID NO: 200) | 116754658 | | | |
| sgRNA_14 | CAAGGCACCCCAGGTTTCCA | chr1: | CD2 | CD2 | 92.70 |
| | (SEQ ID NO: 201) | 116754663 | | | |
| sgRNA_15 | TTACGAATGCCTTGGAAACC | chr1: | CD2 | CD2 | 92.82 |
| | (SEQ ID NO: 202) | 116754666 | | | |
| sgRNA_16 | CAGAGACGCATCTGACCCTC | chr11: | CD3E | CD3E | 90.96 |
| | (SEQ ID NO: 203) | 118315540 | | | |
| sgRNA_17 | CATGCAGTTCTCACACACTG | chr11: | CD3E | CD3E | 87.47 |
| | (SEQ ID NO: 204) | 118313715 | | | |
| sgRNA_18 | GTGTGAGAACTGCATGGAGA | chr11: | CD3E | CD3E | 86.65 |
| | (SEQ ID NO: 205) | 118313715 | | | |
| sgRNA_19 | TCTCATTTCAGGAAACCACT | chr11: | CD3G | CD3G | 87.24 |
| | (SEQ ID NO: 206) | 118349748 | | | |
| sgRNA_20 | AGTCATACACCTTAACCAAG | chr11: | CD3G | CD3G | 87.99 |
| | (SEQ ID NO: 207) | 118349754 | | | |
| sgRNA_21 | TTCAAGGAAACCAGTTGAGG | chr11: | CD3G | CD3G | 86.55 |
| | (SEQ ID NO: 208) | 118352458 | | | |
| sgRNA_22 | GAGCCTTGCCTGGAAATCTG | chr11: | CD5 | CD5 | 84.03 |
| | (SEQ ID NO: 209) | 61118177 | | | |
| sgRNA_23 | AAGCGTCAAAAGTCTGCCAG | chr11: | CD5 | CD5 | 89.19 |
| | (SEQ ID NO: 210) | 61118324 | | | |
| sgRNA_24 | CGTTCCAACTCGAAGTGCCA | chr11: | CD5 | CD5 | 83.11 |
| | (SEQ ID NO: 211) | 61118121 | | | |
| sgRNA_25 | GAGCGACTGGGACACGGTGA | chr9: | EDF1 | EDF1 | 88.84 |
| | (SEQ ID NO: 212) | 136866246 | | | |
| sgRNA_26 | GCTGCGCAAGAAGGGCCCTA | chr9: | EDF1 | EDF1 | 91.04 |
| | (SEQ ID NO: 213) | 136866211 | | | |
| sgRNA_27 | TTGTTCTGGCCAGCAGCCCC | chr9: | EDF1 | EDF1 | 85.98 |
| | (SEQ ID NO: 214) | 136863433 | | | |
| sgRNA_28 | CTTCCAGAGCCACATCATCG | chr19: | FTL | FTL | 93.10 |
| | (SEQ ID NO: 215) | 48965791 | | | |
| sgRNA_29 | GGGACTCACCAGAGAGAGGT | chr19: | FTL | FTL | 88.86 |
| | (SEQ ID NO: 216) | 48965601 | | | |
| sgRNA_30 | CGGTCGAAATAGAAGCCCTA | chr19: | FTL | FTL | 93.14 |
| | (SEQ ID NO: 217) | 48965770 | | | |
| sgRNA_31 | AAAAGGATATTGTGCAACTG | chr10: | PTEN | PTEN | 92.37 |
| | (SEQ ID NO: 218) | 87933015 | | | |
| sgRNA_32 | TGTGCATATTTATTACATCG | chr10: | PTEN | PTEN | 90.64 |
| | (SEQ ID NO: 219) | 87933183 | | | |
| sgRNA_33 | TTTGTGAAGATCTTGACCAA | chr10: | PTEN | PTEN | 85.36 |
| | (SEQ ID NO: 220) | 87933087 | | | |
| sgRNA_34 | TGTCATGCTGAACCGCATTG | chr18: | PTPN2 | PTPN2 | 87.94 |
| | (SEQ ID NO: 221) | 12830972 | | | |
| sgRNA_35 | CCACTCTATGAGGATAGTCA | chr18: | PTPN2 | PTPN2 | 92.45 |
| | (SEQ ID NO: 222) | 12859219 | | | |
| sgRNA_36 | TTGACATAGAAGAGGCACAA | chr18: | PTPN2 | PTPN2 | 93.96 |
| | (SEQ ID NO: 223) | 12836828 | | | |
| sgRNA_37 | GAGTACTACACTCAGCAGCA | chr12: | PTPN6 | PTPN6 | 89.61 |
| | (SEQ ID NO: 224) | 6952098 | | | |
| sgRNA_38 | TCACGCACAAGAAACGTCCA | chr12: | PTPN6 | PTPN6 | 82.74 |
| | (SEQ ID NO: 225) | 6954872 | | | |
| sgRNA_39 | AGGTCTCGGTGAAACCACCT | chr12: | PTPN6 | PTPN6 | 91.27 |
| | (SEQ ID NO: 226) | 6951610 | | | |
| sgRNA_40 | AGCATTATCCAAAGAGTCCG | chr1: | PTPRC | PTPRC | 88.88 |
| | (SEQ ID NO: 227) | 198696873 | | | |
| sgRNA_41 | ATATTAATTCTTACCAGTGG | chr1: | PTPRC | PTPRC | 88.95 |
| | (SEQ ID NO: 228) | 198692370 | | | |
| sgRNA_42 | AGCTTTAAATCAAGGTTCAT | chr1: | PTPRC | PTPRC | 96.89 |
| | (SEQ ID NO: 229) | 198756176 | | | |
| sgRNA_43 | ATCCCGAGCCCTAAGGTGCA | chr11: | PTPRCAP | PTPRCAP | 84.08 |
| | (SEQ ID NO: 230) | 67436325 | | | |
| sgRNA_44 | GGCAGCGCGGAGGACAGCGT | chr11: | PTPRCAP | PTPRCAP | 97.74 |
| | (SEQ ID NO: 231) | 67436285 | | | |
| sgRNA_45 | CTCAGGGGGCTACTACCACC | chr11: | PTPRCAP | PTPRCAP | 91.50 |
| | (SEQ ID NO: 232) | 67436170 | | | |
| sgRNA_46 | GTCACCGACGAGACCAGAAG | chr5: | RPS23 | RPS23 | 79.40 |
| | (SEQ ID NO: 233) | 82277810 | | | |
| sgRNA_47 | GTCGTGGACTTCGTACTGCT | chr5: | RPS23 | RPS23 | 83.07 |
| | (SEQ ID NO: 234) | 82277843 | | | |
| sgRNA_48 | TAATTTTTAGGCAAGTGTCG | chr5: | RPS23 | RPS23 | 61.94 |
| | (SEQ ID NO: 235) | 82277860 | | | |
| sgRNA_49 | TTAGCTGTTAGACTTGAATA | chr14: | RTRAF | RTRAF | 85.50 |
| | (SEQ ID NO: 236) | 51993810 | | | |
| sgRNA_50 | CGAGAGCCGTCAACTTGCGT | chr14: | RTRAF | RTRAF | 85.64 |
| | (SEQ ID NO: 237) | 51989652 | | | |
| sgRNA_51 | CGGCTTCAACTGCAAAGGTG | chr14: | RTRAF | RTRAF | 88.77 |
| | (SEQ ID NO: 238) | 51989700 | | | |
| sgRNA_52 | TATGAAAAAGCAGAGCGACT | chr15: | SERF2 | SERF2 | 89.61 |
| | (SEQ ID NO: 239) | 43793025 | | | |
| sgRNA_53 | TCTGGCGGGCGAGCTCACGC | chr15: | SERF2 | SERF2 | 86.73 |
| | (SEQ ID NO: 240) | 43792989 | | | |
| sgRNA_54 | CTCACGCTGGTTACCGCCTA | chr15: | SERF2 | SERF2 | 80.57 |
| | (SEQ ID NO: 241) | 43792977 | | | |
| sgRNA_55 | AAAGATTACGAACTTCCCTG | chr12: | SLC38A1 | SLC38A1 | 92.24 |
| | (SEQ ID NO: 242) | 46207559 | | | |
| sgRNA_56 | GTTAAAAACAGACATGCCTA | chr12: | SLC38A1 | SLC38A1 | 91.51 |
| | (SEQ ID NO: 243) | 46229232 | | | |
| sgRNA_57 | ATGCCTAAGGAGGTTGTACC | chr12: | SLC38A1 | SLC38A1 | 79.48 |
| | (SEQ ID NO: 244) | 46229246 | | | |
| sgRNA_58 | CTCCAGGTATCCCATCGAAA | chr18: | SMAD2 | SMAD2 | 79.53 |
| | (SEQ ID NO: 245) | 47869418 | | | |
| sgRNA_59 | CACCAAATACGATAGATCAG | chr18: | SMAD2 | SMAD2 | 86.61 |
| | (SEQ ID NO: 246) | 47870532 | | | |
| sgRNA_60 | TGGCGGCGTGAATGGCAAGA | chr18: | SMAD2 | SMAD2 | 82.91 |
| | (SEQ ID NO: 247) | 47896729 | | | |
| sgRNA_61 | TAGGATGGTAGCACACAACC | chr16: | SOCS1 | SOCS1 | 92.25 |
| | (SEQ ID NO: 248) | 11255478 | | | |
| sgRNA_62 | CAGCAGCAGAGCCCCGACGG | chr16: | SOCS1 | SOCS1 | 83.79 |
| | (SEQ ID NO: 249) | 11255432 | | | |
| sgRNA_63 | CGGCGTGCGAACGGAATGTG | chr16: | SOCS1 | SOCS1 | 84.24 |
| | (SEQ ID NO: 250) | 11255296 | | | |
| sgRNA_64 | TATAGACGCTGCCCGACGTC | chr15: | SRP14 | SRP14 | 95.12 |
| | (SEQ ID NO: 251) | 40038895 | | | |
| sgRNA_65 | TCCAAAGAAGGGTACTGTGG | chr15: | SRP14 | SRP14 | 92.14 |
| | (SEQ ID NO: 252) | 40038368 | | | |
| sgRNA_66 | ACAGTACCCTTCTTTGGAAT | chr15: | SRP14 | SRP14 | 65.82 |
| | (SEQ ID NO: 253) | 40038358 | | | |
| sgRNA_67 | GCGACGGGCGCATCTACGTG | chr12: | SRSF9 | SRSF9 | 83.68 |
| | (SEQ ID NO: 254) | 120469572 | | | |
| sgRNA_68 | CCCGACCTCCATAAGTCCTG | chr12: | SRSF9 | SRSF9 | 92.56 |
| | (SEQ ID NO: 255) | 120465700 | | | |
| sgRNA_69 | GGGGTCCTCGAAGCGCACGA | chr12: | SRSF9 | SRSF9 | 89.94 |
| | (SEQ ID NO: 256) | 120469426 | | | |
| sgRNA_70 | TGCTCTGTTTAGAAGATGAC | chr5: | SUB1 | SUB1 | 79.36 |
| | (SEQ ID NO: 257) | 32591641 | | | |
| sgRNA_71 | ATATTCTTTTCTAGTTAAAG | chr5: | SUB1 | SUB1 | 70.93 |
| | (SEQ ID NO: 258) | 32591566 | | | |
| sgRNA_72 | CCTGTAAAGAAACAAAAGAC | chr5: | SUB1 | SUB1 | 93.66 |
| | (SEQ ID NO: 259) | 32591614 | | | |
| sgRNA_73 | TGGAGAAAGACGTAACTTCG | chr4: | TET2 | TET2 | 83.53 |
| | (SEQ ID NO: 260) | 105234315 | | | |
| sgRNA_74 | TCTGCCCTGAGGTATGCGAT | chr4: | TET2 | TET2 | 90.97 |
| | (SEQ ID NO: 261) | 105234747 | | | |
| sgRNA_75 | ATTCCGCTTGGTGAAAACGA | chr4: | TET2 | TET2 | 89.62 |
| | (SEQ ID NO: 262) | 105235656 | | | |
| sgRNA_76 | CAGGCACAATAGAAACAACG | chr3: | TIGIT | TIGIT | 92.65 |
| | (SEQ ID NO: 263) | 114295571 | | | |
| sgRNA_77 | CCATTTGTAATGCTGACTTG | chr3: | TIGIT | TIGIT | 60.75 |
| | (SEQ ID NO: 264) | 114295700 | | | |
| sgRNA_78 | CTGGGTCACTTGTGCCGTGG | chr3: | TIGIT | TIGIT | 87.99 |
| | (SEQ ID NO: 265) | 114295634 | | | |
| sgRNA_79 | GTCAGGGTTCTGGATATCTG | chr14: | TRAC | TRAC | 98.20 |
| | (SEQ ID NO: 266) | 22547508 | | | |
| sgRNA_80 | TGGATTTAGAGTCTCTCAGC | chr14: | TRAC | TRAC | 88.15 |
| | (SEQ ID NO: 267) | 22547541 | | | |
| sgRNA_81 | CTGCGGCTGTGGTCCAGCTG | chr14: | TRAC | TRAC | 94.77 |
| | (SEQ ID NO: 268) | 22550661 | | | |
| sgRNA_82 | ACAAAACTGTGCTAGACATG | chr14: | TRAC | TRAC | 87.86 |
| | (SEQ ID NO: 269) | 22547658 | | | |
| sgRNA_83 | TTCTTCCCCAGCCCAGGTAA | chr14: | TRAC | TRAC | 89.85 |
| | (SEQ ID NO: 270) | 22547778 | | | |
| sgRNA_84 | CGTCATGAGCAGATTAAACC | chr14: | TRAC | TRAC | 95.81 |
| | (SEQ ID NO: 271) | 22550625 | | | |
| sgRNA_85 | GAGAGCGCCTGCGACCCGAG | chr19: | TRIM28 | TRIM28 | 89.44 |
| | (SEQ ID NO: 272) | 58544980 | | | |
| sgRNA_86 | CCAGCGGGTGAAGTACACCA | chr19: | TRIM28 | TRIM28 | 94.79 |
| | (SEQ ID NO: 273) | 58544869 | | | |
| sgRNA_87 | GGAGCGCTTTTCGCCGCCAG | chr19: | TRIM28 | TRIM28 | 91.81 |
| | (SEQ ID NO: 274) | 58544839 | | | |
| sgRNA_88 | TGAGGCCTGGACCTTATGCA | chr10: | chr10: | desert_1 | 69.44 |
| | (SEQ ID NO: 275) | 33134193 | 33130000- | (GS88) | |
| | | | 33140000 | | |
| sgRNA_89 | CCTGGTGGAGTGAACCATGA | chr10: | chr10: | desert_1 | 95.25 |
| | (SEQ ID NO: 276) | 33132917 | 33130000- | (GS89) | |
| | | | 33140000 | | |
| sgRNA_90 | CAAGCACTTAGGTTCCCCTG | chr10: | chr10: | desert_1 | 91.13 |
| | (SEQ ID NO: 277) | 33134633 | 33130000- | (GS90) | |
| | | | 33140000 | | |
| sgRNA_91 | GGTCTCCCTACAATTCAGCG | chr10: | chr10: | desert_2 | 92.02 |
| | (SEQ ID NO: 278) | 72294568 | 72290000- | (GS91) | |
| | | | 72300000 | | |
| sgRNA_92 | CACAGCGCGTGACTGCAATG | chr10: | chr10: | desert_2 | 90.22 |
| | (SEQ ID NO: 279) | 72298268 | 72290000- | (GS92) | |
| | | | 72300000 | | |
| sgRNA_93 | TCTGGGGCACCAATTCTAGG | chr10: | chr10: | desert_2 | 86.35 |
| | (SEQ ID NO: 280) | 72292786 | 72290000- | (GS93) | |
| | | | 72300000 | | |
| sgRNA_94 | GAGCCATGCTTGGCTTACGA | chr11: | chr11: | desert_3 | 91.24 |
| | (SEQ ID NO: 281) | 128342576 | 128340000- | (GS94) | |
| | | | 128350000 | | |
| sgRNA_95 | GTACAAGTACTTATCTCATG | chr11: | chr11: | desert_3 | 89.02 |
| | (SEQ ID NO: 282) | 128343592 | 128340000- | (GS95) | |
| | | | 128350000 | | |
| sgRNA_96 | GAGATAACAACATAACAACA | chr11: | chr11: | desert_3 | 96.47 |
| | (SEQ ID NO: 283) | 128347170 | 128340000- | (GS96) | |
| | | | 128350000 | | |
| sgRNA_97 | CATATTCCATAGTCTTTGGG | chr11: | chr11: | desert_4 | 88.54 |
| | (SEQ ID NO: 284) | 65425000 | 65425000- | (GS97) | |
| | | | 65427000 | | |
| | | | (NEAT1) | | |
| sgRNA_98 | CTGCCCCTTAGCAACTTAGG | chr11: | chr11: | desert_4 | 92.76 |
| | (SEQ ID NO: 285) | 65425507 | 65425000- | (GS98) | |
| | | | 65427000 | | |
| | | | (NEAT1) | | |
| sgRNA_99 | TGTTTAAAAATATGTTGACA | chr11: | chr11: | desert_4 | 90.76 |
| | (SEQ ID NO: 286) | 65426264 | 65425000- | (GS99) | |
| | | | 65427000 | | |
| | | | (NEAT1) | | |
| sgRNA_100 | CCAGGAATGGAAACTCACGC | chr15: | chr15: | desert_5 | 87.84 |
| | (SEQ ID NO: 287) | 92830315 | 92830000- | (GS100) | |
| | | | 92840000 | | |
| sgRNA_101 | GAGGCCGCTGAATTAACCCG | chr15: | chr15: | desert_5 | 85.32 |
| | (SEQ ID NO: 288) | 92831850 | 92830000- | (GS101) | |
| | | | 92840000 | | |
| sgRNA_102 | ATACACGCACACTTGCAGAA | chr15: | chr15: | desert_5 | 99.92 |
| | (SEQ ID NO: 289) | 92831131 | 92830000- | (GS102) | |
| | | | 92840000 | | |
| sgRNA_103 | GAGCAGACAGAAACCCAGGG | chr16: | chr16: | desert_6 | 87.92 |
| | (SEQ ID NO: 290) | 11225670 | 11220000- | (GS103) | |
| | | | 11230000 | | |
| sgRNA_104 | TGAGTCTCCAAACAGAACAG | chr16: | chr16: | desert_6 | 88.53 |
| | (SEQ ID NO: 291) | 11226284 | 11220000- | (GS104) | |
| | | | 11230000 | | |
| sgRNA_105 | TAATATCACTGACTTCACGG | chr16: | chr16: | desert_6 | 87.65 |
| | (SEQ ID NO: 292) | 11225029 | 11220000- | (GS105) | |
| | | | 11230000 | | |
| sgRNA_106 | TACACACAATGTAAGCAGCA | chr2: | chr2: | desert_7 | 71.79 |
| | (SEQ ID NO: 293) | 87467461 | 87460000- | (GS106) | |
| | | | 87470000 | | |
| sgRNA_107 | GGGAGCTCAATTCGAAACCA | chr2: | chr2: | desert_7 | 65.89 |
| | (SEQ ID NO: 294) | 87468809 | 87460000- | (GS107) | |
| | | | 87470000 | | |
| sgRNA_108 | TTGGACAGGTGAGACAGTCG | chr2: | chr2: | desert_7 | 72.64 |
| | (SEQ ID NO: 295) | 87467001 | 87460000- | (GS108) | |
| | | | 87470000 | | |
| sgRNA_109 | AAGCTCACTCAGATAGTGTG | chr3: | chr3: | desert_8 | 76.89 |
| | (SEQ ID NO: 296) | 186511316 | 186510000- | (GS109) | |
| | | | 186520000 | | |
| sgRNA_110 | CAGGAGAACCACCTTACACG | chr3: | chr3: | desert_8 | 86.31 |
| | (SEQ ID NO: 297) | 186515260 | 186510000- | (GS110) | |
| | | | 186520000 | | |
| sgRNA_111 | GGACAGACCCTGATTCACAA | chr3: | chr3: | desert_8 | 85.47 |
| | (SEQ ID NO: 298) | 186519655 | 186510000- | (GS111) | |
| | | | 186520000 | | |
| sgRNA_112 | ACATGGCAGTCTATGAACAG | chr3: | chr3: | desert_9 | 87.77 |
| | (SEQ ID NO: 299) | 59451154 | 59450000- | (GS112) | |
| | | | 59460000 | | |
| sgRNA_113 | CCTATAGAGAGTACTACTTG | chr3: | chr3: | desert_9 | 79.33 |
| | (SEQ ID NO: 300) | 59456416 | 59450000- | (GS113) | |
| | | | 59460000 | | |
| sgRNA_114 | CCAACCGGGTCTTCATTACG | chr3: | chr3: | desert_9 | 92.21 |
| | (SEQ ID NO: 301) | 59457029 | 59450000- | (GS114) | |
| | | | 59460000 | | |
| sgRNA_115 | TCAAGCGTAGAGTTCCGAGT | chr8: | chr8: | desert_10 | 93.07 |
| | (SEQ ID NO: 302) | 127993006 | 127980000- | (GS115) | |
| | | | 128000000 | | |
| sgRNA_116 | TCATGCAATTATGGACCCAG | chr8: | chr8: | desert_10 | 89.40 |
| | (SEQ ID NO: 303) | 127994663 | 127980000- | (GS116) | |
| | | | 128000000 | | |
| sgRNA_117 | CGGGAAAGTGACTGGCCATG | chr8: | chr8: | desert_10 | 87.45 |
| | (SEQ ID NO: 304) | 127996766 | 127980000- | (GS117) | |
| | | | 128000000 | | |
| sgRNA_118 | TGAGATTGAAATCAAATCGG | chr9: | chr9: | desert_11 | 84.84 |
| | (SEQ ID NO: 305) | 7974159 | 7970000- | (GS118) | |
| | | | 7980000 | | |
| sgRNA_119 | TATGCAATATTCATCACGCG | chr9: | chr9: | desert_11 | 85.44 |
| | (SEQ ID NO: 306) | 7977914 | 7970000- | (GS119) | |
| | | | 7980000 | | |
| sgRNA_120 | AATGTGTTAAATCAAATGCA | chr9: | chr9: | desert_11 | 83.48 |
| | (SEQ ID NO: 307) | 7976895 | 7970000- | (GS120) | |
| | | | 7980000 | | |

### CRISPR-Cas Editing

One effective example of gene editing is the CRISPR-Cas approach (e.g., CRISPR-Cas9). This approach incorporates the use of a guide polynucleotide (e.g., guide ribonucleic acid or gRNA) and a Cas endonuclease (e.g., Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g., Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g., Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540 (7631), 144-149, the relevant disclosures of which are herein incorporated in their entirety).

Methods for conducing gene editing using CRISPR-Cas approaches are known to those of ordinary skill in the art. (See, for example, US Application Nos. U.S. Ser. No. 16/312,676, U.S. Ser. No. 15/303,722, and U.S. Ser. No. 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or tricthylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount. The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of cancer. The appropriate dosage of the immune cell comprising the system may be determined based on the type of cancer to be treated, the type of the immune cell comprising the system, the severity and course of the cancer, the clinical condition of the individual, the individual's clinical history and response to the treatment, and the discretion of the attending physician.

### Pharmaceutical Compositions

The engineered recombinant cells provided herein can be administered as part of a pharmaceutical compositions. These compositions can comprise, in addition to one or more of the recombinant cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the system or cell compositions described herein along with instructions for use. The instructions for use can be present in the kits as a package insert, in the labeling of the container of the kit or components thereof, or can be in digital form (e.g., on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### Additional Embodiments

Embodiment 1 A system comprising:
a. a first chimeric polypeptide comprises a priming receptor;
b. a second chimeric polypeptide comprises a chimeric antigen receptor (CAR); and
c. a cytokine.

Embodiment 2 A system comprising:
a. a first chimeric polypeptide comprising a priming receptor;
b. a second chimeric polypeptide comprising a chimeric antigen receptor (CAR); and
c. a third chimeric polypeptide comprising a synthetic pathway activator (SPA).

Embodiment 3 A system comprising:
a. a first chimeric polypeptide comprising a priming receptor;
b. a second chimeric polypeptide comprising a chimeric antigen receptor (CAR);
c. a third chimeric polypeptide comprising a synthetic pathway activator (SPA); and
d. a cytokine

Embodiment 4 A system comprising:
a. a first chimeric polypeptide comprising a priming receptor;
b. a second chimeric polypeptide comprising a chimeric antigen receptor (CAR);
c. a suppressor of gene expression, and
d. one or both of:
   i. a third chimeric polypeptide comprising a synthetic pathway activator (SPA); and/or
   ii. a cytokine.

Embodiment 5 The system of embodiments 1-4, wherein the priming receptor comprises, from N-terminus to C-terminus,
a. a first extracellular antigen-binding domain;
b. a first transmembrane domain comprising one or more ligand -inducible proteolytic cleavage sites; and
c. an intracellular domain comprising a human or humanized transcriptional effector.

Embodiment 6 The system of embodiment 5, wherein the first extracellular antigen-binding domain specifically binds to Alkaline Phosphatase, Germ Cell (ALPG/P).

Embodiment 7 The system of embodiment 5 or 6, wherein the first extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein:
a. CDR-H1 comprises the sequence set forth in SEQ ID NO: 1, 39, 40, 41, or 42,
b. CDR-H2 comprises the sequence set forth in SEQ ID NO: 2, 43, 44, 45, or 46,
c. CDR-H3 comprises the sequence set forth in SEQ ID NO: 3, 47, or 48,
d. CDR-L1 comprises the sequence set forth in SEQ ID NO: 4, 49, or 50,
e. CDR-L2 comprises the sequence set forth in SEQ ID NO: 5 or 51; and
f. CDR-L3 comprises the sequence set forth in SEQ ID NO: 6 or 53.

Embodiment 8 The system of embodiment 7, wherein the VH chain sequence comprises the sequence set forth in SEQ ID NO: 7.

Embodiment 9 The system of embodiment 7 or 8, wherein the VL chain sequence comprises the sequence set forth in SEQ ID NO: 8.

Embodiment 10 The system of any one of embodiments 5-9, wherein the first extracellular antigen-binding domain comprises the sequence set forth in SEQ ID NO: 9.

Embodiment 11 The system of any one of embodiments 5-10, wherein binding of ALPG/P by the first extracellular antigen-binding domain results in cleavage at the one or more ligand -inducible proteolytic cleavage sites within the intracellular domain.

Embodiment 12 The system of any one of embodiments 5-11, wherein the priming receptor further comprises a first hinge domain positioned between the first extracellular antigen-binding domain and the first transmembrane domain.

Embodiment 13 The system of embodiment 12, wherein the first hinge domain comprises a CD8a or truncated CD8a hinge domain.

Embodiment 14 The system of embodiment 13, wherein the first hinge comprises the sequence as set forth in SEQ ID NO: 18.

Embodiment 15 The system of any one of embodiments 5-14, wherein the first transmembrane domain comprises a Notch1 transmembrane domain.

Embodiment 16 The system of embodiment 15, wherein the first transmembrane domain comprises the sequence as set forth in SEQ ID NO: 19.

Embodiment 17 The system of any one of embodiments 5-16, wherein the intracellular domain comprises an HNF1a/p65 domain or a Gal4/VP64 domain.

Embodiment 18 The system of embodiment 17, wherein the intracellular domain comprises the sequence as set forth in SEQ ID NO: 23.

Embodiment 19 The system of any one of embodiments 5-18, wherein the priming receptor further comprises a stop-transfer-sequence between the first transmembrane domain and the intracellular domain.

Embodiment 20 The system of embodiment 19, wherein the stop-transfer-sequence comprises the sequence as set forth in SEQ ID NO: 20.

Embodiment 21 The system of any one of embodiments 1-20, wherein the priming receptor comprises a sequence as set forth in SEQ ID NO: 24.

Embodiment 22 The system of any one of embodiments 1-21, wherein the CAR comprises, from N-terminus to C-terminus,
a. a second extracellular antigen-binding domain;
b. a second transmembrane domain;
c. an intracellular co-stimulatory domain; and
d. an intracellular activation domain.

Embodiment 23 The system of embodiment 22, wherein the second extracellular antigen-binding domain specifically binds to mesothelin (MSLN), wherein the second extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein:
a. CDR-H1 comprises the sequence set forth in SEQ ID NO: 10, 54, 56, 57, or 71,
b. CDR-H2 comprises the sequence set forth in SEQ ID NO: 11, 58, 59, 60, or 61,
c. CDR-H3 comprises the sequence set forth in SEQ ID NO: 12, 62, or 63,
d. CDR-L1 comprises the sequence set forth in SEQ ID NO: 14, 64, 65, 66, or 67,
c. CDR-L2 comprises the sequence set forth in SEQ ID NO: 15, 68, 69, or 70, and
f. CDR-L3 comprises the sequence set forth in SEQ ID NO: 16, 72, or 73.

Embodiment 24 The system of embodiment 23, wherein the VH chain sequence comprises the sequence set forth in SEQ ID NO: 13.

Embodiment 25 The system of embodiment 23 or 24, wherein the VL chain sequence comprises the sequence set forth in SEQ ID NO: 17.

Embodiment 26 The system of any one of embodiments 23-25, wherein the second extracellular antigen-binding domain comprises the amino acid sequence set forth in SEQ ID NO: 30.

Embodiment 27 The system of any one of embodiments 1-26, wherein the CAR comprises a second hinge domain.

Embodiment 28 The system of embodiment 27, wherein the second hinge domain comprises a CD8a or truncated CD8a hinge domain.

Embodiment 29 The system of any one of embodiments 22-28, wherein the second transmembrane domain comprises a CD8a transmembrane domain.

Embodiment 30 The system of any one of embodiments 22-29, wherein the intracellular co-stimulatory domain comprises a 4-1BB domain.

Embodiment 31 The system of any one of embodiments 22-30, wherein the intracellular activation domain comprises a CD32 domain.

Embodiment 32 The system of any one of embodiments 1-31, wherein the CAR comprises a sequence as set forth in SEQ ID NO: 31 or 32.

Embodiment 33 The system of any one of embodiments 2-32, wherein the SPA is an activator of STAT phosphorylation, optionally STAT1, STAT3, and/or STAT5 phosphorylation.

Embodiment 34 The system of any one of embodiments 2-33, wherein the SPA comprises an extracellular domain linked to an intracellular signaling domain.

Embodiment 35 The system of embodiment 34, wherein the intracellular signaling domain comprises an intracellular signaling region derived from a cytokine receptor.

Embodiment 36 The system of embodiment 34 or 35, wherein the intracellular signaling domain comprises a polypeptide sequence derived from an interleukin receptor.

Embodiment 37 The system of embodiment 34 or 35, wherein the cytokine receptor comprises interleukin-6 signal transducer (IL6ST).

Embodiment 38 The system of any one of embodiments 34-37, wherein the extracellular domain conveys constitutive activity to the intracellular signaling domain.

Embodiment 39 The system of any one of embodiment 34-38, wherein the extracellular domain comprises a dimerization region, optionally wherein the dimerization region comprises at least one of a cysteine residue and a leucine zipper.

Embodiment 40 The system of embodiment 39, wherein the dimerization region forms a homodimer.

Embodiment 41 The system of any one of embodiments 2-40, wherein the SPA comprises a leucine zipper-gp130 (L-gp130).

Embodiment 42 The system of any one of embodiments 2-41, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74.

Embodiment 43 The system of any one of embodiments 2-42, wherein the SPA comprises the amino acid sequence of SEQ ID NO: 74.

Embodiment 44 The system of any one of embodiments 2-43, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75.

Embodiment 45 The system of any one of embodiments 2-44, wherein the SPA comprises the amino acid sequence of SEQ ID NO: 75.

Embodiment 46 The system of embodiment 38, wherein the extracellular domain comprises a polypeptide derived from a cytokine and mimics receptor agonism.

Embodiment 47 The system of any one of embodiments 2-36, 38, or 46, wherein the SPA comprises a membrane-bound interleukin-15 (mbIL-15).

Embodiment 48 The system of any one of embodiments 2-38, 46, or 47, wherein the SPA comprises an amino acid sequence at least 90%, at least 91%, at least 92%, at least 93%, at least 94%, at least 95%, at least 96%, at least 97%, at least 98%, or at least 99% % identical to the sequence set forth in SEQ ID NO: 76.

Embodiment 49 The system of embodiment 2-36, 38 or 46-48, wherein the SPA comprises the amino acid sequence of SEQ ID NO: 76.

Embodiment 50 The system of any one of embodiments 2-36 or 38, wherein the SPA comprises a CD34-interleukin-7 receptor (C7R).

Embodiment 51 The system of embodiment 2-36, 38, or 50, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 77.

Embodiment 52 The system of embodiment 2-36, 38, 50, or 51, wherein the SPA comprises an amino acid sequence of SEQ ID NO: 77.

Embodiment 53 The system of embodiment 2-36, 38, or 50-52, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 78.

Embodiment 54 The system of embodiment 2-36, 38, or 50-53, wherein the SPA comprises an amino acid sequence of SEQ ID NO: 78.

Embodiment 55 The system of any one of embodiments 1-32, wherein the cytokine is a secreted cytokine.

Embodiment 56 The system of any one of embodiments 1-32, wherein the cytokine is a membrane-bound cytokine.

Embodiment 57 The system of any one of embodiments 1-32, 55, and 56, wherein the cytokine is an interleukin.

Embodiment 58 The system of any one of embodiments 1-32, and 55-57, wherein the cytokine comprises at least one of interleukin (IL)-2, Super-2, IL-12, IL-12/23p40, IL-7, IL-15, IL-21, and IL-18.

Embodiment 59 The system of any one of embodiments 1-32, and 55-58, wherein the cytokine is IL-2.

Embodiment 60 The system of any one of embodiments 1-32, and 55-58, wherein the cytokine is Super-2.

Embodiment 61 The system of any one of embodiments 1-32, and 55-58, wherein the cytokine is IL-12.

Embodiment 62 The system of any one of embodiments 1-32, and 55-58, wherein the cytokine is IL-12/23p40.

Embodiment 63 The system of any one of embodiments 1-32, and 55-58, wherein the cytokine is IL-7.

Embodiment 64 The system of any one of embodiments 1-32, and 55-58, wherein the cytokine is IL-15.

Embodiment 65 The system of any one of embodiments 1-32, and 55-58, wherein the cytokine is IL-21.

Embodiment 66 The system of any one of embodiments 1-32, and 55-58, wherein the cytokine is IL-18.

Embodiment 67 The system of any one of embodiments 1-32, and 55-66, wherein the cytokine comprises a non-native signal peptide.

Embodiment 68 The system of embodiment 67, wherein the non-native signal peptide comprises a signal peptide from at least one of CD44, CD3E, CD5, IGTAL, IL-2, GMCSF, chymotrypsinogen, trypsinogen, IgK, IgKVIII, IgE, OSM, IgG2H, BM40, secrecon, and tPA.

Embodiment 69 The system of embodiment 67 or 68, wherein the non-native signal peptide comprises or consists of the amino acid sequence set forth in SEQ ID NO: 100, 102, 104, 106, 108, 110, 112, 114, 116, 118, 120, 122, 124, 126, 128, or 130.

Embodiment 70 The system of any one of embodiments 1-32, and 55-69, wherein the cytokine comprises the amino acid sequence set forth in SEQ ID NO: 86, 88, 90, 92, 94, 96, 98, or 132.

Embodiment 71 The system of any one of embodiments 3-32, wherein the suppressor of gene expression is an sgRNA or an shRNA.

Embodiment 72 The system of any one of embodiments 3-32 and 71, wherein the suppressor of gene expression is an sgRNA.

Embodiment 73 The system of embodiment 72, wherein the sgRNA suppresses the expression of a gene selected from PTPN2, RASA2, SOCS1, ZC3H12A, and CISH.

Embodiment 74 The system of embodiment 72 or 73, wherein the sgRNA suppresses the expression of PTPN2.

Embodiment 75 The system of embodiment 72 or 73, wherein the sgRNA suppresses the expression of RASA2.

Embodiment 76 The system of embodiment 72 or 73, wherein the sgRNA suppresses the expression of SOCS1.

Embodiment 77 The system of embodiment 72 or 73, wherein the sgRNA suppresses the expression of ZC3H12A.

Embodiment 78 The system of embodiment 72 or 73, wherein the sgRNA suppresses the expression of CISH.

Embodiment 79 The system of embodiment 72 or 73, wherein the sgRNA comprises a nucleic acid sequence set forth in any one of SEQ ID NOs: 160-164.

Embodiment 80 The system of any one of embodiments 3-32 and 71, wherein the suppressor of gene expression is an shRNA.

Embodiment 81 The system of embodiment 80, wherein the shRNA suppresses the expression of a gene selected from RASA2, SOCS1, ZC3H12A, TGFBR1, and CISH.

Embodiment 82 The system of embodiment 80 or 81, wherein the shRNA suppresses the expression of RASA2.

Embodiment 83 The system of embodiment 80 or 81, wherein the shRNA suppresses the expression of SOCS1.

Embodiment 84 The system of embodiment 80 or 81, wherein the shRNA suppresses the expression of ZC3H12A.

Embodiment 85 The system of embodiment 80 or 81, wherein the shRNA suppresses the expression of TGFBR1.

Embodiment 86 The system of embodiment 80 or 81, wherein the shRNA suppresses the expression of CISH.

Embodiment 87 The system of embodiment 80 or 81, wherein the shRNA comprises a nucleic acid sequence set forth in any one of SEQ ID NOs: 165-172.

Embodiment 88 The system of any one of embodiments 3-32, wherein the system comprises two or more suppressors of gene expression.

Embodiment 89 The system of embodiment 88, wherein the system comprises an shRNA that suppresses the expression of TNFRSF6 (Fas) and an additional suppressor of gene expression.

Embodiment 90 The system of embodiment 88, wherein the system comprises an shRNA that suppresses the expression of TNFRSF6 (Fas), an shRNA that suppresses the expression of TGFBR2 and an additional suppressor of gene expression.

Embodiment 91 The system of embodiment 88, wherein the system comprises an shRNA that suppresses the expression of TNFRSF6 (Fas), an shRNA that suppresses the expression of PTPN2 and an additional suppressor of gene expression.

Embodiment 92 The system of any one of embodiments 3-32, wherein the system comprises an sgRNA that suppresses CISH expression and a cytokine that is IL-2.

Embodiment 93 The system of any one of embodiments 3-32, wherein the system comprises an sgRNA that suppresses PTPN2 expression and a cytokine that is IL-2.

Embodiment 94 The system of any one of embodiments 3-32, wherein the system comprises an sgRNA that suppresses RASA2 expression and a cytokine that is IL-2.

Embodiment 95 The system of any one of embodiments 3-32, wherein the system comprises an sgRNA that suppresses SOCS1 expression and a cytokine that is IL-2.

Embodiment 96 The system of any one of embodiments 3-32, wherein the system comprises an sgRNA that suppresses ZC3H12A expression and a cytokine that is IL-2.

Embodiment 97 The system of any one of embodiments 3-32, wherein the system comprises an sgRNA that suppresses PTPN2 expression and a cytokine that is IL-21.

Embodiment 98 The system of any one of embodiments 3-32, wherein the system comprises an sgRNA that suppresses ZC3H12A expression and a cytokine that is IL-21.

Embodiment 99 The system of any one of embodiments 3-32, wherein the system comprises an shRNA that suppresses RASA2 expression and a cytokine that is IL-2.

Embodiment 100 The system of any one of embodiments 3-32, wherein the system comprises an sgRNA that suppresses CISH expression and an SPA that is C7R.

Embodiment 101 The system of any one of embodiments 3-32, wherein the system comprises an sgRNA that suppresses PTPN2 expression and an SPA that is C7R.

Embodiment 102 The system of any one of embodiments 3-32, wherein the system comprises an sgRNA that suppresses RASA2 expression and an SPA that is C7R.

Embodiment 103 The system of any one of embodiments 3-32, wherein the system comprises an sgRNA that suppresses SOCS1 expression and an SPA that is C7R.

Embodiment 104 The system of any one of embodiments 3-32, wherein the system comprises an sgRNA that suppresses ZC3H12A expression and an SPA that is C7R.

Embodiment 105 The system of any one of embodiments 3-32, wherein the system comprises an sgRNA that suppresses CISH expression and an SPA that is L-gp130.

Embodiment 106 The system of any one of embodiments 3-32, wherein the system comprises an sgRNA that suppresses RASA2 expression and an SPA that is L-gp130.

Embodiment 107 The system of any one of embodiments 3-32, wherein the system comprises an sgRNA that suppresses ZC3H12A expression and an SPA that is L-gp130.

Embodiment 108 The system of any one of embodiments 3-32, wherein the system comprises an shRNA that suppresses RASA2 expression, a cytokine that is IL-2, and an SPA that is L-gp130.

Embodiment 109 The system of any one of embodiments 3-32, wherein the system comprises an sgRNA that suppresses RASA2 expression, a cytokine that is IL-2, and an SPA that is L-gp130.

Embodiment 110 The system of any one of embodiments 3-32, wherein the system comprises an shRNA that suppresses RASA2 expression, a cytokine that is IL-15, and an SPA that is L-gp130.

Embodiment 111 The system of any one of embodiments 3-32, wherein the system comprises an sgRNA that suppresses RASA2 expression, a cytokine that is IL-15, and an SPA that is L-gp130.

Embodiment 112 The system of any one of embodiments 3-112, wherein the priming receptor and the CAR are capable of binding to a same target cell.

Embodiment 113 The system of embodiment 112, wherein the target cell is a human cell.

Embodiment 114 The system of embodiment 112 or 113, wherein the target cell is a cancer cell.

Embodiment 115 The system of embodiment 114, wherein the cancer cell is a solid cancer cell or a liquid cancer cell.

Embodiment 116 The system of embodiment 114 or 115, wherein the cancer cell is ovarian cancer, fallopian cancer, primary peritoneal cancer, uterine cancer, mesothelioma, cervical cancer, or pancreatic cancer.

Embodiment 117 One or more recombinant nucleic acids comprising at least one nucleic acid fragment comprising a nucleotide sequence encoding the system of one of embodiments 1-116.

Embodiment 118 One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising a second extracellular antigen-binding domain; and
c. a nucleotide sequence encoding a cytokine.

Embodiment 119 One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain; and
c. a nucleotide sequence encoding a synthetic pathway activator.

Embodiment 120 One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain;
c. a nucleotide sequence encoding a synthetic pathway activator; and
d. a nucleotide sequence encoding a cytokine.

Embodiment 121 One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain; and
c. a nucleotide sequence of a suppressor of gene expression; and
d. one or both of:
   i. a nucleotide sequence encoding a synthetic pathway activator; and/or
   ii. a nucleotide sequence encoding a cytokine.

Embodiment 122 The recombinant nucleic acid of any one of embodiments 118-121, wherein the first extracellular antigen-binding domain specifically binds to ALPG/P.

Embodiment 123 The recombinant nucleic acid of any one of embodiments 118-122, wherein the second extracellular antigen-binding domain specifically binds to MSLN.

Embodiment 124 The recombinant nucleic acid of embodiment 117-123, wherein the recombinant nucleic acid comprises two or more nucleic acid fragments.

Embodiment 125 The recombinant nucleic acid of any one of embodiments 117-124, wherein the recombinant nucleic acid further comprises an inducible promoter operably linked to the nucleotide sequence encoding the CAR.

Embodiment 126 The recombinant nucleic acid of any one of embodiments 117-125, wherein the recombinant nucleic acid further comprises an inducible promoter operably linked to the nucleotide sequence encoding the priming receptor.

Embodiment 127 The recombinant nucleic acid of any one of embodiments 117-125, wherein the recombinant nucleic acid further comprises a constitutive promoter operably linked to the nucleotide sequence encoding the priming receptor.

Embodiment 128 The recombinant nucleic acid of any one of embodiments 117-125, wherein the recombinant nucleic acid further comprises a constitutive promoter operably linked to the nucleotide sequence encoding the synthetic pathway activator.

Embodiment 129 The recombinant nucleic acid of any one of embodiments 117-127, wherein the recombinant nucleic acid further comprises an inducible promoter operably linked to the nucleotide sequence encoding the synthetic pathway activator.

Embodiment 130 The recombinant nucleic acid of embodiment 127 or 128, wherein the priming receptor and the synthetic pathway activator are under the control of the same constitutive promoter.

Embodiment 131 The recombinant nucleic acid of any one of embodiments 117-124, wherein the recombinant nucleic acid further comprises an inducible promoter operably linked to the nucleotide sequence encoding the chimeric antigen receptor and a constitutive promoter operably linked to the nucleotide sequence encoding the priming receptor and the nucleotide sequence encoding the synthetic pathway activator.

Embodiment 132 The recombinant nucleic acid of embodiments 131, wherein the nucleic acid comprises, in a 5′ to 3′ direction,
a. the constitutive promoter;
b. the nucleotide sequence encoding the synthetic pathway activator;
c. the nucleotide sequence encoding priming receptor;
d. the inducible promoter; and
e. the nucleotide sequence encoding chimeric antigen receptor.

Embodiment 133 The recombinant nucleic acid of any one of embodiments 131, wherein the nucleic acid comprises, in a 5′ to 3′ direction,
a. the inducible promoter;
b. the nucleotide sequence encoding chimeric antigen receptor;
c. the constitutive promoter;
d. the nucleotide sequence encoding priming receptor; and
e. the nucleotide sequence encoding the synthetic pathway activator.

Embodiment 134 The recombinant nucleic acid of any one of embodiments 117-127, wherein the recombinant nucleic acid further comprises an inducible promoter operably linked to the nucleotide sequence encoding the cytokine.

Embodiment 135 The recombinant nucleic acid of any one of embodiments 117-127, and 134, wherein the recombinant nucleic acid further comprises:
a. an inducible promoter operably linked to the nucleotide sequence encoding the chimeric antigen receptor and the nucleotide sequence encoding the cytokine; and
b. a constitutive promoter operably linked to the nucleotide sequence encoding the priming receptor.

Embodiment 136 The recombinant nucleic acid of embodiment 135, wherein the nucleic acid comprises, in a 5′ to 3′ direction,
a. the constitutive promoter;
b. the nucleotide sequence encoding the priming receptor;
c. the inducible promoter;
d. the nucleotide sequence encoding the chimeric antigen receptor; and
e. the nucleic acid sequence encoding the cytokine and/or the nucleic acid sequence encoding synthetic pathway activator.

Embodiment 137 The recombinant nucleic acid of embodiment 135, wherein the nucleic acid comprises, in a 5′ to 3′ direction,
a. the constitutive promoter;
b. the nucleotide sequence encoding the priming receptor;
c. the inducible promoter;
d. the nucleotide sequence encoding the cytokine and/or the nucleic acid sequence encoding synthetic pathway activator; and
e. the nucleic acid sequence encoding the chimeric antigen receptor.

Embodiment 138 The recombinant nucleic acid of embodiment 135, wherein the nucleic acid comprises, in a 5′ to 3′ direction,
a. the inducible promoter;
b. the nucleotide sequence encoding the chimeric antigen receptor;
c. the nucleic acid sequence encoding the cytokine and/or the nucleic acid sequence encoding synthetic pathway activator;
d. the constitutive promoter; and
e. the nucleotide sequence encoding the priming receptor.

Embodiment 139 The recombinant nucleic acid of embodiment 135, wherein the nucleic acid comprises, in a 5′ to 3′ direction,
a. the inducible promoter;
b. the nucleotide sequence encoding the cytokine and/or the nucleic acid sequence encoding synthetic pathway activator;
c. the nucleic acid sequence encoding the chimeric antigen receptor;
d. the constitutive promoter; and
e. the nucleotide sequence encoding the priming receptor.

Embodiment 140 The recombinant nucleic acid of any one of embodiments 117-127, wherein the recombinant nucleic acid further comprises:
a. a first inducible promoter operably linked to the nucleotide sequence encoding the chimeric antigen receptor;
b. a second inducible promoter operably linked to the nucleotide sequence encoding the cytokine or synthetic pathway activator; and
c. a constitutive promoter operably linked to the nucleotide sequence encoding the priming receptor.

Embodiment 141 The recombinant nucleic acid of embodiment 140, wherein the nucleic acid comprises, in a 5′ to 3′ direction,
a. the constitutive promoter;
b. the nucleotide sequence encoding the priming receptor;
c. the first inducible promoter;
d. the nucleotide sequence encoding the chimeric antigen receptor;
e. the second inducible promoter; and
f. the nucleic acid sequence encoding the cytokine and/or the nucleic acid sequence encoding synthetic pathway activator.

Embodiment 142 The recombinant nucleic acid of embodiment 140, wherein the nucleic acid comprises, in a 5′ to 3′ direction,
a. the constitutive promoter;
b. the nucleotide sequence encoding the priming receptor;
c. the second inducible promoter;
d. the nucleic acid sequence encoding the cytokine and/or the nucleic acid sequence encoding synthetic pathway activator;
e. the first inducible promoter; and
f. the nucleotide sequence encoding the chimeric antigen receptor.

Embodiment 143 The recombinant nucleic acid of embodiment 140, wherein the nucleic acid comprises, in a 5′ to 3′ direction,
a. the first inducible promoter;
b. the nucleotide sequence encoding the chimeric antigen receptor;
c. the second inducible promoter;
d. the nucleic acid sequence encoding the cytokine and/or the nucleic acid sequence encoding synthetic pathway activator;
e. the constitutive promoter; and
f. the nucleotide sequence encoding the priming receptor.

Embodiment 144 The recombinant nucleic acid of embodiment 140, wherein the nucleic acid comprises, in a 5′ to 3′ direction,
a. the first inducible promoter;
b. the nucleotide sequence encoding the chimeric antigen receptor;
c. the constitutive promoter;
d. the nucleotide sequence encoding the priming receptor;
e. the second inducible promoter; and
f. the nucleic acid sequence encoding the cytokine and/or the nucleic acid sequence encoding synthetic pathway activator.

Embodiment 145 The recombinant nucleic acid of embodiment 140, wherein the nucleic acid comprises, in a 5′ to 3′ direction,
a. the second inducible promoter;
b. the nucleic acid sequence encoding the cytokine and/or synthetic pathway activator;
c. the first inducible promoter;
d. the nucleotide sequence encoding the chimeric antigen receptor;
e. the constitutive promoter; and
f. the nucleotide sequence encoding the priming receptor.

Embodiment 146 The recombinant nucleic acid of embodiment 140, wherein the nucleic acid comprises, in a 5′ to 3′ direction,
a. the second inducible promoter;
b. the nucleic acid sequence encoding the cytokine and/or the nucleic acid sequence encoding synthetic pathway activator;
c. the constitutive promoter;
d. the nucleotide sequence encoding priming receptor;
e. the first inducible promoter; and
f. the nucleotide sequence encoding chimeric antigen receptor.

Embodiment 147 The recombinant nucleic acid of any one of embodiments 117-146, wherein the nucleotide sequence encoding the priming receptor comprises the sequence set forth in SEQ ID NO: 35.

Embodiment 148 The recombinant nucleic acid of any one of embodiments 117-147, wherein the nucleotide sequence encoding the chimeric antigen receptor comprises the sequence set forth in SEQ ID NO: 36.

Embodiment 149 The recombinant nucleic acid of any one of embodiments 117-133, 147, and 148, wherein the nucleotide sequence encoding the synthetic pathway activator comprises the sequence set forth in SEQ ID NO: 79, 80, 81, 82, or 83.

Embodiment 150 The recombinant nucleic acid of any one of embodiments 117-127 and 134-148, wherein nucleotide sequence encoding the cytokine comprises the sequence set forth in SEQ ID NO: 87, 89, 91, 93, 95, 97, 99, or 133.

Embodiment 151 The recombinant nucleic acid of any one of embodiments 117-150, wherein the suppressor of gene expression comprises a nucleic acid sequence set forth in any one of SEQ ID NOs: 160-172.

Embodiment 152 The recombinant nucleic acid of embodiment of any one of embodiment 117-151, wherein the nucleic acid further comprises a 5′ homology directed repair arm and a 3′ homology directed repair arm complementary to an insertion site in a host cell chromosome.

Embodiment 153 The recombinant nucleic acid of any one of embodiments 117-152, wherein the recombinant nucleic acid further comprises a nucleotide sequence encoding a self-excising 2A peptide (P2A).

Embodiment 154 The recombinant nucleic acid of embodiment 153, wherein the P2A is at the 3′ end of the nucleotide sequence encoding chimeric antigen receptor.

Embodiment 155 The recombinant nucleic acid of embodiment 153, wherein the P2A is at the 3′ end of the nucleotide sequence encoding priming receptor.

Embodiment 156 The recombinant nucleic acid of any one of embodiments 117-155, wherein the recombinant nucleic acid further comprises a woodchuck hepatitis virus post-translational regulatory element (WPRE).

Embodiment 157 The recombinant nucleic acid of embodiment 156, wherein the WPRE is at the 3′ end of the nucleotide sequence encoding chimeric antigen receptor and at the 5′ end of the nucleotide sequence encoding priming receptor or wherein the WPRE is at the 3′ end of the nucleotide sequence encoding priming receptor and at the 5′ end of the nucleotide sequence encoding chimeric antigen receptor.

Embodiment 158 The recombinant nucleic acid of any one of embodiments 117-157, wherein the recombinant nucleic acid further comprises an SV40 polyA element.

Embodiment 159 The recombinant nucleic acid of any one of embodiments 117 to 158, wherein the nucleic acid is incorporated into an expression cassette or an expression vector.

Embodiment 160 The recombinant nucleic acid of embodiment 159, wherein the expression vector is a non-viral vector.

Embodiment 161 An expression vector comprising the recombinant nucleic acid of any one of embodiments 117-160.

Embodiment 162 The vector of embodiment 161, wherein the 5′ and 3′ ends of the recombinant nucleic acid comprise nucleotide sequences that are homologous to genomic sequences flanking an insertion site in a genome of a primary cell.

Embodiment 163 The vector of embodiment 162, wherein the insertion site is located at a T Cell Receptor Alpha Constant (TRAC) locus or a genomic safe harbor (GSH) locus.

Embodiment 164 An immune cell comprising:
a. the system of any one of embodiments 1-116;
b. at least one recombinant nucleic acid of any one of embodiments 117-160; and/or
c. the vector of any one of embodiments 161-163.

Embodiment 165 The immune cell of embodiment 164, wherein the immune cell is a primary human immune cell.

Embodiment 166 The immune cell of any one of embodiments 164 or 165, wherein the immune cell is an allogeneic immune cell.

Embodiment 167 The immune cell of any one of embodiments 164 or 165, wherein the immune cell is an autologous immune cell.

Embodiment 168 The immune cell of any one of embodiments 165-167, wherein the primary immune cell is a natural killer (NK) cell, a T cell, a CD8+ T cell, a CD4+ T cell, a primary T cell, or a T cell progenitor.

Embodiment 169 The immune cell of any one of embodiments 165-168, wherein the primary immune cell is a primary T cell.

Embodiment 170 The immune cell of any one of embodiments 165-169, wherein the primary immune cell is a primary human T cell.

Embodiment 171 The immune cell of any one of embodiments 165-170, wherein the primary immune cell is virus-free.

Embodiment 172 A primary immune cell comprising at least one recombinant nucleic acid comprising:
a. a nucleic acid sequence encoding a priming receptor comprising a first extracellular antigen-binding domain;
b. a nucleic acid sequence encoding a chimeric antigen receptor comprising a second extracellular antigen-binding domain; and
c. a nucleic acid sequence encoding a synthetic pathway activator and/or a nucleic acid sequence encoding a cytokine;
wherein the recombinant nucleic acid is inserted into a target region of the genome of the primary immune cell, wherein the primary immune cell does not comprise a viral vector for introducing the recombinant nucleic acid into the primary immune cell.

Embodiment 173 The primary immune cell of embodiment 172, wherein the first extracellular antigen-binding domain specifically binds to ALPG/P.

Embodiment 174 The primary immune cell of embodiment 172 or 173, wherein the second extracellular antigen-binding domain specifically binds to MSLN.

Embodiment 175 A viable, virus-free, primary cell comprising a ribonucleoprotein complex (RNP)-recombinant nucleic acid complex, wherein the RNP comprises a nuclease domain and a guide RNA, wherein recombinant nucleic acid comprises:
a. a nucleic acid sequence encoding a priming receptor comprising a first extracellular antigen-binding domain that specifically binds to ALPG/P;
b. a nucleic acid sequence encoding a chimeric antigen receptor comprising a second extracellular antigen-binding domain that specifically binds to MSLN; and
c. a nucleic acid sequence encoding a synthetic pathway activator that constitutively activates cytokine signaling and/or a nucleic acid sequence encoding a cytokine;
wherein the 5′ and 3′ ends of the recombinant nucleic acid comprise nucleotide sequences that are homologous to genomic sequences flanking an insertion site in the genome of the primary cell.

Embodiment 176 The primary cell of embodiment 175, wherein the first extracellular antigen-binding domain specifically binds to ALPG/P.

Embodiment 177 The primary cell of embodiment 175 or 176, wherein the second extracellular antigen-binding domain specifically binds to MSLN.

Embodiment 178 A population of cells comprising a plurality of immune cells of any one of embodiments 164-171 or primary cells of any one of embodiments 172-177.

Embodiment 179 A pharmaceutical composition comprising the immune cell of any one of embodiments 164 to 177 or the population of cells of embodiment 178, and a pharmaceutically acceptable excipient.

Embodiment 180 A pharmaceutical composition comprising the recombinant nucleic acid of any one of embodiments 117-160 or the vector of any one of embodiments 161-163, and a pharmaceutically acceptable excipient.

Embodiment 181 A method of editing an immune cell, comprising:
a. providing a ribonucleoprotein complex (RNP)-recombinant nucleic acid complex, wherein the RNP comprises a nuclease domain and a guide RNA, wherein the recombinant nucleic acid comprises the recombinant nucleic acid of any one of embodiments 117-160, and wherein the 5′ and 3′ ends of the recombinant nucleic acid comprise nucleotide sequences that are homologous to genomic sequences flanking an insertion site in the genome of the immune cell;
b. non-virally introducing the RNP-recombinant nucleic acid complex into the immune cell, wherein the guide RNA specifically hybridizes to a target region of the genome of the primary immune cell, and wherein the nuclease domain cleaves the target region to create the insertion site in the genome of the immune cell; and
c. editing the immune cell via insertion of the recombinant nucleic acid of any one of embodiments 117-160 into the insertion site in the genome of the immune cell.

Embodiment 182 The method of embodiment 181, wherein non-virally introducing comprises electroporation.

Embodiment 183 The method of embodiment 181 or 182, wherein the nuclease domain comprises a CRISPR-associated endonuclease (Cas), optionally a Cas9 nuclease.

Embodiment 184 The method of any one of embodiments 181-183, wherein the target region of the genome of the cell is a T Cell Receptor Alpha Constant (TRAC) locus or a genomic safe harbor (GSH) locus.

Embodiment 185 The method of any one of embodiments 181-184, wherein the recombinant nucleic acid is a double-stranded recombinant nucleic acid or a single-stranded recombinant nucleic acid.

Embodiment 186 The method of any one of embodiments 181-185, wherein the recombinant nucleic acid is a linear recombinant nucleic acid or a circular recombinant nucleic acid, optionally wherein the circular recombinant nucleic acid is a plasmid.

Embodiment 187 The method of any one of embodiments 181-186, wherein the immune cell is a primary human immune cell.

Embodiment 188 The method of any one of embodiments 181-187, wherein the immune cell is an autologous immune cell.

Embodiment 189 The method of any one of embodiments 181-187, wherein the immune cell is an allogeneic immune cell.

Embodiment 190 The method of any one of embodiments 181-189, wherein the immune cell is a natural killer (NK) cell, a T cell, a CD8+ T cell, a CD4+ T cell, a primary T cell, or a T cell progenitor.

Embodiment 191 The method of any one of embodiments 181-190, wherein the immune cell is a primary T cell.

Embodiment 192 The method of any one of embodiments 181-191, wherein the immune cell is a primary human T cell.

Embodiment 193 The method of any one of embodiments 181-192, wherein the immune cell is virus-free.

Embodiment 194 The method of any one of embodiments 181-193, further comprising obtaining the immune cell from a patient and introducing the recombinant nucleic acid in vitro.

Embodiment 195 A method of treating a disease in a subject comprising administering the immune cell of any one of embodiments 164-171 or primary cells of any one of embodiments 172-177 or the pharmaceutical composition of embodiments 179 or 180 to the subject.

Embodiment 196 The method of embodiment 195, wherein the disease is cancer.

Embodiment 197 The method of embodiment 196, wherein the cancer is a solid cancer or a liquid cancer.

Embodiment 198 The method of embodiment 196 or 197, wherein the cancer is ovarian cancer, fallopian cancer, primary peritoneal cancer, uterine cancer, mesothelioma, cervical cancer, or pancreatic cancer.

Embodiment 199 The method of any one of embodiments 196-198, wherein the administration of the immune cell enhances an immune response in the subject.

Embodiment 200 The method of embodiment 199, wherein the enhanced immune response is an adaptive immune response.

Embodiment 201 The method of embodiment 199, wherein the enhanced immune response is an innate immune response.

Embodiment 202 The method of any one of embodiments 196-201, wherein the enhanced immune response is an increased expression of at least one cytokine or chemokine.

Embodiment 203 The method of embodiment 202, wherein the at least one cytokine or chemokine is IL-2 or IFNγ.

Embodiment 204 The method of any one of embodiments 196-201, wherein the enhanced immune response is an increased lysis of target cells as compared to administration of a control cell.

Embodiment 205 The method of any one of embodiments 195-198, further comprising administering an immunotherapy to the subject concurrently with the immune cell or subsequently to the immune cell.

Embodiment 206 A method of inhibiting a target cell in a subject comprising administering the immune cell of any one of embodiments 164-171 or primary cells of any one of embodiments 172-177 to the subject, wherein the immune cell inhibits the target cell.

Embodiment 207 The method of embodiment 206, wherein the target cell expresses ALPG/P and MSLN.

Embodiment 208 The method of embodiment 206 or 207, wherein the target cell is a cancer cell.

Embodiment 209 A method of modulating the activity of an immune cell comprising:
a. obtaining an immune cell comprising
   i. the system of any one of embodiments 2-116;
   ii. the recombinant nucleic acid of any one of embodiments 117-160; and/or
   iii. the vector of any one of embodiments 161-163; and
b. contacting the immune cell with a target cell expressing a priming receptor antigen and a CAR antigen, wherein binding of the priming receptor to the priming receptor antigen on the target cell induces activation of the priming receptor and expression of the chimeric antigen receptor, wherein binding of the chimeric antigen receptor to the CAR antigen on the target cell modulates the activity of the immune cell, and wherein the synthetic pathway activator and/or cytokine also modulates the activity of the immune cell.

Embodiment 210 A method of modulating the activity of an immune cell comprising:
a. obtaining an immune cell comprising
   i. the system of any one of embodiments 2-116;
   ii. the recombinant nucleic acid of any one of embodiments 117-160; and/or
   iii. the vector of any one of embodiments 161-163; and
b. contacting the immune cell with a target cell expressing ALPG/P and MSLN, wherein binding of the priming receptor to ALPG/P on the target cell induces activation of the priming receptor and expression of the chimeric antigen receptor, wherein binding of the chimeric antigen receptor to MSLN on the target cell modulates the activity of the immune cell, and wherein the synthetic pathway activator and/or cytokine also modulates the activity of the immune cell.

Embodiment 211 The method of embodiment 210, wherein the modulation of the immune cell activity comprises enhancing an immune response.

Embodiment 212 The method of embodiment 211, wherein the enhanced immune response is an adaptive immune response.

Embodiment 213 The method of embodiment 211, wherein the enhanced immune response is an innate immune response.

Embodiment 214 The method of any one of embodiments 210-213, wherein the immune cell activity is an increased expression of at least one cytokine or chemokine.

Embodiment 215 The method of embodiment 214, wherein the at least one cytokine or chemokine is IL-2 or IFNγ.

Embodiment 216 The method of any one of embodiments 210-213, wherein the immune cell activity is lysis of target cells.

Embodiment 217 A system comprising:
a. at least one of a priming receptor, a CAR, a SPA, and a cytokine; and
b. a suppressor of RASA2 expression.

Embodiment 218 The system of embodiment 217, wherein the SPA comprises a leucine zipper-gp130 (L-gp130).

Embodiment 219 The system of embodiment 217 or 218, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74.

Embodiment 220 The system of any one of embodiments 217-219, wherein the SPA comprises the amino acid sequence of SEQ ID NO: 74.

Embodiment 221 The system of any one of embodiments 217-219, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75.

Embodiment 222 The system of any one of embodiments 217-221, wherein the SPA comprises the amino acid sequence of SEQ ID NO: 75.

Embodiment 223 The system of any one of embodiments 217-222, wherein the cytokine is IL-2.

Embodiment 224 The system of any one of embodiments 217-223, wherein the cytokine comprises the amino acid sequence set forth in SEQ ID NO: 86.

Embodiment 225 The system of any one of embodiments 217-222, wherein the cytokine is IL-15.

Embodiment 226 The system of any one of embodiments 217-223, wherein the cytokine comprises the amino acid sequence set forth in SEQ ID NO: 96.

Embodiment 227 The system of any one of embodiments 217-224, wherein the suppressor of RASA2 expression is an shRNA or an sgRNA.

Embodiment 228 The system of any one of embodiments 217-227, wherein the suppressor of RASA2 expression is an shRNA.

Embodiment 229 The system of embodiment 228, wherein the shRNA comprises the nucleic acid sequence of SEQ ID NO: 165.

Embodiment 230 The system of any one of embodiments 217-227, wherein the suppressor of RASA2 expression is an sgRNA.

Embodiment 231 The system of embodiment 230, wherein the sgRNA comprises the nucleic acid sequence of SEQ ID NO: 161.

Embodiment 232 The system of any one of embodiments 217-231, further comprising an shRNA that suppresses the expression of TNFRSF6 (Fas).

Embodiment 233 The system of embodiment 232, further comprising an shRNA that suppresses the expression of TGFBR2.

Embodiment 234 The system of embodiment 232, further comprising an shRNA that suppresses the expression of PTPN2.

Embodiment 235 One or more recombinant nucleic acids comprising at least one nucleic acid fragment comprising a nucleotide sequence encoding the system of one of embodiments 217-234.

Embodiment 236 An expression vector comprising the recombinant nucleic acid sequence of embodiment 235.

Embodiment 237 An engineered immune cell comprising:
a. the system of any one of embodiments 217-234;
b. the recombinant nucleic acid sequence of embodiment 235; or
c. the expression vector of embodiment 236.

Embodiment 238 In an engineered immune cell, the improvement comprising:
a. at least one of a a priming receptor, a CAR, an SPA, and a cytokine; and
b. a suppressor of RASA2 expression.

Embodiment 239 The engineered immune cell of embodiment 238, wherein the SPA comprises a leucine zipper-gp130 (L-gp130).

Embodiment 240 The engineered immune cell of embodiment 238 or 239, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 74.

Embodiment 241 The engineered immune cell of any one of embodiments 238-240, wherein the SPA comprises the amino acid sequence of SEQ ID NO: 74.

Embodiment 242 The engineered immune cell of any one of embodiments 238-240, wherein the SPA comprises an amino acid sequence at least 90%, at least 91%, at least 92%, at least 93%, at least 94%, at least 95%, at least 96%, at least 97%, at least 98%, or at least 99% identical to the sequence set forth in SEQ ID NO: 75.

Embodiment 243 The engineered immune cell of any one of embodiments 238-242, wherein the SPA comprises the amino acid sequence of SEQ ID NO: 75.

Embodiment 244 The engineered immune cell of any one of embodiments 238-243, wherein the cytokine is IL-2.

Embodiment 245 The engineered immune cell of any one of embodiments 238-244, wherein the cytokine comprises the amino acid sequence set forth in SEQ ID NO: 86.

Embodiment 246 The engineered immune cell of any one of embodiments 238-245, wherein the suppressor of RASA2 expression is an shRNA or an sgRNA.

Embodiment 247 The engineered immune cell of any one of embodiments 238-246, wherein the suppressor of RASA2 expression is an shRNA.

Embodiment 248 The engineered immune cell of embodiment 247, wherein the shRNA comprises the nucleic acid sequence of SEQ ID NO: 165.

Embodiment 249 The engineered immune cell of any one of embodiments 238-246, wherein the suppressor of RASA2 expression is an sgRNA.

Embodiment 250 The engineered immune cell of embodiment 249, wherein the sgRNA comprises the nucleic acid sequence of SEQ ID NO: 161.

Embodiment 251 The engineered immune cell of any one of embodiments 238-250, further comprising an shRNA that suppresses the expression of TNFRSF6 (Fas).

Embodiment 252 The engineered immune cell of embodiment 251, further comprising an shRNA that suppresses the expression of TGFBR2.

Embodiment 253 The engineered immune cell of embodiment 251, further comprising an shRNA that suppresses the expression of PTPN2.

Embodiment 254 The engineered immune cell of any one of embodiments 237-253, wherein the engineered immune cell is a primary human immune cell.

Embodiment 255 The engineered immune cell of any one of any one of embodiments 237-254, wherein the engineered immune cell is an allogeneic immune cell.

Embodiment 256 The engineered immune cell of any one of any one of embodiments 237-254, wherein the engineered immune cell is an autologous immune cell.

Embodiment 257 The engineered immune cell of any one of embodiments 254-256, wherein the primary immune cell is a natural killer (NK) cell, a T cell, a CD8+ T cell, a CD4+ T cell, a primary T cell, or a T cell progenitor.

Embodiment 258 The engineered immune cell of any one of embodiments 254-257, wherein the primary immune cell is a primary T cell.

Embodiment 259 The engineered immune cell of any one of embodiments 254-258, wherein the primary immune cell is a primary human T cell.

Embodiment 260 The engineered immune cell of any one of embodiments 254-259, wherein the primary immune cell is virus-free.

Embodiment 261 A population of cells comprising a plurality of engineered immune cells of any one of embodiments 237-260.

Embodiment 262 A pharmaceutical composition comprising the engineered immune cell of any one of embodiments 237-260 or the population of cells of embodiment 261 and a pharmaceutically acceptable excipient.

Embodiment 263 A method of treating a disease in a subject comprising administering the engineered immune cell of any one of embodiments 237-260 or the pharmaceutical composition of embodiment 262 to the subject.

Embodiment 264 The method of embodiment 263, wherein the disease is cancer.

Embodiment 265 The method of embodiment 264, wherein the cancer is a solid cancer or a liquid cancer.

Embodiment 266 The method of embodiment 264 or 265, wherein the cancer is ovarian cancer, fallopian cancer, primary peritoneal cancer, uterine cancer, mesothelioma, cervical cancer, or pancreatic cancer.

Embodiment 267 The method of any one of embodiments 264-266, wherein the administration of the engineered immune cell enhances an immune response in the subject.

Embodiment 268 The method of embodiment 267, wherein the enhanced immune response is an adaptive immune response.

Embodiment 269 The method of embodiment 267, wherein the enhanced immune response is an innate immune response.

Embodiment 270 The method of any one of embodiments 264-269, wherein the enhanced immune response is an increased expression of at least one cytokine or chemokine.

Embodiment 271 The method of embodiment 270, wherein the at least one cytokine or chemokine is IL-2 or IFNγ.

Embodiment 272 The method of any one of embodiments 264-271, wherein the enhanced immune response is an increased lysis of target cells as compared to administration of a control cell.

Embodiment 273 The method of any one of embodiments 263-272, further comprising administering an immunotherapy to the subject concurrently with the engineered immune cell or subsequently to the engineered immune cell.

Embodiment 274 A method of inhibiting a target cell in a subject comprising administering the engineered immune cell of any one of embodiments 237-260 or the pharmaceutical composition of embodiment 262 to the subject, wherein the engineered immune cell inhibits the target cell.

Embodiment 275 The method of embodiment 274, wherein the target cell is a cancer cell.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Enhanced Logic Gate T Cell Survival Using Synthetic Pathway Activators

### Materials and Methods

T cells were engineered to express the ALPG/MSLN logic gate including the APLG PrimeR and the MSLN CAR (LG T cells). LG T cells were further engineered to express one of three synthetic pathway activators (SPAs): leucine zipper-gp130 (L-gp130) (SEQ ID NO: 75), membrane-bound IL-15 (mbIL-15) (SEQ ID NO: 76), CD34-IL-7 receptor (C7R) (SEQ ID NO: 78) or a control: truncated EGFR (EGFRt, SEQ ID NO: 84) or c-Jun (SEQ ID NO: 85).

### Cytokine-Free Survival

T cells from a single donor were engineered to express ALPG/MSLN circuits containing genes encoding SPA accessory proteins using the CiTE manufacturing process and were frozen and cryobanked at Day 9 after initial activation. Prior to the assay, engineered T cells were thawed and rested overnight in media including 12.5 ng/ml human IL-7 and IL-15. On the day of the assay, T cells were counted, spun down, washed 1× in cytokine-free media, and then resuspended in cytokine-free media. x total T cells were then plated per well of a 96-well v-bottom plate in 100 uL media without IL-7 and IL-15. Duplicate plates were prepared. One of the plates was immediately analyzed for cell counts and % total edited cells. The second plate was placed in a cell incubator for 6 days, at which time cell count and % editing data were collected.

Cell count and % editing were determined by pelleting cells at 300×g for 5 min, and resuspending in FACS buffer containing anti-Myc PE antibodies at 1:50 dilution (for detection of surface PrimeR expression) and anti-FLAG BV421 (for surface CAR expression). Following a 20 min staining period at room temperature, cells were spun down and washed 1× with FACS Buffer. Following a spin down, cells were resuspended in 50 μL of FACS buffer, then topped with 50 μL of CountBright Plus counting beads. Data were acquired on an Attune NxT flow cytometer. FSC and SSC paramters were used to specify gates for counting beads versus T cells. Absolute cell count was derived by using the formula: Cells/μL=(Cell count/Counting beads count)×Counting beads concentration from bottle. Fold change of edited T cell number and % edited T cells was determined by the formula: T-cell fold change from DO=(T cell count at D6/T cell count at DO).

### Analysis of STAT Phosphorylation

T cells from one donor CITE-edited with ALPG/MSLN circuits containing genes encoding SPA accessory proteins were profiled for their STAT phosphorylation status by intracellular antibody staining. Cryopreserved CITE-edited T cells were thawed and rested for 20 hours in media containing IL-7 and IL-15 at 12.5 ng/mL, respectively. Following the resting period T cells were washed with cytokine-free, serum-free media and then spun down and resuspended in cytokine-free, serum-free media. Following a 20-hr serum starvation period at 37° C., Zombie-Near Infrared dye was added to the cell media at 1:100 dilutiuon. After a 15 min. incubation, equivalent volume BD Cytofix was added to the cells and the plate was incubated at 37° C. for 15 min. After fixation, cells were spun down at 400×g for 5 min and resuspended in 200 μL of BD Perm Buffer III, and incubated on ice for 30 min. Cells were then washed 2× with BD FACS buffer and resuspended in FACS staining buffer containing anti-Myc PE antibodies at 1:50 (For detecting PrimeR-expressing cells), anti-pSTAT5 A488 at 1:10, and anti-pSTAT3 A647 at 1:10 or anti-pSTATI A647. Following a 30 min incubation at room temperature, cells were washed with 200 μL FACS Buffer and resuspended in 100 μL. Data were acquired on an Attune NxT flow cytometer. FSC and SSC parameters were used to specify gates for T cells. pSTAT data was stratified by Myc+ (edited) T cells.

### Results

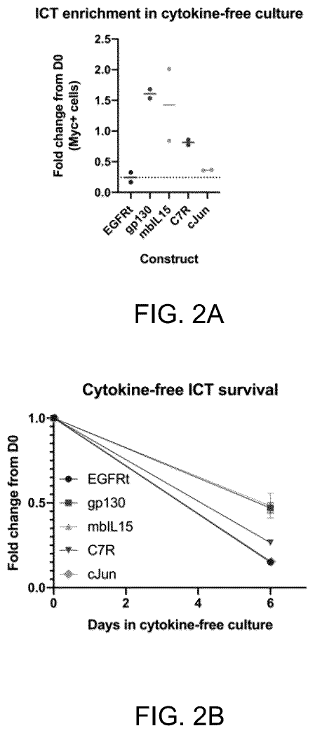

To analyze the ability of SPAs to enhance T cell proliferation in cytokine-free conditions, LG T cells expressing a SPA or control protein were cultured in cytokine-free media (e.g., no extraneous antigen or cytokine stimulation). Knock-in levels and total edited T cell count were compared at Day 0 and Day 6. LG T cells expressing a SPA (L-gp130, mbIL-15, or C7R) exhibited about 3-6-fold enrichment compared to EGFRt or cJun control (FIGS. 2A and 2B). All cell counts ultimately declined, indicating that no transformation occurs, but LG T cells expressing SPAs persisted longer in the absence of antigen stimulation relative to control.

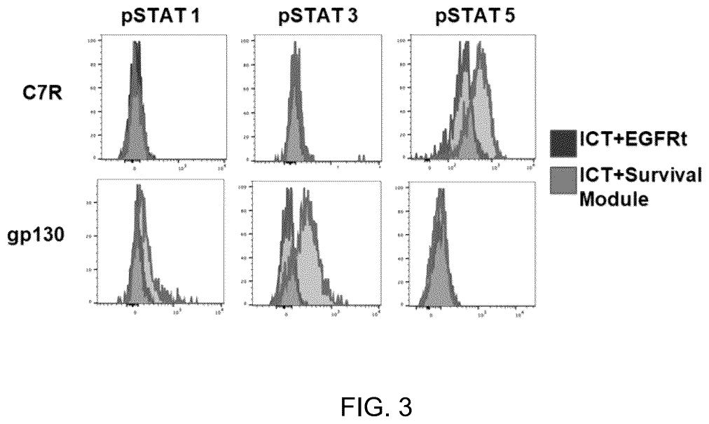

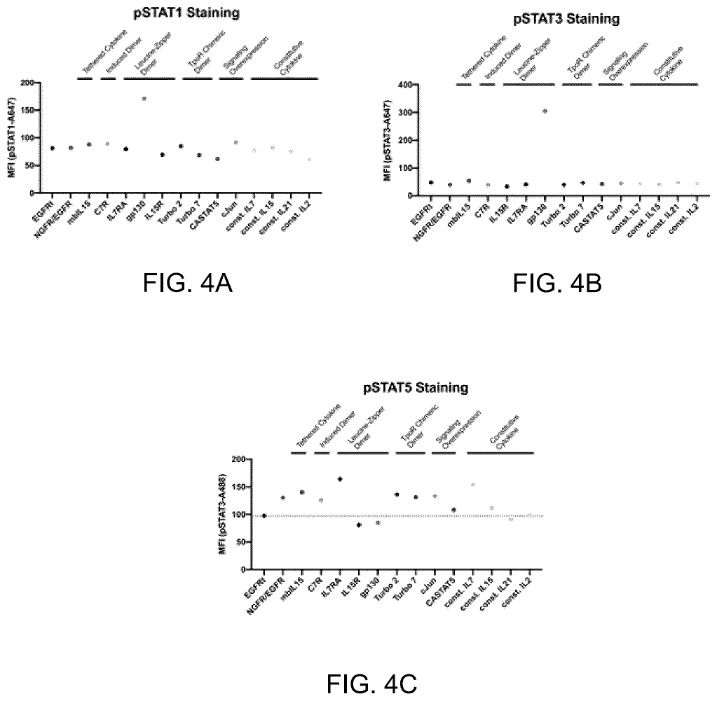

To assess the ability of SPAs to drive cytokine signaling in LG T cells, edited cells were starved for 24 hours with no antigen stimulation or cytokine support, then fixed and stained for pSTAT proteins. LG T cells expressing a SPA exhibited increased phosphorylation of STAT1, STAT3, and STAT5 proteins under baseline (non-stimulated) conditions relative to control (FIG. 3). Activation of STAT phosphorylation under identical conditions using various types of SPAs are also shown (FIGS. 4A, 4B, and 4C). These data indicate that pSTAT activity can be tailored through the use of distinct SPA molecules.

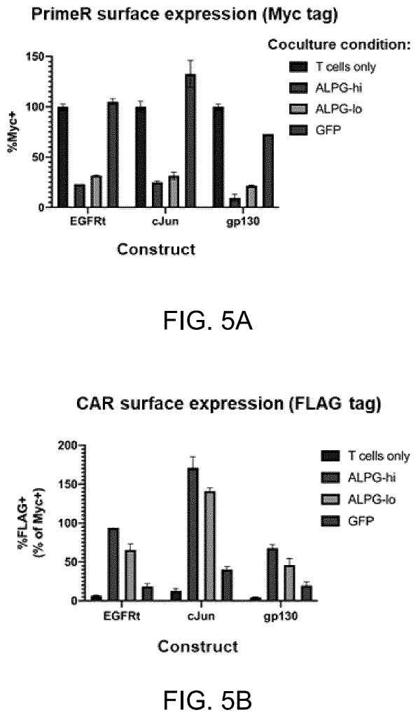

To verify that SPA expression does not alter expression of logic gate proteins, LG T cells were cocultured with K562 tumor cells expressing no cognate antigens (GFP), PrimeR antigen at high concentrations (ALPG-hi), or low concentrations (ALPG-lo). Following a 72 hr incubation, activation of the logic gate was measured by PrimeR (Myc tag) and CAR (FLAG tag) surface expression. L-gp130 LG T cells cocultured with tumor cells demonstrated intact cytolytic CAR logic gate. LG T cells cultured alone or with antigen-negative tumor cells (ALPG-GFP) expressed primeR on the surface (FIG. 5A), but did not express a cytolytic CAR (FIG. 5B). When cocultured with tumor cells expressing the cognate PrimeR antigen (ALPG-hi or ALPG-lo), the LG T cells downregulated PrimeR (FIG. 5A), and upregulated cytolytic CAR (FIG. 5B). These results demonstrate that addition of constitutive accessory molecules (represented in the data by the exemplary SPA L-gp130 and control cJun) to the circuit does not disrupt sensitivity or integrity of the logic gate.

### Example 2: In vitro Assessment of LG T Cells Expressing SPAs

### Materials and Methods

### Endpoint Luciferase Assay

T cells from two donors were engineered to express ALPG/MSLN logic gates (LG) containing genes encoding SPA accessory proteins using the CiTE manufacturing process and were frozen and cryobanked at Day 9 after initial activation. Prior to the assay, engineered T cells and RNP only control T cells from the same donors were thawed and rested overnight in media including 12.5 ng/ml human IL-7 and IL-15. On the day of the assay, engineered T cells were counted and stained for PrimeR and CAR expression using anti-Myc PE and anti-FLAG BV421, respectively, and analyzed by flow cytometry. For each donor, all engineered T cell populations were normalized to the lowest KI % within that donor by adding RNP only cells to dilute engineered populations that were above the lowest KI %. After normalization, T cells were resuspended in medium without IL-7 and IL-15 and serially diluted prior to being added to 96-well flat-bottom, white-walled assay plates. The serial dilution of T cells resulted in the following co-culture KI+ effector: target (E: T) ratios once 1e4 target cells are added/well: 1:1, 1:3, and 1:9, in technical duplicates. Each T cell population was co-cultured with Luciferase+K562 tumor cell lines that either express only CAR antigen (K562-MSLN), or both PrimeR and CAR antigens (ALPG/MSLN). Cytotoxicity at the end of the 72 hour co-culture was measured using an end-point luciferase assay. Following addition of the luciferase substrate directly to the wells data was collected in a PHERAstar plate reader. Data was normalized by setting the emission from wells containing only K562 cells as the baseline, and plotting reduction in luciferase as “percent lysis.”

### Incucyte Assay

T cells from two donors were engineered to express ALPG/MSLN circuits containing genes encoding SPA accessory proteins using the CiTE manufacturing process and were frozen and cryobanked at Day 9 after initial activation. Prior to the assay, engineered T cells and RNP only control T cells from the same donors were thawed and rested overnight in media including 12.5 ng/ml human IL-7 and IL-15. On the day of the assay, engineered T cells were counted and stained for PrimeR and CAR expression using anti-Myc PE and anti-FLAG BV421, respectively, and analyzed by flow cytometry. For each donor, all engineered T cell populations were normalized to the lowest KI % within that donor by adding RNP only cells to dilute engineered populations that were above the lowest KI %. After normalization, T cells were resuspended in medium without IL-7 and IL-15 and serially diluted prior to being added to 96-well flat-bottom, white-walled assay plates. The serial dilution of T cells resulted in the following co-culture KI+ effector: target (E: T) ratios once 1e4 target cells are added/well: 1:1, 1:3, 1:9, and 1:27 in technical duplicates. Each T cell population was co-cultured with GFP+K562 tumor cell lines that express both PrimeR and CAR antigens (ALPG/MSLN). Imaging of GFP and AnnexinV co-culture was conducted throughout a 72 hr. period to derive live target cell and live T cell numbers.

### Continuous Stimulation Assay

T cells from two donors were engineered to express ALPG/MSLN logic gate (LG) in addition to genes encoding SPA accessory proteins using the CiTE manufacturing process. At Day 9 following initial activation, edited cells were isolated by pretreatment with ADAMI0 and Gamma-Secretase inhibitors for 30 minutes followed by incubation with Fc-conjugated recombinant ALPG protein for 30 min on ice. Cells were then spun down, and resuspended in PBS+BSA containing ProteinG Dynabeads. The Cell/Bead mixture was incubated at room temperature for 15 minutes, and then spun down at 400×g for 5 min. The cell/bead fraction was washed 1× in PBS/BSA and resuspended in media including 12.5 ng/ml human IL-7 and IL-15 and seeded in a 24-well GREX plate. Following a 24-hour incubation period cells were debeaded and resuspended in T cell media with IL-7/15. 24 hours later, T cells were counted and stained for PrimeR using anti-Myc PE antibodies and analyzed by flow cytometry to acquire the final percentage of edited cells. LG T cells were then cocultured with RPMI cells expressing both PrimeR and CAR antigens (ALPG/MSLN) at a 1:100 E:T ratio. Cells were cocultured either with or without IL-2 supplementation at 100 IU/mL. Every 2-3 days after the initial coculture setup, media was changed and cells were stained with anti-Myc PE antibodies and measured by flow cytometry with CountBright beads to attain the total number of T cells and RPMI cells/well. Once cell counts were acquired, cell concentration was re-normalized to the initial 1:100 ratio. This process was repeated until all T cells exhibited loss of tumor control or the study was terminated.

### Results

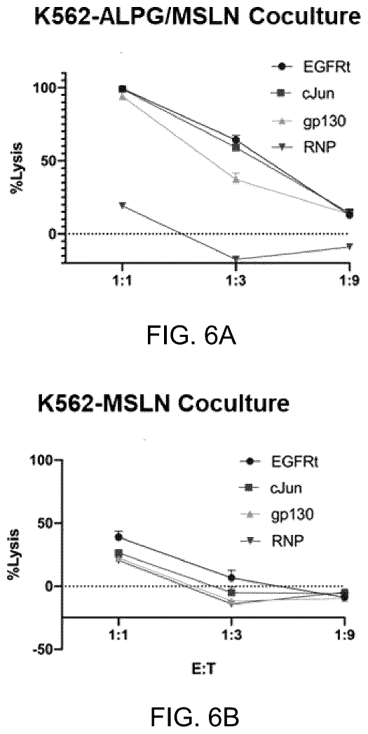

To assess the effects of SPA expression on LG T cell-induced cytotoxicity, LG T cells expressing a SPA or control were cocultured at three different E: T ratios with K562 tumor cells expressing both PrimeR and CAR cognate antigens (ALPG/MSLN), or CAR antigen only (MSLN). Following a 72 hr incubation, tumor cytotoxicity was measured by luciferase activity of the target K562 cells. The results show that LG T cells expressing SPA accessory molecules were able to specifically kill tumor cells expressing both PrimeR and CAR cognate antigens (FIGS. 6A and 6B).

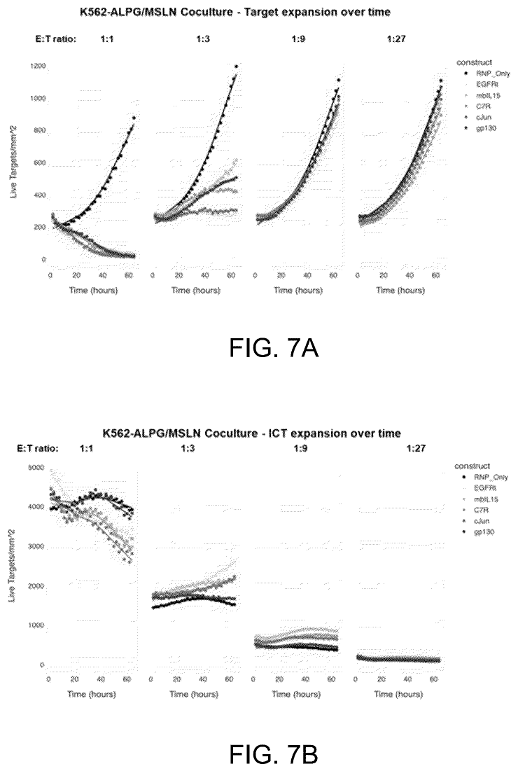

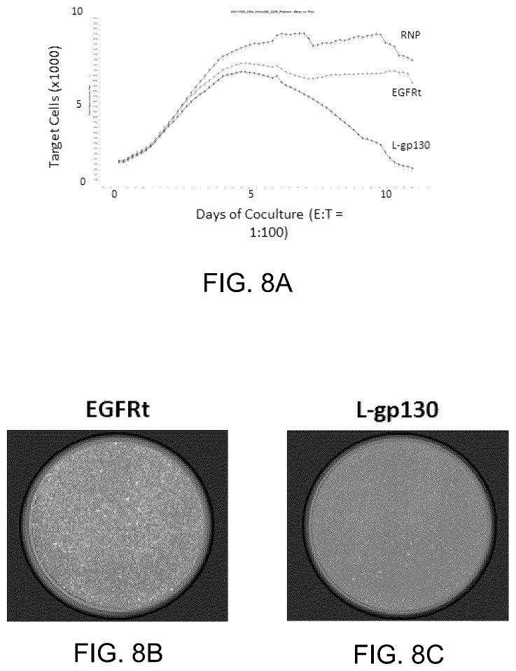

To assess the ability of SPA-expressing LG T cells to sustain cytotoxic effects over an extended period of time, LG T cells expressing a SPA or control protein were cocultured at four different E: T ratios with K562 tumor cells expressing both PrimeR and CAR cognate antigens (ALPG/MSLN). Cocultures were continually imaged throughout a 72-hour period via Incucyte, and T cell and K562 cell counts were derived. LG T cells expressing SPA accessory molecules killed tumor cells at similar levels to an EGFRt control circuit throughout a 72 hr period. (FIG. 7A). LG T cells expressing SPA accessory molecules exhibited similar T cell expansion to an EGFRt control circuit throughout a 72 hr period (FIG. 7B). In a separate experiment, L-gp130-enhanced LG T cells were cocultured with RPMI cells expressing both ALPG and MSLN at a 1:100 E: T. Cocultures were continually imaged throughout a 10-day period via Incucyte, and tumor cell counts were derived. L-gp130 LG T cells demonstrated vastly improved tumor control compared to EGFRt LG T cells, leading to complete tumor clearance (FIG. 8A). Representative well images from the Day 10 are shown (FIGS. 8B and 8C).

### Continuous Stimulation Assay

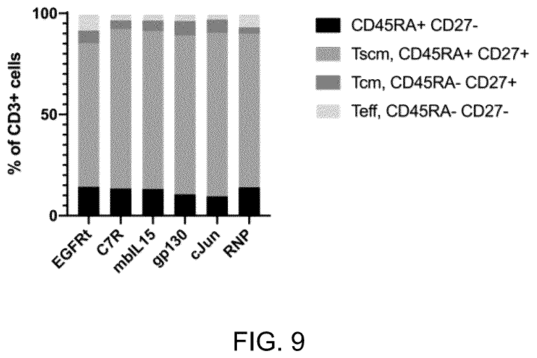

To measure prolonged targeted killing, SPA-expressing LG T cells were subjected to repetitive stimulation with IL-2. Edited LG T cells expressing a SPA or control protein in the absence of antigen stimulation were stained with a panel of antibodies to characterize their cell differentiation state and analyzed by flow cytometry. At baseline levels (in absence of stimulatory antigens), LG T cells expressing SPAs did not exhibit a modified cell differentiation state compared to LG T cells expressing an inert EGFRt molecule (FIG. 9).

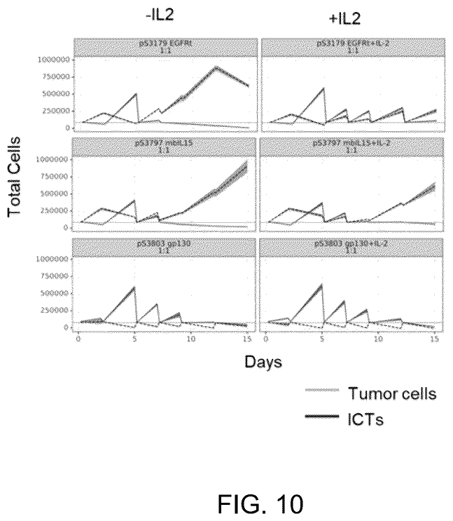

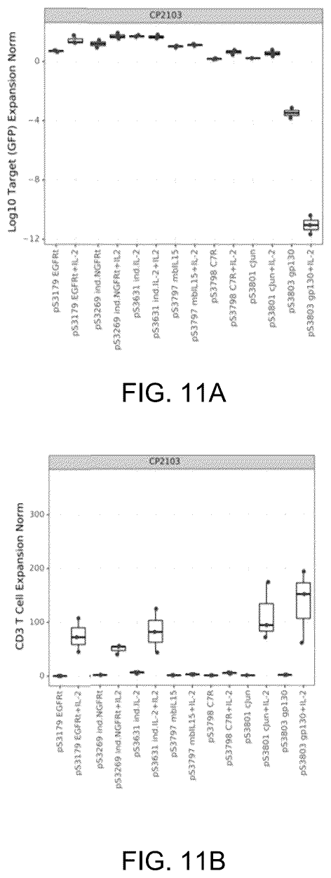

Subsequently, LG T cells and RPMI target cells were cocultured throughout a 15-day continuous stimulation assay with or without IL-2. Cells were re-normalized to a fixed ratio every 2-3 days (1 tumor cells: 100 LG Ts). Prior to each normalization the number of tumor cells and LG T cells were calculated in each well and plotted. L-gp130-expressing LG T cells controlled tumor cell growth throughout the assay period while the EGFRt control LG T cells lost control (FIG. 10). These data indicate L-gp130 LG T cells do not require IL-2 support compared to control tumor cells. Cumulative LG T cell outgrowth throughout a 31-day period of repetitive stimulation shows that L-gp130 LG T cell's ability to control tumor cell growth was vastly superior to a control EGFRt LG T cells. (FIG. 11A). L-gp130 LG T cells expanded at similar levels as control EGFRt LG T cells in the presence of IL-2. In absence of IL-2, LG T cell expansion was superior in L-gp130-expressing LG T cells compared to all other LG T cells tested (FIG. 11B).

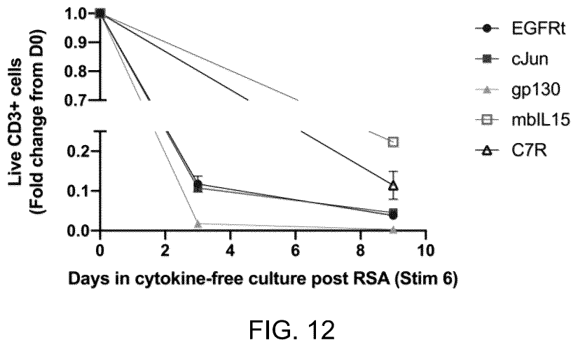

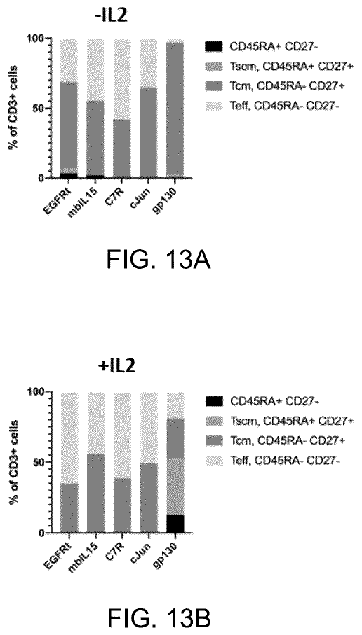

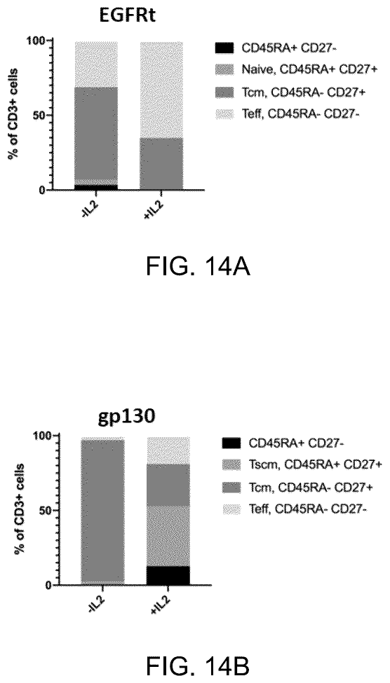

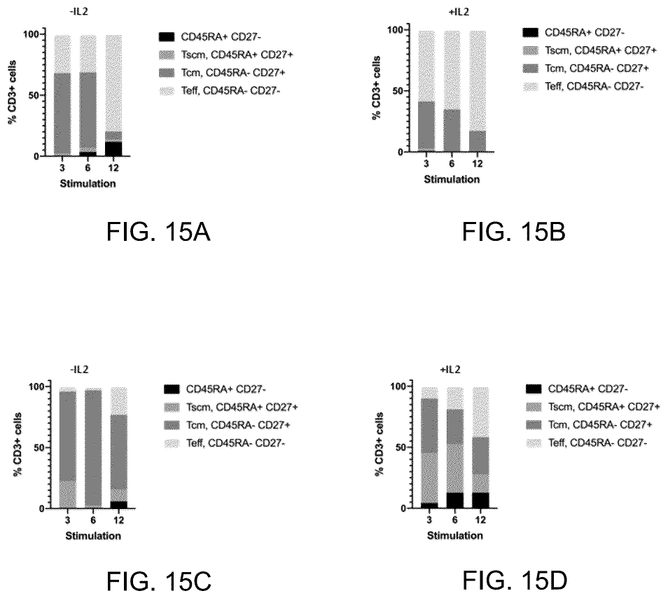

Following 15 days of repetitive stimulation, LG T cells were removed from culture and incubated in cytokine-free media over a nine day period. LG T cell count was measured throughout the incubation period. In absence of supportive cytokines, LG T cells expressing SPAs that have previously been stimulated by tumor antigen did not grow out independently of supportive cytokines or additional antigen stimulation (FIG. 12). Following 15 days of repetitive stimulation in absence or presence of IL-2, T cell differentiation phenotype was assessed by flow cytometry. L-gp130 LG T cells exhibited a more naïve phenotype compared to EGFRt-expressing LG T cells (FIGS. 13A and 13B). The differentiation state of L-gp130-expressing LG T cells was more sensitive to IL-2 supplementation than control LG T cells. (FIGS. 14A and 14B). IL-2 drove differentiation of an effector phenotype in control EGFRt LG T cells while L-gp 130 LG T cells developed a less differentiated stem-memory T cell (Tscm) phenotype. The differentiation state of L-gp130 LG T cells was more sensitive to IL-2 supplementation than control LG T cells (FIGS. 15A-15D).

### Example 3: Modulation of SPA Activity with FAS/PTPN2 Knockdown

### Materials and Methods

pSTAT Signaling

T cells from multiple donors CITE-edited with ALPG/MSLN circuits containing genes encoding SPA accessory proteins as well as shRNA modules targeting FAS and PTPN2 genes, respectively, were profiled for their STAT phosphorylation status by intracellular antibody staining. At day 7 post electroporation, cells were washed and cultured in cytokine-free, serum-free media. Following a 20-hr serum starvation period at 37° C., Zombie-Near Infrared dye was added to the cell media at 1:100 dilution. After a 15 min. incubation, equivalent volume BD Cytofix was added to the cells and the plate was incubated at 37° C. for 15 min. After fixation, cells were spun down at 400×g for 5 min and resuspended in 200 μL of BD Perm Buffer III, and incubated on ice for 30 min. Cells were then washed 2× with BD FACS buffer and resuspended in FACS staining buffer containing anti-Myc PE antibodies at 1:50 (For detecting PrimeR-expressing cells), anti-pSTAT5 A488 at 1:10, and anti-pSTAT3 A647 at 1:10 or anti-pSTATI A647. Following a 30 min incubation at room temperature, cells were washed with 200 μL FACS Buffer and resuspended in 100 μL. Data were acquired on an Attune NxT flow cytometer. FSC and SSC parameters were used to specify gates for T cells. pSTAT data was stratified by Myc+ (edited) T cells.

### Repetitive Stimulation Assay

T cells from two donors were engineered to express ALPG/MSLN logic gate in addition to genes encoding SPA accessory proteins using the CiTE manufacturing process. At Day 9 following initial activation, edited cells were isolated by pretreatment with ADAM10 and Gamma-Secretase inhibitors for 30 minutes followed by incubation with Fc-conjugated recombinant ALPG protein for 30 min on ice. Cells were then spun down, and resuspended in PBS+BSA containing ProteinG Dynabeads. The Cell/Bead mixture was incubated at room temperature for 15 minutes, and then spun down at 400×g for 5 min. The cell/bead fraction was washed 1× in PBS/BSA and resuspended in media including 12.5 ng/ml human IL-7 and IL-15 and seeded in a 24-well GREX plate. Following a 24-hour incubation period cells were debeaded and resuspended in T cell media with IL-7/15. 24 hours later, T cells were counted and stained for PrimeR using anti-Myc PE antibodies and analyzed by flow cytometry to acquire the final percentage of edited cells. T cells were then cocultured with K562 cells expressing both PrimeR and CAR antigens (ALPG/MSLN) at a 1:1 E:T ratio. Every 2-3 days after the initial coculture setup, media was changed and cells were stained with anti-Myc PE antibodies and measured by flow cytometry with CountBright beads to attain the total number of T cells and RPMI cells/well. Once cell counts were acquired, cell concentration was re-normalized to the initial 1:1 ratio. This process was repeated for 14 days.

### Results

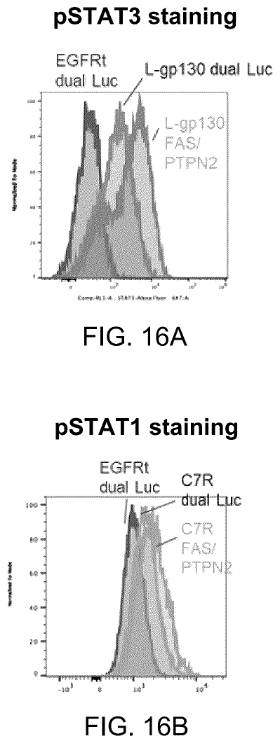

Edited T cells expressing a SPA together with an inert, dual-luciferase targeting shRNA module exhibited pSTAT levels above T cells encoding a gene encoding truncated EGFRt (FIGS. 16A-16B). T cells expressing both a SPA and a FAS/PTPN shRNA module exhibited an additional increase in pSTAT levels. The additive effect was seen in both a STAT3-driving SPA (L-gp130) and a STAT5-driving SPA (C7R).

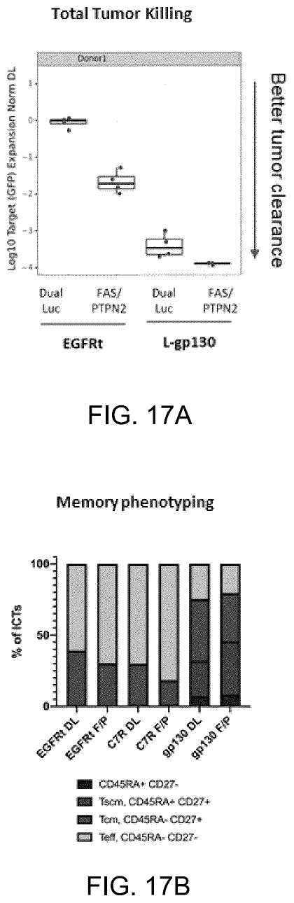

To measure the effect of a Fas/PTPN2 module combined with a SPA on prolonged targeted killing, SPA-expressing T cells with an shRNA module were subjected to repetitive stimulation in IL-2. Edited T cells expressing a SPA or control protein were cocultured with K562 cells at a 1:1 ratio throughout a 14-day repetitive stimulation assay in the presence of IL-2. Cells were re-normalized to a fixed ratio every 2-3 days. Prior to each normalization the number of tumor cells and T cells were calculated in each well and plotted. L-gp130-expressing T cells controlled tumor cell growth throughout the assay period while the EGFRt control T cells lost control (FIG. 17A). Edited T cells expressing the Fas/PTPN2 module exhibited increased antitumor activity with and without a SPA. T cells expressing both an L-gp130 SPA and a Fas/PTPN2 shRNA module demonstrated the most potent killing. Following 15 days of repetitive stimulation, T cell differentiation phenotype was assessed by flow cytometry. L-gp130 T cells exhibited a more naïve phenotype compared to EGFRt-expressing cells, and this trend was preserved in the presence of the Fas/PTPN2 shRNA module (FIG. 17B).

### Example 4: Gene Expression Profiling of LG T Cells Expressing SPAs

### Materials and Methods

### Repetitive Stimulation Assay

ICTs (Integrated circuit T cells) expressing SPAs with or without FAS/PTPN2 modules were challenged in a repetitive stimulation assay as described above. Following 14-days of the RSA, ICTs were separated from target K562 cells by flow cytometry, using PrimeR (Myc) and CAR (Flag) surface expression to distinguish from the target cells.

### ATAC-Seq

ATAC-Seq (Assay for Transposase-Accessible Chromatin with high-throughput sequencing) was carried out according to a previously published report (Corces et al. Nature Methods 2017). Briefly, 50,000 viable cells were centrifuged at 500 RCF at 4° C. for 5 min. To isolate nuclei, the cell pellet was resuspended in 50 μL cold ATAC-Resuspension Buffer (RSB) containing 0.1% NP40, 0.1% Tween 20, and 0.01% Digitonin and incubated on ice for 3 minutes. Then, 1 mL of cold ATAC-RSB containing 0.1% Tween-20 only was added. The isolated nuclei were centrifuged at 500 RCF for 10 min at 4° C. and the supernatant was removed carefully. For transposition of accessible chromatin genomic regions, the nuclei pellet was resuspended in 25 μL 2× TD buffer, 100 nM final Tn5 transposase, 16.5 μL PBS, 0.5 μL 1% digitonin, 0.5 μL 10% Tween-20 and incubated at 37° C. for 30 minutes with 1000 RPM shaking. The transposition reaction was cleaned up using a Zymo DNA Clean and Concentrator-5 Kit and the cluted transposed DNA was amplified using Illumina barcoded Nextera primers for 5 cycles. Quantitative PCR (qPCR) was performed on 10% of the first PCR reaction to determine additional PCR cycles to amplify the barcoded libraries without saturation. Additional PCR cycles were performed to achieve final ATAC-Seq libraries. Typical number of PCR cycles for 50,000 cells is about 12 cycles. ATAC-Seq libraries were then quantified and qualified on the Agilent Tapestation D5000 assay focusing on the size range of 200 bp-1000 bp. ATAC-Seq libraries were pooled and sequenced on the Illumina NovaSeq 6000 SP flowcell (v1.5) at paired-end 150 bp, index 1 and 2 at 8 bp for dual-indexed samples. Each ATAC-Seq libraries were targeted to be sequenced for 20-30M reads. The fastq files were generated and demultiplexed using bc12fastq script (v2.20.0.422). ATAC-seq reads were first trimmed of adapter sequences using cutadapt (v3.5) and then aligned to GRCh38 with BWA (0.7.17). PCR duplicates were identified using Picard's (v2.26.10) MarkDuplicates procedure and removed prior to downstream analysis. ArchR (v1.0.1) was used to count pileup of ATAC-seq reads across the genome and producc plots of ATAC peaks.

### RNAseq

For bulk RNA isolation, RNA was isolated using RNEasy Plus Micro kit following manufacturer's protocol. RNA was quantified using Qubit RNA HS Assay Kit, and RNA quality was assessed using Tape station instrument with RNA screen tape. RNA libraries were prepared using the TruSeq RNA library prep kit v2 (Illumina). 100 ng of RNA samples were used unless limited by sample concentration where the totality of RNA samples were used (≤50 ul) for library prep, following supplier's protocol. Library concentrations were normalized to 2 nM and pooled. Sequencing was performed using Novaseq 6000 instruments using 100/200 cycle Novaseq SP Kit (Illumina). RNA-seq reads were aligned to GRCh38 using STAR (v2.7.7a). STAR was additionally used to quantify gene expression. Normalization and differential expression tests were performed using edgeR (3.28.1) with a cutoff of FDR<0.05 with model coefficients for donor and collection day added in addition to the presence/absence of gp130.

### Results

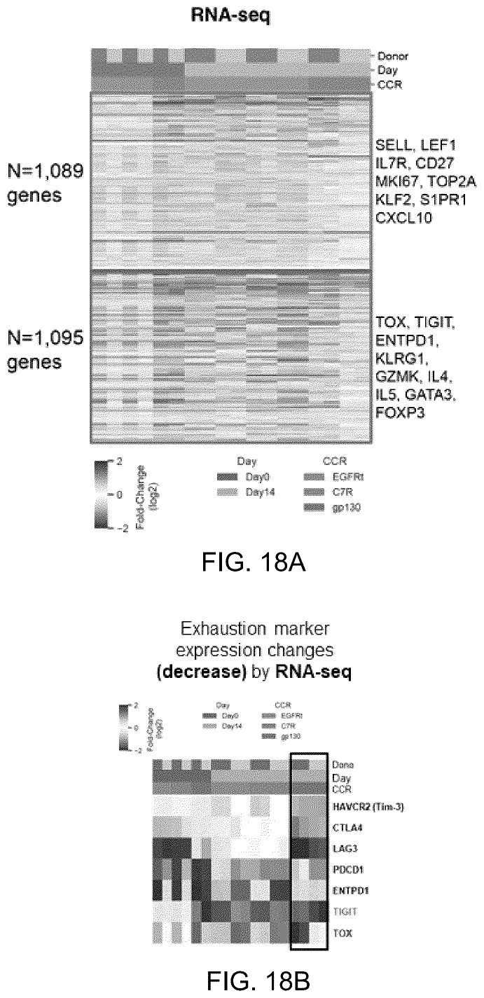

RNAseq analysis identified over 2000 genes that were differentially expressed in gp130 ICTs compared to control EGFRt ICTs following the 14-day repetitive stimulation assay (FIG. 18A). The differentially regulated genes represent various categories, including checkpoint molecules, cell-surface receptors, T cell memory and exhaustion markers, as well as other notable T cell transcription factors (FIG. 18B). A noteworthy trend in the data is that gp130 ICTs at Day 14 more closely resemble Day 0 samples than the other groups tested.

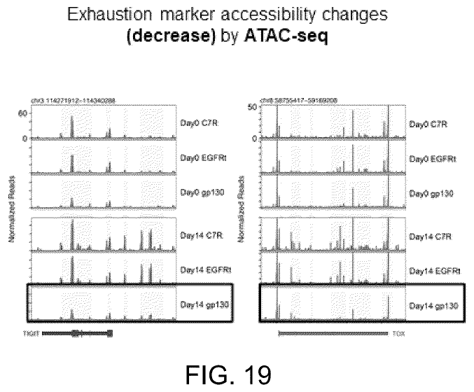

ATAC-seq analysis revealed differential chromatin accessibility across various loci in gp130 ICTs compared to EGFRt or C7R ICTs (FIG. 19). Logic gate ICTs expressing L-gp130 were also analyzed by ATAC-seq to evaluate chromatin accessibility changes of two exhaustion markers, TIGIT and TOX, at day 0 and after a 14 day repetitive stimulation assay. ICTs expressing L-gp130 displayed restricted accessibility of key exhaustion markers TIGIT and TOX (FIG. 19), indicating that L-gp130 expression preserved a stem T cell phenotype. Some specific examples include diminished transcriptional activity at the TIGIT and TOX loci, indicated by reduced reads across those gene loci. This reduction in transcription is corroborated by reduced detection of those transcripts at the RNA level by bulk RNA seq analysis.

### Example 5: In vivo Characterization of LG T cells Expressing SPAs

### Materials and Methods

Characterization of mbIL-15- or C7R-Expressing LG T Cells

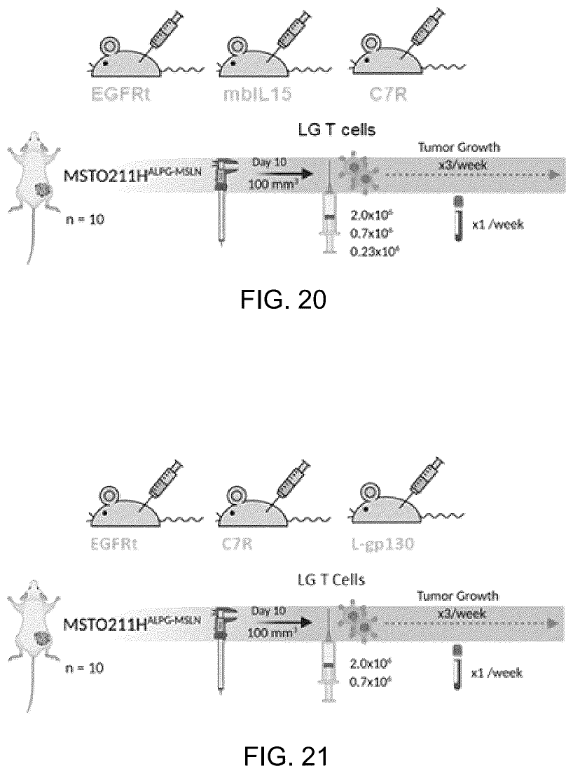

Details of the in vivo interrogation of LG T cells expressing SPAs are detailed in FIG. 20. NSG MHC-DKO mice (Jackson Laboratories, 025216) were engrafted subcutaneously with 1×10⁶ MSTO cells expressing ALPG and MSLN antigens. (n=6-10/arm). When tumors reached 100 mm3, LG T cells, prepared as detailed above, were administered IV. Tumors were measured 3 times weekly by caliper and peripheral blood was drawn once weekly for PK studies. LG T cells equipped with mbIL-15 or C7R were compared to LG T cells expressing control EGFRt. Tumor volume was measured by caliper along with body weight. Cell counting was performed as in Example 3 above.

### Characterization of L-gp130- or C7R-Expressing LG T Cells

Details of the in vivo interrogation of LG T cells expressing SPAs are detailed in FIG. 21. NSG MHC-DKO (Jackson Laboratories, 025216) mice were engrafted subcutaneously with 1×10⁶ MSTO-211H cells expressing ALPG and MSLN antigens (n=10/arm). When tumors reached 100 mm³, LG T cells, prepared as detailed above, were administered IV. Tumors were measured 3 times weekly by caliper and peripheral blood was drawn once weekly for PK studies. LG T cells equipped with C7R or L-gp130 were compared to LG T cells expressing control EGFRt/NGFRt molecules. Tumor volume was measured by caliper along with body weight. Cell counting was performed as in Example 3 above.

### Characterization of L-gp130- or C7R-Expressing LG T Cells in a Renal Cell Carcinoma Model

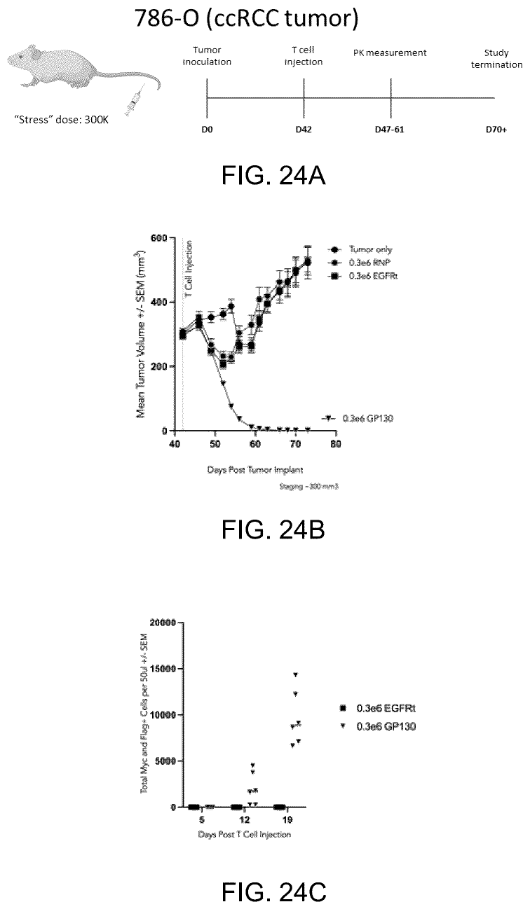

Details of the in vivo interrogation of LG T cells expressing SPAs are detailed in FIG. 24A. To evaluate the therapeutic effects of ICTs, 6-7 week old NSG MHC DKO female mice were subcutaneously injected on the right dorsal flank with 2e6 cells of 786-O-EFG-CA9-ALPG/MSLN_MCB tumor cells in 50% Sigma-Aldrich Extracellular Matrix E1270 to PBS suspension. On day 10 mice were checked for palpable tumors. Day 32 onwards, mice were measured twice a week until they demonstrated the mean tumor volume of approximately 300 mm³. On day 42 post tumor implantation, mice reached the average tumor volume of 299 mm³ and were randomized into 10 groups consisting of 7-8 mice. Following randomization, the T cells were thawed and rested overnight for intravenous injection (I.V.). On day 42, the T cells were harvested and normalized for I.V. injections. The T cells were suspended in 100 μL of PBS at doses of 0.3e6 and 0.05e6. Following treatment, mice were monitored twice a week for tumor volumes, body weights and clinical observations. Each mouse was bled on days 5, 12, 19, 40 post treatment and 100 μL of blood was collected via submandibular vein. Blood was processed for immune phenotyping of ICTs and Total T cells and edited T cells were quantified.

### Results

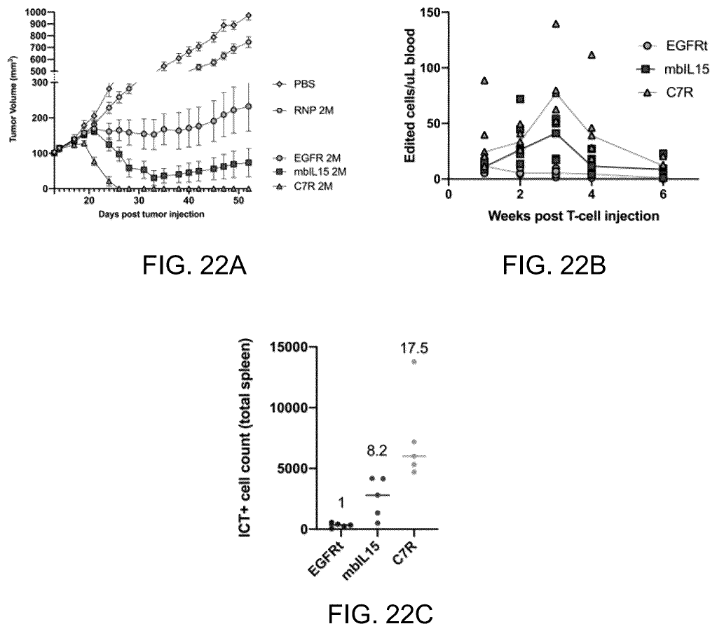

SPAs were compared with control EGFRt in LG T cells. C7R-expressing LG T cells achieved a complete response (CR) in 100% of mice, which was a ˜9-fold higher rate than EGFRt-expressing cells (3/10). mbIL-15-expressing LG T cells achieved a CR in 7/10 mice, which was a ˜5-fold higher rate than EGFRt-expressing cells (FIG. 22A). CR results of all groups are provided in Table E. Anti-tumor efficacy correlated with LG T cell expansion detected in the blood, with eventual contraction of the LG T cells (FIG. 22B). At experiment endpoint splenic LG T cells were isolated and enumerated. Total edited cell count was plotted and the fold-increase in SPA LG T cells compared to the EGFRt LG T cell control group is indicated (FIG. 22C).

Table E shows CR results in mice treated with LG T cells expressing indicated SPA molecule or control

TABLE E

| Group | CR |
|---|---|
| PBS | 0/10 |
| RNP | 0/10 |
| EGFRt | 3/10 |
| mbIL15 | 7/10 |
| C7R | 8/8 |

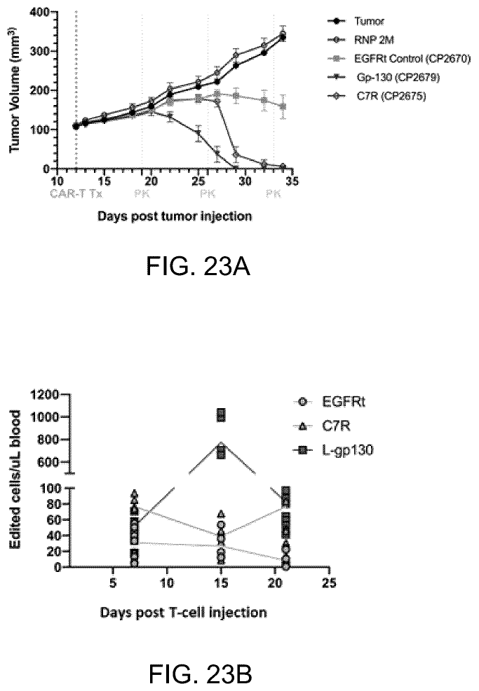

L-gp130 and C7R-expressing LG T cells cleared tumors better than EGFRt LG T cells, with 100% CR in all mice compared to 0% in control mice (FIG. 23A). Anti-tumor efficacy correlated with LG T expansion detected in the blood (FIG. 23B).

786-O-engrafted mice treated with ICTs expressing the L-gp130 SPA controlled tumor better than mice treated with control ICTs expressing EGFRt, with 10/10 mice achieving CR versus 0/10 in the control group (FIG. 24B). Blood PK demonstrated improved proliferation in gp130-expressing T cells, with maximum expansion observed at 19-days post T-cell injection, while 0/6 mice examined from the control group had detectable edited T cell levels in the blood (FIG. 24C). The data demonstrate improved tumor clearance by SPA-equipped ICTs in an alternative cancer model (Renal Cell Carcinoma).

### Example 6: In vitro Analysis of LG T Cells with Inducible Cytokine Expression

### Materials and Methods

### Analysis of Cytokine Secretion

T cells were engineered to express the ALPG priming receptor (primeR) (SEQ ID NO: 24) and MSLN CAR (SEQ ID NO: 31), as well as inducible expression of IL-2 (SEQ ID NO: 74) upon activation of the primeR (“LG T cells”). Engineered T cells were co-cultured with K562 target cells expressing no antigen, primeR antigen (ALPG) only, or both primeR and CAR antigens (MSLN/ALPG) for 72 hours. Supernatants were collected for cytokine quantification by Milliplex MAP Human High Sensitivity T cell Panel Premixed 21-plex.

### Endpoint Luciferase Assay

T cells were engineered to express the ALPG priming receptor (primeR) (SEQ ID NO: 24) and MSLN CAR (SEQ ID NO: 31), as well as inducible expression of IL-2 (SEQ ID NO: 86) upon activation of the primeR. Engineered T cells, T cells expressing only the logic gate, and RNP-only control cells were co-cultured with K562 target cells expressing no antigen or both ALPG and MSLN for 72 hours. T cells were resuspended in medium and serially diluted prior to being added to 96-well flat-bottom, white-walled assay plates. The serial dilution of T cells resulted in the following co-culture effector: target (E: T) ratios once 1×10⁴ target cells are added/well: 1:1, 1:3, and 1:9, in technical duplicates. Each T cell population was co-cultured with Luciferase+ K562 tumor cell lines that either express no antigen or both PrimeR and CAR antigens (ALPG/MSLN). Cytotoxicity at the end of the 72 hour co-culture was measured using an end-point luciferase assay. Following addition of the luciferase substrate directly to the wells, data was collected in a PHERAstar plate reader. Data was normalized by setting the emission from wells containing only K562 cells as the baseline, and plotting reduction in luciferase as “percent lysis.”

### Repetitive Stimulation Assay

T cells from two or three donors were engineered to express the ALPG priming receptor (primeR) (SEQ ID NO: 24) and MSLN CAR (SEQ ID NO: 31), as well as inducible expression of IL-2 (SEQ ID NO: 86) upon activation of the primeR with two different orientations of cytokine placement within the expression construct: payload-2A (self-cleaving peptide)-CAR or CAR-2A-payload. LG T cells were co-cultured in media containing 12.5 ng/ml human IL-7 and IL-15. Seven days after electroporation, logic gate CAR T cells were stained for PrimeR and CAR expression using anti-myc PE and anti-Flag APC respectively, and analyzed by flow cytometry. Leakiness (antigen independent expression of CAR) is calculated by dividing the % CAR positive population by the total primeR+ population.

### Repetitive Stimulation Assay

T cells were engineered to express the ALPG priming receptor (primeR) (SEQ ID NO: 24) and MSLN CAR (SEQ ID NO: 31), as well as inducible expression of IL-2 (SEQ ID NO: 86) or Super-2 (SEQ ID NO: 132) upon activation of the primeR. Edited cells were co-cultured with K562 target cells expressing PrimeR and CAR antigens (MSLN/ALPG) at a 1:1 E:T ratio. Control cells that were not engineered with inducible IL-2 expression were cultured in medium containing exogenous IL-2 or control medium. Following a 2-3 day co-culture period, T cell media was changed and cells were stained with anti-Myc PE antibodies and measured by flow cytometry with CountBright beads to attain the total number of T cells and RPMI cells/well. Once cell counts were acquired, cell concentration was re-normalized to the initial 1:1 ratio. This process was repeated over a 14-day period.

### Results

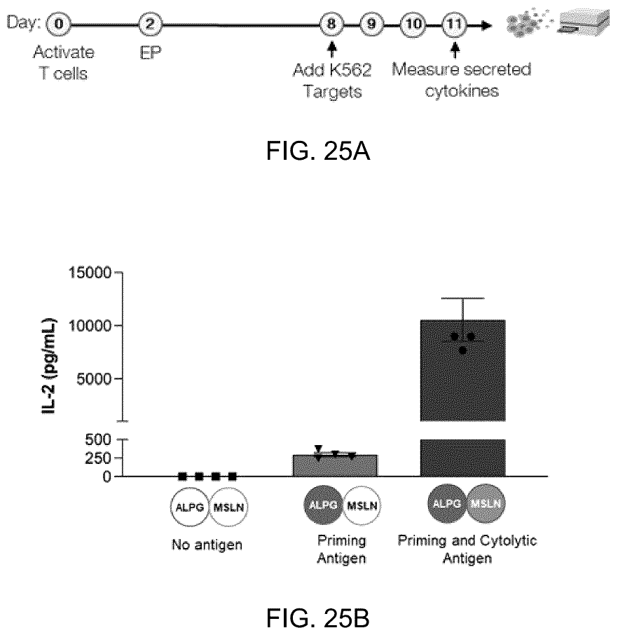

LG T cells were engineered to have inducible IL-2 expression upon stimulation of primeR. Engineered cells were co-cultured with targets cells expressing no antigen, primeR antigen (ALPG), or both primeR and CAR antigens (ALPG/MSLN) at a 1:1 E: T Ratio (FIG. 25A). After a 72-hour co-culture, the levels of IL-2 secretion after engagement of the priming antigen indicated that cytokine secretion was induced by activation of the primeR. Furthermore, co-culture with both primeR and CAR antigens enhanced IL-2 secretion (FIG. 25B). This indicates that inducible cytokine expression in LG T cells can provide a method of delivering high levels of cytokine payloads to the tumor microenvironment.

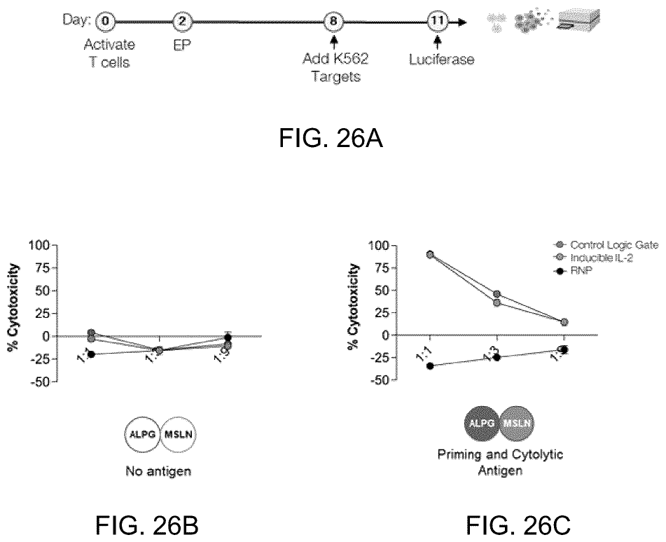

To assess the effects of inducible cytokine expression on the function of logic gate-expressing T cells, LG T cells were engineered to have inducible IL-2 expression upon stimulation of the priming receptor primeR or control. Both groups of engineered T cells, as well as RNP-only control cells, were incubated over 72 hours with target cells expressing no antigen or both primeR and CAR antigens (FIG. 26A). Cytotoxicity of target cells expressing no antigen was not observed for LG1 T cells with inducible IL-2 expression, control LG T cells, or RNP-only T cells (FIG. 26B). However, both control LG T cells and LG T cells with inducible IL-2 expression induced dose-dependent cytotoxicity of target cells expressing both primeR and CAR antigens (FIG. 26C). These results indicate that the addition of inducible cytokine expression to LG T cells does not impair their anti-tumor activity.

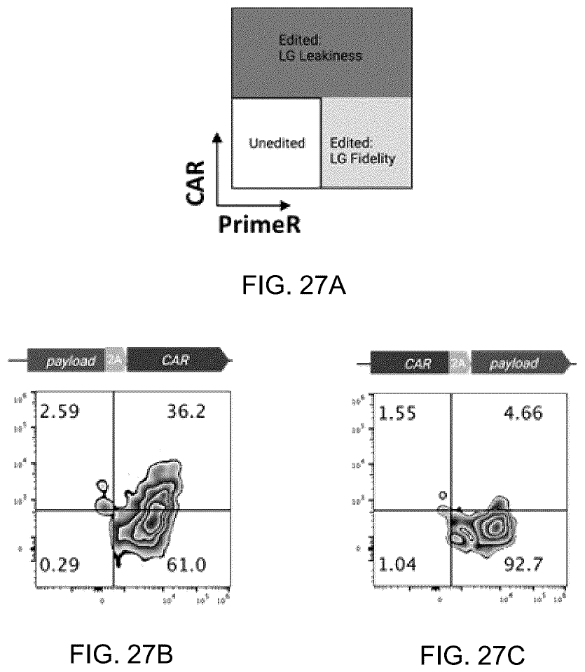

To assess the effects of the orientation of the cytokine with respect to the CAR, LG T cells were engineered to express the cytokine either immediately upstream or downstream of the CAR. In the absence of priming antigen (ALPG), all of the T cells should be either unedited (PrimeR-CAR−) or PrimeR+ CAR−. Leakiness is defined as the priming antigen independent expression of CAR+ T cells (FIG. 27A). When the payload is placed upstream of the CAR (payload-2A-CAR), there is an increase in the leakiness of the SSI logic gate (FIG. 27B) compared to the orientation in which the payload is placed downstream of the CAR (CAR-2A-payload) (FIG. 27C).

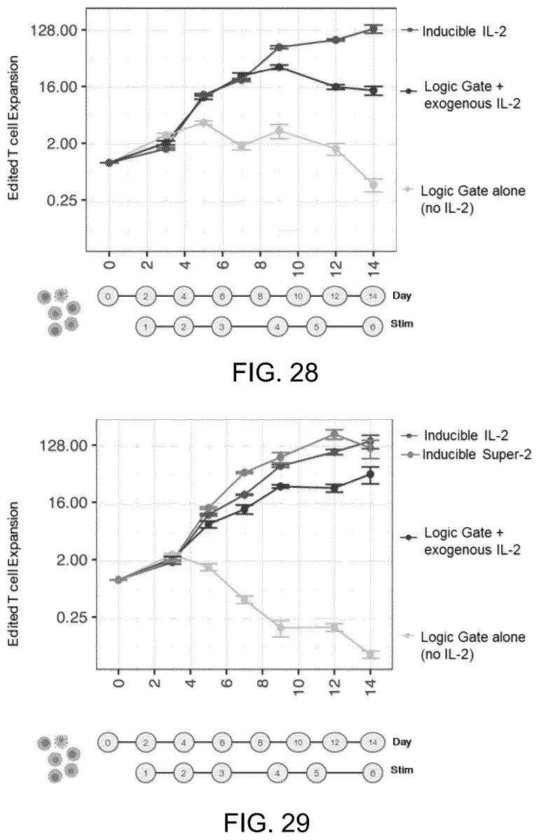

To assess whether inducible cytokine expression can enhance the activity of LG T cells, LG T cells were engineered to have inducible IL-2 expression or control upon stimulation of primeR. LG T cells were then repeatedly stimulated with target cells expressing primeR and CAR antigens every 2-3 days over a 14 day period, and cell counts were taken continuously. Control LG T cells were also incubated in the presence of exogenous IL-2 or control medium. LG T cells with inducible IL-2 expression showed the highest degree of expansion over the 14-day stimulation, with control cells incubated with exogenous IL-2 also showing expansion over the course of the experiment (FIG. 28). By contrast LG T cells stimulated in the absence of cytokines did not substantially expand over the course of the stimulation. Furthermore, inducible expression of “Super-2,” a modified IL-2 variant that has enhanced activation of IL-2 receptors, showed similar effects on LG T cell expansion as wild-type IL-2 in a separate experiment (FIG. 29). These results indicate that inducible cytokine expression enhances T cell persistence under constant antigen stimulation, which can be similar to the conditions present in the tumor microenvironment.

### Example 7: Assessment of Tunability of Inducible Cytokine Expression

### Materials and Methods

### Analysis of Cytokine Secretion

T cells were engineered to express the ALPG priming receptor (primeR) (SEQ ID NO: 24) and MSLN CAR (SEQ ID NO: 31), as well as to have inducible expression of IL-2 (SEQ ID NO: 86), IL-12/23p40 (SEQ ID NO: 94), IL-18 (SEQ ID NO: 98), or IL-21 (SEQ ID NO: 90) upon activation of primeR. Engineered T cells were co-cultured with K562 target cells expressing no antigen, primeR antigen (ALPG) only, or both PrimeR and CAR antigens (MSLN/ALPG) for 72 hours. Supernatants were collected for cytokine quantification by Milliplex MAP Human High Sensitivity T cell Panel Premixed 21-plex.

### Repetitive Stimulation Assay

T cells were engineered to express the ALPG priming receptor (primeR) (SEQ ID NO: 24) and MSLN CAR (SEQ ID NO: 31) in addition to inducible expression of IL-2 (SEQ ID NO: 86), IL-12/23p40 (SEQ ID NO: 94), IL-18 (SEQ ID NO: 98), or IL-21 (SEQ ID NO: 90). Edited cells were co-cultured with K562 target cells expressing primeR and CAR antigens (MSLN/ALPG) at a 1:1 E:T ratio. Control cells that were not engineered with inducible IL-2 expression were cultured in medium containing exogenous IL-2 or control medium. Following a 2-3 day co-culture period. T cells, media was changed and cells were stained with anti-Myc PE antibodies and measured by flow cytometry with CountBright beads to attain the total number of T cells and RPMI cells/well. Once cell counts were acquired, cell concentration was re-normalized to the initial 1:1 ratio. This process was repeated over a 14-day period.

### Analysis of Signal Peptide Effects on Cytokine Secretion

Constructs were prepared to operably link IL-7 (SEQ ID NO: 88) to signal peptides from IgKVIII (SEQ ID NO: 118), CD44 (SEQ ID NO: 100), tPA (SEQ ID NO: 130), trypsinogen (SEQ ID NO: 114), CD5 (SEQ ID NO: 104), IL-2 (SEQ ID NO: 108), secrecon (SEQ ID NO: 128), CD3E (SEQ ID NO: 102), IgE (SEQ ID NO: 120), OSM (SEQ ID NO: 122), ITGAL (SEQ ID NO: 106). GMCSF (SEQ ID NO: 110). chymotryspinogen (SEQ ID NO: 112), IgK (SEQ ID NO: 116), IgG2H (SEQ ID NO: 124), or BM40 (SEQ ID NO: 126). LG1 T cells were engineered to have inducible expression of IL-7 with each of the signal peptides upon activation of primeR. Engineered T cells were co-cultured with K562 target cells expressing both ALPG and MSLN for 72 hours. Supernatants were collected for cytokine quantification by Milliplex MAP Human High Sensitivity T cell Panel Premixed 21-plex.

### Results

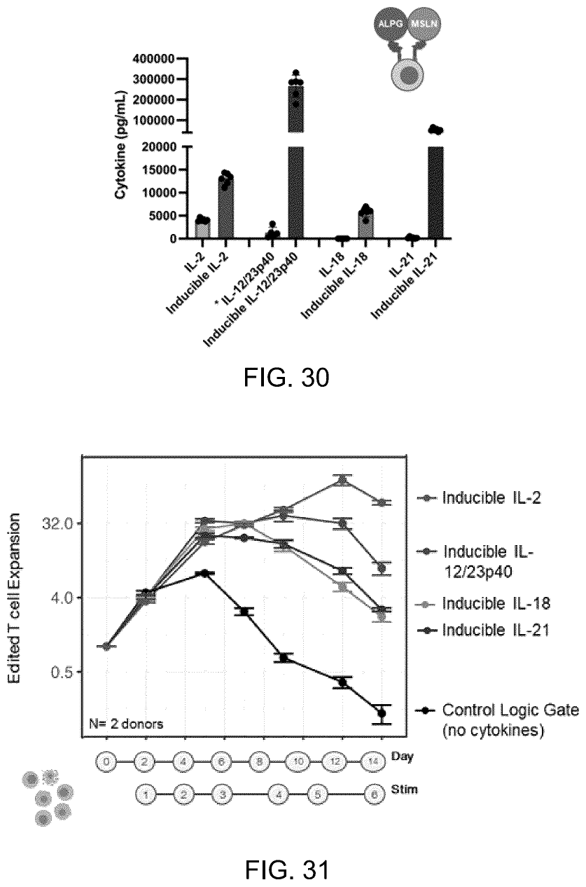

LG T cells were engineered to have inducible expression of IL-2, IL-12/23p40, IL-18, or IL-21 upon activation of primeR. Engineered cells were co-cultured with target cells expressing both primeR and CAR antigens. Robust inducible secretion was observed for all cytokines as compared to control LG T cells (FIG. 30). Furthermore, LG T cells with inducible expression of each of these cytokines showed enhanced expansion over 14 days of repetitive stimulation as compared to control LG T cells (FIG. 31). These results indicate that a wide variety of cytokines can be inducibly expressed by LG T cells to elicit a variety of desirable effects on the T cells.

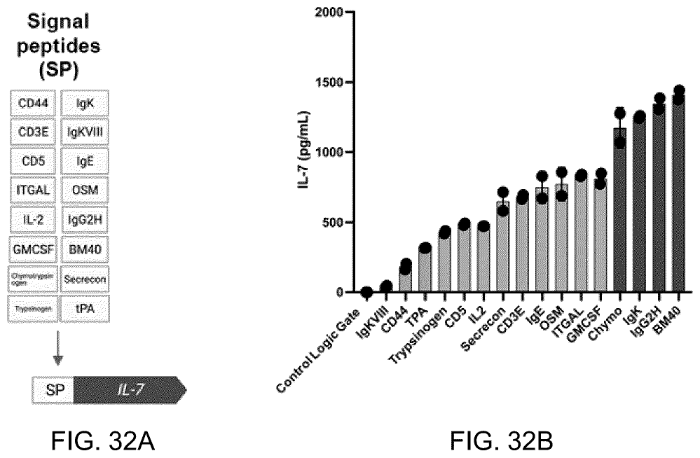

To further assess the tunability of inducible cytokine expression, IL-7 was engineered to be linked to a variety of non-native signal peptides (FIG. 32A). LG T cells expressing each of these constructs with inducible expression upon stimulation of primeR were created. Engineered cells expressing each of these constructs were co-cultured with target cells expressing both primeR and CAR antigens. Levels of secreted IL-7 were shown to vary based on the specific signal peptide used (FIG. 32B). Signal peptides from IgGKVIII and CD44 yielded the lowest relative levels of secreted IL-7, whereas BM40 and IgG2H yielded the highest IL-7 relative secretion levels. These results indicate that inducible cytokine secretion (and subsequent activation of signaling) can be tuned by selection of particular non-native signal peptides.

### Example 8: In vivo Characterization of LG T Cells with Inducible Cytokine Expression

### Materials and Methods

Female 6 to 7-week-old NSG MHC I/II DKO mice (Jax) were implanted subcutaneously with 1×10⁶ MSTO-211H-MSLN-ALPG cells in 50% Matrigel. Mice were staged at tumor volumes of approximately 80-150 mm³. 2×10⁶ of T cells expressing the ALPG priming receptor (primeR) (SEQ ID NO: 24) and MSLN CAR (SEQ ID NO: 31) with inducible expression of EGFRt control (SEQ ID NO: 84), IL-2 (SEQ ID NO: 86), IL-7 (SEQ ID NO: 88), or IL-21 (SEQ ID NO: 90) upon activation of the primeR were injected via the tail vein. Tumor volumes were measured via caliper over the course of the study. 100 μL of whole blood from the mice were collected by retro-orbital bleeding 7, 14, 21, 28 and 42 days post LG T cell administration and T cell titers were measured by flow cytometry.

### Results

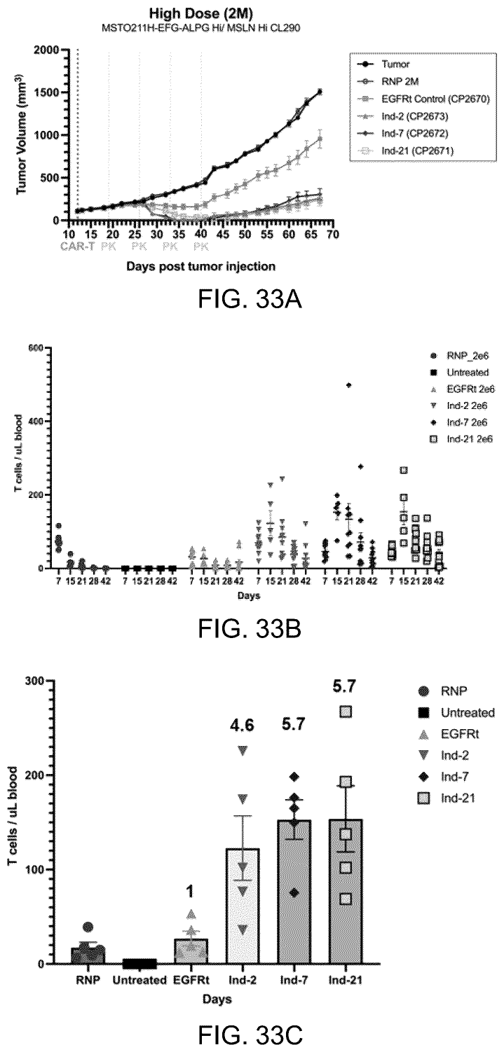

Tumor-bearing mice treated with RNP-only control T cells, EGFRt control-expressing LG T cells, and LG T cells with inducible expression of IL-2, IL-7 or IL-21 were assessed for their anti-tumor responses. Mice treated with LG T cells inducibly expressing any of the cytokines showed robust suppression of tumor growth, whereas mice treated with LG T cells expressing EGFRt control showed more a relatively more limited suppression of tumor growth, and mice treated with RNP-only T cells showed no improvement in tumor suppression compared to no-treatment control (FIG. 33A). Additionally, LG T cells expressing any of the inducible cytokines showed increased expansion compared to control T cells (FIG. 33B). Peak T cell expansion was observed at 15-days after treatment, where LG T cells inducibly expressing IL-2, IL-7, or IL-21 showed 4.6-fold, 5.7-fold, and 5.7-fold increased expansion, respectively, over LG T cells expressing EGFRt control (FIG. 33C). These results indicate that combining inducible expression of cytokines with the primeR/CAR logic gate leads to enhanced anti-tumor activity of LG T cells relative to controls.

### Example 9: High-Throughput Screening of LG T Cells Expressing a Combination Including a Suppressor of Gene Expression and an SPA and/or a Cytokine

### Materials and Methods

### Editing and Normalization of T Cells:

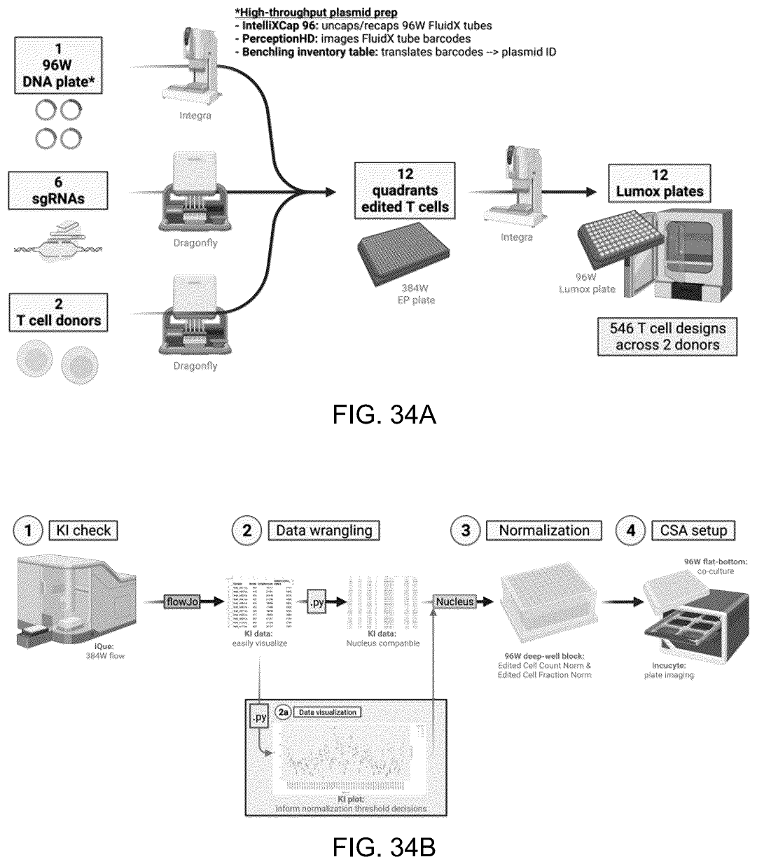

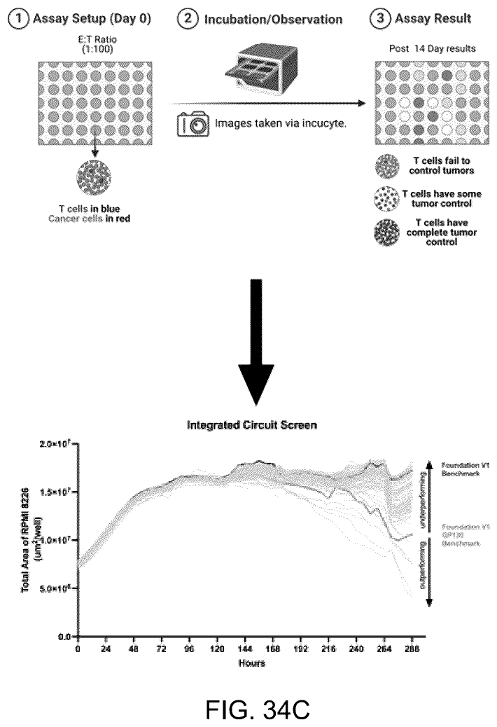

To generate a library of T cells for screening T cells from 2 donors were incubated in a 384-well plate with plasmids encoding the CAR, PrimeR, and combinations including a synthetic pathway activator, a cytokine, and an sgRNA for gene silencing. A diagram of the production of edited cells is shown in FIG. 34A. Edited cells were stained with Ki-67 to measure cell count. Ki-67 data was wrangled and cells were normalized for both overall count and fraction of edited cells prior to commencement of the continuous stimulation assay. A detailed schematic of the normalization workflow is shown in FIG. 34B.

### Continuous Stimulation Assay:

To evaluate the cytolytic and proliferative capacity of the engineered T cells, continuous stimulation assay was performed. Briefly, the engineered T cells and tumor cells were seeded at 1:100 (250 edited T cells: 25K RPMI 8226 target cells) with complete RPMI medium (RPMI 1640+glutamax, 10% fetal bovine serum, 15 ng/ul gentamicin). Media exchange occurred in every co-culture 3 times a week (every 2-3 days) with the RPMI medium. A split and re-challenge was performed at day 12 of co-culture. To split the co-culture, each well was mixed to achieve a homogenous mixture and 25% of the volume (50 ul) was acquired and replated to a new plate. To re-challenge the cells, 25K tumor cells were introduced to the new plate containing the split co-culture cells. The assay was concluded at 14 days of co-culture and target cell count and T cell count were measured via Incucyte using the manufacturer's protocol. A detailed schematic of the continuous stimulation assay setup and output is shown in FIG. 34C.

### Results

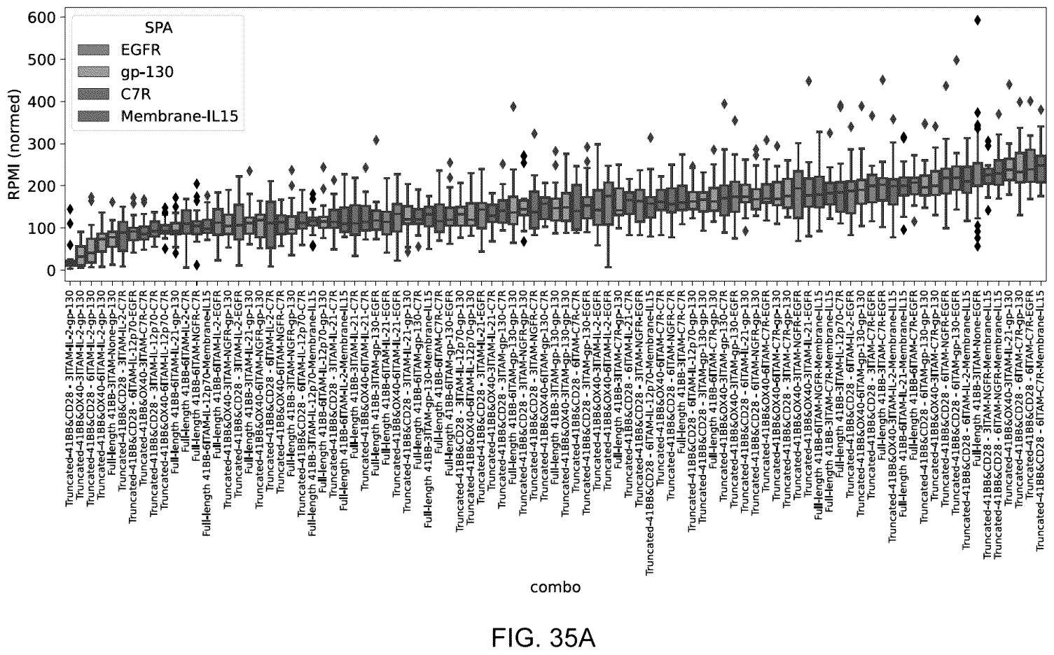

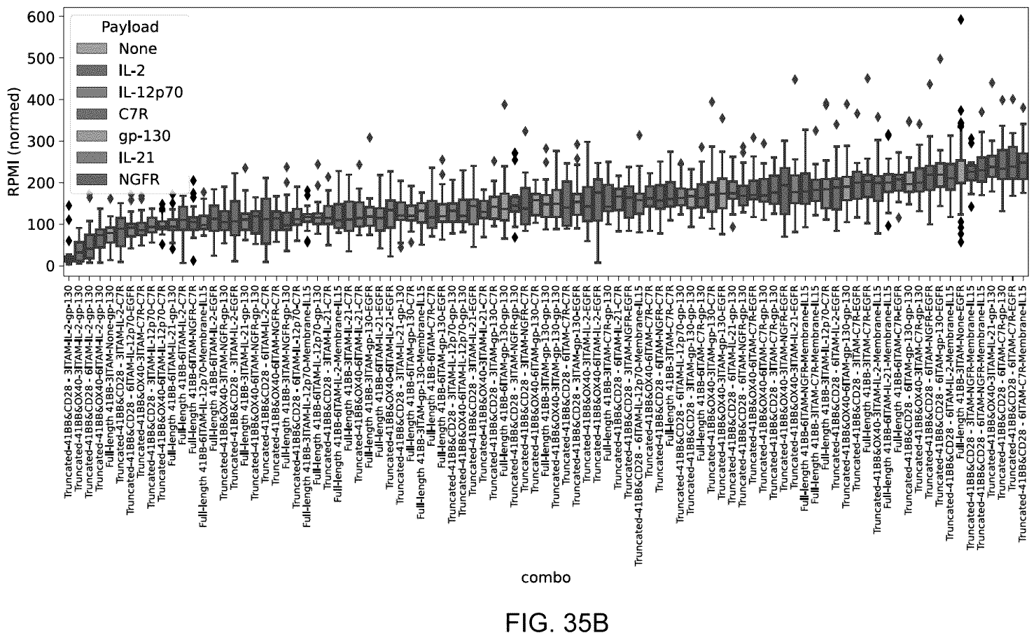

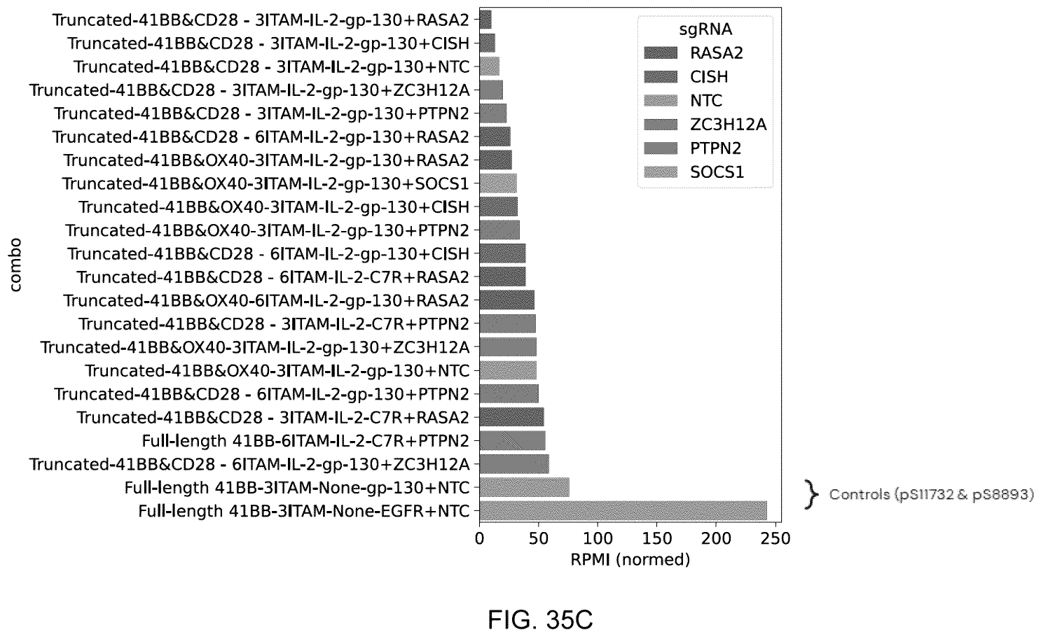

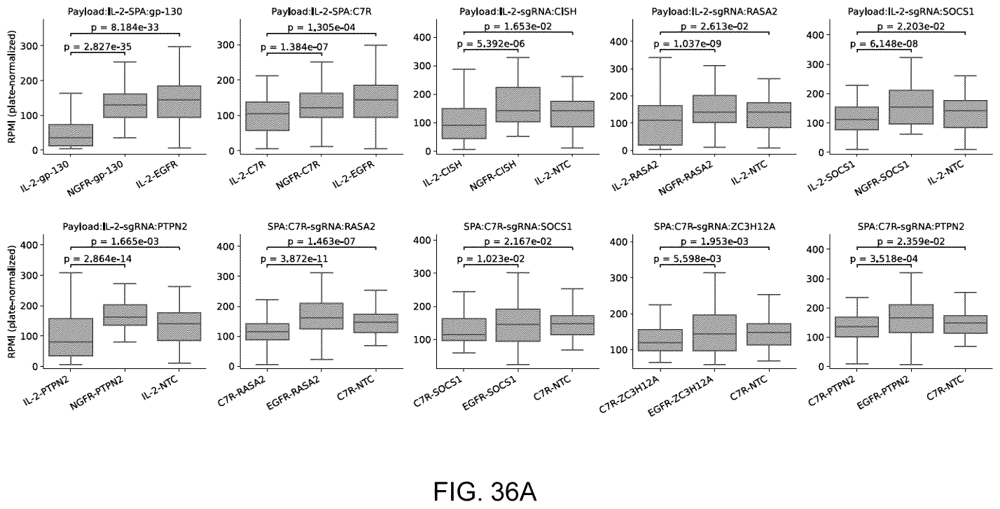

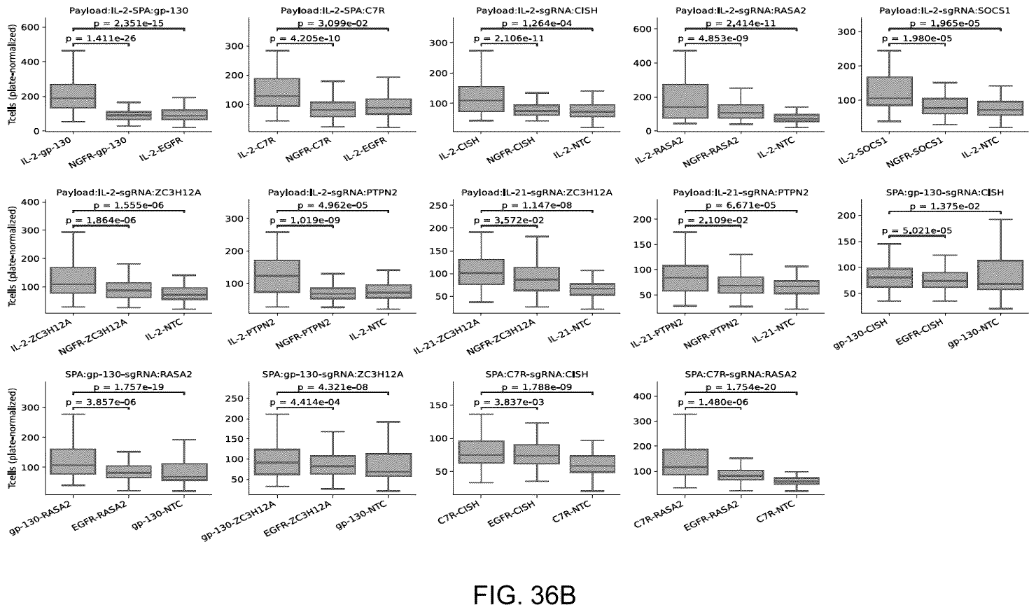

Normalized results of the continuous stimulation assay screen are shown in FIGS. 35A and 35B. A wide range of RPMI control cell counts was observed throughout the various tested combinations, indicating that different module combinations lead to different functional output. From the combinations high performance was consistently observed with L-gp130 SPA and IL-2 modules. The top 20 combinations are shown in comparison to positive controls pS11732 and pS8893 in FIG. 35C, with some combinations leading to a greater than 8-fold increase in suppression of target cell counts. Overall, 18% of combinations performed better than single-module circuits in increased LG T cell activity based on decreased target cell count (FIG. 36A), and 25% of combinations performed better than single-module circuits in increased LG T cell counts (FIG. 36B).

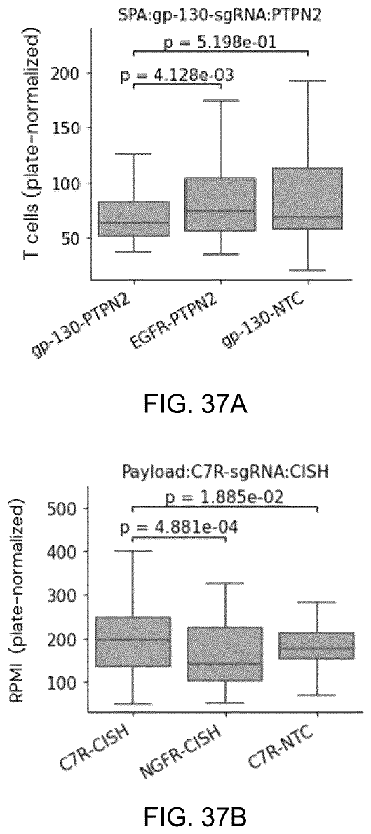

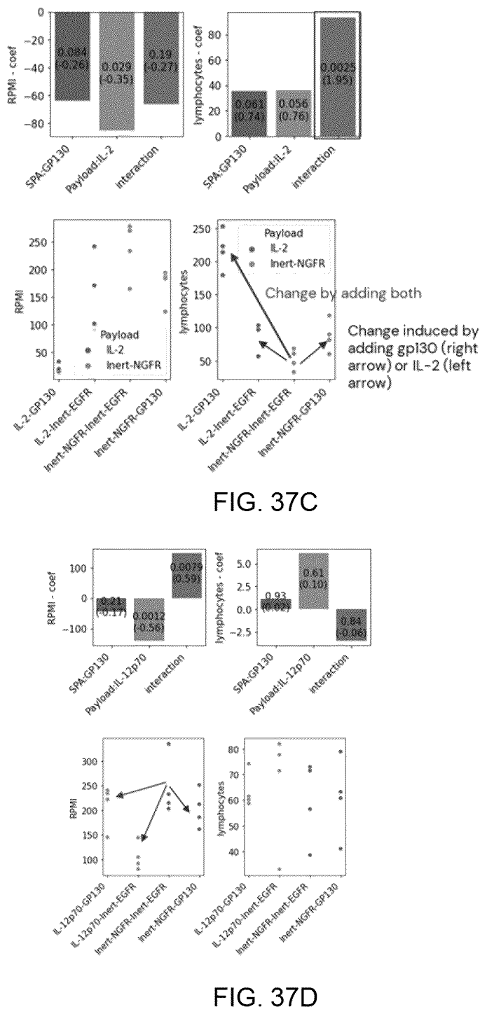

Not all combinations yielded an additive effect, however. For example, the combination of L-gp130 with an sgRNA targeting PTPN2 yielded no improvement in LG T cell expansion compared to either L-gp130 or the sgRNA alone (FIG. 37A). Similarly, the combination of C7R with an sgRNA targeting CISH yielded no additional decrease in target cell count compared to cither C7R or the sgRNA alone (FIG. 37B).

To further assess the potential super-additive effect of L-gp13 and IL-2, the ANOVA analysis was carried out with a fixed CAR module and across every tested background. Briefly, the ANOVA model coefficients of interaction terms with nominal a p-value <0.05. A coefficient >0 suggests more RPMI (worse) than expected by an additive effect, whereas a coefficient <0 suggests fewer RPMI (better) than expected by an additive effect. The analysis demonstrated that the co-expression of L-gp130 and IL-2 in LG T cells leads to a 195% increase in T cell expansion and a 27% decrease in target cell count compared to the expected values based on an additive effect, indicating an interaction between the two modules in LG T cells (FIG. 37C).

An additional observation of the screen was the redundancy found between L-gp130 and IL-12. An ANOVA analysis carried out as described above with a fixed CAR module and across every tested sgRNA demonstrated that both L-gp130 and IL-12 independently increased LG T cell activity based on a decreased target cell count, this effect almost completely disappeared when the two were combined (FIG. 37D).

### Example 10: Analysis of LG T Cells Expressing a Combination Including One or More shRNAs, Additional Suppressors of Gene Expression, and an SPA and/or a Cytokine

### Materials and Methods

### Editing and Normalization of T Cells:

To generate a library of T cells for screening T cells from 2 donors were incubated in a 384-well plate with plasmids encoding the CAR, PrimeR, and combinations including one or more shRNAs, a synthetic pathway activator, a cytokine, and an sgRNA for gene silencing. Edited cells were stained with Ki-67 to measure cell count. Ki-67 data was wrangled and cells were normalized for both overall count and fraction of edited cells prior to commencement of the continuous stimulation assay.

### Continuous Stimulation Assay:

To evaluate the cytolytic and proliferative capacity of the engineered T cells, continuous stimulation assay was performed. Briefly, the engineered T cells and tumor cells were seeded at 1:50 (500 edited T cells: 25K RPMI 8226 target cells) with complete RPMI medium (RPMI 1640+glutamax, 10% fetal bovine serum, 15 ng/ul gentamicin) with or without 5 ng/ml TGF-beta treatment. Media exchange occurred in every co-culture 3 times a week (every 2-3 days) with the RPMI medium with or without 5 ng/ml TGF-beta treatment. A split and re-challenge was performed at day 9 and 12 of co-culture. To split the co-culture, each well was mixed to achieve a homogenous mixture and 25% of the volume (50 ul) was acquired and replated to a new plate. To re-challenge the cells, 25K tumor cells were introduced to the new plate containing the split co-culture cells. The assay was concluded at 14 days of co-culture and target cell count and T cell count were measured via Incucyte using the manufacturer's protocol.

### Results

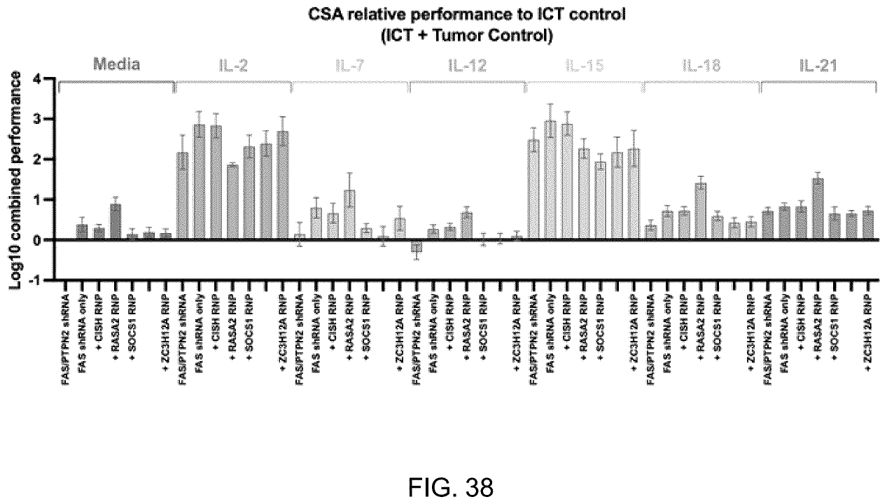

To establish cytokine backgrounds, LG T cells were prepared to express shRNAs targeting Fas (SEQ ID NO: 171) and PTPN2 (SEQ ID NO: 167), Fas alone, or Fas and PTPN2 in combination with an sgRNA targeting CISH (SEQ ID NO: 164), RASA2 (SEQ ID NO: 161), SOCSI (SEQ ID NO: 162), or ZC3H12A (SEQ ID NO: 163). Cells were then run on a continuous stimulation assay in control medium or medium supplemented with one of IL-2, IL-7, IL-12, IL-15, IL-18, or IL-21. Results showed that IL-2 and IL-15 supplementation had the best tumor control and ICT expansion regardless of genetic background (FIG. 38). With IL-2 and IL-15 supplementation, Fas knock-down (KD)/CISH knockout (KO), Fas KD/ZC3H12A KO and Fas KD-only showed the most potency of those tested in the experiment. Without cytokine treatment or in non-IL-2/IL-15 conditions Fas KD/RASA2 KO was the most potent combination tested in the experiment.

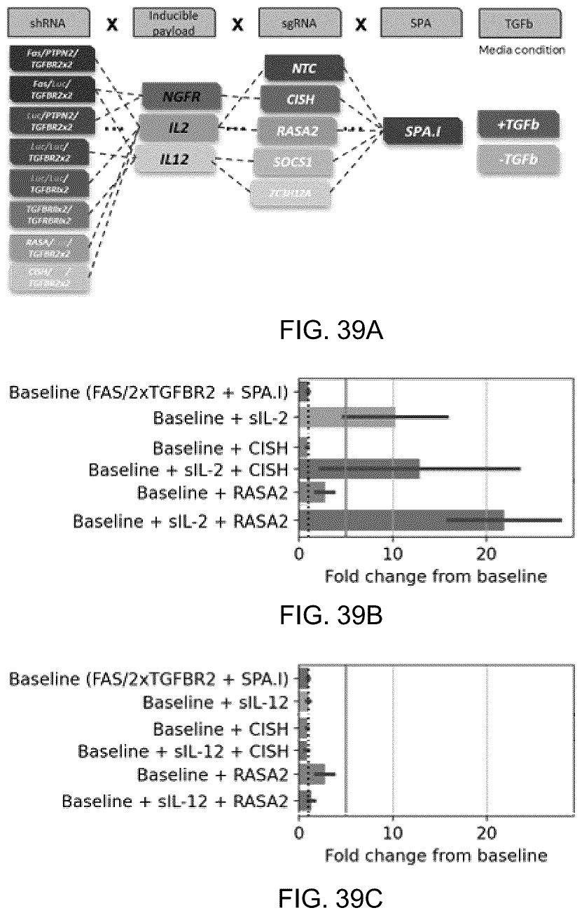

To screen for shRNA cassettes, ICT cells were prepared with combinations of shRNAs, inducible payloads, sgRNAs, and SPAs as detailed in FIG. 39A. Table F. details constructs used in the screen.

TABLE F

| Construct | SEQ ID NO(s) |
|---|---|
| Luciferase shRNA (control) | 173 and 174 |
| Fas shRNA | 168 |
| PTPN2 shRNA | 166 |
| TGFBR2 shRNA | 171 and 172 |
| TGFBR1 shRNA | 169 and 170 |
| RASA2 shRNA | 165 |
| CISH shRNA | 167 |
| NGFR payload (control) | 175 |
| IL-2 | 86 |
| IL-12 | 92 |
| Non-targeting control sgRNA | 159 |
| CISH-targeting sgRNA | 164 |
| RASA2-targeting sgRNA | 161 |
| SOCS1-targeting sgRNA | 162 |
| ZC3H12A-targeting sgRNA | 163 |
| L-gp130 (SPA.I) | 75 |

Engineered cells were then run on a continuous stimulation assay with or without TGF-β added to the medium. Results showed that all tested conditions where IL-2 was inducibly expressed showed a greater than 5-fold increase in potency (FIG. 39B). It was also shown that inducible IL-2 improved the potency of ICTs co-expressing an SPA, which was further improved by RASA2 KO.

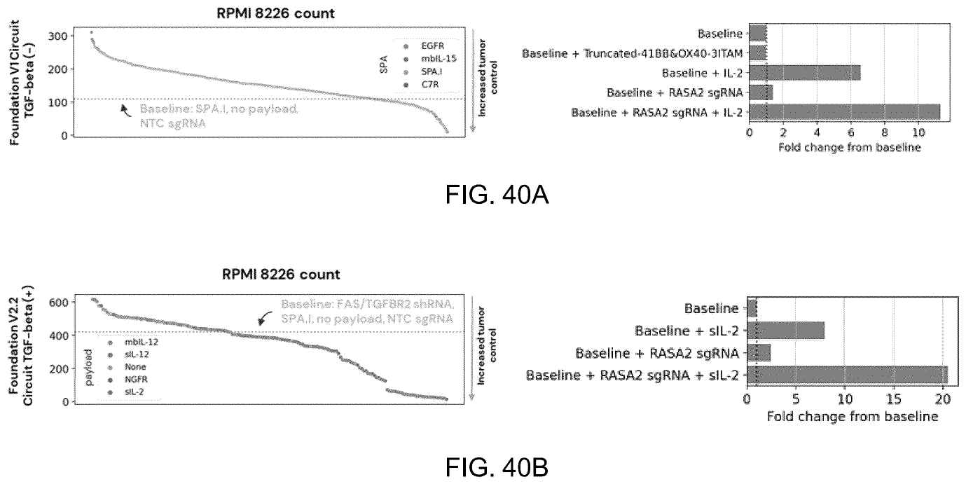

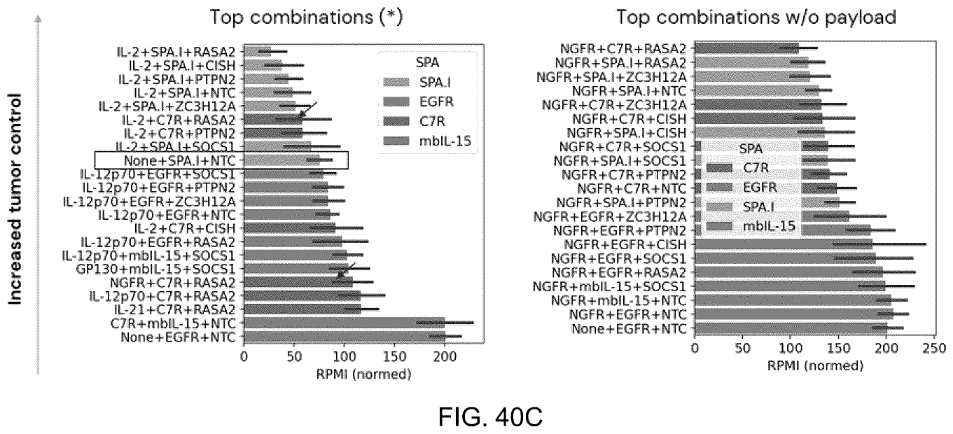

To further assess the effects of the combination of inducible IL-2 expression and RASA2 suppression, ICT cells were prepared as above with additional SPAs and payloads and run on a continuous stimulation assay in the presence (FIG. 40A) or absence (FIG. 40B) of TGF-β. Analysis of combinations with or without expression of a cytokine payload showed that inducible IL-2 expression plus L-gp130 expression with RASA2, CISH, PTPN2, or NTC sgRNAs were the top performing constructs, with the combination of IL-2 expression, L-gp130, and RASA2 knockout showing the greatest tumor control. (FIG. 40C). Additionally, C7R+RASA2, CISH, or PTPN2 KO had comparable performance to L-gp130 with or without an IL-2 payload.

### Example 11: In Vivo Analysis of LG T Cells Expressing a Synthetic Pathway Activator and Cytokine Payload

### Materials and Methods

To determine the anti-cancer efficacy of L-gp130 SPA+IL-2 combination modules, a late stage MSTO-211H-EFG-ALPG^{h}/MSLN^{h} subcutaneous xenograft model was utilized. 1×10⁶ MSTO-211H-EFG-ALPG^{h}/MSLN^{h} cells in 50% matrigel were inoculated into the right dorsal flank of six-to-seven-weeks old, female NSG MHC I/II DKO mice. Day 28 post tumor inoculation, mean tumor volume of 300 mm³ was reached, and tumor-bearing animals were randomized into various treatment groups such that mean tumor volume per group was within 10% of the overall mean. Mice were injected intravenously with a single dose of 0.05, 0.1, 0.3 and 0.7 ×10⁶ L-gp130 or L-gp130+IL-2 containing ICT and the negative control RNP T cells were administered at the highest dose equivalent to the total T cells administered in the cohort of the L-gp130+IL2. Mice were dosed individually by fixed volume (200 μL) on the day of treatment as described above. Tumor growth and body weight were monitored twice per week. Whole blood samples were collected at day 7, 14, 21, 42, 56 and 70 in K²EDTA tubes through survival technique and processed for staining of total and receptor positive T cells. Whole blood samples were collected on day 1, 3, 7, 14, 21, 42, 56 into serum separator tubes, processed to serum and snap frozen. Serum samples were assessed for human cytokine concentrations via a Luminex 13-plex assay per manufacturer's protocol. At the end of study or upon reaching euthanasia criteria, animals were necropsied for liver, lungs, spleen, kidneys, heart, brain and tumor. Tissues were either snap frozen or fixed in 10% neutral buffered formalin, 24 hrs and stored in 70% reagent grade ethanol for mounting and sectioning

### Results

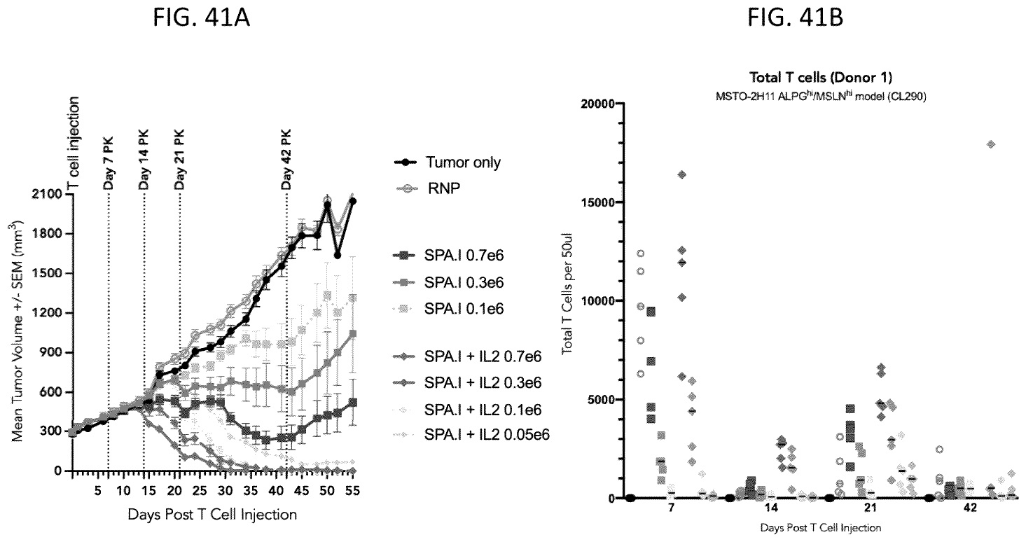

The in vivo efficacy and potency of ICT cells expressing a cytokine IL-2 payload and L-gp130 were assessed as compared to ICT cells expressing L-gp130 and a control cytokine NGFR. As shown in FIGS. 41A and 41B, ICT cells expressing L-gp130 (SPA.I) and an IL2 payload had a greater than 14 fold enhanced potency as compared to ICT cells expressing L-gp130 (SPA.I) alone, in the MSTO (mesothelioma) in vivo model. FIG. 41A shows the mean tumor volume in mice after treatment with the indicated ICT cells. FIG. 41B shows the total T cells at 7, 14, 21, and 42 days post T cell injection. The 0.05e6 SPA.I plus IL2 ICT cell dose outperformed the 0.7c6 dose of SPA.I only T cells in controlling the tumor volume. Thus, in the late stage MSTO tumor model, the IL-2 payload module significantly increased the SPA.I ICT cell potency.

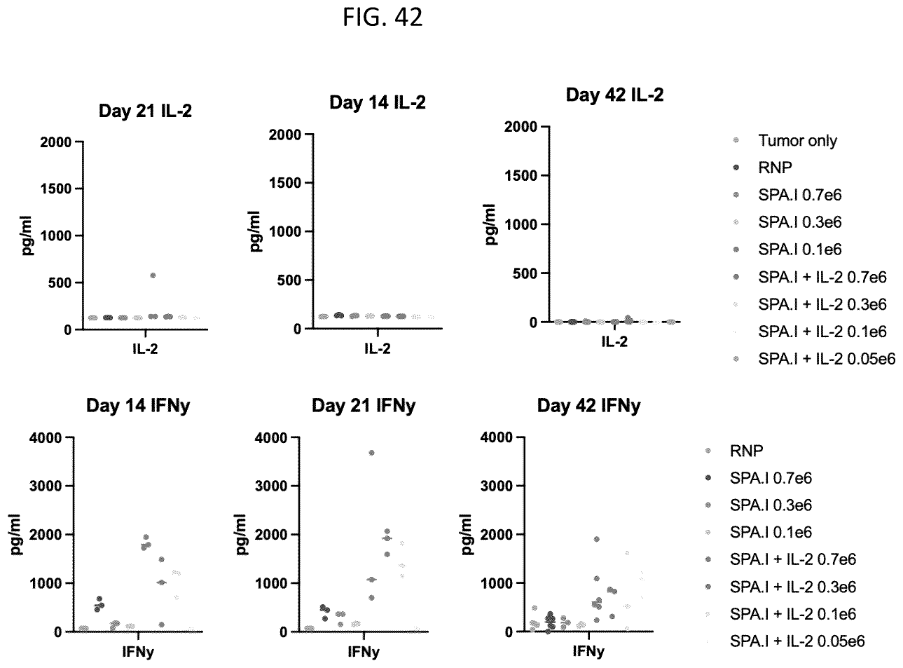

Serum IL-2 was detected in only one animal in the 0.7e6 high dose SPA.I plus IL-2 ICT cell group (FIG. 42). In a late stage MSTO tumor model, the IL-2 payload module did not correspond to detectable human IL-2 in mouse serum at time points assessed (Day 1, 3, 7, 14, 21, and 42) with the exception of one animal from the high dose group (0.7e6 edited T cells/mouse) (FIG. 42). Thus, the IL-2 payload was not released into circulation in vivo.

Inclusion of the IL-2 payload module did correspond with increased detectable human IFN-γ in mouse serum at time points assessed. IFN-γ was detected in the mouse serum at days 14, 21, and 42 post injection in the animals dosed with 0.7c6, 0.3c6, and 0.1c6 L-gp130 (SPA.I) plus IL-2 ICT cells (FIG. 42). IFN-γ was also detected in the mouse serum at day 42 post injection in the animals dosed with the lowest dose of 0.05c6 L-gp130 (SPA.I) plus IL-2 ICT cells (FIG. 42). In contrast, the ICT cells expressing only L-gp130 (SPA.I) induced very low levels of detectable IFN-γ on Days 14 and 21 only in the higher cell dosages (0.7c6 and 0.3c6).

### Example 12: In Vitro Validation of Payload Enhancement of Other Immune Cells

### Materials and Methods

Cryopreserved T cells were activated and cultured in preparation for DNA knock-in as described previously. After 2 days of activation T cells were removed from CD3/CD28 beads and electroporated with a Cas9 RNP targeting the TRAC locus and a DNA plasmid encoding the NY-ESO TCR. Post electroporation T cells were cultured for an additional 5 days in IL-7 and IL-15 and then assessed via flow cytometry for successful TRAC knockout and knock in the NY-ESO TCR via tetramer and TCR VB13.1 antibody staining. TCR knock in T cells were then enriched via a PE-TCR VB13.1 antibody, a biotinylated anti-PE antibody, and streptavidin beads. After enrichment TCR knock in T cells were seeded at 10,000 TCR+ T cells per well in a 96 well with 10,000 mCherry expressing A375 target cells. Tumor cell killing was assessed by fluorescent imaging over time in the incucyte instrument according to the manufacturer's instructions. Cultures were maintained in RPMI1640 media+10% FBS+/−1 ng/ml IL-2 or IL-15. Every 3 days non-adherent cells in each well were split 1:1 and half were reseeded with 10,000 fresh A375 target cells for a total of 4 target cell stimulations. In between stimulations, the remaining half of the non-adherent cells were stained with the TCR VB13.1 antibody to quantify TCR expansion.

Readouts: “Endogenous” T cell function, TCR knock in T cell proliferation, TCR target cell control, activation of non-engineered T cells

Assay objective was to identify payload designs which enhance endogenous T cell anti-tumor function and result in minimal activation of Treg cells with minimal cytokine production in order to reduce CRS while maintaining high cytolytic activity.

### Results

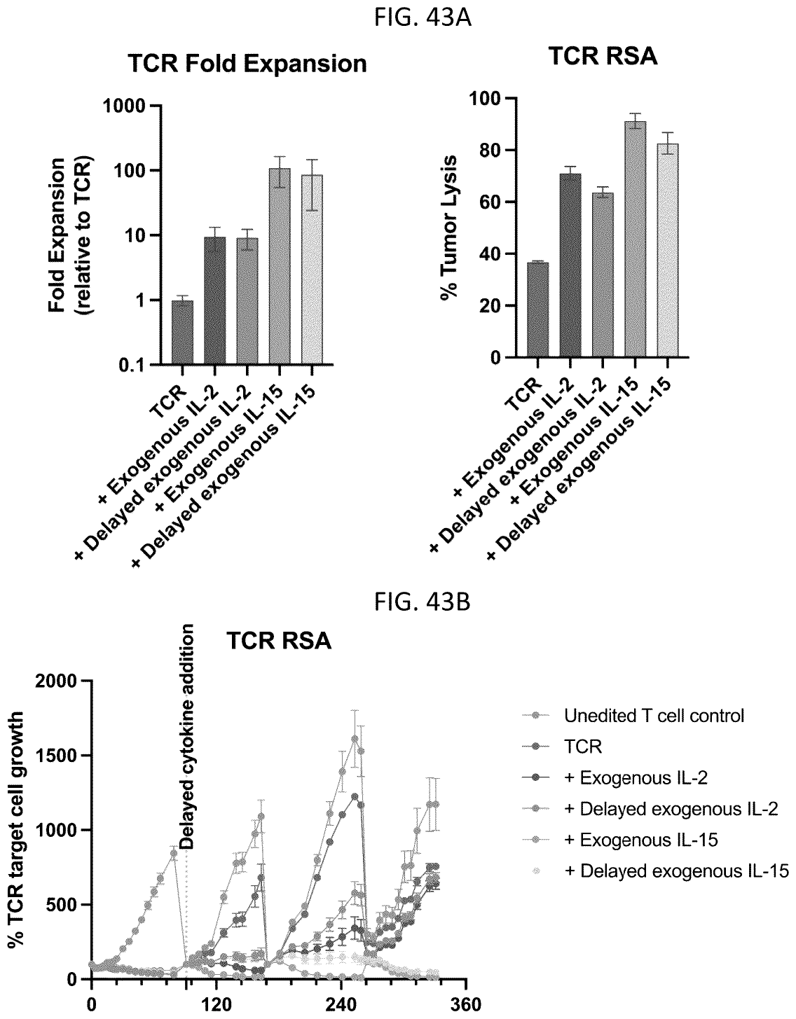

Exogenous IL-2 or IL-15 treatment improved TCR T cell tumor control and T cell proliferation (FIGS. 43A and 43B). FIG. 43A shows TCR expansion and % tumor lysis after incubation of T cells with exogenous IL-2 or IL-15 or delayed addition of exogenous IL-2 or IL-15. As shown in FIG. 43A, the addition of IL-2 or IL-15 increased the TCR expansion and tumor lysis as compared to no cytokine addition (TCR group). As shown in FIG. 43B, the delayed addition of exogenous IL2 or IL-15 still resulted in improved TCR T cell tumor control. Thus, exogenous IL-2 or IL-15 significantly improved TCR T cell tumor control. IL-15 showed full tumor control in four target cell stimulations every 14 days, even after delayed addition of IL-15.

### Example 13: In Vitro ICT Cytolytic Assay with IL-15

### Materials and Methods

### Continuous Stimulation Assay

To evaluate the cytolytic and proliferative capacity of the engineered T cells, a continuous stimulation assay was performed as in Example 2 and Example 10 with ICT cells expressing IL-15. Briefly, the engineered T cells and tumor cells were seeded at 1:50 (500 edited T cells: 25K RPMI 8226 target cells) with complete RPMI medium (RPMI 1640+glutamax, 10% fetal bovine serum, 15 ng/ml gentamicin, 5 ng/ml TGFb). Media exchange occurred in every co-culture 3 times a week (every 2-3 days) with the RPMI medium mentioned above. The assay was concluded at 7 days of co-culture and target cell count and T cell count were measured via incucyte using the manufacturer's protocol.

### Results

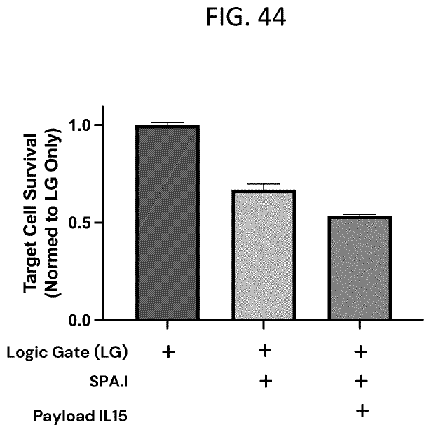

Addition of IL-15 expression to the ICT cells expressing L-gp130 (SPA.I) resulted in increased killing by the T cells, as shown by the reduced target survival (FIG. 44). Target cell survival was normalized to ICT cells expressing a logic gate only.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ | | |
|---|---|---|
| ID | | |
| NO | Name | Sequence |
| 1 | ALPG/P CDR | GFSLTSY |
| | H1 | |
| 2 | ALPG/P CDR | WEDGS |
| | H2 | |
| 3 | ALPG/P CDR | PHYGSSYVGAMEY |
| | H3 | |
| 4 | ALPG/P CDR | RASENIYSYVA |
| | L1 | |
| 5 | ALPG/P CDR | NAKSLAS |
| | L2 | |
| 6 | ALPG/P CDR | QHHYVSPWT |
| | L3 | |
| 7 | ALPG/P VH | QVQLQESGPGLVKPSETLSLTCTVSGFSLTSYGVSWIRQPAG |
| | | KGLEWIGVIWEDGSTNYHSALISRVTMSVDTSKNQFSLKLSS |
| | | VTAADTAVYYCARPHYGSSYVGAMEYWGAGTTVTVSS |
| 8 | ALPG/P VL | DIQMTQSPSSLSASVGDRVTITCRASENIYSYVAWYQQKPGK |
| | | APKLLIYNAKSLASGVPSRFSGSGSGTDFTLTISSLQPEDFAT |
| | | YYCQHHYVSPWTFGGGTKLEIK |
| 9 | primeR | DIQMTQSPSSLSASVGDRVTITCRASENIYSYVAWYQQKPGK |
| | APLG/P full | APKLLIYNAKSLASGVPSRFSGSGSGTDFTLTISSLQPEDFAT |
| | binder ECD | YYCQHHYVSPWTFGGGTKLEIKGGGGSGGGGSGGGGSQVQ |
| | scFv | LQESGPGLVKPSETLSLTCTVSGFSLTSYGVSWIRQPAGKGL |
| | | EWIGVIWEDGSTNYHSALISRVTMSVDTSKNQFSLKLSSVTA |
| | | ADTAVYYCARPHYGSSYVGAMEYWGAGTTVTVSS |
| 10 | MSLN CDR | GYTMN |
| | H1 | |
| 11 | MSLN CDR | LITPYNGASSYNQKFRG |
| | H2 | |
| 12 | MSLN CDR | GGYDGRGFDY |
| | H3 | |
| 13 | MSLN VH | QVQLQQSGPELEKPGASVKISCKASGYSFTGYTMNWVKQSH |
| | | GKSLEWIGLITPYNGASSYNQKFRGKATLTVDKSSSTAYMD |
| | | LLSLTSEDSAVYFCARGGYDGRGFDYWGQGTTVTVSS |
| 14 | MSLN CDR | SASSSVSYMH |
| | L1 | |
| 15 | MSLN CDR | DTSKLAS |
| | L2 | |
| 16 | MSLN CDR | QQWSGYPLT |
| | L3 | |
| 17 | MSLN VL | DIELTQSPAIMSASPGEKVTMTCSASSSVSYMHWYQQKSGT |
| | | SPKRWIYDTSKLASGVPGRFSGSGSGNSYSLTISSVEAEDDA |
| | | TYYCQQWSGYPLTFGAGTKLEIK |
| 18 | primeR CD8α | TTTPAPRPPTPAPTIASQPLSLRPEAC |
| | Hinge | |
| 19 | primeR TMD | FMYVAAAAFVLLFFVGCGVLLS |
| 20 | primeR STS | RKRRRQHGQLWFPEGFKVSEASKKKRREPLGEDSVGLKPLK |
| | | NA |
| 21 | primeR Gal4 | KLLSSIEQACDICRLKKLKCSKEKPKCAKCLKNNWECRYSP |
| | DBD | KTKRSPLTRAHLTEVESRLERLEQLFLLIFPREDLDMILKMDS |
| | | LQDIKALLTGLFVQDNVNKDAVTDRLASVETDMPLTLRQHR |
| | | ISATSSSEESSNKGQRQLTVS |
| 22 | primeR VP64 | AAAGGSGGSGGSDALDDFDLDMLGSDALDDFDLDMLGSDA |
| | domain | LDDFDLDMLGSDALDDFDLDMLGS |
| 23 | primeR TAD | KLLSSIEQACDICRLKKLKCSKEKPKCAKCLKNNWECRYSP |
| | | KTKRSPLTRAHLTEVESRLERLEQLFLLIFPREDLDMILKMDS |
| | | LQDIKALLTGLFVQDNVNKDAVTDRLASVETDMPLTLRQHR |
| | | ISATSSSEESSNKGQRQLTVSAAAGGSGGSGGSDALDDFDLD |
| | | MLGSDALDDFDLDMLGSDALDDFDLDMLGSDALDDFDLD |
| | | MLGS |
| 24 | ALPG primeR | DIQMTQSPSSLSASVGDRVTITCRASENIYSYVAWYQQKPGK |
| | protein full | APKLLIYNAKSLASGVPSRFSGSGSGTDFTLTISSLQPEDFAT |
| | sequence | YYCQHHYVSPWTFGGGTKLEIKGGGGSGGGGSGGGGSQVQ |
| | | LQESGPGLVKPSETLSLTCTVSGFSLTSYGVSWIRQPAGKGL |
| | | EWIGVIWEDGSTNYHSALISRVTMSVDTSKNQFSLKLSSVTA |
| | | ADTAVYYCARPHYGSSYVGAMEYWGAGTTVTVSS |
| 25 | ALPG primeR | MALPVTALLLPLALLLHAARPEQKLISEEDLDIQMTQSPSSLS |
| | protein full | ASVGDRVTITCRASENIYSYVAWYQQKPGKAPKLLIYNAKS |
| | sequence with | LASGVPSRFSGSGSGTDFTLTISSLQPEDFATYYCQHHYVSP |
| | CD8 signal | WTFGGGTKLEIKGGGGSGGGGSGGGGSQVQLQESGPGLVK |
| | sequence | PSETLSLTCTVSGFSLTSYGVSWIRQPAGKGLEWIGVIWEDG |
| | | STNYHSALISRVTMSVDTSKNQFSLKLSSVTAADTAVYYCA |
| | | RPHYGSSYVGAMEYWGAGTTVTVSS |
| 26 | MSLN Hinge | TTTPAPRPPTPAPTIASQPLSLRPEACRPAAGGAVHTRGLDFA |
| | CD8a | CD |
| 27 | MSLN TMD | IYIWAPLAGTCGVLLLSLVITLYC |
| | CD8a | |
| 28 | MSLN Co- | KRGRKKLLYIFKQPFMRPVQTTQEEDGCSCRFPEEEEGGCEL |
| | stim 4-1BB | |
| 29 | MSLN | RVKFSRSADAPAYQQGQNQLYNELNLGRREEYDVLDKRRG |
| | Activation | RDPEMGGKPRRKNPQEGLYNELQKDKMAEAYSEIGMKGER |
| | CD3z | RRGKGHDGLYQGLSTATKDTYDALHMQALPPR |
| 30 | MSLN scFv | QVQLQQSGPELEKPGASVKISCKASGYSFTGYTMNWVKQSH |
| | protein full | GKSLEWIGLITPYNGASSYNQKFRGKATLTVDKSSSTAYMD |
| | sequence | LLSLTSEDSAVYFCARGGYDGRGFDYWGQGTTVTVSSGGG |
| | | GSGGGGSGGGGSDIELTQSPAIMSASPGEKVTMTCSASSSVS |
| | | YMHWYQQKSGTSPKRWIYDTSKLASGVPGRFSGSGSGNSYS |
| | | LTISSVEAEDDATYYCQQWSGYPLTFGAGTKLEIK |
| 31 | MSLN CAR | QVQLQQSGPELEKPGASVKISCKASGYSFTGYTMNWVKQSH |
| | protein full | GKSLEWIGLITPYNGASSYNQKFRGKATLTVDKSSSTAYMD |
| | sequence | LLSLTSEDSAVYFCARGGYDGRGFDYWGQGTTVTVSSGGG |
| | | GSGGGGSGGGGSDIELTQSPAIMSASPGEKVTMTCSASSSVS |
| | | YMHWYQQKSGTSPKRWIYDTSKLASGVPGRFSGSGSGNSYS |
| | | LTISSVEAEDDATYYCQQWSGYPLTFGAGTKLEIKAAATTTP |
| | | APRPPTPAPTIASQPLSLRPEACRPAAGGAVHTRGLDFACDIY |
| | | IWAPLAGTCGVLLLSLVITLYCKRGRKKLLYIFKQPFMRPVQ |
| | | TTQEEDGCSCRFPEEEEGGCELRVKFSRSADAPAYQQGQNQ |
| | | LYNELNLGRREEYDVLDKRRGRDPEMGGKPRRKNPQEGLY |
| | | NELQKDKMAEAYSEIGMKGERRRGKGHDGLYQGLSTATKD |
| | | TYDALHMQALPPR |
| 32 | MSLN CAR | MALPVTALLLPLALLLHAARPDYKDDDDKQVQLQQSGPELE |
| | protein full | KPGASVKISCKASGYSFTGYTMNWVKQSHGKSLEWIGLITP |
| | sequence with | YNGASSYNQKFRGKATLTVDKSSSTAYMDLLSLTSEDSAVY |
| | signal | FCARGGYDGRGFDYWGQGTTVTVSSGGGGSGGGGSGGGG |
| | sequence | SDIELTQSPAIMSASPGEKVTMTCSASSSVSYMHWYQQKSG |
| | | TSPKRWIYDTSKLASGVPGRFSGSGSGNSYSLTISSVEAEDD |
| | | ATYYCQQWSGYPLTFGAGTKLEIKAAATTTPAPRPPTPAPTI |
| | | ASQPLSLRPEACRPAAGGAVHTRGLDFACDIYIWAPLAGTC |
| | | GVLLLSLVITLYCKRGRKKLLYIFKQPFMRPVQTTQEEDGCS |
| | | CRFPEEEEGGCELRVKFSRSADAPAYQQGQNQLYNELNLGR |
| | | REEYDVLDKRRGRDPEMGGKPRRKNPQEGLYNELQKDKM |
| | | AEAYSEIGMKGERRRGKGHDGLYQGLSTATKDTYDALHMQ |
| | | ALPPR |
| 33 | CD8α signal | MALPVTALLLPLALLLHAARP |
| | peptide | |
| | sequence | |
| 34 | Myc Tag | EQKLISEEDL |
| 35 | ALPG primeR | atggccttaccagtgaccgccttgctcctgccg |
| | nucleic acid | ctggccttgctgctccacgccgccaggcct |
| | full sequence | GAACAAAAACTCATTAGCGAAGAGGATCTCGACATACAG |
| | | ATGACACAGAGCCCTAGCAGTCTGAGCGCCAGTGTGGGC |
| | | GATAGAGTTACTATCACTTGTAGAGCATCCGAGAACATA |
| | | TACAGTTACGTGGCCTGGTATCAGCAAAAACCTGGCAAA |
| | | GCTCCCAAGTTATTGATTTACAATGCTAAGAGCTTGGCCT |
| | | CTGGGGTGCCATCGAGGTTCAGCGGTAGCGGGAGCGGGA |
| | | CCGACTTCACTCTGACCATCTCGAGTCTCCAGCCGGAGGA |
| | | CTTTGCGACATACTATTGTCAACACCATTACGTATCACCC |
| | | TGGACCTTCGGCGGCGGGACTAAGTTAGAGATCAAGGGT |
| | | GGAGGAGGATCAGGCGGCGGTGGATCAGGAGGAGGAGG |
| | | GTCACAAGTGCAGTTACAGGAATCAGGGCCCGGCCTGGT |
| | | GAAGCCAAGTGAAACCCTGAGTCTGACGTGCACGGTTTC |
| | | AGGATTTAGCCTCACTTCCTACGGTGTCTCTTGGATTCGG |
| | | CAGCCAGCCGGCAAAGGGCTCGAGTGGATTGGGGTGATC |
| | | TGGGAAGATGGCTCAACAAACTATCATTCTGCACTAATCT |
| | | CTCGCGTGACAATGTCGGTGGACACGTCCAAGAATCAAT |
| | | TTTCCCTTAAACTGTCCTCCGTGACCGCAGCCGATACAGC |
| | | GGTATATTATTGCGCGCGACCTCACTACGGATCTAGCTAT |
| | | GTCGGCGCGATGGAGTATTGGGGCGCTGGCACAACCGTC |
| | | ACCGTTTCTTCC |
| 36 | MSLN CAR | ATGGCCCTGCCAGTAACGGCTCTGCTGCTGCCACTTGCTC |
| | nucleic acid | TGCTCCTCCATGCAGCCAGGCCTGACTACAAAGACGATG |
| | full sequence | ACGACAAGCAAGTCCAGCTCCAGCAGTCGGGCCCAGAGT |
| | | TGGAGAAGCCTGGGGCGAGCGTGAAGATCTCATGCAAAG |
| | | CCTCAGGCTACTCCTTTACTGGATACACGATGAATTGGGT |
| | | GAAACAGTCGCATGGAAAGTCACTGGAATGGATCGGTCT |
| | | GATTACGCCCTACAACGGCGCCTCCAGCTACAACCAGAA |
| | | GTTCAGGGGAAAGGCGACCCTTACTGTCGACAAGTCGTC |
| | | AAGCACCGCCTACATGGACCTCCTGTCCCTGACCTCCGAA |
| | | GATAGCGCGGTCTACTTTTGTGCACGCGGAGGTTACGATG |
| | | GACGGGGATTCGACTACTGGGGCCAGGGAACCACTGTCA |
| | | CCGTGTCGAGCGGAGGCGGAGGGAGCGGAGGAGGAGGC |
| | | AGCGGAGGTGGAGGGTCGGATATCGAACTCACTCAGTCC |
| | | CCAGCAATCATGTCCGCTTCACCGGGAGAAAAGGTGACC |
| | | ATGACTTGCTCGGCCTCCTCGTCCGTGTCATACATGCACT |
| | | GGTACCAACAAAAATCGGGGACCTCCCCTAAGAGATGGA |
| | | TCTACGATACCAGCAAACTGGCTTCAGGCGTGCCGGGAC |
| | | GCTTCTCGGGTTCGGGGAGCGGAAATTCGTATTCGTTGAC |
| | | CATTTCGTCCGTGGAAGCCGAGGACGACGCAACTTATTA |
| | | CTGCCAACAGTGGTCAGGCTACCCGCTCACTTTCGGAGCC |
| | | GGCACTAAGCTGGAGATCAAGGCGGCAGCAaccacgacg |
| | | ccagcgccgcgaccaccaacaccggcgcccaccatcgcgt |
| | | cgcagCCActgTCActgcgcccaGAAgcgtgccggccagc |
| | | ggcggggggcgcagtgcacacgagggggctggacttcgcc |
| | | tgtgatatctacatctgggcgcccttggccgggacttgt |
| | | ggggtccttctcctgtcactggttatcaccctttactgca |
| | | aacggggcagaaagaaactcctgtatatattcaaacaacc |
| | | atttatgagaccagtacaaactactcaagaagaggacggc |
| | | tgtagctgccgatttccagaagaagaagaaggaggatgtg |
| | | aactgAGAGTGAAGTTCAGCAGGAGCGCAGACGCCCCC |
| | | GCGTACCAGCAGGGCCAGAACCAGCTCTATAACGAGCTC |
| | | AATCTAGGACGAAGAGAGGAGTACGATGTTTTGGACAAG |
| | | AGGcgtGGCCGGGACCCTGAGATGGGGGGAAAGCCGAGA |
| | | AGGAAGAACCCTCAGGAAGGCCTGTACAATGAACTGCAG |
| | | AAAGATAAGATGGCGGAGGCCTACAGTGAGATTGGGATG |
| | | AAAGGCGAGCGCCGGAGGGGCAAGGGGCACGATGGCCT |
| | | TTACCAGGGTCTCAGTACAGCCACCAAGGACACCTACGA |
| | | CGCCCTTCACATGCAGGCCCTGCCCCCTAGG |
| 37 | primeR TMD | FMYVAAAAFVLLFFVGCGVLLS |
| 74 | L-gp130 | NKRDLIKKHIWPNVPDPSKSHIAQWSPHTPPRHNFNSKDQM |
| | intracellular | YSDGNFTDVSVVEIEANDKKPFPEDLKSLDLFKKEKINTEGH |
| | signaling | SSGIGGSSCMSSSRPSISSSDENESSQNTSSTVQYSTVVHSGY |
| | domain amino | RHQVPSVQVFSRSESTQPLLDSEERPEDLQLVDHVDGGDGIL |
| | acid sequence | PRQQYFKQNCSQHESSPDISHFERSKQVSSVNEEDFVRLKQQ |
| | | ISDHISQSCGSGQMKMFQEVSAADAFGPGTEGQVERFETVG |
| | | MEAATDEGMPKSYLPQTVRQGGYMPQ |
| 75 | L-gp130 | MALPVTALLLPLALLLHAARPDYKDDDDKELCGGRIARLEE |
| | amino acid | KVKTLKAQNSELASTANMLREQVAQLKQKVMNAQGEIEAI |
| | sequence | VVPVCLAFLLTTLLGVLFCFNKRDLIKKHIWPNVPDPSKSHIA |
| | | QWSPHTPPRHNFNSKDQMYSDGNFTDVSVVEIEANDKKPFP |
| | | EDLKSLDLFKKEKINTEGHSSGIGGSSCMSSSRPSISSSDENES |
| | | SQNTSSTVQYSTVVHSGYRHQVPSVQVFSRSESTQPLLDSEE |
| | | RPEDLQLVDHVDGGDGILPRQQYFKQNCSQHESSPDISHFER |
| | | SKQVSSVNEEDFVRLKQQISDHISQSCGSGQMKMFQEVSAA |
| | | DAFGPGTEGQVERFETVGMEAATDEGMPKSYLPQTVRQGG |
| | | YMPQ |
| 76 | mbIL-15 | MDWTWILFLVAAATRVHSGIHVFILGCFSAGLPKTEANWVN |
| | amino acid | VISDLKKIEDLIQSMHIDATLYTESDVHPSCKVTAMKCFLLEL |
| | sequence | QVISLESGDASIHDTVENLIILANNSLSSNGNVTESGCKECEE |
| | | LEEKNIKEFLQSFVHIVQMFINTSSGGGSGGGGSGGGGSGGG |
| | | GSGGGSLQITCPPPMSVEHADIWVKSYSLYSRERYICNSGFK |
| | | RKAGTSSLTECVLNKATNVAHWTTPSLKCIRDPALVHQRPA |
| | | PPSTVTTAGVTPQPESLSPSGKEPAASSPSSNNTAATTAAIVP |
| | | GSQLMPSKSPSTGTTEISSHESSHGTPSQTTAKNWELTASAS |
| | | HQPPGVYPQGHSDTTVAISTSTVLLCGLSAVSLLACYLKSRQ |
| | | TPPLASVEMEAMEALPVTWGTSSRDEDLENCSHHL |
| 77 | C7R | KKRIKPIVWPSLPDHKKTLEHLCKKPRKNLNVSFNPESFLDC |
| | intracellular | QIHRVDDIQARDEVEGFLQDTFPQQLEESEKQRLGGDVQSPN |
| | signaling | CPSEDVVITPESFGRDSSLTCLAGNVSACDAPILSSSRSLDCR |
| | domain amino | ESGKNGPHVYQDLLLSLGTTNSTLPPPFSLQSGILTLNPVAQG |
| | acid sequence | QPILTSLGSNQEEAYVTMSSFYQNQ |
| 78 | C7R amino | MLVRRGARAGPRMPRGWTALCLLSLLPSGFMSLDNNGTAT |
| | acid sequence | PELPTQGTFSNVSTNVSYQETTTPSTLGSTSLHPVSQHGNEA |
| | | TTNITETTVKFTSTSVITSVYGNTNSSVQSQTSVISTVFTTPAN |
| | | VSTPETTLKPSLSPGNVSDLSTTSTSLATSPTKPYTSSSPILSDI |
| | | KAEIKCSGIREVKLTQGICLEQNKTSSCAEFKKDRGEGLARV |
| | | LCGEEQADADAGAQVCSLLLAQSEVRPQCLLLVLANRTEISS |
| | | KLQLMKKHQSDLKKLGILDFTEQDVASHQSYSQKTPILLTCP |
| | | TISILSFFSVALLVILACVLWKKRIKPIVWPSLPDHKKTLEHLC |
| | | KKPRKNLNVSFNPESFLDCQIHRVDDIQARDEVEGFLQDTFP |
| | | QQLEESEKQRLGGDVQSPNCPSEDVVITPESFGRDSSLTCLA |
| | | GNVSACDAPILSSSRSLDCRESGKNGPHVYQDLLLSLGTTNS |
| | | TLPPPFSLQSGILTLNPVAQGQPILTSLGSNQEEAYVTMSSFY |
| | | QNQ |
| 79 | L-gp130 | AACAAGCGGGACCTTATCAAAAAGCACATCTGGCCCAAC |
| | intracellular | GTGCCAGATCCCAGCAAATCTCATATTGCTCAATGGAGTC |
| | signaling | CACATACGCCACCACGACACAACTTCAATAGCAAGGACC |
| | domain | AGATGTACTCTGACGGGAATTTTACCGACGTGTCAGTAGT |
| | nucleic acid | AGAGATCGAAGCCAATGACAAGAAGCCCTTCCCGGAGGA |
| | sequence | CCTCAAGAGCCTAGATCTCTTCAAAAAGGAGAAAATTAA |
| | | CACGGAGGGACACAGCTCAGGAATTGGCGGTAGCTCCTG |
| | | TATGTCAAGCTCAAGGCCGTCAATAAGCAGTAGTGATGA |
| | | GAACGAGTCCTCCCAGAACACCAGTTCAACAGTGCAGTA |
| | | CAGTACCGTCGTGCACAGTGGGTATAGGCATCAAGTTCC |
| | | CAGCGTGCAAGTGTTCAGCCGATCCGAAAGTACACAGCC |
| | | ATTATTGGACTCCGAGGAGAGGCCTGAGGACTTGCAGCT |
| | | CGTGGACCACGTGGACGGTGGGGATGGGATTCTGCCAAG |
| | | ACAACAATACTTTAAGCAAAACTGCTCCCAGCACGAGTC |
| | | TAGCCCGGATATATCACATTTCGAAAGATCTAAACAGGT |
| | | CAGCTCGGTGAATGAGGAAGATTTTGTTCGGCTGAAGCA |
| | | GCAGATAAGTGACCACATCAGTCAGAGTTGTGGTTCAGG |
| | | CCAGATGAAGATGTTTCAGGAGGTATCTGCTGCGGATGC |
| | | CTTTGGCCCTGGCACAGAGGGTCAAGTTGAAAGGTTCGA |
| | | AACCGTCGGAATGGAAGCTGCCACAGATGAGGGAATGCC |
| | | TAAGTCTTATTTGCCACAAACTGTCCGGCAGGGCGGGTAT |
| | | ATGCCTCAG |
| 80 | L-gp130 | ATGGCCCTGCCAGTAACGGCTCTGCTGCTGCCACTTGCTC |
| | nucleic acid | TGCTCCTCCATGCAGCCAGGCCTGACTACAAAGACGATG |
| | sequence | ACGACAAGGAACTGTGCGGAGGGCGAATCGCCCGCCTGG |
| | | AAGAGAAAGTCAAAACCCTGAAAGCCCAAAACTCTGAAC |
| | | TGGCATCAACTGCGAATATGCTCCGTGAGCAGGTCGCTC |
| | | AGCTTAAGCAGAAAGTGATGAATGCACAAGGCGAAATCG |
| | | AGGCCATAGTGGTTCCAGTTTGTTTGGCATTTCTCCTCAC |
| | | CACTCTGCTGGGCGTCCTTTTCTGCTTCAACAAGCGGGAC |
| | | CTTATCAAAAAGCACATCTGGCCCAACGTGCCAGATCCC |
| | | AGCAAATCTCATATTGCTCAATGGAGTCCACATACGCCAC |
| | | CACGACACAACTTCAATAGCAAGGACCAGATGTACTCTG |
| | | ACGGGAATTTTACCGACGTGTCAGTAGTAGAGATCGAAG |
| | | CCAATGACAAGAAGCCCTTCCCGGAGGACCTCAAGAGCC |
| | | TAGATCTCTTCAAAAAGGAGAAAATTAACACGGAGGGAC |
| | | ACAGCTCAGGAATTGGCGGTAGCTCCTGTATGTCAAGCTC |
| | | AAGGCCGTCAATAAGCAGTAGTGATGAGAACGAGTCCTC |
| | | CCAGAACACCAGTTCAACAGTGCAGTACAGTACCGTCGT |
| | | GCACAGTGGGTATAGGCATCAAGTTCCCAGCGTGCAAGT |
| | | GTTCAGCCGATCCGAAAGTACACAGCCATTATTGGACTCC |
| | | GAGGAGAGGCCTGAGGACTTGCAGCTCGTGGACCACGTG |
| | | GACGGTGGGGATGGGATTCTGCCAAGACAACAATACTTT |
| | | AAGCAAAACTGCTCCCAGCACGAGTCTAGCCCGGATATA |
| | | TCACATTTCGAAAGATCTAAACAGGTCAGCTCGGTGAAT |
| | | GAGGAAGATTTTGTTCGGCTGAAGCAGCAGATAAGTGAC |
| | | CACATCAGTCAGAGTTGTGGTTCAGGCCAGATGAAGATG |
| | | TTTCAGGAGGTATCTGCTGCGGATGCCTTTGGCCCTGGCA |
| | | CAGAGGGTCAAGTTGAAAGGTTCGAAACCGTCGGAATGG |
| | | AAGCTGCCACAGATGAGGGAATGCCTAAGTCTTATTTGC |
| | | CACAAACTGTCCGGCAGGGCGGGTATATGCCTCAG |
| 81 | mbIL-15 | ATGGATTGGACTTGGATACTGTTTCTGGTGGCAGCCGCTA |
| | nucleic acid | CCCGGGTACACAGTGGCATCCACGTGTTTATCCTGGGTTG |
| | sequence | CTTCTCTGCCGGCCTGCCCAAGACCGAAGCCAACTGGGTC |
| | | AACGTAATCTCAGACCTCAAGAAGATCGAGGATTTGATT |
| | | CAGTCGATGCACATTGACGCCACTCTCTACACCGAGTCCG |
| | | ACGTCCATCCCTCCTGTAAAGTTACCGCGATGAAGTGTTT |
| | | CCTGCTAGAGCTTCAAGTGATAAGCCTGGAGAGTGGCGA |
| | | CGCCTCCATACACGATACCGTGGAGAATCTCATCATCCTG |
| | | GCTAACAACTCCCTAAGTTCCAATGGGAATGTCACCGAA |
| | | AGCGGCTGTAAAGAGTGCGAGGAACTGGAGGAAAAGAA |
| | | CATCAAGGAGTTCCTCCAGTCTTTCGTGCACATCGTGCAG |
| | | ATGTTTATTAATACGTCATCAGGCGGCGGAAGTGGTGGA |
| | | GGAGGATCTGGAGGAGGAGGGTCTGGTGGAGGCGGATCT |
| | | GGCGGCGGGTCCTTACAGATCACCTGTCCACCACCAATG |
| | | AGCGTGGAACATGCAGACATTTGGGTGAAATCATATAGT |
| | | CTGTACTCACGAGAGCGATACATCTGTAACTCTGGGTTTA |
| | | AGAGAAAGGCTGGAACAAGTAGCCTGACCGAGTGCGTTC |
| | | TGAACAAGGCTACCAACGTAGCTCACTGGACAACACCCA |
| | | GCCTTAAATGTATTAGAGATCCGGCCCTGGTCCATCAGCG |
| | | TCCTGCACCTCCGTCTACAGTCACAACGGCTGGGGTGACA |
| | | CCACAGCCAGAATCCCTGTCACCCAGCGGTAAAGAACCC |
| | | GCCGCCTCCTCACCTAGTTCTAATAACACGGCTGCGACTA |
| | | CCGCCGCGATAGTGCCTGGGAGCCAGTTGATGCCCTCTA |
| | | AATCGCCCTCAACTGGGACTACAGAAATTAGCAGTCACG |
| | | AGTCCTCACACGGCACACCTAGCCAGACCACCGCAAAAA |
| | | ATTGGGAATTAACAGCCAGCGCCTCCCATCAGCCTCCTGG |
| | | GGTCTATCCACAAGGTCACAGCGATACTACGGTTGCAATT |
| | | AGCACTAGCACTGTTCTGTTGTGCGGCCTGAGCGCAGTCA |
| | | GTTTGCTCGCCTGCTATCTCAAGTCAAGGCAAACACCACC |
| | | GTTAGCTAGTGTGGAAATGGAGGCAATGGAGGCCTTGCC |
| | | CGTGACTTGGGGCACCAGCTCCCGCGACGAAGATCTCGA |
| | | GAATTGCTCACATCATCTG |
| 82 | C7R | AAAAAACGTATCAAGCCCATTGTGTGGCCCTCTCTGCCCG |
| | intracellular | ACCACAAAAAGACCCTCGAACACCTCTGCAAGAAGCCAC |
| | signaling | GGAAGAATCTTAATGTCTCCTTCAACCCTGAATCATTCCT |
| | domain | AGACTGCCAGATTCATCGAGTTGACGACATTCAGGCTCG |
| | nucleic acid | GGACGAGGTGGAAGGGTTTCTCCAGGATACCTTCCCACA |
| | sequence | GCAACTGGAGGAGAGCGAGAAACAGCGCCTGGGTGGGG |
| | | ACGTCCAGAGTCCAAATTGTCCCAGCGAGGATGTGGTTA |
| | | TCACTCCTGAATCGTTTGGAAGAGATTCCAGTCTGACCTG |
| | | TCTGGCAGGCAACGTTTCAGCGTGTGACGCACCGATACT |
| | | GTCCAGCTCTAGGTCCTTGGATTGTAGGGAGAGCGGCAA |
| | | AAATGGGCCACACGTGTATCAAGATCTCCTGTTAAGTCTG |
| | | GGAACAACAAACTCTACACTCCCACCGCCATTTAGCCTGC |
| | | AATCCGGAATTTTAACACTTAACCCTGTAGCTCAAGGCCA |
| | | GCCTATATTGACCAGCCTTGGTAGCAACCAGGAAGAGGC |
| | | CTACGTGACTATGTCAAGTTTTTACCAGAACCAA |
| 83 | C7R nucleic | ATGTTAGTACGGCGTGGGGCAAGGGCTGGACCAAGGATG |
| | acid sequence | CCACGCGGCTGGACAGCCTTGTGCCTGCTGTCACTGCTTC |
| | | CCTCGGGCTTTATGAGCCTCGACAATAACGGCACGGCAA |
| | | CACCCGAGTTGCCTACTCAAGGGACCTTTTCCAACGTGTC |
| | | CACCAATGTCAGTTATCAGGAGACAACTACTCCCTCCACC |
| | | CTGGGCTCTACGTCTTTGCATCCCGTCAGTCAACACGGTA |
| | | ACGAGGCTACCACGAACATCACGGAGACAACGGTCAAAT |
| | | TCACTTCTACAAGCGTTATAACTAGCGTCTACGGCAATAC |
| | | TAACAGTTCAGTTCAGTCTCAGACCAGCGTGATCTCAACC |
| | | GTATTTACTACACCGGCGAACGTTTCAACACCTGAAACA |
| | | ACCCTTAAACCGTCCCTGTCACCCGGGAATGTCTCCGACC |
| | | TGAGCACAACCTCCACTAGCTTGGCCACCTCTCCTACAAA |
| | | GCCCTATACCAGCAGCTCACCGATACTGAGCGACATTAA |
| | | GGCGGAAATCAAGTGCTCGGGTATTCGAGAAGTGAAACT |
| | | CACCCAAGGAATCTGTCTTGAACAGAACAAGACCAGTTC |
| | | CTGCGCTGAGTTCAAAAAGGACCGTGGGGAAGGCCTTGC |
| | | AAGAGTGCTGTGTGGGGAGGAACAGGCCGACGCCGATGC |
| | | CGGAGCCCAAGTGTGTAGTCTGCTATTAGCACAATCCGA |
| | | GGTGCGGCCACAGTGCCTGTTGCTCGTGCTGGCCAATCGA |
| | | ACCGAGATCTCTTCTAAGCTCCAGTTGATGAAGAAGCAC |
| | | CAGTCCGATCTCAAAAAGCTAGGAATTTTAGATTTCACTG |
| | | AGCAGGATGTGGCTTCACATCAGAGTTACTCTCAGAAAA |
| | | CCCCCATCTTGTTAACTTGCCCTACGATCTCGATACTAAG |
| | | TTTCTTCTCCGTCGCCCTCCTGGTAATCTTGGCCTGCGTGC |
| | | TGTGGAAAAAACGTATCAAGCCCATTGTGTGGCCCTCTCT |
| | | GCCCGACCACAAAAAGACCCTCGAACACCTCTGCAAGAA |
| | | GCCACGGAAGAATCTTAATGTCTCCTTCAACCCTGAATCA |
| | | TTCCTAGACTGCCAGATTCATCGAGTTGACGACATTCAGG |
| | | CTCGGGACGAGGTGGAAGGGTTTCTCCAGGATACCTTCC |
| | | CACAGCAACTGGAGGAGAGCGAGAAACAGCGCCTGGGT |
| | | GGGGACGTCCAGAGTCCAAATTGTCCCAGCGAGGATGTG |
| | | GTTATCACTCCTGAATCGTTTGGAAGAGATTCCAGTCTGA |
| | | CCTGTCTGGCAGGCAACGTTTCAGCGTGTGACGCACCGAT |
| | | ACTGTCCAGCTCTAGGTCCTTGGATTGTAGGGAGAGCGG |
| | | CAAAAATGGGCCACACGTGTATCAAGATCTCCTGTTAAG |
| | | TCTGGGAACAACAAACTCTACACTCCCACCGCCATTTAGC |
| | | CTGCAATCCGGAATTTTAACACTTAACCCTGTAGCTCAAG |
| | | GCCAGCCTATATTGACCAGCCTTGGTAGCAACCAGGAAG |
| | | AGGCCTACGTGACTATGTCAAGTTTTTACCAGAACCAA |
| 84 | Truncated | MDWTWILFLVAAATRVHSRKVCNGIGIGEFKDSLSINATNIK |
| | EGFR | HFKNCTSISGDLHILPVAFRGDSFTHTPPLDPQELDILKTVKEI |
| | (EGFRt) + | TGFLLIQAWPENRTDLHAFENLEIIRGRTKQHGQFSLAVVSL |
| | IgE signal | NITSLGLRSLKEISDGDVIISGNKNLCYANTINWKKLFGTSGQ |
| | peptide amino | KTKIISNRGENSCKATGQVCHALCSPEGCWGPEPRDCVSCR |
| | acid | NVSRGRECVDKCNLLEGEPREFVENSECIQCHPECLPQAMNI |
| | sequenced | TCTGRGPDNCIQCAHYIDGPHCVKTCPAGVMGENNTLVWK |
| | | YADAGHVCHLCHPNCTYGCTGPGLEGCPTNGPKIPSIATGM |
| | | VGALLLLLVVALGIGLFM |
| 85 | cJun amino | MTAKMETTFYDDALNASFLPSESGPYGYSNPKILKQSMTLN |
| | acid sequence | LADPVGSLKPHLRAKNSDLLTSPDVGLLKLASPELERLIIQSS |
| | | NGHITTTPTPTQFLCPKNVTDEQEGFAEGFVRALAELHSQNT |
| | | LPSVTSAAQPVNGAGMVAPAVASVAGGSGSGGFSASLHSEP |
| | | PVYANLSNFNPGALSSGGGAPSYGAAGLAFPAQPQQQQQPP |
| | | HHLPQQMPVQHPRLQALKEEPQTVPEMPGETPPLSPIDMESQ |
| | | ERIKAERKRMRNRIAASKCRKRKLERIARLEEKVKTLKAQN |
| | | SELASTANMLREQVAQLKQKVMNHVNSGCQLMLTQQLQTF |
| 86 | IL-2 | APTSSSTKKTQLQLEHLLLDLQMILNGINNYKNPKLTRMLTF |
| | polypeptide | KFYMPKKATELKHLQCLEEELKPLEEVLNLAQSKNFHLRPR |
| | sequence | DLISNINVIVLELKGSETTFMCEYADETATIVEFLNRWITFCQ |
| | | SIISTLT |
| 87 | IL-2 nucleic | GCACCAACGTCATCATCAACCAAAAAGACCCAGCTTCAG |
| | acid sequence | CTAGAGCATCTACTGCTGGACCTCCAAATGATTTTGAACG |
| | | GGATCAACAACTATAAGAATCCCAAACTAACAAGGATGC |
| | | TGACGTTCAAGTTTTACATGCCGAAAAAGGCAACCGAAC |
| | | TTAAACACCTCCAATGTTTAGAGGAGGAACTCAAACCTCT |
| | | CGAGGAGGTATTGAATCTGGCGCAATCCAAGAATTTCCA |
| | | CCTGAGGCCACGCGATCTGATCAGCAACATCAATGTGAT |
| | | CGTGCTTGAATTGAAGGGAAGCGAAACGACTTTCATGTG |
| | | CGAATACGCCGATGAGACTGCCACAATCGTGGAGTTCCT |
| | | TAATCGGTGGATAACCTTTTGTCAGTCAATCATTTCTACA |
| | | CTGACC |
| 88 | IL-7 | DCDIEGKDGKQYESVLMVSIDQLLDSMKEIGSNCLNNEFNFF |
| | polypeptide | KRHICDANKEGMFLFRAARKLRQFLKMNSTGDFDLHLLKVS |
| | sequence | EGTTILLNCTGQVKGRKPAALGEAQPTKSLEENKSLKEQKK |
| | | LNDLCFLKRLLQEIKTCWNKILMGTKEH |
| 89 | IL-7 nucleic | GATTGTGACATTGAAGGGAAAGATGGGAAACAGTACGAG |
| | acid sequence | TCCGTCCTGATGGTATCTATAGACCAGTTACTCGATAGCA |
| | | TGAAGGAGATCGGCTCCAACTGCCTTAACAACGAGTTTA |
| | | ATTTCTTCAAACGCCATATTTGCGATGCCAATAAGGAAGG |
| | | AATGTTCTTATTCCGAGCTGCCCGGAAGCTGAGGCAGTTT |
| | | CTGAAGATGAACAGCACTGGCGACTTTGACCTGCACCTC |
| | | CTTAAGGTGTCTGAGGGCACCACAATTCTGCTGAATTGTA |
| | | CCGGCCAAGTGAAGGGACGAAAACCTGCTGCGCTAGGAG |
| | | AAGCACAACCCACAAAATCACTCGAGGAGAATAAGAGTC |
| | | TTAAGGAACAGAAGAAGCTGAACGACCTCTGCTTTTTGA |
| | | AGAGGCTGTTGCAAGAGATCAAAACCTGTTGGAATAAGA |
| | | TCTTGATGGGGACCAAAGAACAC |
| 90 | IL-21 | QGQDRHMIRMRQLIDIVDQLKNYVNDLVPEFLPAPEDVETN |
| | polypeptide | CEWSAFSCFQKAQLKSANTGNNERIINVSIKKLKRKPPSTNA |
| | sequence | GRRQKHRLTCPSCDSYEKKPPKEFLERFKSLLQKMIHQHLSS |
| | | RTHGSEDS |
| 91 | IL-21 nucleic | CAAGGACAGGACCGACATATGATAAGGATGAGGCAATTA |
| | acid sequence | ATTGATATTGTGGACCAATTGAAAAACTACGTCAATGAC |
| | | CTCGTGCCAGAGTTCCTGCCCGCTCCCGAAGATGTAGAA |
| | | ACGAATTGCGAATGGTCAGCCTTTAGCTGTTTCCAAAAGG |
| | | CACAGCTCAAAAGCGCGAATACCGGAAACAACGAGCGTA |
| | | TAATTAACGTTTCAATCAAAAAACTTAAGAGGAAGCCTC |
| | | CTTCTACAAATGCCGGCAGGCGCCAGAAGCACCGCCTGA |
| | | CCTGCCCGTCTTGCGACAGTTATGAGAAGAAGCCACCCA |
| | | AAGAATTTCTCGAGAGATTCAAGTCTTTACTTCAGAAAAT |
| | | GATCCATCAGCACCTGAGCAGCCGAACTCACGGGAGTGA |
| | | AGATTCC |
| 92 | IL-12 | IWELKKDVYVVELDWYPDAPGEMVVLTCDTPEEDGITWTL |
| | polypeptide | DQSSEVLGSGKTLTIQVKEFGDAGQYTCHKGGEVLSHSLLLL |
| | sequence | HKKEDGIWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSF |
| | | FIRDIIKPDPPKNLQLKPLKNSRQVEVSWEYPDTWSTPHSYFS |
| | | LTFCVQVQGKSKREKKDRVFTDKTSATVICRKNASISVRAQ |
| | | DRYYSSSWSEWASVPCSGGGGSGGGGSGGGGSRNLPVATP |
| | | DPGMFPCLHHSQNLLRAVSNMLQKARQTLEFYPCTSEEIDH |
| | | EDITKDKTSTVEACLPLELTKNESCLNSRETSFITNGSCLASR |
| | | KTSFMMALCLSSIYEDLKMYQVEFKTMNAKLLMDPKRQIFL |
| | | DQNMLAVIDELMQALNFNSETVPQKSSLEEPDFYKTKIKLCI |
| | | LLHAFRIRAVTIDRVMSYLNAS |
| 93 | IL-12 nucleic | ATTTGGGAACTGAAGAAAGACGTATATGTCGTGGAACTG |
| | acid sequence | GACTGGTATCCCGATGCCCCAGGGGAAATGGTGGTCCTT |
| | | ACTTGCGACACCCCCGAAGAGGACGGCATTACATGGACC |
| | | CTTGACCAGTCCAGTGAGGTCCTCGGGAGTGGAAAGACA |
| | | CTGACAATTCAGGTTAAGGAGTTCGGCGATGCCGGTCAG |
| | | TACACCTGTCATAAAGGGGGAGAGGTTCTGTCCCATTCAC |
| | | TCCTGCTGCTGCACAAGAAGGAAGATGGTATCTGGTCCA |
| | | CGGACATTTTGAAAGATCAAAAGGAGCCCAAGAACAAAA |
| | | CTTTCCTGCGGTGTGAAGCCAAAAATTACAGCGGGCGAT |
| | | TTACTTGTTGGTGGCTCACCACTATTAGTACAGATCTCAC |
| | | CTTCAGCGTTAAGAGTTCTAGGGGCTCCAGTGATCCGCAG |
| | | GGAGTGACGTGTGGCGCTGCGACCCTGTCCGCTGAGCGA |
| | | GTGAGGGGCGATAATAAGGAGTATGAGTATAGTGTGGAG |
| | | TGTCAGGAGGATTCTGCTTGCCCTGCTGCCGAGGAGTCCC |
| | | TTCCTATCGAAGTGATGGTCGATGCTGTACACAAACTGAA |
| | | ATACGAGAATTATACTAGCTCCTTTTTCATAAGGGACATC |
| | | ATAAAGCCAGATCCCCCCAAGAACCTTCAGCTGAAACCG |
| | | CTGAAAAATTCACGCCAGGTGGAGGTATCTTGGGAGTAC |
| | | CCAGACACATGGAGCACACCTCATAGTTACTTCAGCCTG |
| | | ACCTTCTGTGTCCAGGTCCAGGGGAAGTCTAAAAGGGAA |
| | | AAAAAGGACAGAGTTTTCACCGACAAGACATCCGCAACC |
| | | GTAATCTGTAGGAAAAACGCCTCAATTTCCGTTAGAGCG |
| | | CAAGATAGGTATTATTCCTCTTCCTGGTCTGAATGGGCCT |
| | | CTGTACCCTGCAGCGGGGGTGGCGGTAGCGGTGGGGGAG |
| | | GGAGCGGAGGTGGAGGCAGCCGAAACCTGCCTGTGGCCA |
| | | CACCTGATCCTGGGATGTTCCCTTGCTTGCACCACAGTCA |
| | | GAATCTGCTCAGGGCCGTTAGCAACATGCTGCAGAAGGC |
| | | GCGGCAGACACTTGAGTTCTACCCTTGCACAAGTGAAGA |
| | | GATCGATCACGAAGATATCACAAAGGATAAAACGTCTAC |
| | | TGTCGAGGCCTGCCTCCCCCTCGAACTCACAAAGAACGA |
| | | GAGTTGCCTCAATTCCCGGGAAACCAGCTTCATCACTAAT |
| | | GGGTCCTGCCTGGCAAGCAGAAAAACTTCTTTCATGATG |
| | | GCATTGTGTTTGAGTAGCATCTATGAGGACCTCAAAATGT |
| | | ACCAAGTTGAATTTAAGACTATGAACGCTAAGCTCCTCAT |
| | | GGACCCTAAGCGGCAGATCTTCCTGGATCAGAACATGTT |
| | | GGCTGTCATCGACGAACTCATGCAAGCCCTGAACTTTAAT |
| | | TCCGAAACAGTTCCCCAGAAGTCCAGTCTGGAGGAACCC |
| | | GATTTCTACAAAACCAAAATTAAGCTGTGTATCTTGCTGC |
| | | ATGCTTTCCGGATCAGGGCCGTTACGATCGATCGGGTAAT |
| | | GAGCTACCTGAATGCCAGT |
| 94 | IL-12/23p40 | IWELKKDVYVVELDWYPDAPGEMVVLTCDTPEEDGITWTL |
| | polypeptide | DQSSEVLGSGKTLTIQVKEFGDAGQYTCHKGGEVLSHSLLLL |
| | sequence | HKKEDGIWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSF |
| | | FIRDIIKPDPPKNLQLKPLKNSRQVEVSWEYPDTWSTPHSYFS |
| | | LTFCVQVQGKSKREKKDRVFTDKTSATVICRKNASISVRAQ |
| | | DRYYSSSWSEWASVPCS |
| 95 | IL-12/23p40 | ATTTGGGAACTCAAAAAGGACGTGTATGTAGTCGAGCTA |
| | nucleic acid | GATTGGTATCCCGATGCACCCGGTGAAATGGTCGTTCTGA |
| | sequence | CCTGCGACACACCAGAAGAGGATGGAATCACCTGGACTC |
| | | TGGATCAAAGCTCCGAGGTGCTGGGGAGCGGCAAAACCC |
| | | TGACAATTCAGGTCAAAGAGTTCGGCGACGCGGGACAGT |
| | | ACACTTGCCATAAGGGTGGGGAGGTTCTGTCACACTCTCT |
| | | GCTACTGCTGCACAAAAAGGAGGACGGAATATGGTCAAC |
| | | GGACATCCTGAAGGACCAGAAGGAGCCAAAAAACAAGA |
| | | CCTTCCTGAGGTGCGAGGCCAAAAACTACTCAGGCCGCT |
| | | TTACTTGTTGGTGGCTGACAACAATCTCCACAGACCTCAC |
| | | CTTTAGTGTGAAGAGTAGTCGCGGATCTAGCGATCCTCAA |
| | | GGCGTCACTTGCGGTGCCGCTACCCTGAGTGCTGAACGA |
| | | GTGCGTGGGGACAACAAGGAATACGAGTACAGTGTGGAA |
| | | TGCCAAGAGGACAGCGCTTGCCCGGCAGCCGAGGAATCA |
| | | CTACCCATTGAAGTGATGGTCGATGCCGTCCATAAGTTAA |
| | | AATACGAGAATTACACGTCCTCTTTCTTCATCCGCGATAT |
| | | AATCAAGCCTGACCCGCCAAAGAATTTGCAGTTGAAGCC |
| | | CTTGAAAAACAGCCGGCAGGTTGAAGTGTCGTGGGAATA |
| | | TCCCGACACTTGGAGCACACCACATAGCTATTTCTCACTG |
| | | ACATTCTGTGTGCAAGTTCAGGGAAAGTCAAAACGGGAG |
| | | AAGAAGGATAGAGTATTTACCGACAAAACGTCAGCGACA |
| | | GTGATATGTCGAAAAAATGCGTCGATTAGTGTGCGGGCA |
| | | CAGGATAGATACTATTCCAGTTCTTGGTCCGAGTGGGCTT |
| | | CTGTACCTTGTTCA |
| 96 | IL-15 | GIHVFILGCFSAGLPKTEANWVNVISDLKKIEDLIQSMHIDAT |
| | polypeptide | LYTESDVHPSCKVTAMKCFLLELQVISLESGDASIHDTVENLI |
| | sequence | ILANNSLSSNGNVTESGCKECEELEEKNIKEFLQSFVHIVQMF |
| | | INTS |
| 97 | IL-15 nucleic | GGCATCCACGTGTTTATCCTGGGTTGCTTCTCTGCCGGCC |
| | acid sequence | TGCCCAAGACCGAAGCCAACTGGGTCAACGTAATCTCAG |
| | | ACCTCAAGAAGATCGAGGATTTGATTCAGTCGATGCACA |
| | | TTGACGCCACTCTCTACACCGAGTCCGACGTCCATCCCTC |
| | | CTGTAAAGTTACCGCGATGAAGTGTTTCCTGCTAGAGCTT |
| | | CAAGTGATAAGCCTGGAGAGTGGCGACGCCTCCATACAC |
| | | GATACCGTGGAGAATCTCATCATCCTGGCTAACAACTCCC |
| | | TAAGTTCCAATGGGAATGTCACCGAAAGCGGCTGTAAAG |
| | | AGTGCGAGGAACTGGAGGAAAAGAACATCAAGGAGTTCC |
| | | TCCAGTCTTTCGTGCACATCGTGCAGATGTTTATTAATAC |
| | | GTCA |
| 98 | IL-18 | YFGKLESKLSVIRNLNDQVLFIDQGNRPLFEDMTDSDCRDNA |
| | polypeptide | PRTIFIISMYKDSQPRGMAVTISVKCEKISTLSCENKIISFKEM |
| | sequence | NPPDNIKDTKSDIIFFQRSVPGHDNKMQFESSSYEGYFLACEK |
| | | ERDLFKLILKKEDELGDRSIMFTVQNED |
| 99 | IL-18 nucleic | TATTTTGGAAAACTCGAATCAAAACTTTCTGTGATCCGGA |
| | acid sequence | ACCTCAACGACCAGGTTTTATTCATCGACCAAGGGAATC |
| | | GACCGCTGTTCGAGGACATGACAGATAGCGACTGTAGAG |
| | | ATAATGCTCCCAGGACCATTTTTATAATCTCCATGTACAA |
| | | AGATAGCCAGCCTCGCGGGATGGCAGTGACTATTAGCGT |
| | | GAAATGCGAGAAAATCTCTACCCTGAGTTGCGAAAACAA |
| | | AATAATCTCCTTTAAGGAGATGAATCCACCAGACAACAT |
| | | TAAGGACACAAAGTCCGACATAATTTTCTTTCAACGAAGT |
| | | GTCCCTGGACACGATAACAAAATGCAGTTCGAAAGTAGT |
| | | AGTTACGAGGGTTATTTCCTGGCCTGTGAGAAGGAGCGG |
| | | GATCTGTTTAAGCTAATCCTTAAGAAGGAAGATGAATTG |
| | | GGCGATAGATCGATAATGTTCACGGTCCAGAATGAGGAC |
| 100 | CD44 signal | MDKFWWHAAWGLCLVPLSLA |
| | peptide | |
| | sequence | |
| 101 | CD44 signal | ATGGACAAGTTTTGGTGGCACGCAGCCTGGGGACTCTGC |
| | peptide | CTCGTGCCGCTGAGCCTGGCG |
| | nucleic acid | |
| | sequence | |
| 102 | CD3E signal | MQSGTHWRVLGLCLLSVGVWGQ |
| | peptide | |
| | sequence | |
| 103 | CD3E signal | ATGCAGTCGGGCACTCACTGGAGAGTTCTGGGCCTCTGCC |
| | peptide | TCTTATCAGTTGGCGTTTGGGGGCAA |
| | nucleic acid | |
| | sequence | |
| 104 | CD5 signal | MPMGSLQPLATLYLLGMLVASCLG |
| | peptide | |
| | sequence | |
| 105 | CD5 signal | ATGCCCATGGGGTCTCTGCAACCGCTGGCCACCTTGTACC |
| | peptide | TGCTGGGGATGCTGGTCGCTTCCTGCCTCGGA |
| | nucleic acid | |
| | sequence | |
| 106 | IGTAL signal | MKDSCITVMAMALLSGFFFFAPASS |
| | peptide | |
| | sequence | |
| 107 | IGTAL signal | ATGAAGGATTCCTGCATCACTGTGATGGCCATGGCGCTGC |
| | peptide | TGTCTGGGTTCTTTTTCTTCGCGCCGGCCTCGAGC |
| | nucleic acid | |
| | sequence | |
| 108 | IL-2 signal | MYRMQLLSCIALSLALVTNS |
| | peptide | |
| | sequence | |
| 109 | IL-2 signal | ATGTACAGGATGCAACTCCTGTCTTGCATTGCACTAAGTC |
| | peptide | TTGCACTTGTCACAAACAGT |
| | nucleic acid | |
| | sequence | |
| 110 | GMCSF | MWLQSLLLLGTVACSIS |
| | signal peptide | |
| | sequence | |
| 111 | GMCSF | ATGTGGCTGCAGAGCCTGCTGCTCTTGGGCACTGTGGCCT |
| | signal peptide | GCAGCATCTCT |
| | nucleic acid | |
| | sequence | |
| 112 | Chymotrypsinogen | MAFLWLLSCWALLGTTFG |
| | signal | |
| | peptide | |
| | sequence | |
| 113 | Chymotrypsinogen | ATGGCTTTCCTCTGGCTCCTCTCCTGCTGGGCCCTCCTGG |
| | signal | GTACCACCTTCGGC |
| | peptide | |
| | nucleic acid | |
| | sequence | |
| 114 | Trypsinogen | MNLLLILTFVAAAVA |
| | signal peptide | |
| | sequence | |
| 115 | Trypsinogen | ATGAATCTACTTCTGATCCTTACCTTTGTTGCAGCTGCTGT |
| | signal peptide | TGCT |
| | nucleic acid | |
| | sequence | |
| 116 | IgK signal | MDMRVLAQLLGLLLLCFPGARC |
| | peptide | |
| | sequence | |
| 117 | IgK signal | ATGGACATGAGAGTCCTCGCTCAGCTCCTGGGGCTCCTGC |
| | peptide | TGCTCTGTTTCCCAGGTGCCAGATGT |
| | nucleic acid | |
| | sequence | |
| 118 | IgKVIII signal | MDMRVPAQLLGLLLLWLRGARC |
| | peptide | |
| | sequence | |
| 119 | IgKVIII signal | ATGGACATGAGGGTCCCCGCTCAGCTCCTGGGGCTCCTGC |
| | peptide | TACTCTGGCTCCGAGGTGCCAGATGT |
| | nucleic acid | |
| | sequence | |
| 120 | IgE signal | MDWTWILFLVAAATRVHS |
| | peptide | |
| | sequence | |
| 121 | IgE signal | ATGGACTGGACCTGGATCCTCTTCTTGGTGGCAGCAGCCA |
| | peptide | CGCGAGTCCACTCC |
| | nucleic acid | |
| | sequence | |
| 122 | OSM signal | MGVLLTQRTLLSLVLALLFPSMASM |
| | peptide | |
| | sequence | |
| 123 | OSM signal | ATGGGGGTACTGCTCACACAGAGGACGCTGCTCAGTCTG |
| | peptide | GTCCTTGCACTCCTGTTTCCAAGCATGGCGAGCATG |
| | nucleic acid | |
| | sequence | |
| 124 | IgG2H signal | MGWSCIILFLVATATGVHS |
| | peptide | |
| | sequence | |
| 125 | IgG2H signal | ATGGGCTGGTCCTGCATCATCCTGTTTCTGGTGGCCACAG |
| | peptide | CCACCGGTGTCCATTCT |
| | nucleic acid | |
| | sequence | |
| 126 | BM40 signal | MRAWIFFLLCLAGRALA |
| | peptide | |
| | sequence | |
| 127 | BM40 signal | ATGAGGGCCTGGATCTTCTTTCTCCTTTGCCTGGCCGGGA |
| | peptide | GGGCCTTGGCA |
| | nucleic acid | |
| | sequence | |
| 128 | Secrecon | MWWRLWWLLLLLLLLWPMVWA |
| | signal peptide | |
| | sequence | |
| 129 | Secrecon | ATGTGGTGGAGGCTTTGGTGGCTATTACTCCTGTTGCTCC |
| | signal peptide | TGCTGTGGCCTATGGTGTGGGCC |
| | nucleic acid | |
| | sequence | |
| 130 | tPA signal | MDAMKRGLCCVLLLCGAVFVSPS |
| | peptide | |
| | sequence | |
| 131 | tPA signal | ATGGATGCAATGAAGAGAGGGCTCTGCTGTGTGCTGCTG |
| | peptide | CTGTGTGGAGCAGTCTTCGTTTCGCCCAGC |
| | nucleic acid | |
| | sequence | |
| 132 | Super-2 | APTSSSTKKTQLQLEHLLLDLQMILNGINNYKNPKLTRMLTF |
| | polypeptide | KFYMPKKATELKHLQCLEEELKPLEEVLNLAQSKNFHFDPR |
| | sequence | DVVSNINVFVLELKGSETTFMCEYADETATIVEFLNRWITFC |
| | | QSIISTL |
| 133 | Super-2 | GCACCAACGTCATCATCAACCAAAAAGACCCAGCTTCAG |
| | nucleic acid | CTAGAGCATCTACTGCTGGACCTCCAAATGATTTTGAACG |
| | sequence | GGATCAACAACTATAAGAATCCCAAACTAACAAGGATGC |
| | | TGACGTTCAAGTTTTACATGCCGAAAAAGGCAACCGAAC |
| | | TTAAACACCTCCAATGTTTAGAGGAGGAACTCAAACCTCT |
| | | CGAGGAGGTATTGAATCTGGCGCAATCCAAGAATTTCCA |
| | | CTTTGACCCACGCGATGTGGTTAGCAACATCAATGTGTTT |
| | | GTGCTTGAATTGAAGGGAAGCGAAACGACTTTCATGTGC |
| | | GAATACGCCGATGAGACTGCCACAATCGTGGAGTTCCTT |
| | | AATCGGTGGATAACCTTTTGTCAGTCAATCATTTCTACAC |
| | | TGACC |
| 134 | IL-10 | SPGQGTQSENSCTHFPGNLPNMLRDLRDAFSRVKTFFQMKD |
| | polypeptide | QLDNLLLKESLLEDFKGYLGCQALSEMIQFYLEEVMPQAEN |
| | sequence | QDPDIKAHVNSLGENLKTLRLRLRRCHRFLPCENKSKAVEQ |
| | | VKNAFNKLQEKGIYKAMSEFDIFINYIEAYMTMKIRN |
| 135 | IL-10 nucleic | AGGCAACCTGCCTAACATGCTTCGAGATCTCCGAGATGC |
| | acid sequence | CTTCAGCAGAGTGAAGACTTTCTTTCAAATGAAGGATCA |
| | | GCTGGACAACTTGTTGTTAAAGGAGTCCTTGCTGGAGGA |
| | | CTTTAAGGGTTACCTGGGTTGCCAAGCCTTGTCTGAGATG |
| | | ATCCAGTTTTACCTGGAGGAGGTGATGCCCCAAGCTGAG |
| | | AACCAAGACCCAGACATCAAGGCGCATGTGAACTCCCTG |
| | | GGGGAGAACCTGAAGACCCTCAGGCTGAGGCTACGGCGC |
| | | TGTCATCGATTTCTTCCCTGTGAAAACAAGAGCAAGGCCG |
| | | TGGAGCAGGTGAAGAATGCCTTTAATAAGCTCCAAGAGA |
| | | AAGGCATCTACAAAGCCATGAGTGAGTTTGACATCTTCAT |
| | | CAACTACATAGAAGCCTACATGACAATGAAGATACGAAA |
| | | C |
| 136 | TGF-β | LSTCKTIDMELVKRKRIEAIRGQILSKLRLASPPSQGEVPPGPL |
| | polypeptide | PEAVLALYNSTRDRVAGESAEPEPEPEADYYAKEVTRVLMV |
| | sequence | ETHNEIYDKFKQSTHSIYMFFNTSELREAVPEPVLLSRAELRL |
| | | LRLKLKVEQHVELYQKYSNNSWRYLSNRLLAPSDSPEWLSF |
| | | DVTGVVRQWLSRGGEIEGFRLSAHCSCDSRDNTLQVDINGF |
| | | TTGRRGDLATIHGMNRPFLLLMATPLERAQHLQSSRHRRAL |
| | | DTNYCFSSTEKNCCVRQLYIDFRKDLGWKWIHEPKGYHANF |
| | | CLGPCPYIWSLDTQYSKVLALYNQHNPGASAAPCCVPQALE |
| | | PLPIVYYVGRKPKVEQLSNMIVRSCKCS |
| 137 | TGF-β | CTATCCACCTGCAAGACTATCGACATGGAGCTGGTGAAG |
| | nucleic acid | CGGAAGCGCATCGAGGCCATCCGCGGCCAGATCCTGTCC |
| | sequence | AAGCTGCGGCTCGCCAGCCCCCCGAGCCAGGGGGAGGTG |
| | | CCGCCCGGCCCGCTGCCCGAGGCCGTGCTCGCCCTGTACA |
| | | ACAGCACCCGCGACCGGGTGGCCGGGGAGAGTGCAGAAC |
| | | CGGAGCCCGAGCCTGAGGCCGACTACTACGCCAAGGAGG |
| | | TCACCCGCGTGCTAATGGTGGAAACCCACAACGAAATCT |
| | | ATGACAAGTTCAAGCAGAGTACACACAGCATATATATGT |
| | | TCTTCAACACATCAGAGCTCCGAGAAGCGGTACCTGAAC |
| | | CCGTGTTGCTCTCCCGGGCAGAGCTGCGTCTGCTGAGGCT |
| | | CAAGTTAAAAGTGGAGCAGCACGTGGAGCTGTACCAGAA |
| | | ATACAGCAACAATTCCTGGCGATACCTCAGCAACCGGCT |
| | | GCTGGCACCCAGCGACTCGCCAGAGTGGTTATCTTTTGAT |
| | | GTCACCGGAGTTGTGCGGCAGTGGTTGAGCCGTGGAGGG |
| | | GAAATTGAGGGCTTTCGCCTTAGCGCCCACTGCTCCTGTG |
| | | ACAGCAGGGATAACACACTGCAAGTGGACATCAACGGGT |
| | | TCACTACCGGCCGCCGAGGTGACCTGGCCACCATTCATG |
| | | GCATGAACCGGCCTTTCCTGCTTCTCATGGCCACCCCGCT |
| | | GGAGAGGGCCCAGCATCTGCAAAGCTCCCGGCACCGCCG |
| | | AGCCCTGGACACCAACTATTGCTTCAGCTCCACGGAGAA |
| | | GAACTGCTGCGTGCGGCAGCTGTACATTGACTTCCGCAA |
| | | GGACCTCGGCTGGAAGTGGATCCACGAGCCCAAGGGCTA |
| | | CCATGCCAACTTCTGCCTCGGGCCCTGCCCCTACATTTGG |
| | | AGCCTGGACACGCAGTACAGCAAGGTCCTGGCCCTGTAC |
| | | AACCAGCATAACCCGGGCGCCTCGGCGGCGCCGTGCTGC |
| | | GTGCCGCAGGCGCTGGAGCCGCTGCCCATCGTGTACTAC |
| | | GTGGGCCGCAAGCCCAAGGTGGAGCAGCTGTCCAACATG |
| | | ATCGTGCGCTCCTGCAAGTGCAGC |
| 139 | IL-2 precursor | MYRMQLLSCIALSLALVTNSAPTSSSTKKTQLQLEHLLLDLQ |
| | polypeptide | MILNGINNYKNPKLTRMLTFKFYMPKKATELKHLQCLEEEL |
| | sequence | KPLEEVLNLAQSKNFHLRPRDLISNINVIVLELKGSETTFMCE |
| | | YADETATIVEFLNRWITFCQSIISTLT |
| 140 | IL-2 precursor | ATGTATCGAATGCAACTGCTGTCGTGCATAGCACTATCAC |
| | nucleic acid | TGGCTCTGGTGACAAACAGTGCACCAACGTCATCATCAA |
| | sequence | CCAAAAAGACCCAGCTTCAGCTAGAGCATCTACTGCTGG |
| | | ACCTCCAAATGATTTTGAACGGGATCAACAACTATAAGA |
| | | ATCCCAAACTAACAAGGATGCTGACGTTCAAGTTTTACAT |
| | | GCCGAAAAAGGCAACCGAACTTAAACACCTCCAATGTTT |
| | | AGAGGAGGAACTCAAACCTCTCGAGGAGGTATTGAATCT |
| | | GGCGCAATCCAAGAATTTCCACCTGAGGCCACGCGATCT |
| | | GATCAGCAACATCAATGTGATCGTGCTTGAATTGAAGGG |
| | | AAGCGAAACGACTTTCATGTGCGAATACGCCGATGAGAC |
| | | TGCCACAATCGTGGAGTTCCTTAATCGGTGGATAACCTTT |
| | | TGTCAGTCAATCATTTCTACACTGACC |
| 141 | IL-7 precursor | MFHVSFRYIFGLPPLILVLLPVASSDCDIEGKDGKQYESVLM |
| | polypeptide | VSIDQLLDSMKEIGSNCLNNEFNFFKRHICDANKEGMFLFRA |
| | sequence | ARKLRQFLKMNSTGDFDLHLLKVSEGTTILLNCTGQVKGRK |
| | | PAALGEAQPTKSLEENKSLKEQKKLNDLCFLKRLLQEIKTC |
| | | WNKILMGTKEH |
| 142 | IL-7 precursor | ATGTTCCATGTGTCGTTTCGATATATCTTCGGCCTCCCACC |
| | nucleic acid | TCTGATTCTTGTGCTGTTGCCCGTTGCCTCCAGCGATTGTG |
| | sequence | ACATTGAAGGGAAAGATGGGAAACAGTACGAGTCCGTCC |
| | | TGATGGTATCTATAGACCAGTTACTCGATAGCATGAAGG |
| | | AGATCGGCTCCAACTGCCTTAACAACGAGTTTAATTTCTT |
| | | CAAACGCCATATTTGCGATGCCAATAAGGAAGGAATGTT |
| | | CTTATTCCGAGCTGCCCGGAAGCTGAGGCAGTTTCTGAAG |
| | | ATGAACAGCACTGGCGACTTTGACCTGCACCTCCTTAAGG |
| | | TGTCTGAGGGCACCACAATTCTGCTGAATTGTACCGGCCA |
| | | AGTGAAGGGACGAAAACCTGCTGCGCTAGGAGAAGCACA |
| | | ACCCACAAAATCACTCGAGGAGAATAAGAGTCTTAAGGA |
| | | ACAGAAGAAGCTGAACGACCTCTGCTTTTTGAAGAGGCT |
| | | GTTGCAAGAGATCAAAACCTGTTGGAATAAGATCTTGAT |
| | | GGGGACCAAAGAACAC |
| 143 | IL-21 | MRSSPGNMERIVICLMVIFLGTLVHKSSSQGQDRHMIRMRQ |
| | precursor | LIDIVDQLKNYVNDLVPEFLPAPEDVETNCEWSAFSCFQKAQ |
| | polypeptide | LKSANTGNNERIINVSIKKLKRKPPSTNAGRRQKHRLTCPSC |
| | sequence | DSYEKKPPKEFLERFKSLLQKMIHQHLSSRTHGSEDS |
| 144 | IL-21 | ATGAGAAGCTCACCCGGGAACATGGAACGTATCGTTATC |
| | precursor | TGTCTTATGGTGATTTTTCTGGGTACTCTCGTACACAAGA |
| | nucleic acid | GTAGTAGTCAAGGACAGGACCGACATATGATAAGGATGA |
| | sequence | GGCAATTAATTGATATTGTGGACCAATTGAAAAACTACG |
| | | TCAATGACCTCGTGCCAGAGTTCCTGCCCGCTCCCGAAGA |
| | | TGTAGAAACGAATTGCGAATGGTCAGCCTTTAGCTGTTTC |
| | | CAAAAGGCACAGCTCAAAAGCGCGAATACCGGAAACAA |
| | | CGAGCGTATAATTAACGTTTCAATCAAAAAACTTAAGAG |
| | | GAAGCCTCCTTCTACAAATGCCGGCAGGCGCCAGAAGCA |
| | | CCGCCTGACCTGCCCGTCTTGCGACAGTTATGAGAAGAA |
| | | GCCACCCAAAGAATTTCTCGAGAGATTCAAGTCTTTACTT |
| | | CAGAAAATGATCCATCAGCACCTGAGCAGCCGAACTCAC |
| | | GGGAGTGAAGATTCC |
| 145 | IL-12 | MCHQQLVISWFSLVFLASPLVAIWELKKDVYVVELDWYPD |
| | polypeptide | APGEMVVLTCDTPEEDGITWTLDQSSEVLGSGKTLTIQVKEF |
| | sequence | GDAGQYTCHKGGEVLSHSLLLLHKKEDGIWSTDILKDQKEP |
| | | KNKTFLRCEAKNYSGRFTCWWLTTISTDLTFSVKSSRGSSDP |
| | | QGVTCGAATLSAERVRGDNKEYEYSVECQEDSACPAAEESL |
| | | PIEVMVDAVHKLKYENYTSSFFIRDIIKPDPPKNLQLKPLKNS |
| | | RQVEVSWEYPDTWSTPHSYFSLTFCVQVQGKSKREKKDRVF |
| | | TDKTSATVICRKNASISVRAQDRYYSSSWSEWASVPCSGGG |
| | | GSGGGGSGGGGSRNLPVATPDPGMFPCLHHSQNLLRAVSN |
| | | MLQKARQTLEFYPCTSEEIDHEDITKDKTSTVEACLPLELTK |
| | | NESCLNSRETSFITNGSCLASRKTSFMMALCLSSIYEDLKMY |
| | | QVEFKTMNAKLLMDPKRQIFLDQNMLAVIDELMQALNFNS |
| | | ETVPQKSSLEEPDFYKTKIKLCILLHAFRIRAVTIDRVMSYLN |
| | | AS |
| 146 | IL-12 | ATGTGTCATCAGCAACTGGTCATTAGCTGGTTTTCCTTGG |
| | precursor | TGTTCCTGGCCTCACCTCTCGTTGCAATTTGGGAACTGAA |
| | nucleic acid | GAAAGACGTATATGTCGTGGAACTGGACTGGTATCCCGA |
| | sequence | TGCCCCAGGGGAAATGGTGGTCCTTACTTGCGACACCCCC |
| | | GAAGAGGACGGCATTACATGGACCCTTGACCAGTCCAGT |
| | | GAGGTCCTCGGGAGTGGAAAGACACTGACAATTCAGGTT |
| | | AAGGAGTTCGGCGATGCCGGTCAGTACACCTGTCATAAA |
| | | GGGGGAGAGGTTCTGTCCCATTCACTCCTGCTGCTGCACA |
| | | AGAAGGAAGATGGTATCTGGTCCACGGACATTTTGAAAG |
| | | ATCAAAAGGAGCCCAAGAACAAAACTTTCCTGCGGTGTG |
| | | AAGCCAAAAATTACAGCGGGCGATTTACTTGTTGGTGGC |
| | | TCACCACTATTAGTACAGATCTCACCTTCAGCGTTAAGAG |
| | | TTCTAGGGGCTCCAGTGATCCGCAGGGAGTGACGTGTGG |
| | | CGCTGCGACCCTGTCCGCTGAGCGAGTGAGGGGCGATAA |
| | | TAAGGAGTATGAGTATAGTGTGGAGTGTCAGGAGGATTC |
| | | TGCTTGCCCTGCTGCCGAGGAGTCCCTTCCTATCGAAGTG |
| | | ATGGTCGATGCTGTACACAAACTGAAATACGAGAATTAT |
| | | ACTAGCTCCTTTTTCATAAGGGACATCATAAAGCCAGATC |
| | | CCCCCAAGAACCTTCAGCTGAAACCGCTGAAAAATTCAC |
| | | GCCAGGTGGAGGTATCTTGGGAGTACCCAGACACATGGA |
| | | GCACACCTCATAGTTACTTCAGCCTGACCTTCTGTGTCCA |
| | | GGTCCAGGGGAAGTCTAAAAGGGAAAAAAAGGACAGAG |
| | | TTTTCACCGACAAGACATCCGCAACCGTAATCTGTAGGA |
| | | AAAACGCCTCAATTTCCGTTAGAGCGCAAGATAGGTATT |
| | | ATTCCTCTTCCTGGTCTGAATGGGCCTCTGTACCCTGCAG |
| | | CGGGGGTGGCGGTAGCGGTGGGGGAGGGAGCGGAGGTG |
| | | GAGGCAGCCGAAACCTGCCTGTGGCCACACCTGATCCTG |
| | | GGATGTTCCCTTGCTTGCACCACAGTCAGAATCTGCTCAG |
| | | GGCCGTTAGCAACATGCTGCAGAAGGCGCGGCAGACACT |
| | | TGAGTTCTACCCTTGCACAAGTGAAGAGATCGATCACGA |
| | | AGATATCACAAAGGATAAAACGTCTACTGTCGAGGCCTG |
| | | CCTCCCCCTCGAACTCACAAAGAACGAGAGTTGCCTCAA |
| | | TTCCCGGGAAACCAGCTTCATCACTAATGGGTCCTGCCTG |
| | | GCAAGCAGAAAAACTTCTTTCATGATGGCATTGTGTTTGA |
| | | GTAGCATCTATGAGGACCTCAAAATGTACCAAGTTGAAT |
| | | TTAAGACTATGAACGCTAAGCTCCTCATGGACCCTAAGC |
| | | GGCAGATCTTCCTGGATCAGAACATGTTGGCTGTCATCGA |
| | | CGAACTCATGCAAGCCCTGAACTTTAATTCCGAAACAGTT |
| | | CCCCAGAAGTCCAGTCTGGAGGAACCCGATTTCTACAAA |
| | | ACCAAAATTAAGCTGTGTATCTTGCTGCATGCTTTCCGGA |
| | | TCAGGGCCGTTACGATCGATCGGGTAATGAGCTACCTGA |
| | | ATGCCAGT |
| 147 | IL-12/23p40 | MCHQQLVISWFSLVFLASPLVAIWELKKDVYVVELDWYPD |
| | precursor | APGEMVVLTCDTPEEDGITWTLDQSSEVLGSGKTLTIQVKEF |
| | polypeptide | GDAGQYTCHKGGEVLSHSLLLLHKKEDGIWSTDILKDQKEP |
| | sequence | KNKTFLRCEAKNYSGRFTCWWLTTISTDLTFSVKSSRGSSDP |
| | | QGVTCGAATLSAERVRGDNKEYEYSVECQEDSACPAAEESL |
| | | PIEVMVDAVHKLKYENYTSSFFIRDIIKPDPPKNLQLKPLKNS |
| | | RQVEVSWEYPDTWSTPHSYFSLTFCVQVQGKSKREKKDRVF |
| | | TDKTSATVICRKNASISVRAQDRYYSSSWSEWASVPCS |
| 148 | IL-12/23p40 | ATGTGTCACCAGCAACTCGTGATCTCATGGTTTTCACTCG |
| | precursor | TTTTTCTGGCAAGTCCGCTTGTGGCAATTTGGGAACTCAA |
| | nucleic acid | AAAGGACGTGTATGTAGTCGAGCTAGATTGGTATCCCGA |
| | sequence | TGCACCCGGTGAAATGGTCGTTCTGACCTGCGACACACC |
| | | AGAAGAGGATGGAATCACCTGGACTCTGGATCAAAGCTC |
| | | CGAGGTGCTGGGGAGCGGCAAAACCCTGACAATTCAGGT |
| | | CAAAGAGTTCGGCGACGCGGGACAGTACACTTGCCATAA |
| | | GGGTGGGGAGGTTCTGTCACACTCTCTGCTACTGCTGCAC |
| | | AAAAAGGAGGACGGAATATGGTCAACGGACATCCTGAAG |
| | | GACCAGAAGGAGCCAAAAAACAAGACCTTCCTGAGGTGC |
| | | GAGGCCAAAAACTACTCAGGCCGCTTTACTTGTTGGTGGC |
| | | TGACAACAATCTCCACAGACCTCACCTTTAGTGTGAAGA |
| | | GTAGTCGCGGATCTAGCGATCCTCAAGGCGTCACTTGCG |
| | | GTGCCGCTACCCTGAGTGCTGAACGAGTGCGTGGGGACA |
| | | ACAAGGAATACGAGTACAGTGTGGAATGCCAAGAGGACA |
| | | GCGCTTGCCCGGCAGCCGAGGAATCACTACCCATTGAAG |
| | | TGATGGTCGATGCCGTCCATAAGTTAAAATACGAGAATT |
| | | ACACGTCCTCTTTCTTCATCCGCGATATAATCAAGCCTGA |
| | | CCCGCCAAAGAATTTGCAGTTGAAGCCCTTGAAAAACAG |
| | | CCGGCAGGTTGAAGTGTCGTGGGAATATCCCGACACTTG |
| | | GAGCACACCACATAGCTATTTCTCACTGACATTCTGTGTG |
| | | CAAGTTCAGGGAAAGTCAAAACGGGAGAAGAAGGATAG |
| | | AGTATTTACCGACAAAACGTCAGCGACAGTGATATGTCG |
| | | AAAAAATGCGTCGATTAGTGTGCGGGCACAGGATAGATA |
| | | CTATTCCAGTTCTTGGTCCGAGTGGGCTTCTGTACCTTGTT |
| | | CA |
| 149 | IL-15 | MRISKPHLRSISIQCYLCLLLNSHFLTEAGIHVFILGCFSAGLP |
| | precursor | KTEANWVNVISDLKKIEDLIQSMHIDATLYTESDVHPSCKVT |
| | polypeptide | AMKCFLLELQVISLESGDASIHDTVENLIILANNSLSSNGNVT |
| | sequence | ESGCKECEELEEKNIKEFLQSFVHIVQMFINTS |
| 150 | IL-15 | ATGAGGATAAGTAAGCCTCATCTGAGATCAATCTCAATC |
| | precursor | CAATGTTATCTGTGCTTATTGCTGAACTCGCATTTCTTGAC |
| | nucleic acid | GGAGGCGGGCATCCACGTGTTTATCCTGGGTTGCTTCTCT |
| | sequence | GCCGGCCTGCCCAAGACCGAAGCCAACTGGGTCAACGTA |
| | | ATCTCAGACCTCAAGAAGATCGAGGATTTGATTCAGTCG |
| | | ATGCACATTGACGCCACTCTCTACACCGAGTCCGACGTCC |
| | | ATCCCTCCTGTAAAGTTACCGCGATGAAGTGTTTCCTGCT |
| | | AGAGCTTCAAGTGATAAGCCTGGAGAGTGGCGACGCCTC |
| | | CATACACGATACCGTGGAGAATCTCATCATCCTGGCTAAC |
| | | AACTCCCTAAGTTCCAATGGGAATGTCACCGAAAGCGGC |
| | | TGTAAAGAGTGCGAGGAACTGGAGGAAAAGAACATCAA |
| | | GGAGTTCCTCCAGTCTTTCGTGCACATCGTGCAGATGTTT |
| | | ATTAATACGTCA |
| 151 | IL-18 | MYRMQLLSCIALSLALVTNSYFGKLESKLSVIRNLNDQVLFI |
| | precursor | DQGNRPLFEDMTDSDCRDNAPRTIFIISMYKDSQPRGMAVTI |
| | polypeptide | SVKCEKISTLSCENKIISFKEMNPPDNIKDTKSDIIFFQRSVPG |
| | sequence | HDNKMQFESSSYEGYFLACEKERDLFKLILKKEDELGDRSIM |
| | | FTVQNED |
| 152 | IL-18 | ATGTATCGAATGCAACTGCTGTCGTGCATAGCACTATCAC |
| | precursor | TGGCTCTGGTGACAAACAGTTATTTTGGAAAACTCGAATC |
| | nucleic acid | AAAACTTTCTGTGATCCGGAACCTCAACGACCAGGTTTTA |
| | sequence | TTCATCGACCAAGGGAATCGACCGCTGTTCGAGGACATG |
| | | ACAGATAGCGACTGTAGAGATAATGCTCCCAGGACCATT |
| | | TTTATAATCTCCATGTACAAAGATAGCCAGCCTCGCGGGA |
| | | TGGCAGTGACTATTAGCGTGAAATGCGAGAAAATCTCTA |
| | | CCCTGAGTTGCGAAAACAAAATAATCTCCTTTAAGGAGA |
| | | TGAATCCACCAGACAACATTAAGGACACAAAGTCCGACA |
| | | TAATTTTCTTTCAACGAAGTGTCCCTGGACACGATAACAA |
| | | AATGCAGTTCGAAAGTAGTAGTTACGAGGGTTATTTCCTG |
| | | GCCTGTGAGAAGGAGCGGGATCTGTTTAAGCTAATCCTT |
| | | AAGAAGGAAGATGAATTGGGCGATAGATCGATAATGTTC |
| | | ACGGTCCAGAATGAGGAC |
| 153 | Super-2 | MYRMQLLSCIALSLALVTNSAPTSSSTKKTQLQLEHLLLDLQ |
| | precursor | MILNGINNYKNPKLTRMLTFKFYMPKKATELKHLQCLEEEL |
| | polypeptide | KPLEEVLNLAQSKNFHFDPRDVVSNINVFVLELKGSETTFMC |
| | sequence | EYADETATIVEFLNRWITFCQSIISTL |
| 154 | Super-2 | ATGTATCGAATGCAACTGCTGTCGTGCATAGCACTATCAC |
| | precursor | TGGCTCTGGTGACAAACAGTGCACCAACGTCATCATCAA |
| | nucleic acid | CCAAAAAGACCCAGCTTCAGCTAGAGCATCTACTGCTGG |
| | sequence | ACCTCCAAATGATTTTGAACGGGATCAACAACTATAAGA |
| | | ATCCCAAACTAACAAGGATGCTGACGTTCAAGTTTTACAT |
| | | GCCGAAAAAGGCAACCGAACTTAAACACCTCCAATGTTT |
| | | AGAGGAGGAACTCAAACCTCTCGAGGAGGTATTGAATCT |
| | | GGCGCAATCCAAGAATTTCCACTTTGACCCACGCGATGTG |
| | | GTTAGCAACATCAATGTGTTTGTGCTTGAATTGAAGGGAA |
| | | GCGAAACGACTTTCATGTGCGAATACGCCGATGAGACTG |
| | | CCACAATCGTGGAGTTCCTTAATCGGTGGATAACCTTTTG |
| | | TCAGTCAATCATTTCTACACTGACC |
| 155 | IL-10 | MHSSALLCCLVLLTGVRASPGQGTQSENSCTHFPGNLPNML |
| | precursor | RDLRDAFSRVKTFFQMKDQLDNLLLKESLLEDFKGYLGCQA |
| | polypeptide | LSEMIQFYLEEVMPQAENQDPDIKAHVNSLGENLKTLRLRLR |
| | sequence | RCHRFLPCENKSKAVEQVKNAFNKLQEKGIYKAMSEFDIFIN |
| | | YIEAYMTMKIRN |
| 156 | IL-10 | ATGCACTCATCAGCCTTGCTGTGCTGCCTCGTGTTGCTGA |
| | precursor | CCGGCGTGCGGGCTTCTCCAGGTCAAGGCACACAGAGTG |
| | nucleic acid | AAAATAGCTGTACTCATTTTCCCGGGAATCTGCCAAATAT |
| | sequence | GCTCCGGGATCTGAGGGACGCATTTTCCAGAGTGAAGAC |
| | | ATTTTTCCAAATGAAGGATCAGCTTGATAATCTCCTGCTG |
| | | AAGGAATCTCTGCTAGAGGACTTTAAGGGCTATCTCGGG |
| | | TGTCAGGCCCTTAGTGAGATGATCCAGTTCTATCTGGAGG |
| | | AAGTAATGCCTCAAGCCGAAAATCAAGATCCTGACATCA |
| | | AAGCTCATGTTAACAGCCTTGGAGAAAATCTGAAAACGC |
| | | TACGCTTACGATTAAGACGCTGCCACAGGTTTTTGCCCTG |
| | | TGAGAACAAATCCAAAGCTGTGGAGCAGGTCAAGAACGC |
| | | ATTCAACAAACTCCAGGAGAAAGGAATTTACAAGGCGAT |
| | | GAGCGAGTTCGACATATTCATCAACTACATTGAGGCCTAC |
| | | ATGACTATGAAGATTCGTAAC |
| 157 | TGF-β | MPPSGLRLLPLLLPLLWLLVLTPGRPAAGLSTCKTIDMELVK |
| | precursor | RKRIEAIRGQILSKLRLASPPSQGEVPPGPLPEAVLALYNSTR |
| | polypeptide | DRVAGESAEPEPEPEADYYAKEVTRVLMVETHNEIYDKFKQ |
| | sequence | STHSIYMFFNTSELREAVPEPVLLSRAELRLLRLKLKVEQHV |
| | | ELYQKYSNNSWRYLSNRLLAPSDSPEWLSFDVTGVVRQWLS |
| | | RGGEIEGFRLSAHCSCDSRDNTLQVDINGFTTGRRGDLATIH |
| | | GMNRPFLLLMATPLERAQHLQSSRHRRALDTNYCFSSTEKN |
| | | CCVRQLYIDFRKDLGWKWIHEPKGYHANFCLGPCPYIWSLD |
| | | TQYSKVLALYNQHNPGASAAPCCVPQALEPLPIVYYVGRKP |
| | | KVEQLSNMIVRSCKCS |
| 158 | TGF-β | ATGCCACCGTCCGGACTGCGGTTGCTGCCTCTGCTCCTGC |
| | precursor | CTCTCCTCTGGCTGCTGGTTCTGACACCAGGGAGGCCTGC |
| | nucleic acid | TGCGGGCCTGTCCACTTGCAAAACCATTGATATGGAACTG |
| | sequence | GTGAAACGCAAGCGGATTGAGGCAATCCGCGGGCAGATC |
| | | CTGTCCAAGCTACGCCTGGCCTCTCCACCCTCTCAAGGCG |
| | | AAGTGCCTCCTGGTCCTCTGCCTGAGGCAGTCCTGGCACT |
| | | CTATAATAGCACTCGAGATCGCGTTGCTGGGGAGAGCGC |
| | | CGAACCTGAACCCGAACCCGAGGCCGATTACTATGCTAA |
| | | AGAGGTCACGCGCGTACTGATGGTTGAAACTCACAACGA |
| | | GATCTACGACAAGTTCAAGCAGAGCACCCACAGTATCTA |
| | | CATGTTCTTTAACACCTCTGAGCTGAGGGAGGCAGTACCA |
| | | GAACCCGTCCTGCTGAGCAGGGCCGAGTTACGGCTTCTTC |
| | | GGCTCAAGCTGAAGGTCGAGCAGCACGTTGAGCTTTACC |
| | | AGAAGTATTCAAACAACTCCTGGCGATACTTGTCCAACC |
| | | GGCTTCTGGCGCCCAGCGACTCACCCGAGTGGCTCTCTTT |
| | | TGACGTGACCGGAGTAGTCCGACAATGGCTCTCTCGCGG |
| | | CGGCGAGATTGAAGGCTTTCGGTTGTCCGCCCATTGTAGC |
| | | TGCGATAGTAGGGATAATACCCTCCAGGTGGACATTAAT |
| | | GGATTTACAACCGGAAGGCGTGGGGACCTTGCTACAATC |
| | | CACGGCATGAACAGACCGTTTTTGCTCCTAATGGCCACTC |
| | | CACTGGAGCGCGCTCAACATCTTCAGTCAAGCAGACACA |
| | | GACGAGCCTTGGACACAAACTATTGCTTCAGTAGTACGG |
| | | AGAAGAATTGTTGCGTGAGACAGTTATATATCGACTTCCG |
| | | TAAAGACTTAGGTTGGAAATGGATACACGAACCAAAGGG |
| | | CTATCATGCAAATTTCTGTCTCGGACCCTGTCCATACATA |
| | | TGGAGTTTGGATACACAGTACTCGAAAGTGCTGGCTCTAT |
| | | ACAATCAGCATAATCCCGGCGCCTCAGCCGCGCCGTGTT |
| | | GTGTGCCACAAGCCCTTGAACCTTTACCCATCGTGTACTA |
| | | TGTGGGCAGAAAACCCAAGGTGGAACAGTTGTCTAACAT |
| | | GATTGTGCGTTCCTGCAAATGCTCA |
| 159* | Non-targeting | GGUUCUUGACUACCGUAAUU |
| | control | |
| | sgRNA | |
| 160* | PTPN2- | UUGACAUAGAAGAGGCACAA |
| | targeting | |
| | sgRNA | |
| 161* | RASA2- | CUAGAGAAACUGUUGCAUAA |
| | targeting | |
| | sgRNA | |
| 162* | SOCS1- | GCGCCCGCGGCCGUGCCCCG |
| | targeting | |
| | sgRNA | |
| 163* | ZC3H12A- | CAGCUCCCUCUAGUCCCGCG |
| | targeting | |
| | sgRNA | |
| 164* | CISH- | GCGAAAGGUGCUGGACCCAG |
| | targeting | |
| | sgRNA | |
| 165 | RASA2- | TCTTTTATACATCTCACCTTCT |
| | targeting | |
| | shRNA | |
| 166 | PTPN2- | TCTGACAAGAGCTTCACACTGA |
| | targeting | |
| | shRNA | |
| 167 | CISH- | TTCATGAAGTCTTATCAGACGT |
| | targeting | |
| | shRNA | |
| 168 | TNFRSF6 | TTAAGAATCTTTTCAAACACTA |
| | (Fas)- | |
| | targeting | |
| | shRNA | |
| 169 | TGFBR1- | TAGAATTACATTTTGATGCCTT |
| | targeting | |
| | shRNA #1 | |
| 170 | TGFBR1- | TTAGTAATAAGACATGTTTCAT |
| | targeting | |
| | shRNA #2 | |
| 171 | TGFBR2- | TAAAAATCAATCTCATTTCCTG |
| | targeting | |
| | shRNA #1 | |
| 172 | TGFBR2- | TTATGTAAAAGACAAACAATGC |
| | targeting | |
| | shRNA #2 | |
| 173 | fLuciferase- | TAGATAAGCATTATAATTCCTA |
| | targeting | |
| | shRNA | |
| 174 | rLuciferase- | TTAATCAGAGACTTCAGGCGGT |
| | targeting | |
| | shRNA | |
| 175 | NGFR | KEACPTGLYTHSGECCKACNLGEGVAQPCGANQTVCEPCL |
| | polypeptide | DSVTFSDVVSATEPCKPCTECVGLQSMSAPCVEADDAVCRC |
| | sequence | AYGYYQDETTGRCEACRVCEAGSGLVFSCQDKQNTVCEEC |
| | | PDGTYSDEANHVDPCLPCTVCEDTERQLRECTRWADAECEE |
| | | IPGRWITRSTPPEGSDSTAPSTQEPEAPPEQDLIASTVAGVVT |
| | | TVMGSSQPVVTRGTTDNLIPVYCSILAAVVVGLVAYIAFKR |
| | | WNS |
| 176 | ALPP UniProt | MLGPCMLLLLLLLGLRLQLSLGIIPVEEENPDFWNREAAEAL |
| | P05187 | GAAKKLQPAQTAAKNLIIFLGDGMGVSTVTAARILKGQKKD |
| | | KLGPEIPLAMDRFPYVALSKTYNVDKHVPDSGATATAYLCG |
| | | VKGNFQTIGLSAAARFNQCNTTRGNEVISVMNRAKKAGKS |
| | | VGVVTTTRVQHASPAGTYAHTVNRNWYSDADVPASARQE |
| | | GCQDIATQLISNMDIDVILGGGRKYMFRMGTPDPEYPDDYS |
| | | QGGTRLDGKNLVQEWLAKRQGARYVWNRTELMQASLDPS |
| | | VTHLMGLFEPGDMKYEIHRDSTLDPSLMEMTEAALRLLSRN |
| | | PRGFFLFVEGGRIDHGHHESRAYRALTETIMFDDAIERAGQL |
| | | TSEEDTLSLVTADHSHVFSFGGYPLRGSSIFGLAPGKARDRK |
| | | AYTVLLYGNGPGYVLKDGARPDVTESESGSPEYRQQSAVPL |
| | | DEETHAGEDVAVFARGPQAHLVHGVQEQTFIAHVMAFAAC |
| | | LEPYTACDLAPPAGTTDAAHPGRSVVPALLPLLAGTLLLLET |
| | | ATAP |
| 177 | ALPG | MQGPWVLLLLGLRLQLSLGIIPVEEENPDFWNRQAAEALGA |
| | UniProt | AKKLQPAQTAAKNLIIFLGDGMGVSTVTAARILKGQKKDKL |
| | P10696 | GPETFLAMDRFPYVALSKTYSVDKHVPDSGATATAYLCGV |
| | | KGNFQTIGLSAAARFNQCNTTRGNEVISVMNRAKKAGKSV |
| | | GVVTTTRVQHASPAGAYAHTVNRNWYSDADVPASARQEG |
| | | CQDIATQLISNMDIDVILGGGRKYMFPMGTPDPEYPDDYSQ |
| | | GGTRLDGKNLVQEWLAKHQGARYVWNRTELLQASLDPSV |
| | | THLMGLFEPGDMKYEIHRDSTLDPSLMEMTEAALLLLSRNP |
| | | RGFFLFVEGGRIDHGHHESRAYRALTETIMFDDAIERAGQLT |
| | | SEEDTLSLVTADHSHVFSFGGYPLRGSSIFGLAPGKARDRKA |
| | | YTVLLYGNGPGYVLKDGARPDVTESESGSPEYRQQSAVPLD |
| | | GETHAGEDVAVFARGPQAHLVHGVQEQTFIAHVMAFAACL |
| | | EPYTACDLAPRAGTTDAAHPGPSVVPALLPLLAGTLLLLGTA |
| | | TAP |
| 178 | MSLN | MALPTARPLLGSCGTPALGSLLFLLFSLGWVQPSRTLAGETG |
| | UniProt | QEAAPLDGVLANPPNISSLSPRQLLGFPCAEVSGLSTERVREL |
| | Q13421 | AVALAQKNVKLSTEQLRCLAHRLSEPPEDLDALPLDLLLFLN |
| | | PDAFSGPQACTRFFSRITKANVDLLPRGAPERQRLLPAALAC |
| | | WGVRGSLLSEADVRALGGLACDLPGRFVAESAEVLLPRLVS |
| | | CPGPLDQDQQEAARAALQGGGPPYGPPSTWSVSTMDALRG |
| | | LLPVLGQPIIRSIPQGIVAAWRQRSSRDPSWRQPERTILRPRFR |
| | | REVEKTACPSGKKAREIDESLIFYKKWELEACVDAALLATQ |
| | | MDRVNAIPFTYEQLDVLKHKLDELYPQGYPESVIQHLGYLFL |
| | | KMSPEDIRKWNVTSLETLKALLEVNKGHEMSPQAPRRPLPQ |
| | | VATLIDRFVKGRGQLDKDTLDTLTAFYPGYLCSLSPEELSSV |
| | | PPSSIWAVRPQDLDTCDPRQLDVLYPKARLAFQNMNGSEYF |
| | | VKIQSFLGGAPTEDLKALSQQNVSMDLATFMKLRTDAVLPL |
| | | TVAEVQKLLGPHVEGLKAEERHRPVRDWILRQRQDDLDTL |
| | | GLGLQGGIPNGYLVLDLSMQEALSGTPCLLGPGPVLTVLALL |
| | | LASTLA |
| 179 | Lecine zipepr | MALPVTALLLPLALLLHAARPDYKDDDDKELCGGRIARLEE |
| | domain L- | KVKTLKAQNSELASTANMLREQVAQLKQKVMNAQGEIE |
| | gp130 | |
| 180 | gp130 | AIVVPVCLAFLLTTLLGVLFCF |
| | (IL6ST) | |
| | transmembrane | |
| | domain | |
| 181 | C7R | MLVRRGARAGPRMPRGWTALCLLSLLPSGFMSLDNNGTAT |
| | ectodomain | PELPTQGTFSNVSTNVSYQETTTPSTLGSTSLHPVSQHGNEA |
| | and TMD | TTNITETTVKFTSTSVITSVYGNTNSSVQSQTSVISTVFTTPAN |
| | | VSTPETTLKPSLSPGNVSDLSTTSTSLATSPTKPYTSSSPILSDI |
| | | KAEIKCSGIREVKLTQGICLEQNKTSSCAEFKKDRGEGLARV |
| | | LCGEEQADADAGAQVCSLLLAQSEVRPQCLLLVLANRTEISS |
| | | KLQLMKKHQSDLKKLGILDFTEQDVASHQSYSQKTPILLTCP |
| | | TISILSFFSVALLVILACVLW |

*first 3 nucleotide are 2′-o-methyl analogs and 3′-phosphorothioate internucleotide linkages

1. A system comprising:
a. a first chimeric polypeptide comprising a priming receptor;
b. a second chimeric polypeptide comprising a chimeric antigen receptor (CAR); and
c. a third chimeric polypeptide comprising a synthetic pathway activator (SPA).

2. A system comprising:
a. a first chimeric polypeptide comprising a priming receptor;
b. a second chimeric polypeptide comprising a chimeric antigen receptor (CAR);
c. a third chimeric polypeptide comprising a synthetic pathway activator (SPA); and
d. a cytokine.

3. A system comprising:
a. a first chimeric polypeptide comprises a priming receptor;
b. a second chimeric polypeptide comprises a chimeric antigen receptor (CAR); and
c. a cytokine.

4. A system comprising:
a. a first chimeric polypeptide comprising a priming receptor;
b. a second chimeric polypeptide comprising a chimeric antigen receptor (CAR);
c. a suppressor of gene expression, and
d. one or both of:
i. a third chimeric polypeptide comprising a synthetic pathway activator (SPA); and/or
ii. a cytokine.

5. The system of claims 1-4, wherein the priming receptor comprises, from N-terminus to C-terminus,
a. a first extracellular antigen-binding domain;
b. a first transmembrane domain comprising one or more ligand -inducible proteolytic cleavage sites; and
c. an intracellular domain comprising a human or humanized transcriptional effector.

6. The system of claim 5, wherein the first extracellular antigen-binding domain specifically binds to Alkaline Phosphatase, Germ Cell (ALPG/P).

7. The system of claim 5 or 6, wherein the first extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein:
a. CDR-H1 comprises the sequence set forth in SEQ ID NO: 1, 39, 40, 41, or 42,
b. CDR-H2 comprises the sequence set forth in SEQ ID NO: 2, 43, 44, 45, or 46,
c. CDR-H3 comprises the sequence set forth in SEQ ID NO: 3, 47, or 48,
d. CDR-L1 comprises the sequence set forth in SEQ ID NO: 4, 49, or 50,
e. CDR-L2 comprises the sequence set forth in SEQ ID NO: 5 or 51; and
f. CDR-L3 comprises the sequence set forth in SEQ ID NO: 6 or 53.

8. The system of any one of claims 1-6, wherein the CAR comprises, from N-terminus to C-terminus,
a. a second extracellular antigen-binding domain;
b. a second transmembrane domain;
c. an intracellular co-stimulatory domain; and
d. an intracellular activation domain.

9. The system of claim 8, wherein the second extracellular antigen-binding domain specifically binds to mesothelin (MSLN), wherein the second extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein:
a. CDR-H1 comprises the sequence set forth in SEQ ID NO: 10, 54, 55, 56, or 57,
b. CDR-H2 comprises the sequence set forth in SEQ ID NO: 11, 58, 59, 60, or 61,
c. CDR-H3 comprises the sequence set forth in SEQ ID NO: 12, 62, or 63,
d. CDR-L1 comprises the sequence set forth in SEQ ID NO: 14, 64, 65, 66, or 67,
e. CDR-L2 comprises the sequence set forth in SEQ ID NO: 15, 68, 69, or 70, and
f. CDR-L3 comprises the sequence set forth in SEQ ID NO: 16, 72, or 73.

10. The system of any one of claim 1, 2 or 4-9, wherein the SPA comprises a leucine zipper-gp130 (L-gp130).

11. The system of any one of claim 1, 2 or 4-9, wherein the SPA comprises a membrane-bound interleukin-15 (mbIL-15).

12. The system of any one of claim 1, 2 or 4-9, wherein the SPA comprises a CD34-interleukin-7 receptor (C7R).

13. The system of any one of claims 2-12, wherein the cytokine is a secreted cytokine or a membrane-bound cytokine.

14. The system of any one of claims 2-13, wherein the cytokine comprises at least one of interleukin (IL)-2, Super-2, IL-12, IL-12/23p40, IL-7, IL-15, IL-21, and IL-18.

15. The system of any one of claims 4-14, wherein the suppressor of gene expression is an sgRNA or an shRNA.

16. The system of claim 15, wherein the sgRNA suppresses the expression of a gene selected from PTPN2, RASA2, SOCS1, ZC3H12A, and CISH.

17. The system of claim 15, wherein the shRNA suppresses the expression of a gene selected from RASA2, SOCS1, ZC3H12A, TGFBR1, and CISH.

18. One or more recombinant nucleic acids comprising at least one nucleic acid fragment comprising a nucleotide sequence encoding the system of one of claims 1-17.

19. One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising a second extracellular antigen-binding domain; and
c. a nucleotide sequence encoding a cytokine.

20. One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain; and
c. a nucleotide sequence encoding a synthetic pathway activator.

21. One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain;
c. a nucleotide sequence encoding a synthetic pathway activator; and
d. a nucleotide sequence encoding a cytokine.

22. One or more recombinant nucleic acids comprising at least one nucleic acid fragment comprising:
a. a nucleotide sequence encoding a priming receptor comprising a first extracellular antigen-binding domain;
b. a nucleotide sequence encoding a chimeric antigen receptor comprising an second extracellular antigen-binding domain; and
c. a nucleotide sequence of a suppressor of gene expression; and
d. one or both of:
i. a nucleotide sequence encoding a synthetic pathway activator; and/or
ii. a nucleotide sequence encoding a cytokine.

23. An expression vector comprising the recombinant nucleic acid of any one of claims 18-22.

24. An immune cell comprising:
a. the system of any one of claims 1-17;
b. at least one recombinant nucleic acid of any one of claims 18-22; and/or
c. the vector of claim 23.

25. A primary immune cell comprising at least one recombinant nucleic acid comprising:
a. a nucleic acid sequence encoding a priming receptor comprising a first extracellular antigen-binding domain;
b. a nucleic acid sequence encoding a chimeric antigen receptor comprising a second extracellular antigen-binding domain; and
c. a nucleic acid sequence encoding a synthetic pathway activator and/or a nucleic acid sequence encoding a cytokine;
wherein the recombinant nucleic acid is inserted into a target region of the genome of the primary immune cell, wherein the primary immune cell does not comprise a viral vector for introducing the recombinant nucleic acid into the primary immune cell.

26. A viable, virus-free, primary cell comprising a ribonucleoprotein complex (RNP)-recombinant nucleic acid complex, wherein the RNP comprises a nuclease domain and a guide RNA, wherein recombinant nucleic acid comprises:
a. a nucleic acid sequence encoding a priming receptor comprising a first extracellular antigen-binding domain;
b. a nucleic acid sequence encoding a chimeric antigen receptor comprising a second extracellular antigen-binding domain; and
c. a nucleic acid sequence encoding a synthetic pathway activator that constitutively activates cytokine signaling and/or a nucleic acid sequence encoding a cytokine;
wherein the 5′ and 3′ ends of the recombinant nucleic acid comprise nucleotide sequences that are homologous to genomic sequences flanking an insertion site in the genome of the primary cell.

27. A viable, virus-free, primary cell comprising a ribonucleoprotein complex (RNP)-recombinant nucleic acid complex, wherein the RNP comprises a nuclease domain and a guide RNA, wherein recombinant nucleic acid comprises:
a. a nucleic acid sequence encoding a priming receptor comprising a first extracellular antigen-binding domain that specifically binds to ALPG/P;
b. a nucleic acid sequence encoding a chimeric antigen receptor comprising a second extracellular antigen-binding domain that specifically binds to MSLN; and
c. a nucleic acid sequence encoding a synthetic pathway activator that constitutively activates cytokine signaling and/or a nucleic acid sequence encoding a cytokine;
wherein the 5′ and 3′ ends of the recombinant nucleic acid comprise nucleotide sequences that are homologous to genomic sequences flanking an insertion site in the genome of the primary cell.

28. A population of cells comprising a plurality of immune cells of claim 24 or primary cells of any one of claims 25-27.

29. A pharmaceutical composition comprising the immune cell of claim 24, the primary cells of any one of claims 25-27, or the population of cells of claim 28, and a pharmaceutically acceptable excipient.

30. A pharmaceutical composition comprising the recombinant nucleic acid of any one of claims 18-22 or the vector of claim 23, and a pharmaceutically acceptable excipient.

31. A method of editing an immune cell, comprising:
a. providing a ribonucleoprotein complex (RNP)-recombinant nucleic acid complex, wherein the RNP comprises a nuclease domain and a guide RNA, wherein the recombinant nucleic acid comprises the recombinant nucleic acid of any one of claims 18-22, and wherein the 5′ and 3′ ends of the recombinant nucleic acid comprise nucleotide sequences that are homologous to genomic sequences flanking an insertion site in the genome of the immune cell;
b. non-virally introducing the RNP-recombinant nucleic acid complex into the immune cell, wherein the guide RNA specifically hybridizes to a target region of the genome of the primary immune cell, and wherein the nuclease domain cleaves the target region to create the insertion site in the genome of the immune cell; and
c. editing the immune cell via insertion of the recombinant nucleic acid of any one of claims 18-22 into the insertion site in the genome of the immune cell.

32. A method of treating a disease in a subject comprising administering the immune cell of claim 24, the primary cells of any one of claims 25-27, or the population of cells of claim 28, or the pharmaceutical composition of claim 30 or 30 to the subject.

33. A method of inhibiting a target cell in a subject comprising administering the immune cell of claim 24, the primary cells of any one of claims 25-27, or the population of cells of claim 28 to the subject, wherein the immune cell inhibits the target cell.

34. A method of modulating the activity of an immune cell comprising:
a. obtaining an immune cell comprising
i. the system of any one of claims 1-17;
ii. the recombinant nucleic acid of any one of claims 18-22; and/or
iii. the vector of claim 23; and
b. contacting the immune cell with a target cell expressing a priminge receptor antigen and a CAR antigen, wherein binding of the priming receptor to the priminge receptor antigen on the target cell induces activation of the priming receptor and expression of the chimeric antigen receptor, wherein binding of the chimeric antigen receptor to the CAR antigen on the target cell modulates the activity of the immune cell, and wherein the synthetic pathway activator and/or cytokine also modulates the activity of the immune cell.

35. A method of modulating the activity of an immune cell comprising:
a. obtaining an immune cell comprising
i. the system of any one of claims 1-17;
ii. the recombinant nucleic acid of any one of claims 18-22; and/or
iii. the vector of claim 23; and
b. contacting the immune cell with a target cell expressing ALPG/P and MSLN, wherein binding of the priming receptor to ALPG/P on the target cell induces activation of the priming receptor and expression of the chimeric antigen receptor, wherein binding of the chimeric antigen receptor to MSLN on the target cell modulates the activity of the immune cell, and wherein the synthetic pathway activator and/or cytokine also modulates the activity of the immune cell.


SEQUENCE LISTING (308 sequences)

SEQ ID NO: 1 - protein, 7 aa, synthetic construct
GFSLTSY

SEQ ID NO: 2 - protein, 5 aa, synthetic construct
WEDGS

SEQ ID NO: 3 - protein, 13 aa, synthetic construct
PHYGSSYVGAMEY

SEQ ID NO: 4 - protein, 11 aa, synthetic construct
RASENIYSYVA

SEQ ID NO: 5 - protein, 7 aa, synthetic construct
NAKSLAS

SEQ ID NO: 6 - protein, 9 aa, synthetic construct
QHHYVSPWT

SEQ ID NO: 7 - protein, 121 aa, synthetic construct
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 8 - protein, 107 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIK

SEQ ID NO: 9 - protein, 243 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 10 - protein, 5 aa, synthetic construct
GYTMN

SEQ ID NO: 11 - protein, 17 aa, synthetic construct
LITPYNGASSYNQKFRG

SEQ ID NO: 12 - protein, 10 aa, synthetic construct
GGYDGRGFDY

SEQ ID NO: 13 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 14 - protein, 10 aa, synthetic construct
SASSSVSYMH

SEQ ID NO: 15 - protein, 7 aa, synthetic construct
DTSKLAS

SEQ ID NO: 16 - protein, 9 aa, synthetic construct
QQWSGYPLT

SEQ ID NO: 17 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 18 - protein, 27 aa, synthetic construct
TTTPAPRPPTPAPTIASQPLSLRPEAC

SEQ ID NO: 19 - protein, 22 aa, synthetic construct
FMYVAAAAFVLLFFVGCGVLLS

SEQ ID NO: 20 - protein, 43 aa, synthetic construct
RKRRRQHGQLWFPEGFKVSEASKKKRREPLGEDSVGLKPLKNA

SEQ ID NO: 21 - protein, 146 aa, synthetic construct
KLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 22 - protein, 64 aa, synthetic construct
AAAGGSGGSGGSDALDDFDLDMLGSDALDDFDLDMLGSDALDDFDLDMLGSDALDDFDLDMLGS

SEQ ID NO: 23 - protein, 210 aa, synthetic construct
KLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 24 - protein, 243 aa, synthetic construct; identical to SEQ ID NO: 9

SEQ ID NO: 25 - protein, 274 aa, synthetic construct
MALPVTALLLPLALLLHAARPEQKLISEEDLDIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 26 - protein, 45 aa, synthetic construct
TTTPAPRPPTPAPTIASQPLSLRPEACRPAAGGAVHTRGLDFACD

SEQ ID NO: 27 - protein, 24 aa, synthetic construct
IYIWAPLAGTCGVLLLSLVITLYC

SEQ ID NO: 28 - protein, 42 aa, synthetic construct
KRGRKKLLYIFKQPFMRPVQTTQEEDGCSCRFPEEEEGGCEL

SEQ ID NO: 29 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 240 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 31 - protein, 466 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 495 aa, synthetic construct
MALPVTALLLPLALLLHAARPDYKDDDDKQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 21 aa, synthetic construct
MALPVTALLLPLALLLHAARP

SEQ ID NO: 34 - protein, 10 aa, synthetic construct
EQKLISEEDL

SEQ ID NO: 35 - DNA, 822 bp, synthetic construct
  source 1..822: mol_type = other DNA
atggccttaccagtgaccgccttgctcctgccgctggccttgctgctccacgccgccaggcctgaacaaaaactcattagcgaagaggatctcgacatacagatgacacagagccctagcagtctgagcgccagtgtgggcgatagagttactatcacttgtagagcatccgagaacatatacagttacgtggcctggtatcagcaaaaacctggcaaagctcccaagttattgatttacaatgctaagagcttggcctctggggtgccatcgaggttcagcggtagcgggagcgggaccgacttcactctgaccatctcgagtctccagccggaggactttgcgacatactattgtcaacaccattacgtatcaccctggaccttcggcggcgggactaagttagagatcaagggtggaggaggatcaggcggcggtggatcaggaggaggagggtcacaagtgcagttacaggaatcagggcccggcctggtgaagccaagtgaaaccctgagtctgacgtgcacggtttcaggatttagcctcacttcctacggtgtctcttggattcggcagccagccggcaaagggctcgagtggattggggtgatctgggaagatggctcaacaaactatcattctgcactaatctctcgcgtgacaatgtcggtggacacgtccaagaatcaattttcccttaaactgtcctccgtgaccgcagccgatacagcggtatattattgcgcgcgacctcactacggatctagctatgtcggcgcgatggagtattggggcgctggcacaaccgtcaccgtttcttcc

SEQ ID NO: 36 - DNA, 1485 bp, synthetic construct
  source 1..1485: mol_type = other DNA
atggccctgccagtaacggctctgctgctgccacttgctctgctcctccatgcagccaggcctgactacaaagacgatgacgacaagcaagtccagctccagcagtcgggcccagagttggagaagcctggggcgagcgtgaagatctcatgcaaagcctcaggctactcctttactggatacacgatgaattgggtgaaacagtcgcatggaaagtcactggaatggatcggtctgattacgccctacaacggcgcctccagctacaaccagaagttcaggggaaaggcgacccttactgtcgacaagtcgtcaagcaccgcctacatggacctcctgtccctgacctccgaagatagcgcggtctacttttgtgcacgcggaggttacgatggacggggattcgactactggggccagggaaccactgtcaccgtgtcgagcggaggcggagggagcggaggaggaggcagcggaggtggagggtcggatatcgaactcactcagtccccagcaatcatgtccgcttcaccgggagaaaaggtgaccatgacttgctcggcctcctcgtccgtgtcatacatgcactggtaccaacaaaaatcggggacctcccctaagagatggatctacgataccagcaaactggcttcaggcgtgccgggacgcttctcgggttcggggagcggaaattcgtattcgttgaccatttcgtccgtggaagccgaggacgacgcaacttattactgccaacagtggtcaggctacccgctcactttcggagccggcactaagctggagatcaaggcggcagcaaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctagg

SEQ ID NO: 37 - protein, 22 aa, synthetic construct
FMYVAAAAFVLLFFVGCGVLLS

SEQ ID NO: 38 - skipped

SEQ ID NO: 39 - protein, 10 aa, synthetic construct
GFSLTSYGVS

SEQ ID NO: 40 - protein, 5 aa, synthetic construct
SYGVS

SEQ ID NO: 41 - protein, 6 aa, synthetic construct
TSYGVS

SEQ ID NO: 42 - protein, 8 aa, synthetic construct
GFSLTSYG

SEQ ID NO: 43 - protein, 9 aa, synthetic construct
VIWEDGSTN

SEQ ID NO: 44 - protein, 16 aa, synthetic construct
VIWEDGSTNYHSALIS

SEQ ID NO: 45 - protein, 12 aa, synthetic construct
WIGVIWEDGSTN

SEQ ID NO: 46 - protein, 7 aa, synthetic construct
IWEDGST

SEQ ID NO: 47 - protein, 14 aa, synthetic construct
ARPHYGSSYVGAME

SEQ ID NO: 48 - protein, 15 aa, synthetic construct
ARPHYGSSYVGAMEY

SEQ ID NO: 49 - protein, 7 aa, synthetic construct
YSYVAWY

SEQ ID NO: 50 - protein, 6 aa, synthetic construct
ENIYSY

SEQ ID NO: 51 - protein, 10 aa, synthetic construct
LLIYNAKSLA

SEQ ID NO: 52 - skipped

SEQ ID NO: 53 - protein, 8 aa, synthetic construct
QHHYVSPW

SEQ ID NO: 54 - protein, 10 aa, synthetic construct
GYSFTGYTMN

SEQ ID NO: 55 - protein, 5 aa, synthetic construct
GYTMN

SEQ ID NO: 56 - protein, 6 aa, synthetic construct
TGYTMN

SEQ ID NO: 57 - protein, 8 aa, synthetic construct
GYSFTGYT

SEQ ID NO: 58 - protein, 10 aa, synthetic construct
LITPYNGASS

SEQ ID NO: 59 - protein, 17 aa, synthetic construct
LITPYNGASSYNQKFRG

SEQ ID NO: 60 - protein, 13 aa, synthetic construct
WIGLITPYNGASS

SEQ ID NO: 61 - protein, 8 aa, synthetic construct
ITPYNGAS

SEQ ID NO: 62 - protein, 11 aa, synthetic construct
ARGGYDGRGFD

SEQ ID NO: 63 - protein, 12 aa, synthetic construct
ARGGYDGRGFDY

SEQ ID NO: 64 - protein, 10 aa, synthetic construct
SASSSVSYMH

SEQ ID NO: 65 - protein, 10 aa, synthetic construct
SASSSVSYMH

SEQ ID NO: 66 - protein, 6 aa, synthetic construct
SYMHWY

SEQ ID NO: 67 - protein, 5 aa, synthetic construct
SSVSY

SEQ ID NO: 68 - protein, 7 aa, synthetic construct
DTSKLAS

SEQ ID NO: 69 - protein, 7 aa, synthetic construct
DTSKLAS

SEQ ID NO: 70 - protein, 10 aa, synthetic construct
RWIYDTSKLA

SEQ ID NO: 71 - skipped

SEQ ID NO: 72 - protein, 8 aa, synthetic construct
QQWSGYPL

SEQ ID NO: 73 - protein, 10 aa, synthetic construct
GYSFTGYTMN

SEQ ID NO: 74 - protein, 277 aa, synthetic construct
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 75 - protein, 378 aa, synthetic construct
MALPVTALLLPLALLLHAARPDYKDDDDKELCGGRIARLEEKVKTLKAQNSELASTANMLREQVAQLKQKVMNAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 76 - protein, 414 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 77 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 78 - protein, 513 aa, synthetic construct
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 79 - DNA, 831 bp, synthetic construct
  source 1..831: mol_type = other DNA
aacaagcgggaccttatcaaaaagcacatctggcccaacgtgccagatcccagcaaatctcatattgctcaatggagtccacatacgccaccacgacacaacttcaatagcaaggaccagatgtactctgacgggaattttaccgacgtgtcagtagtagagatcgaagccaatgacaagaagcccttcccggaggacctcaagagcctagatctcttcaaaaaggagaaaattaacacggagggacacagctcaggaattggcggtagctcctgtatgtcaagctcaaggccgtcaataagcagtagtgatgagaacgagtcctcccagaacaccagttcaacagtgcagtacagtaccgtcgtgcacagtgggtataggcatcaagttcccagcgtgcaagtgttcagccgatccgaaagtacacagccattattggactccgaggagaggcctgaggacttgcagctcgtggaccacgtggacggtggggatgggattctgccaagacaacaatactttaagcaaaactgctcccagcacgagtctagcccggatatatcacatttcgaaagatctaaacaggtcagctcggtgaatgaggaagattttgttcggctgaagcagcagataagtgaccacatcagtcagagttgtggttcaggccagatgaagatgtttcaggaggtatctgctgcggatgcctttggccctggcacagagggtcaagttgaaaggttcgaaaccgtcggaatggaagctgccacagatgagggaatgcctaagtcttatttgccacaaactgtccggcagggcgggtatatgcctcag

SEQ ID NO: 80 - DNA, 1134 bp, synthetic construct
  source 1..1134: mol_type = other DNA
atggccctgccagtaacggctctgctgctgccacttgctctgctcctccatgcagccaggcctgactacaaagacgatgacgacaaggaactgtgcggagggcgaatcgcccgcctggaagagaaagtcaaaaccctgaaagcccaaaactctgaactggcatcaactgcgaatatgctccgtgagcaggtcgctcagcttaagcagaaagtgatgaatgcacaaggcgaaatcgaggccatagtggttccagtttgtttggcatttctcctcaccactctgctgggcgtccttttctgcttcaacaagcgggaccttatcaaaaagcacatctggcccaacgtgccagatcccagcaaatctcatattgctcaatggagtccacatacgccaccacgacacaacttcaatagcaaggaccagatgtactctgacgggaattttaccgacgtgtcagtagtagagatcgaagccaatgacaagaagcccttcccggaggacctcaagagcctagatctcttcaaaaaggagaaaattaacacggagggacacagctcaggaattggcggtagctcctgtatgtcaagctcaaggccgtcaataagcagtagtgatgagaacgagtcctcccagaacaccagttcaacagtgcagtacagtaccgtcgtgcacagtgggtataggcatcaagttcccagcgtgcaagtgttcagccgatccgaaagtacacagccattattggactccgaggagaggcctgaggacttgcagctcgtggaccacgtggacggtggggatgggattctgccaagacaacaatactttaagcaaaactgctcccagcacgagtctagcccggatatatcacatttcgaaagatctaaacaggtcagctcggtgaatgaggaagattttgttcggctgaagcagcagataagtgaccacatcagtcagagttgtggttcaggccagatgaagatgtttcaggaggtatctgctgcggatgcctttggccctggcacagagggtcaagttgaaaggttcgaaaccgtcggaatggaagctgccacagatgagggaatgcctaagtcttatttgccacaaactgtccggcagggcgggtatatgcctcag

SEQ ID NO: 81 - DNA, 1242 bp, synthetic construct
  source 1..1242: mol_type = other DNA
atggattggacttggatactgtttctggtggcagccgctacccgggtacacagtggcatccacgtgtttatcctgggttgcttctctgccggcctgcccaagaccgaagccaactgggtcaacgtaatctcagacctcaagaagatcgaggatttgattcagtcgatgcacattgacgccactctctacaccgagtccgacgtccatccctcctgtaaagttaccgcgatgaagtgtttcctgctagagcttcaagtgataagcctggagagtggcgacgcctccatacacgataccgtggagaatctcatcatcctggctaacaactccctaagttccaatgggaatgtcaccgaaagcggctgtaaagagtgcgaggaactggaggaaaagaacatcaaggagttcctccagtctttcgtgcacatcgtgcagatgtttattaatacgtcatcaggcggcggaagtggtggaggaggatctggaggaggagggtctggtggaggcggatctggcggcgggtccttacagatcacctgtccaccaccaatgagcgtggaacatgcagacatttgggtgaaatcatatagtctgtactcacgagagcgatacatctgtaactctgggtttaagagaaaggctggaacaagtagcctgaccgagtgcgttctgaacaaggctaccaacgtagctcactggacaacacccagccttaaatgtattagagatccggccctggtccatcagcgtcctgcacctccgtctacagtcacaacggctggggtgacaccacagccagaatccctgtcacccagcggtaaagaacccgccgcctcctcacctagttctaataacacggctgcgactaccgccgcgatagtgcctgggagccagttgatgccctctaaatcgccctcaactgggactacagaaattagcagtcacgagtcctcacacggcacacctagccagaccaccgcaaaaaattgggaattaacagccagcgcctcccatcagcctcctggggtctatccacaaggtcacagcgatactacggttgcaattagcactagcactgttctgttgtgcggcctgagcgcagtcagtttgctcgcctgctatctcaagtcaaggcaaacaccaccgttagctagtgtggaaatggaggcaatggaggccttgcccgtgacttggggcaccagctcccgcgacgaagatctcgagaattgctcacatcatctg

SEQ ID NO: 82 - DNA, 585 bp, synthetic construct
  source 1..585: mol_type = other DNA
aaaaaacgtatcaagcccattgtgtggccctctctgcccgaccacaaaaagaccctcgaacacctctgcaagaagccacggaagaatcttaatgtctccttcaaccctgaatcattcctagactgccagattcatcgagttgacgacattcaggctcgggacgaggtggaagggtttctccaggataccttcccacagcaactggaggagagcgagaaacagcgcctgggtggggacgtccagagtccaaattgtcccagcgaggatgtggttatcactcctgaatcgtttggaagagattccagtctgacctgtctggcaggcaacgtttcagcgtgtgacgcaccgatactgtccagctctaggtccttggattgtagggagagcggcaaaaatgggccacacgtgtatcaagatctcctgttaagtctgggaacaacaaactctacactcccaccgccatttagcctgcaatccggaattttaacacttaaccctgtagctcaaggccagcctatattgaccagccttggtagcaaccaggaagaggcctacgtgactatgtcaagtttttaccagaaccaa

SEQ ID NO: 83 - DNA, 1539 bp, synthetic construct
  source 1..1539: mol_type = other DNA
atgttagtacggcgtggggcaagggctggaccaaggatgccacgcggctggacagccttgtgcctgctgtcactgcttccctcgggctttatgagcctcgacaataacggcacggcaacacccgagttgcctactcaagggaccttttccaacgtgtccaccaatgtcagttatcaggagacaactactccctccaccctgggctctacgtctttgcatcccgtcagtcaacacggtaacgaggctaccacgaacatcacggagacaacggtcaaattcacttctacaagcgttataactagcgtctacggcaatactaacagttcagttcagtctcagaccagcgtgatctcaaccgtatttactacaccggcgaacgtttcaacacctgaaacaacccttaaaccgtccctgtcacccgggaatgtctccgacctgagcacaacctccactagcttggccacctctcctacaaagccctataccagcagctcaccgatactgagcgacattaaggcggaaatcaagtgctcgggtattcgagaagtgaaactcacccaaggaatctgtcttgaacagaacaagaccagttcctgcgctgagttcaaaaaggaccgtggggaaggccttgcaagagtgctgtgtggggaggaacaggccgacgccgatgccggagcccaagtgtgtagtctgctattagcacaatccgaggtgcggccacagtgcctgttgctcgtgctggccaatcgaaccgagatctcttctaagctccagttgatgaagaagcaccagtccgatctcaaaaagctaggaattttagatttcactgagcaggatgtggcttcacatcagagttactctcagaaaacccccatcttgttaacttgccctacgatctcgatactaagtttcttctccgtcgccctcctggtaatcttggcctgcgtgctgtggaaaaaacgtatcaagcccattgtgtggccctctctgcccgaccacaaaaagaccctcgaacacctctgcaagaagccacggaagaatcttaatgtctccttcaaccctgaatcattcctagactgccagattcatcgagttgacgacattcaggctcgggacgaggtggaagggtttctccaggataccttcccacagcaactggaggagagcgagaaacagcgcctgggtggggacgtccagagtccaaattgtcccagcgaggatgtggttatcactcctgaatcgtttggaagagattccagtctgacctgtctggcaggcaacgtttcagcgtgtgacgcaccgatactgtccagctctaggtccttggattgtagggagagcggcaaaaatgggccacacgtgtatcaagatctcctgttaagtctgggaacaacaaactctacactcccaccgccatttagcctgcaatccggaattttaacacttaaccctgtagctcaaggccagcctatattgaccagccttggtagcaaccaggaagaggcctacgtgactatgtcaagtttttaccagaaccaa

SEQ ID NO: 84 - protein, 353 aa, synthetic construct
MDWTWILFLVAAATRVHSRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 85 - protein, 331 aa, synthetic construct
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 86 - protein, 133 aa, synthetic construct [sequence omitted]

SEQ ID NO: 87 - DNA, 399 bp, synthetic construct
  source 1..399: mol_type = other DNA
gcaccaacgtcatcatcaaccaaaaagacccagcttcagctagagcatctactgctggacctccaaatgattttgaacgggatcaacaactataagaatcccaaactaacaaggatgctgacgttcaagttttacatgccgaaaaaggcaaccgaacttaaacacctccaatgtttagaggaggaactcaaacctctcgaggaggtattgaatctggcgcaatccaagaatttccacctgaggccacgcgatctgatcagcaacatcaatgtgatcgtgcttgaattgaagggaagcgaaacgactttcatgtgcgaatacgccgatgagactgccacaatcgtggagttccttaatcggtggataaccttttgtcagtcaatcatttctacactgacc

SEQ ID NO: 88 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 89 - DNA, 456 bp, synthetic construct
  source 1..456: mol_type = other DNA
gattgtgacattgaagggaaagatgggaaacagtacgagtccgtcctgatggtatctatagaccagttactcgatagcatgaaggagatcggctccaactgccttaacaacgagtttaatttcttcaaacgccatatttgcgatgccaataaggaaggaatgttcttattccgagctgcccggaagctgaggcagtttctgaagatgaacagcactggcgactttgacctgcacctccttaaggtgtctgagggcaccacaattctgctgaattgtaccggccaagtgaagggacgaaaacctgctgcgctaggagaagcacaacccacaaaatcactcgaggagaataagagtcttaaggaacagaagaagctgaacgacctctgctttttgaagaggctgttgcaagagatcaaaacctgttggaataagatcttgatggggaccaaagaacac

SEQ ID NO: 90 - protein, 133 aa, synthetic construct [sequence omitted]

SEQ ID NO: 91 - DNA, 399 bp, synthetic construct
  source 1..399: mol_type = other DNA
caaggacaggaccgacatatgataaggatgaggcaattaattgatattgtggaccaattgaaaaactacgtcaatgacctcgtgccagagttcctgcccgctcccgaagatgtagaaacgaattgcgaatggtcagcctttagctgtttccaaaaggcacagctcaaaagcgcgaataccggaaacaacgagcgtataattaacgtttcaatcaaaaaacttaagaggaagcctccttctacaaatgccggcaggcgccagaagcaccgcctgacctgcccgtcttgcgacagttatgagaagaagccacccaaagaatttctcgagagattcaagtctttacttcagaaaatgatccatcagcacctgagcagccgaactcacgggagtgaagattcc

SEQ ID NO: 92 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 93 - DNA, 1554 bp, synthetic construct
  source 1..1554: mol_type = other DNA
atttgggaactgaagaaagacgtatatgtcgtggaactggactggtatcccgatgccccaggggaaatggtggtccttacttgcgacacccccgaagaggacggcattacatggacccttgaccagtccagtgaggtcctcgggagtggaaagacactgacaattcaggttaaggagttcggcgatgccggtcagtacacctgtcataaagggggagaggttctgtcccattcactcctgctgctgcacaagaaggaagatggtatctggtccacggacattttgaaagatcaaaaggagcccaagaacaaaactttcctgcggtgtgaagccaaaaattacagcgggcgatttacttgttggtggctcaccactattagtacagatctcaccttcagcgttaagagttctaggggctccagtgatccgcagggagtgacgtgtggcgctgcgaccctgtccgctgagcgagtgaggggcgataataaggagtatgagtatagtgtggagtgtcaggaggattctgcttgccctgctgccgaggagtcccttcctatcgaagtgatggtcgatgctgtacacaaactgaaatacgagaattatactagctcctttttcataagggacatcataaagccagatccccccaagaaccttcagctgaaaccgctgaaaaattcacgccaggtggaggtatcttgggagtacccagacacatggagcacacctcatagttacttcagcctgaccttctgtgtccaggtccaggggaagtctaaaagggaaaaaaaggacagagttttcaccgacaagacatccgcaaccgtaatctgtaggaaaaacgcctcaatttccgttagagcgcaagataggtattattcctcttcctggtctgaatgggcctctgtaccctgcagcgggggtggcggtagcggtgggggagggagcggaggtggaggcagccgaaacctgcctgtggccacacctgatcctgggatgttcccttgcttgcaccacagtcagaatctgctcagggccgttagcaacatgctgcagaaggcgcggcagacacttgagttctacccttgcacaagtgaagagatcgatcacgaagatatcacaaaggataaaacgtctactgtcgaggcctgcctccccctcgaactcacaaagaacgagagttgcctcaattcccgggaaaccagcttcatcactaatgggtcctgcctggcaagcagaaaaacttctttcatgatggcattgtgtttgagtagcatctatgaggacctcaaaatgtaccaagttgaatttaagactatgaacgctaagctcctcatggaccctaagcggcagatcttcctggatcagaacatgttggctgtcatcgacgaactcatgcaagccctgaactttaattccgaaacagttccccagaagtccagtctggaggaacccgatttctacaaaaccaaaattaagctgtgtatcttgctgcatgctttccggatcagggccgttacgatcgatcgggtaatgagctacctgaatgccagt

SEQ ID NO: 94 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 95 - DNA, 918 bp, synthetic construct
  source 1..918: mol_type = other DNA
atttgggaactcaaaaaggacgtgtatgtagtcgagctagattggtatcccgatgcacccggtgaaatggtcgttctgacctgcgacacaccagaagaggatggaatcacctggactctggatcaaagctccgaggtgctggggagcggcaaaaccctgacaattcaggtcaaagagttcggcgacgcgggacagtacacttgccataagggtggggaggttctgtcacactctctgctactgctgcacaaaaaggaggacggaatatggtcaacggacatcctgaaggaccagaaggagccaaaaaacaagaccttcctgaggtgcgaggccaaaaactactcaggccgctttacttgttggtggctgacaacaatctccacagacctcacctttagtgtgaagagtagtcgcggatctagcgatcctcaaggcgtcacttgcggtgccgctaccctgagtgctgaacgagtgcgtggggacaacaaggaatacgagtacagtgtggaatgccaagaggacagcgcttgcccggcagccgaggaatcactacccattgaagtgatggtcgatgccgtccataagttaaaatacgagaattacacgtcctctttcttcatccgcgatataatcaagcctgacccgccaaagaatttgcagttgaagcccttgaaaaacagccggcaggttgaagtgtcgtgggaatatcccgacacttggagcacaccacatagctatttctcactgacattctgtgtgcaagttcagggaaagtcaaaacgggagaagaaggatagagtatttaccgacaaaacgtcagcgacagtgatatgtcgaaaaaatgcgtcgattagtgtgcgggcacaggatagatactattccagttcttggtccgagtgggcttctgtaccttgttca

SEQ ID NO: 96 - protein, 133 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 97 - DNA, 399 bp, synthetic construct
  source 1..399: mol_type = other DNA
ggcatccacgtgtttatcctgggttgcttctctgccggcctgcccaagaccgaagccaactgggtcaacgtaatctcagacctcaagaagatcgaggatttgattcagtcgatgcacattgacgccactctctacaccgagtccgacgtccatccctcctgtaaagttaccgcgatgaagtgtttcctgctagagcttcaagtgataagcctggagagtggcgacgcctccatacacgataccgtggagaatctcatcatcctggctaacaactccctaagttccaatgggaatgtcaccgaaagcggctgtaaagagtgcgaggaactggaggaaaagaacatcaaggagttcctccagtctttcgtgcacatcgtgcagatgtttattaatacgtca

SEQ ID NO: 98 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 99 - DNA, 471 bp, synthetic construct
  source 1..471: mol_type = other DNA
tattttggaaaactcgaatcaaaactttctgtgatccggaacctcaacgaccaggttttattcatcgaccaagggaatcgaccgctgttcgaggacatgacagatagcgactgtagagataatgctcccaggaccatttttataatctccatgtacaaagatagccagcctcgcgggatggcagtgactattagcgtgaaatgcgagaaaatctctaccctgagttgcgaaaacaaaataatctcctttaaggagatgaatccaccagacaacattaaggacacaaagtccgacataattttctttcaacgaagtgtccctggacacgataacaaaatgcagttcgaaagtagtagttacgagggttatttcctggcctgtgagaaggagcgggatctgtttaagctaatccttaagaaggaagatgaattgggcgatagatcgataatgttcacggtccagaatgaggac

SEQ ID NO: 100 - protein, 20 aa, synthetic construct
MDKFWWHAAWGLCLVPLSLA

SEQ ID NO: 101 - DNA, 60 bp, synthetic construct
  source 1..60: mol_type = other DNA
atggacaagttttggtggcacgcagcctggggactctgcctcgtgccgctgagcctggcg

SEQ ID NO: 102 - protein, 22 aa, synthetic construct
MQSGTHWRVLGLCLLSVGVWGQ

SEQ ID NO: 103 - DNA, 66 bp, synthetic construct
  source 1..66: mol_type = other DNA
atgcagtcgggcactcactggagagttctgggcctctgcctcttatcagttggcgtttgggggcaa

SEQ ID NO: 104 - protein, 24 aa, synthetic construct
MPMGSLQPLATLYLLGMLVASCLG

SEQ ID NO: 105 - DNA, 72 bp, synthetic construct
  source 1..72: mol_type = other DNA
atgcccatggggtctctgcaaccgctggccaccttgtacctgctggggatgctggtcgcttcctgcctcgga

SEQ ID NO: 106 - protein, 25 aa, synthetic construct
MKDSCITVMAMALLSGFFFFAPASS

SEQ ID NO: 107 - DNA, 75 bp, synthetic construct
  source 1..75: mol_type = other DNA
atgaaggattcctgcatcactgtgatggccatggcgctgctgtctgggttctttttcttcgcgccggcctcgagc

SEQ ID NO: 108 - protein, 20 aa, synthetic construct
MYRMQLLSCIALSLALVTNS

SEQ ID NO: 109 - DNA, 60 bp, synthetic construct
  source 1..60: mol_type = other DNA
atgtacaggatgcaactcctgtcttgcattgcactaagtcttgcacttgtcacaaacagt

SEQ ID NO: 110 - protein, 17 aa, synthetic construct
MWLQSLLLLGTVACSIS

SEQ ID NO: 111 - DNA, 51 bp, synthetic construct
  source 1..51: mol_type = other DNA
atgtggctgcagagcctgctgctcttgggcactgtggcctgcagcatctct

SEQ ID NO: 112 - protein, 18 aa, synthetic construct
MAFLWLLSCWALLGTTFG

SEQ ID NO: 113 - DNA, 54 bp, synthetic construct
  source 1..54: mol_type = other DNA
atggctttcctctggctcctctcctgctgggccctcctgggtaccaccttcggc

SEQ ID NO: 114 - protein, 15 aa, synthetic construct
MNLLLILTFVAAAVA

SEQ ID NO: 115 - DNA, 45 bp, synthetic construct
  source 1..45: mol_type = other DNA
atgaatctacttctgatccttacctttgttgcagctgctgttgct

SEQ ID NO: 116 - protein, 22 aa, synthetic construct
MDMRVLAQLLGLLLLCFPGARC

SEQ ID NO: 117 - DNA, 66 bp, synthetic construct
  source 1..66: mol_type = other DNA
atggacatgagagtcctcgctcagctcctggggctcctgctgctctgtttcccaggtgccagatgt

SEQ ID NO: 118 - protein, 22 aa, synthetic construct
MDMRVPAQLLGLLLLWLRGARC

SEQ ID NO: 119 - DNA, 66 bp, synthetic construct
  source 1..66: mol_type = other DNA
atggacatgagggtccccgctcagctcctggggctcctgctactctggctccgaggtgccagatgt

SEQ ID NO: 120 - protein, 18 aa, synthetic construct
MDWTWILFLVAAATRVHS

SEQ ID NO: 121 - DNA, 54 bp, synthetic construct
  source 1..54: mol_type = other DNA
atggactggacctggatcctcttcttggtggcagcagccacgcgagtccactcc

SEQ ID NO: 122 - protein, 25 aa, synthetic construct
MGVLLTQRTLLSLVLALLFPSMASM

SEQ ID NO: 123 - DNA, 75 bp, synthetic construct
  source 1..75: mol_type = other DNA
atgggggtactgctcacacagaggacgctgctcagtctggtccttgcactcctgtttccaagcatggcgagcatg

SEQ ID NO: 124 - protein, 19 aa, synthetic construct
MGWSCIILFLVATATGVHS

SEQ ID NO: 125 - DNA, 57 bp, synthetic construct
  source 1..57: mol_type = other DNA
atgggctggtcctgcatcatcctgtttctggtggccacagccaccggtgtccattct

SEQ ID NO: 126 - protein, 17 aa, synthetic construct
MRAWIFFLLCLAGRALA

SEQ ID NO: 127 - DNA, 51 bp, synthetic construct
  source 1..51: mol_type = other DNA
atgagggcctggatcttctttctcctttgcctggccgggagggccttggca

SEQ ID NO: 128 - protein, 21 aa, synthetic construct
MWWRLWWLLLLLLLLWPMVWA

SEQ ID NO: 129 - DNA, 63 bp, synthetic construct
  source 1..63: mol_type = other DNA
atgtggtggaggctttggtggctattactcctgttgctcctgctgtggcctatggtgtgggcc

SEQ ID NO: 130 - protein, 23 aa, synthetic construct
MDAMKRGLCCVLLLCGAVFVSPS

SEQ ID NO: 131 - DNA, 69 bp, synthetic construct
  source 1..69: mol_type = other DNA
atggatgcaatgaagagagggctctgctgtgtgctgctgctgtgtggagcagtcttcgtttcgcccagc

SEQ ID NO: 132 - protein, 132 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTL

SEQ ID NO: 133 - DNA, 399 bp, synthetic construct
  source 1..399: mol_type = other DNA
gcaccaacgtcatcatcaaccaaaaagacccagcttcagctagagcatctactgctggacctccaaatgattttgaacgggatcaacaactataagaatcccaaactaacaaggatgctgacgttcaagttttacatgccgaaaaaggcaaccgaacttaaacacctccaatgtttagaggaggaactcaaacctctcgaggaggtattgaatctggcgcaatccaagaatttccactttgacccacgcgatgtggttagcaacatcaatgtgtttgtgcttgaattgaagggaagcgaaacgactttcatgtgcgaatacgccgatgagactgccacaatcgtggagttccttaatcggtggataaccttttgtcagtcaatcatttctacactgacc

SEQ ID NO: 134 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 135 - DNA, 433 bp, synthetic construct
  source 1..433: mol_type = other DNA
aggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 136 - protein, 361 aa, synthetic construct
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 137 - DNA, 1083 bp, synthetic construct
  source 1..1083: mol_type = other DNA
ctatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 138 - skipped

SEQ ID NO: 139 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 140 - DNA, 459 bp, synthetic construct
  source 1..459: mol_type = other DNA
atgtatcgaatgcaactgctgtcgtgcatagcactatcactggctctggtgacaaacagtgcaccaacgtcatcatcaaccaaaaagacccagcttcagctagagcatctactgctggacctccaaatgattttgaacgggatcaacaactataagaatcccaaactaacaaggatgctgacgttcaagttttacatgccgaaaaaggcaaccgaacttaaacacctccaatgtttagaggaggaactcaaacctctcgaggaggtattgaatctggcgcaatccaagaatttccacctgaggccacgcgatctgatcagcaacatcaatgtgatcgtgcttgaattgaagggaagcgaaacgactttcatgtgcgaatacgccgatgagactgccacaatcgtggagttccttaatcggtggataaccttttgtcagtcaatcatttctacactgacc

SEQ ID NO: 141 - protein, 177 aa, synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 142 - DNA, 531 bp, synthetic construct
  source 1..531: mol_type = other DNA
atgttccatgtgtcgtttcgatatatcttcggcctcccacctctgattcttgtgctgttgcccgttgcctccagcgattgtgacattgaagggaaagatgggaaacagtacgagtccgtcctgatggtatctatagaccagttactcgatagcatgaaggagatcggctccaactgccttaacaacgagtttaatttcttcaaacgccatatttgcgatgccaataaggaaggaatgttcttattccgagctgcccggaagctgaggcagtttctgaagatgaacagcactggcgactttgacctgcacctccttaaggtgtctgagggcaccacaattctgctgaattgtaccggccaagtgaagggacgaaaacctgctgcgctaggagaagcacaacccacaaaatcactcgaggagaataagagtcttaaggaacagaagaagctgaacgacctctgctttttgaagaggctgttgcaagagatcaaaacctgttggaataagatcttgatggggaccaaagaacac

SEQ ID NO: 143 - protein, 162 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 144 - DNA, 486 bp, synthetic construct
  source 1..486: mol_type = other DNA
atgagaagctcacccgggaacatggaacgtatcgttatctgtcttatggtgatttttctgggtactctcgtacacaagagtagtagtcaaggacaggaccgacatatgataaggatgaggcaattaattgatattgtggaccaattgaaaaactacgtcaatgacctcgtgccagagttcctgcccgctcccgaagatgtagaaacgaattgcgaatggtcagcctttagctgtttccaaaaggcacagctcaaaagcgcgaataccggaaacaacgagcgtataattaacgtttcaatcaaaaaacttaagaggaagcctccttctacaaatgccggcaggcgccagaagcaccgcctgacctgcccgtcttgcgacagttatgagaagaagccacccaaagaatttctcgagagattcaagtctttacttcagaaaatgatccatcagcacctgagcagccgaactcacgggagtgaagattcc

SEQ ID NO: 145 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 146 - DNA, 1620 bp, synthetic construct
  source 1..1620: mol_type = other DNA
atgtgtcatcagcaactggtcattagctggttttccttggtgttcctggcctcacctctcgttgcaatttgggaactgaagaaagacgtatatgtcgtggaactggactggtatcccgatgccccaggggaaatggtggtccttacttgcgacacccccgaagaggacggcattacatggacccttgaccagtccagtgaggtcctcgggagtggaaagacactgacaattcaggttaaggagttcggcgatgccggtcagtacacctgtcataaagggggagaggttctgtcccattcactcctgctgctgcacaagaaggaagatggtatctggtccacggacattttgaaagatcaaaaggagcccaagaacaaaactttcctgcggtgtgaagccaaaaattacagcgggcgatttacttgttggtggctcaccactattagtacagatctcaccttcagcgttaagagttctaggggctccagtgatccgcagggagtgacgtgtggcgctgcgaccctgtccgctgagcgagtgaggggcgataataaggagtatgagtatagtgtggagtgtcaggaggattctgcttgccctgctgccgaggagtcccttcctatcgaagtgatggtcgatgctgtacacaaactgaaatacgagaattatactagctcctttttcataagggacatcataaagccagatccccccaagaaccttcagctgaaaccgctgaaaaattcacgccaggtggaggtatcttgggagtacccagacacatggagcacacctcatagttacttcagcctgaccttctgtgtccaggtccaggggaagtctaaaagggaaaaaaaggacagagttttcaccgacaagacatccgcaaccgtaatctgtaggaaaaacgcctcaatttccgttagagcgcaagataggtattattcctcttcctggtctgaatgggcctctgtaccctgcagcgggggtggcggtagcggtgggggagggagcggaggtggaggcagccgaaacctgcctgtggccacacctgatcctgggatgttcccttgcttgcaccacagtcagaatctgctcagggccgttagcaacatgctgcagaaggcgcggcagacacttgagttctacccttgcacaagtgaagagatcgatcacgaagatatcacaaaggataaaacgtctactgtcgaggcctgcctccccctcgaactcacaaagaacgagagttgcctcaattcccgggaaaccagcttcatcactaatgggtcctgcctggcaagcagaaaaacttctttcatgatggcattgtgtttgagtagcatctatgaggacctcaaaatgtaccaagttgaatttaagactatgaacgctaagctcctcatggaccctaagcggcagatcttcctggatcagaacatgttggctgtcatcgacgaactcatgcaagccctgaactttaattccgaaacagttccccagaagtccagtctggaggaacccgatttctacaaaaccaaaattaagctgtgtatcttgctgcatgctttccggatcagggccgttacgatcgatcgggtaatgagctacctgaatgccagt

SEQ ID NO: 147 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 148 - DNA, 984 bp, synthetic construct
  source 1..984: mol_type = other DNA
atgtgtcaccagcaactcgtgatctcatggttttcactcgtttttctggcaagtccgcttgtggcaatttgggaactcaaaaaggacgtgtatgtagtcgagctagattggtatcccgatgcacccggtgaaatggtcgttctgacctgcgacacaccagaagaggatggaatcacctggactctggatcaaagctccgaggtgctggggagcggcaaaaccctgacaattcaggtcaaagagttcggcgacgcgggacagtacacttgccataagggtggggaggttctgtcacactctctgctactgctgcacaaaaaggaggacggaatatggtcaacggacatcctgaaggaccagaaggagccaaaaaacaagaccttcctgaggtgcgaggccaaaaactactcaggccgctttacttgttggtggctgacaacaatctccacagacctcacctttagtgtgaagagtagtcgcggatctagcgatcctcaaggcgtcacttgcggtgccgctaccctgagtgctgaacgagtgcgtggggacaacaaggaatacgagtacagtgtggaatgccaagaggacagcgcttgcccggcagccgaggaatcactacccattgaagtgatggtcgatgccgtccataagttaaaatacgagaattacacgtcctctttcttcatccgcgatataatcaagcctgacccgccaaagaatttgcagttgaagcccttgaaaaacagccggcaggttgaagtgtcgtgggaatatcccgacacttggagcacaccacatagctatttctcactgacattctgtgtgcaagttcagggaaagtcaaaacgggagaagaaggatagagtatttaccgacaaaacgtcagcgacagtgatatgtcgaaaaaatgcgtcgattagtgtgcgggcacaggatagatactattccagttcttggtccgagtgggcttctgtaccttgttca

SEQ ID NO: 149 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 150 - DNA, 486 bp, synthetic construct
  source 1..486: mol_type = other DNA
atgaggataagtaagcctcatctgagatcaatctcaatccaatgttatctgtgcttattgctgaactcgcatttcttgacggaggcgggcatccacgtgtttatcctgggttgcttctctgccggcctgcccaagaccgaagccaactgggtcaacgtaatctcagacctcaagaagatcgaggatttgattcagtcgatgcacattgacgccactctctacaccgagtccgacgtccatccctcctgtaaagttaccgcgatgaagtgtttcctgctagagcttcaagtgataagcctggagagtggcgacgcctccatacacgataccgtggagaatctcatcatcctggctaacaactccctaagttccaatgggaatgtcaccgaaagcggctgtaaagagtgcgaggaactggaggaaaagaacatcaaggagttcctccagtctttcgtgcacatcgtgcagatgtttattaatacgtca

SEQ ID NO: 151 - protein, 177 aa, synthetic construct
MYRMQLLSCIALSLALVTNSYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 152 - DNA, 531 bp, synthetic construct
  source 1..531: mol_type = other DNA
atgtatcgaatgcaactgctgtcgtgcatagcactatcactggctctggtgacaaacagttattttggaaaactcgaatcaaaactttctgtgatccggaacctcaacgaccaggttttattcatcgaccaagggaatcgaccgctgttcgaggacatgacagatagcgactgtagagataatgctcccaggaccatttttataatctccatgtacaaagatagccagcctcgcgggatggcagtgactattagcgtgaaatgcgagaaaatctctaccctgagttgcgaaaacaaaataatctcctttaaggagatgaatccaccagacaacattaaggacacaaagtccgacataattttctttcaacgaagtgtccctggacacgataacaaaatgcagttcgaaagtagtagttacgagggttatttcctggcctgtgagaaggagcgggatctgtttaagctaatccttaagaaggaagatgaattgggcgatagatcgataatgttcacggtccagaatgaggac

SEQ ID NO: 153 - protein, 152 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTL

SEQ ID NO: 154 - DNA, 459 bp, synthetic construct
  source 1..459: mol_type = other DNA
atgtatcgaatgcaactgctgtcgtgcatagcactatcactggctctggtgacaaacagtgcaccaacgtcatcatcaaccaaaaagacccagcttcagctagagcatctactgctggacctccaaatgattttgaacgggatcaacaactataagaatcccaaactaacaaggatgctgacgttcaagttttacatgccgaaaaaggcaaccgaacttaaacacctccaatgtttagaggaggaactcaaacctctcgaggaggtattgaatctggcgcaatccaagaatttccactttgacccacgcgatgtggttagcaacatcaatgtgtttgtgcttgaattgaagggaagcgaaacgactttcatgtgcgaatacgccgatgagactgccacaatcgtggagttccttaatcggtggataaccttttgtcagtcaatcatttctacactgacc

SEQ ID NO: 155 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 156 - DNA, 534 bp, synthetic construct
  source 1..534: mol_type = other DNA
atgcactcatcagccttgctgtgctgcctcgtgttgctgaccggcgtgcgggcttctccaggtcaaggcacacagagtgaaaatagctgtactcattttcccgggaatctgccaaatatgctccgggatctgagggacgcattttccagagtgaagacatttttccaaatgaaggatcagcttgataatctcctgctgaaggaatctctgctagaggactttaagggctatctcgggtgtcaggcccttagtgagatgatccagttctatctggaggaagtaatgcctcaagccgaaaatcaagatcctgacatcaaagctcatgttaacagccttggagaaaatctgaaaacgctacgcttacgattaagacgctgccacaggtttttgccctgtgagaacaaatccaaagctgtggagcaggtcaagaacgcattcaacaaactccaggagaaaggaatttacaaggcgatgagcgagttcgacatattcatcaactacattgaggcctacatgactatgaagattcgtaac

SEQ ID NO: 157 - protein, 390 aa, synthetic construct
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 158 - DNA, 1170 bp, synthetic construct
  source 1..1170: mol_type = other DNA
atgccaccgtccggactgcggttgctgcctctgctcctgcctctcctctggctgctggttctgacaccagggaggcctgctgcgggcctgtccacttgcaaaaccattgatatggaactggtgaaacgcaagcggattgaggcaatccgcgggcagatcctgtccaagctacgcctggcctctccaccctctcaaggcgaagtgcctcctggtcctctgcctgaggcagtcctggcactctataatagcactcgagatcgcgttgctggggagagcgccgaacctgaacccgaacccgaggccgattactatgctaaagaggtcacgcgcgtactgatggttgaaactcacaacgagatctacgacaagttcaagcagagcacccacagtatctacatgttctttaacacctctgagctgagggaggcagtaccagaacccgtcctgctgagcagggccgagttacggcttcttcggctcaagctgaaggtcgagcagcacgttgagctttaccagaagtattcaaacaactcctggcgatacttgtccaaccggcttctggcgcccagcgactcacccgagtggctctcttttgacgtgaccggagtagtccgacaatggctctctcgcggcggcgagattgaaggctttcggttgtccgcccattgtagctgcgatagtagggataataccctccaggtggacattaatggatttacaaccggaaggcgtggggaccttgctacaatccacggcatgaacagaccgtttttgctcctaatggccactccactggagcgcgctcaacatcttcagtcaagcagacacagacgagccttggacacaaactattgcttcagtagtacggagaagaattgttgcgtgagacagttatatatcgacttccgtaaagacttaggttggaaatggatacacgaaccaaagggctatcatgcaaatttctgtctcggaccctgtccatacatatggagtttggatacacagtactcgaaagtgctggctctatacaatcagcataatcccggcgcctcagccgcgccgtgttgtgtgccacaagcccttgaacctttacccatcgtgtactatgtgggcagaaaacccaaggtggaacagttgtctaacatgattgtgcgttcctgcaaatgctca

SEQ ID NO: 159 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkage
ggttcttgactaccgtaatt

SEQ ID NO: 160 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkage
ttgacatagaagaggcacaa

SEQ ID NO: 161 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkage
ctagagaaactgttgcataa

SEQ ID NO: 162 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkage
gcgcccgcggccgtgccccg

SEQ ID NO: 163 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkage
cagctccctctagtcccgcg

SEQ ID NO: 164 - RNA, 20 nt, synthetic construct
  source 1..20: mol_type = other RNA
  modified_base 1..3: mod_base = OTHER; note = 2-prime-o-methyl nucleotides
  ncRNA 1..20: ncRNA_class = sgRNA
  modified_base 1..3: mod_base = OTHER; note = 3-prime-phosphorothioate internucleotide linkages
gcgaaaggtgctggacccag

SEQ ID NO: 165 - RNA, 22 nt, synthetic construct
  source 1..22: mol_type = other RNA
  misc_feature 1..22: note = shRNA
tcttttatacatctcaccttct

SEQ ID NO: 166 - RNA, 22 nt, synthetic construct
  source 1..22: mol_type = other RNA
  misc_feature 1..22: note = shRNA
tctgacaagagcttcacactga

SEQ ID NO: 167 - RNA, 22 nt, synthetic construct
  source 1..22: mol_type = other RNA
  misc_feature 1..22: note = shRNA
ttcatgaagtcttatcagacgt

SEQ ID NO: 168 - RNA, 22 nt, synthetic construct
  source 1..22: mol_type = other RNA
  misc_feature 1..22: note = shRNA
ttaagaatcttttcaaacacta

SEQ ID NO: 169 - RNA, 22 nt, synthetic construct
  source 1..22: mol_type = other RNA
  misc_feature 1..22: note = shRNA
tagaattacattttgatgcctt

SEQ ID NO: 170 - RNA, 22 nt, synthetic construct
  source 1..22: mol_type = other RNA
  misc_feature 1..22: note = shRNA
ttagtaataagacatgtttcat

SEQ ID NO: 171 - RNA, 22 nt, synthetic construct
  source 1..22: mol_type = other RNA
  misc_feature 1..22: note = shRNA
taaaaatcaatctcatttcctg

SEQ ID NO: 172 - RNA, 22 nt, synthetic construct
  source 1..22: mol_type = other RNA
  misc_feature 1..22: note = shRNA
ttatgtaaaagacaaacaatgc

SEQ ID NO: 173 - RNA, 22 nt, synthetic construct
  source 1..22: mol_type = other RNA
  misc_feature 1..22: note = shRNA
tagataagcattataattccta

SEQ ID NO: 174 - RNA, 22 nt, synthetic construct
  source 1..22: mol_type = other RNA
  misc_feature 1..22: note = shRNA
ttaatcagagacttcaggcggt

SEQ ID NO: 175 - protein, 249 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 176 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 177 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 178 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 179 - protein, 79 aa, synthetic construct
MALPVTALLLPLALLLHAARPDYKDDDDKELCGGRIARLEEKVKTLKAQNSELASTANMLREQVAQLKQKVMNAQGEIE

SEQ ID NO: 180 - protein, 22 aa, synthetic construct
AIVVPVCLAFLLTTLLGVLFCF

SEQ ID NO: 181 - protein, 318 aa, synthetic construct
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLW

SEQ ID NO: 182 - protein, 7 aa, Tobacco etch virus
ENLYTQS

SEQ ID NO: 183 - protein, 5 aa, synthetic construct
DDDDK

SEQ ID NO: 184 - protein, 4 aa, synthetic construct
LVPR

SEQ ID NO: 185 - protein, 4 aa, synthetic construct
CPPC

SEQ ID NO: 186 - RNA, 83 nt, synthetic construct
  source 1..83: mol_type = other RNA
  ncRNA 1..83: ncRNA_class = guide_RNA
gttttagagctagaaatagcaagttaaaataaggctagtccgttatcaacttgaaaaagtggcaccgagtcggtgcttttttt

SEQ ID NO: 187 - DNA, 20 bp, synthetic construct
  source 1..20: mol_type = other DNA
ggggccactagggacaggat

SEQ ID NO: 188 - RNA, 20 nt, synthetic construct
  source 1..20: mol_type = other RNA
  ncRNA 1..20: ncRNA_class = sgRNA
gcacctgaataccacgcctg

SEQ ID NO: 189 - RNA, 20 nt, synthetic construct
  source 1..20: mol_type = other RNA
  ncRNA 1..20: ncRNA_class = sgRNA
cgcctgcgatgtagtcgatg

SEQ ID NO: 190 - RNA, 20 nt, synthetic construct
  source 1..20: mol_type = other RNA
  ncRNA 1..20: ncRNA_class = sgRNA
caggacgggcgagatgtccc

SEQ ID NO: 191 - RNA, 20 nt, synthetic construct
  source 1..20: mol_type = other RNA
  ncRNA 1..20: ncRNA_class = sgRNA
ctgaatctttggagtacctg

SEQ ID NO: 192 - RNA, 20 nt, synthetic construct
  source 1..20: mol_type = other RNA
  ncRNA 1..20: ncRNA_class = sgRNA
ggccacggagcgagacatct

SEQ ID NO: 193 - RNA, 20 nt, synthetic construct
  source 1..20: mol_type = other RNA
  ncRNA 1..20: ncRNA_class = sgRNA
aagtcaacttcaatgtcgga

SEQ ID NO: 194 - RNA, 20 nt, synthetic construct
  source 1..20: mol_type = other RNA
  ncRNA 1..20: ncRNA_class = sgRNA
gcttggaggcctgatcagcg

SEQ ID NO: 195 - RNA, 20 nt, synthetic construct
  source 1..20: mol_type = other RNA
  ncRNA 1..20: ncRNA_class = sgRNA
cttatctcttcgcagcgagg

SEQ ID NO: 196 - RNA, 20 nt, synthetic construct
  source 1..20: mol_type = other RNA
  ncRNA 1..20: ncRNA_class = sgRNA
cacacattactccaacattg

SEQ ID NO: 197 - RNA, 20 nt, synthetic construct
  source 1..20: mol_type = other RNA
  ncRNA 1..20: ncRNA_class = sgRNA
ttccgcaaaatagagcccca

SEQ ID NO: 198 - RNA, 20 nt, synthetic construct
  source 1..20: mol_type = other RNA
  ncRNA 1..20: ncRNA_class = sgRNA
tgcacagaactatcgtacca

SEQ ID NO: 199 - RNA, 20 nt, synthetic construct
  source 1..20: mol_type = other RNA
  ncRNA 1..20: ncRNA_class = sgRNA
gcaataagactctttaaaga

SEQ ID NO: 200 - RNA, 20 nt, synthetic construct
  source 1..20: mol_type = other RNA
  ncRNA 1..20: ncRNA_class = sgRNA
caaagagattacgaatgcct

SEQ ID NO: 201 - RNA, 20 nt, synthetic construct
  source 1..20: mol_type = other RNA
  ncRNA 1..20: ncRNA_class = sgRNA
caaggcaccccaggtttcca

SEQ ID NO: 202 - RNA, 20 nt, synthetic construct
  source 1..20: mol_type = other RNA
  ncRNA 1..20: ncRNA_class = sgRNA
ttacgaatgccttggaaacc

SEQ ID NO: 203 - RNA, 20 nt, synthetic construct
  source 1..20: mol_type = other RNA
  ncRNA 1..20: ncRNA_class = sgRNA
cagagacgcatctgaccctc

SEQ ID NO: 204 - RNA, 20 nt, synthetic construct
  source 1..20: mol_type = other RNA
  ncRNA 1..20: ncRNA_class = sgRNA
catgcagttctcacacactg

SEQ ID NO: 205 - RNA, 20 nt, synthetic construct
  source 1..20: mol_type = other RNA
  ncRNA 1..20: ncRNA_class = sgRNA
gtgtgagaactgcatggaga

SEQ ID NO: 206 - RNA, 20 nt, synthetic construct
  source 1..20: mol_type = other RNA
  ncRNA 1..20: ncRNA_class = sgRNA
tctcatttcaggaaaccact

SEQ ID NO: 207 - RNA, 20 nt, synthetic construct
  source 1..20: mol_type = other RNA
  ncRNA 1..20: ncRNA_class = sgRNA
agtcatacaccttaaccaag

SEQ ID NO: 208 - RNA, 20 nt, synthetic construct
  source 1..20: mol_type = other RNA
  ncRNA 1..20: ncRNA_class = sgRNA
ttcaaggaaaccagttgagg

SEQ ID NO: 209 - RNA, 20 nt, synthetic construct
  source 1..20: mol_type = other RNA
  ncRNA 1..20: ncRNA_class = sgRNA
gagccttgcctggaaatctg

SEQ ID NO: 210 - RNA, 20 nt, synthetic construct
  source 1..20: mol_type = other RNA
  ncRNA 1..20: ncRNA_class = sgRNA
aagcgtcaaaagtctgccag

SEQ ID NO: 211 - RNA, 20 nt, synthetic construct
  source 1..20: mol_type = other RNA
  ncRNA 1..20: ncRNA_class = sgRNA
cgttccaactcgaagtgcca

SEQ ID NO: 212 - RNA, 20 nt, synthetic construct
  source 1..20: mol_type = other RNA
  ncRNA 1..20: ncRNA_class = sgRNA
gagcgactgggacacggtga

SEQ ID NO: 213 - RNA, 20 nt, synthetic construct
  source 1..20: mol_type = other RNA
  ncRNA 1..20: ncRNA_class = sgRNA
gctgcgcaagaagggcccta

SEQ ID NO: 214 - RNA, 20 nt, synthetic construct
  source 1..20: mol_type = other RNA
  ncRNA 1..20: ncRNA_class = sgRNA
ttgttctggccagcagcccc

SEQ ID NO: 215 - RNA, 20 nt, synthetic construct
  source 1..20: mol_type = other RNA
  ncRNA 1..20: ncRNA_class = sgRNA
cttccagagccacatcatcg

SEQ ID NO: 216 - RNA, 20 nt, synthetic construct
  source 1..20: mol_type = other RNA
  ncRNA 1..20: ncRNA_class = sgRNA
gggactcaccagagagaggt

SEQ ID NO: 217 - RNA, 20 nt, synthetic construct
  source 1..20: mol_type = other RNA
  ncRNA 1..20: ncRNA_class = sgRNA
cggtcgaaatagaagcccta

SEQ ID NO: 218 - RNA, 20 nt, synthetic construct
  source 1..20: mol_type = other RNA
  ncRNA 1..20: ncRNA_class = sgRNA
aaaaggatattgtgcaactg

SEQ ID NO: 219 - RNA, 20 nt, synthetic construct
  source 1..20: mol_type = other RNA
  ncRNA 1..20: ncRNA_class = sgRNA
tgtgcatatttattacatcg

SEQ ID NO: 220 - RNA, 20 nt, synthetic construct
  source 1..20: mol_type = other RNA
  ncRNA 1..20: ncRNA_class = sgRNA
tttgtgaagatcttgaccaa

SEQ ID NO: 221 - RNA, 20 nt, synthetic construct
  source 1..20: mol_type = other RNA
  ncRNA 1..20: ncRNA_class = sgRNA
tgtcatgctgaaccgcattg

SEQ ID NO: 222 - RNA, 20 nt, synthetic construct
  source 1..20: mol_type = other RNA
  ncRNA 1..20: ncRNA_class = sgRNA
ccactctatgaggatagtca

SEQ ID NO: 223 - RNA, 20 nt, synthetic construct
  source 1..20: mol_type = other RNA
  ncRNA 1..20: ncRNA_class = sgRNA
ttgacatagaagaggcacaa

SEQ ID NO: 224 - RNA, 20 nt, synthetic construct
  source 1..20: mol_type = other RNA
  ncRNA 1..20: ncRNA_class = sgRNA
gagtactacactcagcagca

SEQ ID NO: 225 - RNA, 20 nt, synthetic construct
  source 1..20: mol_type = other RNA
  ncRNA 1..20: ncRNA_class = sgRNA
tcacgcacaagaaacgtcca

SEQ ID NO: 226 - RNA, 20 nt, synthetic construct
  source 1..20: mol_type = other RNA
  ncRNA 1..20: ncRNA_class = sgRNA
aggtctcggtgaaaccacct

SEQ ID NO: 227 - RNA, 20 nt, synthetic construct
  source 1..20: mol_type = other RNA
  ncRNA 1..20: ncRNA_class = sgRNA
agcattatccaaagagtccg

SEQ ID NO: 228 - RNA, 20 nt, synthetic construct
  source 1..20: mol_type = other RNA
  ncRNA 1..20: ncRNA_class = sgRNA
atattaattcttaccagtgg

SEQ ID NO: 229 - RNA, 20 nt, synthetic construct
  source 1..20: mol_type = other RNA
  ncRNA 1..20: ncRNA_class = sgRNA
agctttaaatcaaggttcat

SEQ ID NO: 230 - RNA, 20 nt, synthetic construct
  source 1..20: mol_type = other RNA
  ncRNA 1..20: ncRNA_class = sgRNA
atcccgagccctaaggtgca

SEQ ID NO: 231 - RNA, 20 nt, synthetic construct
  source 1..20: mol_type = other RNA
  ncRNA 1..20: ncRNA_class = sgRNA
ggcagcgcggaggacagcgt

SEQ ID NO: 232 - RNA, 20 nt, synthetic construct
  source 1..20: mol_type = other RNA
  ncRNA 1..20: ncRNA_class = sgRNA
ctcagggggctactaccacc

SEQ ID NO: 233 - RNA, 20 nt, synthetic construct
  source 1..20: mol_type = other RNA
  ncRNA 1..20: ncRNA_class = sgRNA
gtcaccgacgagaccagaag

SEQ ID NO: 234 - RNA, 20 nt, synthetic construct
  source 1..20: mol_type = other RNA
  ncRNA 1..20: ncRNA_class = sgRNA
gtcgtggacttcgtactgct

SEQ ID NO: 235 - RNA, 20 nt, synthetic construct
  source 1..20: mol_type = other RNA
  ncRNA 1..20: ncRNA_class = sgRNA
taatttttaggcaagtgtcg

SEQ ID NO: 236 - RNA, 20 nt, synthetic construct
  source 1..20: mol_type = other RNA
  ncRNA 1..20: ncRNA_class = sgRNA
ttagctgttagacttgaata

SEQ ID NO: 237 - RNA, 20 nt, synthetic construct
  source 1..20: mol_type = other RNA
  ncRNA 1..20: ncRNA_class = sgRNA
cgagagccgtcaacttgcgt

SEQ ID NO: 238 - RNA, 20 nt, synthetic construct
  source 1..20: mol_type = other RNA
  ncRNA 1..20: ncRNA_class = sgRNA
cggcttcaactgcaaaggtg

SEQ ID NO: 239 - RNA, 20 nt, synthetic construct
  source 1..20: mol_type = other RNA
  ncRNA 1..20: ncRNA_class = sgRNA
tatgaaaaagcagagcgact

SEQ ID NO: 240 - RNA, 20 nt, synthetic construct
  source 1..20: mol_type = other RNA
  ncRNA 1..20: ncRNA_class = sgRNA
tctggcgggcgagctcacgc

SEQ ID NO: 241 - RNA, 20 nt, synthetic construct
  source 1..20: mol_type = other RNA
  ncRNA 1..20: ncRNA_class = sgRNA
ctcacgctggttaccgccta

SEQ ID NO: 242 - RNA, 20 nt, synthetic construct
  source 1..20: mol_type = other RNA
  ncRNA 1..20: ncRNA_class = sgRNA
aaagattacgaacttccctg

SEQ ID NO: 243 - RNA, 20 nt, synthetic construct
  source 1..20: mol_type = other RNA
  ncRNA 1..20: ncRNA_class = sgRNA
gttaaaaacagacatgccta

SEQ ID NO: 244 - RNA, 20 nt, synthetic construct
  source 1..20: mol_type = other RNA
  ncRNA 1..20: ncRNA_class = sgRNA
atgcctaaggaggttgtacc

SEQ ID NO: 245 - RNA, 20 nt, synthetic construct
  source 1..20: mol_type = other RNA
  ncRNA 1..20: ncRNA_class = sgRNA
ctccaggtatcccatcgaaa

SEQ ID NO: 246 - RNA, 20 nt, synthetic construct
  source 1..20: mol_type = other RNA
  ncRNA 1..20: ncRNA_class = sgRNA
caccaaatacgatagatcag

SEQ ID NO: 247 - RNA, 20 nt, synthetic construct
  source 1..20: mol_type = other RNA
  ncRNA 1..20: ncRNA_class = sgRNA
tggcggcgtgaatggcaaga

SEQ ID NO: 248 - RNA, 20 nt, synthetic construct
  source 1..20: mol_type = other RNA
  ncRNA 1..20: ncRNA_class = sgRNA
taggatggtagcacacaacc

SEQ ID NO: 249 - RNA, 20 nt, synthetic construct
  source 1..20: mol_type = other RNA
  ncRNA 1..20: ncRNA_class = sgRNA
cagcagcagagccccgacgg

SEQ ID NO: 250 - RNA, 20 nt, synthetic construct
  source 1..20: mol_type = other RNA
  ncRNA 1..20: ncRNA_class = sgRNA
cggcgtgcgaacggaatgtg

SEQ ID NO: 251 - RNA, 20 nt, synthetic construct
  source 1..20: mol_type = other RNA
  ncRNA 1..20: ncRNA_class = sgRNA
tatagacgctgcccgacgtc

SEQ ID NO: 252 - RNA, 20 nt, synthetic construct
  source 1..20: mol_type = other RNA
  ncRNA 1..20: ncRNA_class = sgRNA
tccaaagaagggtactgtgg

SEQ ID NO: 253 - RNA, 20 nt, synthetic construct
  source 1..20: mol_type = other RNA
  ncRNA 1..20: ncRNA_class = sgRNA
acagtacccttctttggaat

SEQ ID NO: 254 - RNA, 20 nt, synthetic construct
  source 1..20: mol_type = other RNA
  ncRNA 1..20: ncRNA_class = sgRNA
gcgacgggcgcatctacgtg

SEQ ID NO: 255 - RNA, 20 nt, synthetic construct
  source 1..20: mol_type = other RNA
  ncRNA 1..20: ncRNA_class = sgRNA
cccgacctccataagtcctg

SEQ ID NO: 256 - RNA, 20 nt, synthetic construct
  source 1..20: mol_type = other RNA
  ncRNA 1..20: ncRNA_class = sgRNA
ggggtcctcgaagcgcacga

SEQ ID NO: 257 - RNA, 20 nt, synthetic construct
  source 1..20: mol_type = other RNA
  ncRNA 1..20: ncRNA_class = sgRNA
tgctctgtttagaagatgac

SEQ ID NO: 258 - RNA, 20 nt, synthetic construct
  source 1..20: mol_type = other RNA
  ncRNA 1..20: ncRNA_class = sgRNA
atattcttttctagttaaag

SEQ ID NO: 259 - RNA, 20 nt, synthetic construct
  source 1..20: mol_type = other RNA
  ncRNA 1..20: ncRNA_class = sgRNA
cctgtaaagaaacaaaagac

SEQ ID NO: 260 - RNA, 20 nt, synthetic construct
  source 1..20: mol_type = other RNA
  ncRNA 1..20: ncRNA_class = sgRNA
tggagaaagacgtaacttcg

SEQ ID NO: 261 - RNA, 20 nt, synthetic construct
  source 1..20: mol_type = other RNA
  ncRNA 1..20: ncRNA_class = sgRNA
tctgccctgaggtatgcgat

SEQ ID NO: 262 - RNA, 20 nt, synthetic construct
  source 1..20: mol_type = other RNA
  ncRNA 1..20: ncRNA_class = sgRNA
attccgcttggtgaaaacga

SEQ ID NO: 263 - RNA, 20 nt, synthetic construct
  source 1..20: mol_type = other RNA
  ncRNA 1..20: ncRNA_class = sgRNA
caggcacaatagaaacaacg

SEQ ID NO: 264 - RNA, 20 nt, synthetic construct
  source 1..20: mol_type = other RNA
  ncRNA 1..20: ncRNA_class = sgRNA
ccatttgtaatgctgacttg

SEQ ID NO: 265 - RNA, 20 nt, synthetic construct
  source 1..20: mol_type = other RNA
  ncRNA 1..20: ncRNA_class = sgRNA
ctgggtcacttgtgccgtgg

SEQ ID NO: 266 - RNA, 20 nt, synthetic construct
  source 1..20: mol_type = other RNA
  ncRNA 1..20: ncRNA_class = sgRNA
gtcagggttctggatatctg

SEQ ID NO: 267 - RNA, 20 nt, synthetic construct
  source 1..20: mol_type = other RNA
  ncRNA 1..20: ncRNA_class = sgRNA
tggatttagagtctctcagc

SEQ ID NO: 268 - RNA, 20 nt, synthetic construct
  source 1..20: mol_type = other RNA
  ncRNA 1..20: ncRNA_class = sgRNA
ctgcggctgtggtccagctg

SEQ ID NO: 269 - RNA, 20 nt, synthetic construct
  source 1..20: mol_type = other RNA
  ncRNA 1..20: ncRNA_class = sgRNA
acaaaactgtgctagacatg

SEQ ID NO: 270 - RNA, 20 nt, synthetic construct
  source 1..20: mol_type = other RNA
  ncRNA 1..20: ncRNA_class = sgRNA
ttcttccccagcccaggtaa

SEQ ID NO: 271 - RNA, 20 nt, synthetic construct
  source 1..20: mol_type = other RNA
  ncRNA 1..20: ncRNA_class = sgRNA
cgtcatgagcagattaaacc

SEQ ID NO: 272 - RNA, 20 nt, synthetic construct
  source 1..20: mol_type = other RNA
  ncRNA 1..20: ncRNA_class = sgRNA
gagagcgcctgcgacccgag

SEQ ID NO: 273 - RNA, 20 nt, synthetic construct
  source 1..20: mol_type = other RNA
  ncRNA 1..20: ncRNA_class = sgRNA
ccagcgggtgaagtacacca

SEQ ID NO: 274 - RNA, 20 nt, synthetic construct
  source 1..20: mol_type = other RNA
  ncRNA 1..20: ncRNA_class = sgRNA
ggagcgcttttcgccgccag

SEQ ID NO: 275 - RNA, 20 nt, synthetic construct
  source 1..20: mol_type = other RNA
  ncRNA 1..20: ncRNA_class = sgRNA
tgaggcctggaccttatgca

SEQ ID NO: 276 - RNA, 20 nt, synthetic construct
  source 1..20: mol_type = other RNA
  ncRNA 1..20: ncRNA_class = sgRNA
cctggtggagtgaaccatga

SEQ ID NO: 277 - RNA, 20 nt, synthetic construct
  source 1..20: mol_type = other RNA
  ncRNA 1..20: ncRNA_class = sgRNA
caagcacttaggttcccctg

SEQ ID NO: 278 - RNA, 20 nt, synthetic construct
  source 1..20: mol_type = other RNA
  ncRNA 1..20: ncRNA_class = sgRNA
ggtctccctacaattcagcg

SEQ ID NO: 279 - RNA, 20 nt, synthetic construct
  source 1..20: mol_type = other RNA
  ncRNA 1..20: ncRNA_class = sgRNA
cacagcgcgtgactgcaatg

SEQ ID NO: 280 - RNA, 20 nt, synthetic construct
  source 1..20: mol_type = other RNA
  ncRNA 1..20: ncRNA_class = sgRNA
tctggggcaccaattctagg

SEQ ID NO: 281 - RNA, 20 nt, synthetic construct
  source 1..20: mol_type = other RNA
  ncRNA 1..20: ncRNA_class = sgRNA
gagccatgcttggcttacga

SEQ ID NO: 282 - RNA, 20 nt, synthetic construct
  source 1..20: mol_type = other RNA
  ncRNA 1..20: ncRNA_class = sgRNA
gtacaagtacttatctcatg

SEQ ID NO: 283 - RNA, 20 nt, synthetic construct
  source 1..20: mol_type = other RNA
  ncRNA 1..20: ncRNA_class = sgRNA
gagataacaacataacaaca

SEQ ID NO: 284 - RNA, 20 nt, synthetic construct
  source 1..20: mol_type = other RNA
  ncRNA 1..20: ncRNA_class = sgRNA
catattccatagtctttggg

SEQ ID NO: 285 - RNA, 20 nt, synthetic construct
  source 1..20: mol_type = other RNA
  ncRNA 1..20: ncRNA_class = sgRNA
ctgccccttagcaacttagg

SEQ ID NO: 286 - RNA, 20 nt, synthetic construct
  source 1..20: mol_type = other RNA
  ncRNA 1..20: ncRNA_class = sgRNA
tgtttaaaaatatgttgaca

SEQ ID NO: 287 - RNA, 20 nt, synthetic construct
  source 1..20: mol_type = other RNA
  ncRNA 1..20: ncRNA_class = sgRNA
ccaggaatggaaactcacgc

SEQ ID NO: 288 - RNA, 20 nt, synthetic construct
  source 1..20: mol_type = other RNA
  ncRNA 1..20: ncRNA_class = sgRNA
gaggccgctgaattaacccg

SEQ ID NO: 289 - RNA, 20 nt, synthetic construct
  source 1..20: mol_type = other RNA
  ncRNA 1..20: ncRNA_class = sgRNA
atacacgcacacttgcagaa

SEQ ID NO: 290 - RNA, 20 nt, synthetic construct
  source 1..20: mol_type = other RNA
  ncRNA 1..20: ncRNA_class = sgRNA
gagcagacagaaacccaggg

SEQ ID NO: 291 - RNA, 20 nt, synthetic construct
  source 1..20: mol_type = other RNA
  ncRNA 1..20: ncRNA_class = sgRNA
tgagtctccaaacagaacag

SEQ ID NO: 292 - RNA, 20 nt, synthetic construct
  source 1..20: mol_type = other RNA
  ncRNA 1..20: ncRNA_class = sgRNA
taatatcactgacttcacgg

SEQ ID NO: 293 - RNA, 20 nt, synthetic construct
  source 1..20: mol_type = other RNA
  ncRNA 1..20: ncRNA_class = sgRNA
tacacacaatgtaagcagca

SEQ ID NO: 294 - RNA, 20 nt, synthetic construct
  source 1..20: mol_type = other RNA
  ncRNA 1..20: ncRNA_class = sgRNA
gggagctcaattcgaaacca

SEQ ID NO: 295 - RNA, 20 nt, synthetic construct
  source 1..20: mol_type = other RNA
  ncRNA 1..20: ncRNA_class = sgRNA
ttggacaggtgagacagtcg

SEQ ID NO: 296 - RNA, 20 nt, synthetic construct
  source 1..20: mol_type = other RNA
  ncRNA 1..20: ncRNA_class = sgRNA
aagctcactcagatagtgtg

SEQ ID NO: 297 - RNA, 20 nt, synthetic construct
  source 1..20: mol_type = other RNA
  ncRNA 1..20: ncRNA_class = sgRNA
caggagaaccaccttacacg

SEQ ID NO: 298 - RNA, 20 nt, synthetic construct
  source 1..20: mol_type = other RNA
  ncRNA 1..20: ncRNA_class = sgRNA
ggacagaccctgattcacaa

SEQ ID NO: 299 - RNA, 20 nt, synthetic construct
  source 1..20: mol_type = other RNA
  ncRNA 1..20: ncRNA_class = sgRNA
acatggcagtctatgaacag

SEQ ID NO: 300 - RNA, 20 nt, synthetic construct
  source 1..20: mol_type = other RNA
  ncRNA 1..20: ncRNA_class = sgRNA
cctatagagagtactacttg

SEQ ID NO: 301 - RNA, 20 nt, synthetic construct
  source 1..20: mol_type = other RNA
  ncRNA 1..20: ncRNA_class = sgRNA
ccaaccgggtcttcattacg

SEQ ID NO: 302 - RNA, 20 nt, synthetic construct
  source 1..20: mol_type = other RNA
  ncRNA 1..20: ncRNA_class = sgRNA
tcaagcgtagagttccgagt

SEQ ID NO: 303 - RNA, 20 nt, synthetic construct
  source 1..20: mol_type = other RNA
  ncRNA 1..20: ncRNA_class = sgRNA
tcatgcaattatggacccag

SEQ ID NO: 304 - RNA, 20 nt, synthetic construct
  source 1..20: mol_type = other RNA
  ncRNA 1..20: ncRNA_class = sgRNA
cgggaaagtgactggccatg

SEQ ID NO: 305 - RNA, 20 nt, synthetic construct
  source 1..20: mol_type = other RNA
  ncRNA 1..20: ncRNA_class = sgRNA
tgagattgaaatcaaatcgg

SEQ ID NO: 306 - RNA, 20 nt, synthetic construct
  source 1..20: mol_type = other RNA
  ncRNA 1..20: ncRNA_class = sgRNA
tatgcaatattcatcacgcg

SEQ ID NO: 307 - RNA, 20 nt, synthetic construct
  source 1..20: mol_type = other RNA
  ncRNA 1..20: ncRNA_class = sgRNA
aatgtgttaaatcaaatgca

SEQ ID NO: 308 - protein, 6 aa, synthetic construct
TPYNGA